SEQUENCE LISTING (218 sequences)

SEQ ID NO: 1 - DNA, 54 bp, synthetic construct
  source 1..54: mol_type = other DNA
ccttacctgggcaaacgggaggaccagtggtgtggaagcctgattgggctgact

SEQ ID NO: 2 - protein, 18 aa, unidentified
PYLGKREDQWCGSLIGLT

SEQ ID NO: 3 - DNA, 84 bp, synthetic construct
  source 1..84: mol_type = other DNA
agacggggagacctgcccgtgtggctgtcttacaaggtggccagcgagggcatctcctatacagacagaaagtggtgcttcgat

SEQ ID NO: 4 - protein, 28 aa, unidentified
RRGDLPVWLSYKVASEGISYTDRKWCFD

SEQ ID NO: 5 - DNA, 111 bp, synthetic construct
  source 1..111: mol_type = other DNA
cagcctcactggatcgccgccagcatcatcctggagttctttctgatggtgctgctgatcccagagccagagaagcagaggaccccacaggataaccagctggcctacgtg

SEQ ID NO: 6 - protein, 37 aa, unidentified
QPHWIAASIILEFFLMVLLIPEPEKQRTPQDNQLAYV

SEQ ID NO: 7 - DNA, 120 bp, synthetic construct
  source 1..120: mol_type = other DNA
accctaatcctagcacccacccgggttgtggccagcgagatggcagaggccctgaagggcatgccaatcaggtatcagaccacagccacaaagtccgagcacaccggccgcgagatcgtg

SEQ ID NO: 8 - protein, 40 aa, unidentified
TLILAPTRVVASEMAEALKGMPIRYQTTATKSEHTGREIV

SEQ ID NO: 9 - DNA, 366 bp, synthetic construct
  source 1..366: mol_type = other DNA
gataacatcaatacacctgagggcatcatcccaagcctgttcgagccagagagggagaagtccgccgcaatcgacggagagtacaggctgagaggagaggcacggaagacctttgtggacctgatgaggagaggcgatctgcccgtgtggctggcatacagagtggcagcagagggcatcaactatgccgacaggcgctggtgctttgatggcgagaggaacaatcagatcctggaggagaatatggaggtggagatctggaccaaggagggcgagagaaagaagctgaagccccggtggctggacgccagaatctacagcgatcctctggctctgaaagagtttaaggagttcgcagcaggaagg

SEQ ID NO: 10 - protein, 122 aa, unidentified
DNINTPEGIIPSLFEPEREKSAAIDGEYRLRGEARKTFVDLMRRGDLPVWLAYRVAAEGINYADRRWCFDGERNNQILEENMEVEIWTKEGERKKLKPRWLDARIYSDPLALKEFKEFAAGR

SEQ ID NO: 11 - DNA, 69 bp, synthetic construct
  source 1..69: mol_type = other DNA
ctgaggccaagatggctggatgccagaacatactccgatcccatggctctgaaggacttcaaggagttt

SEQ ID NO: 12 - protein, 23 aa, unidentified
LRPRWLDARTYSDPMALKDFKEF

SEQ ID NO: 13 - DNA, 177 bp, synthetic construct
  source 1..177: mol_type = other DNA
gaccacccctataagacctgggcctaccacggcagctatgagacaaagcagacaggctccgccagctccatggtgaatggcgtggtgaagctgctgaccaagccttgggatgtggtgccaatggtgacacagatggccatgaccgacaccacaccattcggccagcagagggtgttt

SEQ ID NO: 14 - protein, 59 aa, unidentified
DHPYKTWAYHGSYETKQTGSASSMVNGVVKLLTKPWDVVPMVTQMAMTDTTPFGQQRVF

SEQ ID NO: 15 - DNA, 183 bp, synthetic construct
  source 1..183: mol_type = other DNA
accgactgggatttcgtggtgaccacagatatcagcgagatgggcgccaacttcaaggccgaccgcgtgatcgatccacggagatgcctgaagcccgtgatcctgacagacggacctgagagagtgatcctggcaggacctatgccagtgacccactctagcgccgcacagaggaggggccgg

SEQ ID NO: 16 - protein, 61 aa, unidentified
TDWDFVVTTDISEMGANFKADRVIDPRRCLKPVILTDGPERVILAGPMPVTHSSAAQRRGR

SEQ ID NO: 17 - DNA, 369 bp, synthetic construct
  source 1..369: mol_type = other DNA
caccctggcgccggcaagacccggagatacctgccagccatcgtgagagaggccatcaagaggggcctgcgcacactgatcctggcccctaccagggtggtggcagcagagatggaggaggccctgaggggcctgcctatccgctatcagacaccagcaatcagggcagagcacaccggaagagagatcgtggatctgatgtgccacgccaccttcacaatgaggctgctgtccccagtgcgcgtgcccaactacaatctgatcatcatggatgaggcccactttacagacccagcctctatcgccgccagaggctatatcagcaccagagtggagatgggagaggcagcagcaatcttcatgacagca

SEQ ID NO: 18 - protein, 123 aa, unidentified
HPGAGKTRRYLPAIVREAIKRGLRTLILAPTRVVAAEMEEALRGLPIRYQTPAIRAEHTGREIVDLMCHATFTMRLLSPVRVPNYNLIIMDEAHFTDPASIAARGYISTRVEMGEAAAIFMTA

SEQ ID NO: 19 - DNA, 66 bp, synthetic construct
  source 1..66: mol_type = other DNA
ggaccaggacacgaggagccaatccctatgtctacctacggctggaacctggtgaagctgcagagc

SEQ ID NO: 20 - protein, 22 aa, unidentified
GPGHEEPIPMSTYGWNLVKLQS

SEQ ID NO: 21 - DNA, 195 bp, synthetic construct
  source 1..195: mol_type = other DNA
tgcgtgacacaggtgctgatgatgaggaccacatgggcactgtgcgaggccctgacactggcaaccggaccaatcagcaccctgtgggagggctcccctggcaagttctggaacaccacaatcgccgtgtccatggccaatatcttcagaggctcttacctggcaggagcaggcctggcctttagcatcatgaag

SEQ ID NO: 22 - protein, 65 aa, unidentified
CVTQVLMMRTTWALCEALTLATGPISTLWEGSPGKFWNTTIAVSMANIFRGSYLAGAGLAFSIMK

SEQ ID NO: 23 - DNA, 204 bp, synthetic construct
  source 1..204: mol_type = other DNA
tgcgtgtacaatatgatgggcaagagggagaagaagctgggcgagtttggcaaggccaagggctccagggccatctggtacatgtggctgggcgcccgcttcctggagtttgaggccctgggcttcctgaacgaggaccactggttttctcgcgagaattccctgtctggagtggagggagagggcctgcacaagctgggctat

SEQ ID NO: 24 - protein, 68 aa, unidentified
CVYNMMGKREKKLGEFGKAKGSRAIWYMWLGARFLEFEALGFLNEDHWFSRENSLSGVEGEGLHKLGY

SEQ ID NO: 25 - DNA, 231 bp, synthetic construct
  source 1..231: mol_type = other DNA
agggcaagaatctctcagggagcaggatggagcctgagggagacagcctgcctgggcaagtcctacgcccagatgtggagcctgatgtatttccacaggcgcgatctgagactggccgccaatgccatctgttctgccgtgcccagccactgggtgcctacatccaggaccacatggtctatccacgcccaccaccagtggatgaccacagaggacatgctgaccgtgtgg

SEQ ID NO: 26 - protein, 77 aa, unidentified
RARISQGAGWSLRETACLGKSYAQMWSLMYFHRRDLRLAANAICSAVPSHWVPTSRTTWSIHAHHQWMTTEDMLTVW

SEQ ID NO: 27 - DNA, 90 bp, synthetic construct
  source 1..90: mol_type = other DNA
gacatcgatctgaggcccgcctctgcctggaccctgtatgctgtcgccacaaccttcgtcacacctatgctgaggcacaccattgaaaac

SEQ ID NO: 28 - protein, 30 aa, unidentified
DIDLRPASAWTLYAVATTFVTPMLRHTIEN

SEQ ID NO: 29 - DNA, 2463 bp, synthetic construct
  source 1..2463: mol_type = other DNA
atggacccttacctgggcaaacgggaggaccagtggtgtggaagcctgattgggctgactattgatgacgacagacggggagacctgcccgtgtggctgtcttacaaggtggccagcgagggcatctcctatacagacagaaagtggtgcttcgatgagcagcagcctcactggatcgccgccagcatcatcctggagttctttctgatggtgctgctgatcccagagccagagaagcagaggaccccacaggataaccagctggcctacgtgaagtgtaacggcaatgacaccctaatcctagcacccacccgggttgtggccagcgagatggcagaggccctgaagggcatgccaatcaggtatcagaccacagccacaaagtccgagcacaccggccgcgagatcgtggataacatcaatacacctgagggcatcatcccaagcctgttcgagccagagagggagaagtccgccgcaatcgacggagagtacaggctgagaggagaggcacggaagacctttgtggacctgatgaggagaggcgatctgcccgtgtggctggcatacagagtggcagcagagggcatcaactatgccgacaggcgctggtgctttgatggcgagaggaacaatcagatcctggaggagaatatggaggtggagatctggaccaaggagggcgagagaaagaagctgaagccccggtggctggacgccagaatctacagcgatcctctggctctgaaagagtttaaggagttcgcagcaggaagggatgagctgaggccaagatggctggatgccagaacatactccgatcccatggctctgaaggacttcaaggagttttgtaactgctgtaatgaccacccctataagacctgggcctaccacggcagctatgagacaaagcagacaggctccgccagctccatggtgaatggcgtggtgaagctgctgaccaagccttgggatgtggtgccaatggtgacacagatggccatgaccgacaccacaccattcggccagcagagggtgtttaccaaccccacacagcacaccgactgggatttcgtggtgaccacagatatcagcgagatgggcgccaacttcaaggccgaccgcgtgatcgatccacggagatgcctgaagcccgtgatcctgacagacggacctgagagagtgatcctggcaggacctatgccagtgacccactctagcgccgcacagaggaggggccggcaccctggcgccggcaagacccggagatacctgccagccatcgtgagagaggccatcaagaggggcctgcgcacactgatcctggcccctaccagggtggtggcagcagagatggaggaggccctgaggggcctgcctatccgctatcagacaccagcaatcagggcagagcacaccggaagagagatcgtggatctgatgtgccacgccaccttcacaatgaggctgctgtccccagtgcgcgtgcccaactacaatctgatcatcatggatgaggcccactttacagacccagcctctatcgccgccagaggctatatcagcaccagagtggagatgggagaggcagcagcaatcttcatgacagcaggaccaggacacgaggagccaatccctatgtctacctacggctggaacctggtgaagctgcagagcgaggaggacgattgcgtgacacaggtgctgatgatgaggaccacatgggcactgtgcgaggccctgacactggcaaccggaccaatcagcaccctgtgggagggctcccctggcaagttctggaacaccacaatcgccgtgtccatggccaatatcttcagaggctcttacctggcaggagcaggcctggcctttagcatcatgaagtgcatcgacgatgagaactgcatctgcgtgtacaatatgatgggcaagagggagaagaagctgggcgagtttggcaaggccaagggctccagggccatctggtacatgtggctgggcgcccgcttcctggagtttgaggccctgggcttcctgaacgaggaccactggttttctcgcgagaattccctgtctggagtggagggagagggcctgcacaagctgggctatgacgatagggcaagaatctctcagggagcaggatggagcctgagggagacagcctgcctgggcaagtcctacgcccagatgtggagcctgatgtatttccacaggcgcgatctgagactggccgccaatgccatctgttctgccgtgcccagccactgggtgcctacatccaggaccacatggtctatccacgcccaccaccagtggatgaccacagaggacatgctgaccgtgtggtcctgtcagccccctgaggacatcgatctgaggcccgcctctgcctggaccctgtatgctgtcgccacaaccttcgtcacacctatgctgaggcacaccattgaaaactaa

SEQ ID NO: 30 - protein, 820 aa, unidentified
MDPYLGKREDQWCGSLIGLTIDDDRRGDLPVWLSYKVASEGISYTDRKWCFDEQQPHWIAASIILEFFLMVLLIPEPEKQRTPQDNQLAYVKCNGNDTLILAPTRVVASEMAEALKGMPIRYQTTATKSEHTGREIVDNINTPEGIIPSLFEPEREKSAAIDGEYRLRGEARKTFVDLMRRGDLPVWLAYRVAAEGINYADRRWCFDGERNNQILEENMEVEIWTKEGERKKLKPRWLDARIYSDPLALKEFKEFAAGRDELRPRWLDARTYSDPMALKDFKEFCNCCNDHPYKTWAYHGSYETKQTGSASSMVNGVVKLLTKPWDVVPMVTQMAMTDTTPFGQQRVFTNPTQHTDWDFVVTTDISEMGANFKADRVIDPRRCLKPVILTDGPERVILAGPMPVTHSSAAQRRGRHPGAGKTRRYLPAIVREAIKRGLRTLILAPTRVVAAEMEEALRGLPIRYQTPAIRAEHTGREIVDLMCHATFTMRLLSPVRVPNYNLIIMDEAHFTDPASIAARGYISTRVEMGEAAAIFMTAGPGHEEPIPMSTYGWNLVKLQSEEDDCVTQVLMMRTTWALCEALTLATGPISTLWEGSPGKFWNTTIAVSMANIFRGSYLAGAGLAFSIMKCIDDENCICVYNMMGKREKKLGEFGKAKGSRAIWYMWLGARFLEFEALGFLNEDHWFSRENSLSGVEGEGLHKLGYDDRARISQGAGWSLRETACLGKSYAQMWSLMYFHRRDLRLAANAICSAVPSHWVPTSRTTWSIHAHHQWMTTEDMLTVWSCQPPEDIDLRPASAWTLYAVATTFVTPMLRHTIEN

SEQ ID NO: 31 - DNA, 54 bp, synthetic construct
  source 1..54: mol_type = other DNA
gtgaccgacttccagggcaagaccgtgtggttcgtgccctccatcaaggccggc

SEQ ID NO: 32 - protein, 18 aa, unidentified
VTDFQGKTVWFVPSIKAG

SEQ ID NO: 33 - DNA, 39 bp, synthetic construct
  source 1..39: mol_type = other DNA
tccaggaagaccttcgacaccgagtaccagaagaccaag

SEQ ID NO: 34 - protein, 13 aa, unidentified
SRKTFDTEYQKTK

SEQ ID NO: 35 - DNA, 33 bp, synthetic construct
  source 1..33: mol_type = other DNA
aagaccccccgcatgtgcaccagggaggagttc

SEQ ID NO: 36 - protein, 11 aa, unidentified
KTPRMCTREEF

SEQ ID NO: 37 - DNA, 2526 bp, synthetic construct
  source 1..2526: mol_type = other DNA
atggtgaccgacttccagggcaagaccgtgtggttcgtgccctccatcaaggccggcgagaagtccaggaagaccttcgacaccgagtaccagaagaccaagaagaccccccgcatgtgcaccagggaggagttcagagacggagacctgcccgtgtggctgtcttacaaggtggccagcgagggcatctcctatacagacagaaagtggtgcttcgatgagcagcagcctcactggatcgccgccagcatcatcctggagttctttctgatggtgctgctgatcccagagccagagaagcagaggaccccacaggataaccagctggcctacgtgaagtgtaacggcaatgacacactgatcctggcacctacccgggtggtggcaagcgagatggcagaggccctgaagggcatgccaatcaggtatcagaccacagccacaaagtccgagcacaccggccgcgagatcgtggataacatcaatacacctgagggcatcatcccaagcctgttcgagccagagagggagaagtccgccgcaatcgacggagagtacaggctgagaggagaggcacggaagacctttgtggacctgatgaggagaggcgatctgcccgtgtggctggcatacagagtggcagcagagggcatcaactatgccgacaggcgctggtgctttgatggcgagaggaacaatcagatcctggaggagaatatggaggtggagatctggaccaaggagggcgagagaaagaagctgaagccccggtggctggacgccagaatctacagcgatcctctggccctgaaggagttcaaggagtttgcagcaggaagggatgagctgaggccaagatggctggatgccagaacatactccgatcccatggccctgaaggacttcaaggagttttgtaactgctgtaatgaccacccctataagacctgggcctaccacggcagctatgagacaaagcagacaggctccgccagctccatggtgaatggcgtggtgaagctgctgaccaagccttgggatgtggtgccaatggtgacacagatggccatgaccgacaccacaccattcggccagcagagggtgtttaccaaccccacacagcacaccgactgggatttcgtggtgaccacagatatcagcgagatgggcgccaacttcaaggccgaccgcgtgatcgatccacggagatgcctgaagcccgtgatcctgacagacggacctgagagagtgatcctggcaggacctatgccagtgacccactctagcgccgcacagaggaggggccggcaccctggcgccggcaagacccggagatacctgccagccatcgtgagagaggccatcaagaggggcctgcgcacactgatcctggcccctaccagggtggtggcagcagagatggaggaggccctgaggggcctgcctatccgctatcagacaccagcaatcagggcagagcacaccggaagagagatcgtggatctgatgtgccacgccaccttcacaatgaggctgctgtccccagtgcgcgtgcccaactacaatctgatcatcatggatgaggcccactttacagacccagcctctatcgccgccagaggctatatcagcaccagagtggagatgggagaggcagcagcaatcttcatgacagcaggaccaggacacgaggagccaatccctatgtctacctacggctggaacctggtgaagctgcagagcgaggaggacgattgcgtgacacaggtgctgatgatgaggaccacatgggcactgtgcgaggccctgacactggcaaccggaccaatcagcaccctgtgggagggctcccctggcaagttctggaacaccacaatcgccgtgtccatggccaatatcttcagaggctcttacctggcaggagcaggcctggcctttagcatcatgaagtgcatcgacgatgagaactgcatctgcgtgtacaatatgatgggcaagagggagaagaagctgggcgagtttggcaaggccaagggctccagggccatctggtacatgtggctgggcgcccgcttcctggagtttgaggccctgggcttcctgaacgaggaccactggttttctcgcgagaattccctgtctggagtggagggagagggcctgcacaagctgggctatgacgatagggcaagaatctctcagggagcaggatggagcctgagggagacagcctgcctgggcaagtcctacgcccagatgtggagcctgatgtatttccacaggcgcgatctgagactggccgccaatgccatctgttctgccgtgcccagccactgggtgcctacatccaggaccacatggtctatccacgcccaccaccagtggatgaccacagaggacatgctgaccgtgtggtcctgtcagccccctgaggacatcgatctgaggcccgcctctgcctggaccctgtatgctgtcgccacaaccttcgtcacacctatgctgaggcacaccattgaaaactaa

SEQ ID NO: 38 - protein, 841 aa, unidentified
MVTDFQGKTVWFVPSIKAGEKSRKTFDTEYQKTKKTPRMCTREEFRDGDLPVWLSYKVASEGISYTDRKWCFDEQQPHWIAASIILEFFLMVLLIPEPEKQRTPQDNQLAYVKCNGNDTLILAPTRVVASEMAEALKGMPIRYQTTATKSEHTGREIVDNINTPEGIIPSLFEPEREKSAAIDGEYRLRGEARKTFVDLMRRGDLPVWLAYRVAAEGINYADRRWCFDGERNNQILEENMEVEIWTKEGERKKLKPRWLDARIYSDPLALKEFKEFAAGRDELRPRWLDARTYSDPMALKDFKEFCNCCNDHPYKTWAYHGSYETKQTGSASSMVNGVVKLLTKPWDVVPMVTQMAMTDTTPFGQQRVFTNPTQHTDWDFVVTTDISEMGANFKADRVIDPRRCLKPVILTDGPERVILAGPMPVTHSSAAQRRGRHPGAGKTRRYLPAIVREAIKRGLRTLILAPTRVVAAEMEEALRGLPIRYQTPAIRAEHTGREIVDLMCHATFTMRLLSPVRVPNYNLIIMDEAHFTDPASIAARGYISTRVEMGEAAAIFMTAGPGHEEPIPMSTYGWNLVKLQSEEDDCVTQVLMMRTTWALCEALTLATGPISTLWEGSPGKFWNTTIAVSMANIFRGSYLAGAGLAFSIMKCIDDENCICVYNMMGKREKKLGEFGKAKGSRAIWYMWLGARFLEFEALGFLNEDHWFSRENSLSGVEGEGLHKLGYDDRARISQGAGWSLRETACLGKSYAQMWSLMYFHRRDLRLAANAICSAVPSHWVPTSRTTWSIHAHHQWMTTEDMLTVWSCQPPEDIDLRPASAWTLYAVATTFVTPMLRHTIEN

SEQ ID NO: 39 - DNA, 132 bp, synthetic construct
  source 1..132: mol_type = other DNA
atgctgaagaggggggtggccagggtgagccccttcggaggactgaagaggctgcccgccgggctgctgctgggacatggacctatcaggatggtgctggccatcctggccttcctgaggtttacagcaatt

SEQ ID NO: 40 - protein, 44 aa, unidentified
MLKRGVARVSPFGGLKRLPAGLLLGHGPIRMVLAILAFLRFTAI

SEQ ID NO: 41 - DNA, 99 bp, synthetic construct
  source 1..99: mol_type = other DNA
aaggaggccatggagattattaaaaagttcaaaaaggacctggccgccatgctgagaattatcaacgcaaggaaggagaaaaagaggagaggcgccgac

SEQ ID NO: 42 - protein, 33 aa, unidentified
KEAMEIIKKFKKDLAAMLRIINARKEKKRRGAD

SEQ ID NO: 43 - DNA, 198 bp, synthetic construct
  source 1..198: mol_type = other DNA
gtgggactgctgctgaccaccgccatggccgccgaggtgacaaggaggggctccgcttactacatgtacctggacaggaacgacgccggcaaagcaatcagcttcgccaccacactgggcatgaacaaatgctacatccagatcatggatctggggcacatgtgcgacgccaccatgtcctacgagtgcccaatgctg

SEQ ID NO: 44 - protein, 66 aa, unidentified
VGLLLTTAMAAEVTRRGSAYYMYLDRNDAGKAISFATTLGMNKCYIQIMDLGHMCDATMSYECPML

SEQ ID NO: 45 - DNA, 282 bp, synthetic construct
  source 1..282: mol_type = other DNA
ctgaggcccgccagtgcctgggccatctacgccgctctgaccaccttcatcacccccgccgtccagcacgccgtcaccacatcctacaacaactactccctgatggccatggccacccaggccggagtgctgttcggcatgggcaagggcatgcccttctacgcctgggactttggagtgcccctgctgatgatgggctgttactctcagctgacccccctgaccctgattgtggccattattctgctggtggctcactacatgtatctgatccccggcctg

SEQ ID NO: 46 - protein, 94 aa, unidentified
LRPASAWAIYAALTTFITPAVQHAVTTSYNNYSLMAMATQAGVLFGMGKGMPFYAWDFGVPLLMMGCYSQLTPLTLIVAIILLVAHYMYLIPGL

SEQ ID NO: 47 - DNA, 222 bp, synthetic construct
  source 1..222: mol_type = other DNA
ctgtgggagggaagccctaacaaatactggaactcctccaccgccacctccctgtgcaacatcttcaggggctcctatctggccggcgcctccctgatctacactgtgacccgcaacgccggcctggtgaaaagacggggcggcggtaccggcgagacactgggagagaaatggaaggccaggctgaaccagatgtctgccctggagttctactcctacaaa

SEQ ID NO: 48 - protein, 74 aa, unidentified
LWEGSPNKYWNSSTATSLCNIFRGSYLAGASLIYTVTRNAGLVKRRGGGTGETLGEKWKARLNQMSALEFYSYK

SEQ ID NO: 49 - DNA, 288 bp, synthetic construct
  source 1..288: mol_type = other DNA
tggttcttcgatgaaaaccacccctacaggacatgggcctaccacggaagctacgaagcccccacccagggctccgcctcctctctgatcaacggcgtggtgaggctgctgtccaagccctgggacgtggtgaccggcgtgaccggaatcgctatgaccgacacaaccccctacggccagcagcgggtgtttaaggagaaggtggacaccagggtgcccgacccccaggagggaacaaggcaggtgatgaacatggtgagcagttggctgtggaaggagctgggcaaa

SEQ ID NO: 50 - protein, 96 aa, unidentified
WFFDENHPYRTWAYHGSYEAPTQGSASSLINGVVRLLSKPWDVVTGVTGIAMTDTTPYGQQRVFKEKVDTRVPDPQEGTRQVMNMVSSWLWKELGK

SEQ ID NO: 51 - DNA, 210 bp, synthetic construct
  source 1..210: mol_type = other DNA
agggagacagcttgcctggctaagagctacgcccagatgtggcagctgctgtactttcacaggagggacctgaggctgatggccaatgccatctgttctagcgtgcccgtggattgggtgcccacaggaaggactacctggtcaatccacggcaagggcgagtggatgaccaccgaggacatgctggtggtgtggaacagagtgtggatc

SEQ ID NO: 52 - protein, 70 aa, unidentified
RETACLAKSYAQMWQLLYFHRRDLRLMANAICSSVPVDWVPTGRTTWSIHGKGEWMTTEDMLVVWNRVWI

SEQ ID NO: 53 - DNA, 1485 bp, synthetic construct
  source 1..1485: mol_type = other DNA
atgctgaagaggggggtggccagggtgagccccttcggaggactgaagaggctgcccgccgggctgctgctgggacatggacctatcaggatggtgctggccatcctggccttcctgaggtttacagcaattagggataaggaggccatggagattattaaaaagttcaaaaaggacctggccgccatgctgagaattatcaacgcaaggaaggagaaaaagaggagaggcgccgacaagggcaaggtgggactgctgctgaccaccgccatggccgccgaggtgacaaggaggggctccgcttactacatgtacctggacaggaacgacgccggcaaagcaatcagcttcgccaccacactgggcatgaacaaatgctacatccagatcatggatctggggcacatgtgcgacgccaccatgtcctacgagtgcccaatgctgctgaggcccgccagtgcctgggccatctacgccgctctgaccaccttcatcacccccgccgtccagcacgccgtcaccacatcctacaacaactactccctgatggccatggccacccaggccggagtgctgttcggcatgggcaagggcatgcccttctacgcctgggactttggagtgcccctgctgatgatgggctgttactctcagctgacccccctgaccctgattgtggccattattctgctggtggctcactacatgtatctgatccccggcctgggccccggcctgtgggagggaagccctaacaaatactggaactcctccaccgccacctccctgtgcaacatcttcaggggctcctatctggccggcgcctccctgatctacactgtgacccgcaacgccggcctggtgaaaagacggggcggcggtaccggcgagacactgggagagaaatggaaggccaggctgaaccagatgtctgccctggagttctactcctacaaagagcccaccaacgatgatagggggcagtggttcttcgatgaaaaccacccctacaggacatgggcctaccacggaagctacgaagcccccacccagggctccgcctcctctctgatcaacggcgtggtgaggctgctgtccaagccctgggacgtggtgaccggcgtgaccggaatcgctatgaccgacacaaccccctacggccagcagcgggtgtttaaggagaaggtggacaccagggtgcccgacccccaggagggaacaaggcaggtgatgaacatggtgagcagttggctgtggaaggagctgggcaaaagggagacagcttgcctggctaagagctacgcccagatgtggcagctgctgtactttcacaggagggacctgaggctgatggccaatgccatctgttctagcgtgcccgtggattgggtgcccacaggaaggactacctggtcaatccacggcaagggcgagtggatgaccaccgaggacatgctggtggtgtggaacagagtgtggatctaa

SEQ ID NO: 54 - protein, 494 aa, unidentified
MLKRGVARVSPFGGLKRLPAGLLLGHGPIRMVLAILAFLRFTAIRDKEAMEIIKKFKKDLAAMLRIINARKEKKRRGADKGKVGLLLTTAMAAEVTRRGSAYYMYLDRNDAGKAISFATTLGMNKCYIQIMDLGHMCDATMSYECPMLLRPASAWAIYAALTTFITPAVQHAVTTSYNNYSLMAMATQAGVLFGMGKGMPFYAWDFGVPLLMMGCYSQLTPLTLIVAIILLVAHYMYLIPGLGPGLWEGSPNKYWNSSTATSLCNIFRGSYLAGASLIYTVTRNAGLVKRRGGGTGETLGEKWKARLNQMSALEFYSYKEPTNDDRGQWFFDENHPYRTWAYHGSYEAPTQGSASSLINGVVRLLSKPWDVVTGVTGIAMTDTTPYGQQRVFKEKVDTRVPDPQEGTRQVMNMVSSWLWKELGKRETACLAKSYAQMWQLLYFHRRDLRLMANAICSSVPVDWVPTGRTTWSIHGKGEWMTTEDMLVVWNRVWI

SEQ ID NO: 55 - DNA, 1125 bp, synthetic construct
  source 1..1125: mol_type = other DNA
atggctctcgctctcggaggagtgctcatcttcctgtccaccgctgtgtccgctgatgtgggatgttccgtggatttctccaagaaggagacccgctgcggaaccggagtgttcgtctacaacgacgtggaggcatggagagacaggtacaagtaccaccctgactcccccaggcgcttggctgcagcagtcaagcaggcctgggaagatggaatctgtgggatctcctccgtgtcacggatggagaacatcatgtggcggtccgtggagggggaattgaacgccatccttgaggagaacggggtgcagcttaccgtcgtggtggggtctgtcaaaaaccctatgtggcggggcccacaaagactccctgtgcctgtgaacgaacttcctcacggctggaaggcctgggggaaatcttacttcgtccgggccgccaagacaaacaactccttcgtcgtggatggcgataccttgaaggagtgcccactgaagcaccgggcctggaattcttttctggtggaggatcacgggtttggggtctttcacaccagcgtctggctcaaggtgcgggaggactacagcctggagtgcgatccagccgtgatcgggacagccgtgaaagggaaggaagccgtccatagtgatctgggctactggatcgagagcgagaagaacgacacctggaggttgaagagggcccacctgatcgagatgaagacctgcgagtggccaaagagccacacactgtggacagacgggattgaggagagcgacctgattattcccaagagcctggccgggccactcagccaccacaacacacgggagggctacagaacacagatgaaggggccctggcatagcgaggagctggaaattaggtttgaggagtgccccggcactaaggtccatgtggaggagacatgcggcactcggggccccagtctgagaagtacaactgcctcaggcagagtgattgaggagtggtgctgcagggagtgcacaatgccccccctgtcatttagggccaaggacggctgttggtatggcatggagattcggcccaggaaagaacccgaaagcaacctggtcaggagcatggtcactgccggcagcacttaatga

SEQ ID NO: 56 - protein, 373 aa, unidentified
  SIGNAL 1..20
MALALGGVLIFLSTAVSADVGCSVDFSKKETRCGTGVFVYNDVEAWRDRYKYHPDSPRRLAAAVKQAWEDGICGISSVSRMENIMWRSVEGELNAILEENGVQLTVVVGSVKNPMWRGPQRLPVPVNELPHGWKAWGKSYFVRAAKTNNSFVVDGDTLKECPLKHRAWNSFLVEDHGFGVFHTSVWLKVREDYSLECDPAVIGTAVKGKEAVHSDLGYWIESEKNDTWRLKRAHLIEMKTCEWPKSHTLWTDGIEESDLIIPKSLAGPLSHHNTREGYRTQMKGPWHSEELEIRFEECPGTKVHVEETCGTRGPSLRSTTASGRVIEEWCCRECTMPPLSFRAKDGCWYGMEIRPRKEPESNLVRSMVTAGST

SEQ ID NO: 57 - DNA, 84 bp, synthetic construct
  source 1..84: mol_type = other DNA
ctggtgtccggctggaactccatcaccgtgatgcccctgctgtgcgggatcggctgcgccatgctgcactggtccctgatcctg

SEQ ID NO: 58 - protein, 28 aa, unidentified
LVSGWNSITVMPLLCGIGCAMLHWSLIL

SEQ ID NO: 59 - DNA, 57 bp, synthetic construct
  source 1..57: mol_type = other DNA
aggatggccatgaccgacaccacccccttcggccagcagcgcgtgttcaaggagaag

SEQ ID NO: 60 - protein, 19 aa, unidentified
RMAMTDTTPFGQQRVFKEK

SEQ ID NO: 61 - DNA, 27 bp, synthetic construct
  source 1..27: mol_type = other DNA
agatcccacgccgccatcggtgcctac

SEQ ID NO: 62 - protein, 9 aa, unidentified
RSHAAIGAY

SEQ ID NO: 63 - DNA, 111 bp, synthetic construct
  source 1..111: mol_type = other DNA
ctgctgtggaacggacccatggcagtgtccatgaccggcgtgatgaggggcaaccattacgcattcgtgggcgtgatgtacaacctgtggaaaatgaagaccggaaggagg

SEQ ID NO: 64 - protein, 37 aa, unidentified
LLWNGPMAVSMTGVMRGNHYAFVGVMYNLWKMKTGRR

SEQ ID NO: 65 - DNA, 102 bp, synthetic construct
  source 1..102: mol_type = other DNA
gagtttggcaaggcaaagggcagcagggctatctggtacatgtggctgggcgccaggtacctggagttcgaggctctgggcttcctgaacgaggaccattgg

SEQ ID NO: 66 - protein, 34 aa, unidentified
EFGKAKGSRAIWYMWLGARYLEFEALGFLNEDHW

SEQ ID NO: 67 - DNA, 42 bp, synthetic construct
  source 1..42: mol_type = other DNA
agggacctggccgccatggacggaggaggattctatgccgac

SEQ ID NO: 68 - protein, 14 aa, unidentified
RDLAAMDGGGFYAD

SEQ ID NO: 69 - DNA, 105 bp, synthetic construct
  source 1..105: mol_type = other DNA
aggaggaggctgaggacactggtgctggctcctactagggtggtgctgagcgaaatgaaggaggcctttcacggactggatgtgaagtttcatacccaggctttc

SEQ ID NO: 70 - protein, 35 aa, unidentified
RRRLRTLVLAPTRVVLSEMKEAFHGLDVKFHTQAF

SEQ ID NO: 71 - DNA, 42 bp, synthetic construct
  source 1..42: mol_type = other DNA
atcgacgcaatgtgccacgccaccctgacctacaggatgctg

SEQ ID NO: 72 - protein, 14 aa, unidentified
IDAMCHATLTYRML

SEQ ID NO: 73 - DNA, 30 bp, synthetic construct
  source 1..30: mol_type = other DNA
cacgccgtgcccttcggactggtgagtatg

SEQ ID NO: 74 - protein, 10 aa, unidentified
HAVPFGLVSM

SEQ ID NO: 75 - DNA, 30 bp, synthetic construct
  source 1..30: mol_type = other DNA
aagctgaccgtggctgtgggactgcacttc

SEQ ID NO: 76 - protein, 10 aa, unidentified
KLTVAVGLHF

SEQ ID NO: 77 - DNA, 27 bp, synthetic construct
  source 1..27: mol_type = other DNA
atgtacatggccctgatcgccgccttc

SEQ ID NO: 78 - protein, 9 aa, unidentified
MYMALIAAF

SEQ ID NO: 79 - DNA, 27 bp, synthetic construct
  source 1..27: mol_type = other DNA
ggagtgatgggaggcctgtggaaatac

SEQ ID NO: 80 - protein, 9 aa, unidentified
GVMGGLWKY

SEQ ID NO: 81 - DNA, 93 bp, synthetic construct
  source 1..93: mol_type = other DNA
gaaccctggaacactggccatgactggatcttggcagataaaaggcctacagcctggttcctgccctctatccgtgccgccaacgtgatggct

SEQ ID NO: 82 - protein, 31 aa, unidentified
EPWNTGHDWILADKRPTAWFLPSIRAANVMA

SEQ ID NO: 83 - DNA, 81 bp, synthetic construct
  source 1..81: mol_type = other DNA
ggcacaagtggctcccccatcgtgaacaggaacggcgaggtgattggcctgtacggaaatggaattctggtgggcgacaac

SEQ ID NO: 84 - protein, 27 aa, unidentified
GTSGSPIVNRNGEVIGLYGNGILVGDN

SEQ ID NO: 85 - DNA, 66 bp, synthetic construct
  source 1..66: mol_type = other DNA
ctggtgggcgcagcactgcaccccttcgccctgctgctggtcctggctggctggctgttccacgtg

SEQ ID NO: 86 - protein, 22 aa, unidentified
LVGAALHPFALLLVLAGWLFHV

SEQ ID NO: 87 - DNA, 45 bp, synthetic construct
  source 1..45: mol_type = other DNA
agcgtgaaggaggacctggtggcttacggcggctcctggaagctg

SEQ ID NO: 88 - protein, 15 aa, unidentified
SVKEDLVAYGGSWKL

SEQ ID NO: 89 - DNA, 147 bp, synthetic construct
  source 1..147: mol_type = other DNA
gaggctatggacaccatctccgtctttctgcattccgaagaaggtagcagggcttataggaacgccctgagcatgatgcccgaagctatgaccatcgtgatgctgttcatcctggccggactgctgacctccggcatggtgattttc

SEQ ID NO: 90 - protein, 49 aa, unidentified
EAMDTISVFLHSEEGSRAYRNALSMMPEAMTIVMLFILAGLLTSGMVIF

SEQ ID NO: 91 - DNA, 66 bp, synthetic construct
  source 1..66: mol_type = other DNA
ctggtggccctgaccctgacctcctacctggggctgacccagccctttctgggcctgtgcgctttc

SEQ ID NO: 92 - protein, 22 aa, unidentified
LVALTLTSYLGLTQPFLGLCAF

SEQ ID NO: 93 - DNA, 27 bp, synthetic construct
  source 1..27: mol_type = other DNA
aggcccattgacgaccgctttgggctg

SEQ ID NO: 94 - protein, 9 aa, unidentified
RPIDDRFGL

SEQ ID NO: 95 - DNA, 33 bp, synthetic construct
  source 1..33: mol_type = other DNA
aaggcctatgctaacatgtggtccctgatgtac

SEQ ID NO: 96 - protein, 11 aa, unidentified
KAYANMWSLMY

SEQ ID NO: 97 - DNA, 54 bp, synthetic construct
  source 1..54: mol_type = other DNA
ggcggcgtgaaacccacccatatctcctacattatgctgatcttctttgtcctg

SEQ ID NO: 98 - protein, 18 aa, unidentified
GGVKPTHISYIMLIFFVL

SEQ ID NO: 99 - DNA, 27 bp, synthetic construct
  source 1..27: mol_type = other DNA
tacctgatcatcggcatcctgaccctg

SEQ ID NO: 100 - protein, 9 aa, unidentified
YLIIGILTL

SEQ ID NO: 101 - DNA, 51 bp, synthetic construct
  source 1..51: mol_type = other DNA
atgcccgccctgtacgagaaaaagctggccctgtacctgctgctggccctg

SEQ ID NO: 102 - protein, 17 aa, unidentified
MPALYEKKLALYLLLAL

SEQ ID NO: 103 - DNA, 72 bp, synthetic construct
  source 1..72: mol_type = other DNA
agcgagtacatgacctcatggttctacgacaacgacaacccctataggacctggcactattgcggcagctac

SEQ ID NO: 104 - protein, 24 aa, unidentified
SEYMTSWFYDNDNPYRTWHYCGSY

SEQ ID NO: 105 - DNA, 1620 bp, synthetic construct
  source 1..1620: mol_type = other DNA
atggccctggtgtccggctggaactccatcaccgtgatgcccctgctgtgcgggatcggctgcgccatgctgcactggtccctgatcctgaaaaaggaggagaggatggccatgaccgacaccacccccttcggccagcagcgcgtgttcaaggagaagagatcccacgccgccatcggtgcctacaaagacaagctgctgtggaacggacccatggcagtgtccatgaccggcgtgatgaggggcaaccattacgcattcgtgggcgtgatgtacaacctgtggaaaatgaagaccggaaggagggacgagtttggcaaggcaaagggcagcagggctatctggtacatgtggctgggcgccaggtacctggagttcgaggctctgggcttcctgaacgaggaccattggagggacctggccgccatggacggaggaggattctatgccgactgtgagaggaggaggctgaggacactggtgctggctcctactagggtggtgctgagcgaaatgaaggaggcctttcacggactggatgtgaagtttcatacccaggctttcaaggagaaagaaatcgacgcaatgtgccacgccaccctgacctacaggatgctgggacactgcaaaggacacgccgtgcccttcggactggtgagtatgaagctgaccgtggctgtgggactgcacttcgacgacatgtacatggccctgatcgccgccttcggagtgatgggaggcctgtggaaatacgaaccctggaacactggccatgactggatcttggcagataaaaggcctacagcctggttcctgccctctatccgtgccgccaacgtgatggctaagggcacaagtggctcccccatcgtgaacaggaacggcgaggtgattggcctgtacggaaatggaattctggtgggcgacaacaaacacagggacctggtgggcgcagcactgcaccccttcgccctgctgctggtcctggctggctggctgttccacgtgggccccggaagcgtgaaggaggacctggtggcttacggcggctcctggaagctgggacccgaggctatggacaccatctccgtctttctgcattccgaagaaggtagcagggcttataggaacgccctgagcatgatgcccgaagctatgaccatcgtgatgctgttcatcctggccggactgctgacctccggcatggtgattttcgagggcgaggatacactggtggccctgaccctgacctcctacctggggctgacccagccctttctgggcctgtgcgctttccgtaggcccattgacgaccgctttgggctgaaggcctatgctaacatgtggtccctgatgtaccgcaacaaggacggcggcgtgaaacccacccatatctcctacattatgctgatcttctttgtcctgagggaggagaggtacctgatcatcggcatcctgaccctgactagggacgagtgtatgcccgccctgtacgagaaaaagctggccctgtacctgctgctggccctgaagttcaaggacgacagcgagtacatgacctcatggttctacgacaacgacaacccctataggacctggcactattgcggcagctactaa

SEQ ID NO: 106 - protein, 539 aa, unidentified
MALVSGWNSITVMPLLCGIGCAMLHWSLILKKEERMAMTDTTPFGQQRVFKEKRSHAAIGAYKDKLLWNGPMAVSMTGVMRGNHYAFVGVMYNLWKMKTGRRDEFGKAKGSRAIWYMWLGARYLEFEALGFLNEDHWRDLAAMDGGGFYADCERRRLRTLVLAPTRVVLSEMKEAFHGLDVKFHTQAFKEKEIDAMCHATLTYRMLGHCKGHAVPFGLVSMKLTVAVGLHFDDMYMALIAAFGVMGGLWKYEPWNTGHDWILADKRPTAWFLPSIRAANVMAKGTSGSPIVNRNGEVIGLYGNGILVGDNKHRDLVGAALHPFALLLVLAGWLFHVGPGSVKEDLVAYGGSWKLGPEAMDTISVFLHSEEGSRAYRNALSMMPEAMTIVMLFILAGLLTSGMVIFEGEDTLVALTLTSYLGLTQPFLGLCAFRRPIDDRFGLKAYANMWSLMYRNKDGGVKPTHISYIMLIFFVLREERYLIIGILTLTRDECMPALYEKKLALYLLLALKFKDDSEYMTSWFYDNDNPYRTWHYCGSY

SEQ ID NO: 107 - DNA, 30 bp, synthetic construct
  source 1..30: mol_type = other DNA
ctgctgatgaccggcggcgtgaccctggtg

SEQ ID NO: 108 - protein, 10 aa, unidentified
LLMTGGVTLV

SEQ ID NO: 109 - DNA, 171 bp, synthetic construct
  source 1..171: mol_type = other DNA
gtgcaggggttcatcttcttcttcctgttcaacatcctgaccgggaagaaaatcaccgcccacctgaagaggctgtggaaaatgctggaccctaggcagggcctggccgtgctgaggaaggtgaagagggtggtggcctctctgatgagggggctgtccagtaggaagcgg

SEQ ID NO: 110 - protein, 57 aa, unidentified
VQGFIFFFLFNILTGKKITAHLKRLWKMLDPRQGLAVLRKVKRVVASLMRGLSSRKR

SEQ ID NO: 111 - DNA, 27 bp, synthetic construct
  source 1..27: mol_type = other DNA
tactggtgccccgactccatggagtac

SEQ ID NO: 112 - protein, 9 aa, unidentified
YWCPDSMEY

SEQ ID NO: 113 - DNA, 141 bp, synthetic construct
  source 1..141: mol_type = other DNA
aggcaggagaagtggatgacaggcaggatgggagaaaggcagctgcagaagattgaaaggtggttcgtgaggaatccctttttcgccgtgactgctctgaccatcgcttacctggtgggcagtaacatgactcagagggtg

SEQ ID NO: 114 - protein, 47 aa, unidentified
RQEKWMTGRMGERQLQKIERWFVRNPFFAVTALTIAYLVGSNMTQRV

SEQ ID NO: 115 - DNA, 78 bp, synthetic construct
  source 1..78: mol_type = other DNA
gagaccgtggccatcgacaggcccgcagaggtgaggaaggtctgttacaatgccgtgctgacacacgtgaaaatcaac

SEQ ID NO: 116 - protein, 26 aa, unidentified
ETVAIDRPAEVRKVCYNAVLTHVKIN

SEQ ID NO: 117 - DNA, 27 bp, synthetic construct
  source 1..27: mol_type = other DNA
ttcagtaacagggtgtggaactccttc

SEQ ID NO: 118 - protein, 9 aa, unidentified
FSNRVWNSF

SEQ ID NO: 119 - DNA, 27 bp, synthetic construct
  source 1..27: mol_type = other DNA
aagtccgcccacggaagccccaccttt

SEQ ID NO: 120 - protein, 9 aa, unidentified
KSAHGSPTF

SEQ ID NO: 121 - DNA, 33 bp, synthetic construct
  source 1..33: mol_type = other DNA
aagctggcctccatcgtgaaggcctcatttgag

SEQ ID NO: 122 - protein, 11 aa, unidentified
KLASIVKASFE

SEQ ID NO: 123 - DNA, 27 bp, synthetic construct
  source 1..27: mol_type = other DNA
atcccagtgatcgtggctgacgacctg

SEQ ID NO: 124 - protein, 9 aa, unidentified
IPVIVADDL

SEQ ID NO: 125 - DNA, 42 bp, synthetic construct
  source 1..42: mol_type = other DNA
aacaagggcatcctggtcaccgtgaacccaatcgcctccacc

SEQ ID NO: 126 - protein, 14 aa, unidentified
NKGILVTVNPIAST

SEQ ID NO: 127 - DNA, 69 bp, synthetic construct
  source 1..69: mol_type = other DNA
aagtccatgtccctgtttgaggtggaccagacaaagatccagtatgtgatccgggcccagctgcacgtc

SEQ ID NO: 128 - protein, 23 aa, unidentified
KSMSLFEVDQTKIQYVIRAQLHV

SEQ ID NO: 129 - DNA, 90 bp, synthetic construct
  source 1..90: mol_type = other DNA
accgccgtcgactttggcaacagctacatcgccgagatggagaccgagagctggattgtggacagacagtgggctcaggacctgacactg

SEQ ID NO: 130 - protein, 30 aa, unidentified
TAVDFGNSYIAEMETESWIVDRQWAQDLTL

SEQ ID NO: 131 - DNA, 27 bp, synthetic construct
  source 1..27: mol_type = other DNA
gtgctgattgaggtgaatccccccttc

SEQ ID NO: 132 - protein, 9 aa, unidentified
VLIEVNPPF

SEQ ID NO: 133 - DNA, 30 bp, synthetic construct
  source 1..30: mol_type = other DNA
gtgagcagccacaaccacatccccggctat

SEQ ID NO: 134 - protein, 10 aa, unidentified
VSSHNHIPGY

SEQ ID NO: 135 - DNA, 129 bp, synthetic construct
  source 1..129: mol_type = other DNA
ggaggactgaactggatcaccaaggtgatcatgggcgccgtgctgatctgggtcggcatcaacaccaggaacatgactatgtccatgtccatgatcctggtgggcgtgatcatgatgtttctgtccctg

SEQ ID NO: 136 - protein, 43 aa, unidentified
GGLNWITKVIMGAVLIWVGINTRNMTMSMSMILVGVIMMFLSL

SEQ ID NO: 137 - DNA, 30 bp, synthetic construct
  source 1..30: mol_type = other DNA
gtgtacatggacgccgtgttcgaatacacc

SEQ ID NO: 138 - protein, 10 aa, unidentified
VYMDAVFEYT

SEQ ID NO: 139 - DNA, 30 bp, synthetic construct
  source 1..30: mol_type = other DNA
accaacggcccctggatgcaggtgcctctg

SEQ ID NO: 140 - protein, 10 aa, unidentified
TNGPWMQVPL

SEQ ID NO: 141 - DNA, 1164 bp, synthetic construct
  source 1..1164: mol_type = other DNA
atggccctgctgatgaccggcggcgtgaccctggtgaaggacaaggtgcaggggttcatcttcttcttcctgttcaacatcctgaccgggaagaaaatcaccgcccacctgaagaggctgtggaaaatgctggaccctaggcagggcctggccgtgctgaggaaggtgaagagggtggtggcctctctgatgagggggctgtccagtaggaagcggtactggtgccccgactccatggagtacaggcaggagaagtggatgacaggcaggatgggagaaaggcagctgcagaagattgaaaggtggttcgtgaggaatccctttttcgccgtgactgctctgaccatcgcttacctggtgggcagtaacatgactcagagggtggagaccgtggccatcgacaggcccgcagaggtgaggaaggtctgttacaatgccgtgctgacacacgtgaaaatcaacttcagtaacagggtgtggaactccttcaaagacagagacaagtccgcccacggaagccccacctttaaggataagcggaagctggcctccatcgtgaaggcctcatttgagaagagggagaagatcccagtgatcgtggctgacgacctgaacaagggcatcctggtcaccgtgaacccaatcgcctccaccggcaggagggataagtccatgtccctgtttgaggtggaccagacaaagatccagtatgtgatccgggcccagctgcacgtccgcgaggtcgaagaaaccgccgtcgactttggcaacagctacatcgccgagatggagaccgagagctggattgtggacagacagtgggctcaggacctgacactggaggaatgcgatgacgtgctgattgaggtgaatccccccttcaaggactgcgagaaagtgagcagccacaaccacatccccggctataggagggaccacggaggactgaactggatcaccaaggtgatcatgggcgccgtgctgatctgggtcggcatcaacaccaggaacatgactatgtccatgtccatgatcctggtgggcgtgatcatgatgtttctgtccctgcccgacgacgacaagtgcaaggtgtacatggacgccgtgttcgaatacaccaggaagaacaagaccaacggcccctggatgcaggtgcctctgtaa

SEQ ID NO: 142 - protein, 387 aa, unidentified
MALLMTGGVTLVKDKVQGFIFFFLFNILTGKKITAHLKRLWKMLDPRQGLAVLRKVKRVVASLMRGLSSRKRYWCPDSMEYRQEKWMTGRMGERQLQKIERWFVRNPFFAVTALTIAYLVGSNMTQRVETVAIDRPAEVRKVCYNAVLTHVKINFSNRVWNSFKDRDKSAHGSPTFKDKRKLASIVKASFEKREKIPVIVADDLNKGILVTVNPIASTGRRDKSMSLFEVDQTKIQYVIRAQLHVREVEETAVDFGNSYIAEMETESWIVDRQWAQDLTLEECDDVLIEVNPPFKDCEKVSSHNHIPGYRRDHGGLNWITKVIMGAVLIWVGINTRNMTMSMSMILVGVIMMFLSLPDDDKCKVYMDAVFEYTRKNKTNGPWMQVPL

SEQ ID NO: 143 - DNA, 4074 bp, synthetic construct
  source 1..4074: mol_type = other DNA
atggtgaccgacttccagggcaagaccgtgtggttcgtgccctccatcaaggccggcgagaagtccaggaagaccttcgacaccgagtaccagaagaccaagaagaccccccgcatgtgcaccagggaggagttcagagacggagacctgcccgtgtggctgtcttacaaggtggccagcgagggcatctcctatacagacagaaagtggtgcttcgatgagcagcagcctcactggatcgccgccagcatcatcctggagttctttctgatggtgctgctgatcccagagccagagaagcagaggaccccacaggataaccagctggcctacgtgaagtgtaacggcaatgacacactgatcctggcacctacccgggtggtggcaagcgagatggcagaggccctgaagggcatgccaatcaggtatcagaccacagccacaaagtccgagcacaccggccgcgagatcgtggataacatcaatacacctgagggcatcatcccaagcctgttcgagccagagagggagaagtccgccgcaatcgacggagagtacaggctgagaggagaggcacggaagacctttgtggacctgatgaggagaggcgatctgcccgtgtggctggcatacagagtggcagcagagggcatcaactatgccgacaggcgctggtgctttgatggcgagaggaacaatcagatcctggaggagaatatggaggtggagatctggaccaaggagggcgagagaaagaagctgaagccccggtggctggacgccagaatctacagcgatcctctggccctgaaggagttcaaggagtttgcagcaggaagggatgagctgaggccaagatggctggatgccagaacatactccgatcccatggccctgaaggacttcaaggagttttgtaactgctgtaatgaccacccctataagacctgggcctaccacggcagctatgagacaaagcagacaggctccgccagctccatggtgaatggcgtggtgaagctgctgaccaagccttgggatgtggtgccaatggtgacacagatggccatgaccgacaccacaccattcggccagcagagggtgtttaccaaccccacacagcacaccgactgggatttcgtggtgaccacagatatcagcgagatgggcgccaacttcaaggccgaccgcgtgatcgatccacggagatgcctgaagcccgtgatcctgacagacggacctgagagagtgatcctggcaggacctatgccagtgacccactctagcgccgcacagaggaggggccggcaccctggcgccggcaagacccggagatacctgccagccatcgtgagagaggccatcaagaggggcctgcgcacactgatcctggcccctaccagggtggtggcagcagagatggaggaggccctgaggggcctgcctatccgctatcagacaccagcaatcagggcagagcacaccggaagagagatcgtggatctgatgtgccacgccaccttcacaatgaggctgctgtccccagtgcgcgtgcccaactacaatctgatcatcatggatgaggcccactttacagacccagcctctatcgccgccagaggctatatcagcaccagagtggagatgggagaggcagcagcaatcttcatgacagcaggaccaggacacgaggagccaatccctatgtctacctacggctggaacctggtgaagctgcagagcgaggaggacgattgcgtgacacaggtgctgatgatgaggaccacatgggcactgtgcgaggccctgacactggcaaccggaccaatcagcaccctgtgggagggctcccctggcaagttctggaacaccacaatcgccgtgtccatggccaatatcttcagaggctcttacctggcaggagcaggcctggcctttagcatcatgaagtgcatcgacgatgagaactgcatctgcgtgtacaatatgatgggcaagagggagaagaagctgggcgagtttggcaaggccaagggctccagggccatctggtacatgtggctgggcgcccgcttcctggagtttgaggccctgggcttcctgaacgaggaccactggttttctcgcgagaattccctgtctggagtggagggagagggcctgcacaagctgggctatgacgatagggcaagaatctctcagggagcaggatggagcctgagggagacagcctgcctgggcaagtcctacgcccagatgtggagcctgatgtatttccacaggcgcgatctgagactggccgccaatgccatctgttctgccgtgcccagccactgggtgcctacatccaggaccacatggtctatccacgcccaccaccagtggatgaccacagaggacatgctgaccgtgtggtcctgtcagccccctgaggacatcgatctgaggcccgcctctgcctggaccctgtatgctgtcgccacaaccttcgtcacacctatgctgaggcacaccattgaaaacggatctggtgccaccaacttttccctgctgaaacaggccggcgatgtggaggaaaacccaggccccatgctgaagaggggggtggccagggtgagccccttcggaggactgaagaggctgcccgccgggctgctgctgggacatggacctatcaggatggtgctggccatcctggccttcctgaggtttacagcaattagggataaggaggccatggagattattaaaaagttcaaaaaggacctggccgccatgctgagaattatcaacgcaaggaaggagaaaaagaggagaggcgccgacaagggcaaggtgggactgctgctgaccaccgccatggccgccgaggtgacaaggaggggctccgcttactacatgtacctggacaggaacgacgccggcaaagcaatcagcttcgccaccacactgggcatgaacaaatgctacatccagatcatggatctggggcacatgtgcgacgccaccatgtcctacgagtgcccaatgctgctgaggcccgccagtgcctgggccatctacgccgctctgaccaccttcatcacccccgccgtccagcacgccgtcaccacatcctacaacaactactccctgatggccatggccacccaggccggagtgctgttcggcatgggcaagggcatgcccttctacgcctgggactttggagtgcccctgctgatgatgggctgttactctcagctgacccccctgaccctgattgtggccattattctgctggtggctcactacatgtatctgatccccggcctgggccccggcctgtgggagggaagccctaacaaatactggaactcctccaccgccacctccctgtgcaacatcttcaggggctcctatctggccggcgcctccctgatctacactgtgacccgcaacgccggcctggtgaaaagacggggcggcggtaccggcgagacactgggagagaaatggaaggccaggctgaaccagatgtctgccctggagttctactcctacaaagagcccaccaacgatgatagggggcagtggttcttcgatgaaaaccacccctacaggacatgggcctaccacggaagctacgaagcccccacccagggctccgcctcctctctgatcaacggcgtggtgaggctgctgtccaagccctgggacgtggtgaccggcgtgaccggaatcgctatgaccgacacaaccccctacggccagcagcgggtgtttaaggagaaggtggacaccagggtgcccgacccccaggagggaacaaggcaggtgatgaacatggtgagcagttggctgtggaaggagctgggcaaaagggagacagcttgcctggctaagagctacgcccagatgtggcagctgctgtactttcacaggagggacctgaggctgatggccaatgccatctgttctagcgtgcccgtggattgggtgcccacaggaaggactacctggtcaatccacggcaagggcgagtggatgaccaccgaggacatgctggtggtgtggaacagagtgtggatctaa

SEQ ID NO: 144 - protein, 1357 aa, unidentified
MVTDFQGKTVWFVPSIKAGEKSRKTFDTEYQKTKKTPRMCTREEFRDGDLPVWLSYKVASEGISYTDRKWCFDEQQPHWIAASIILEFFLMVLLIPEPEKQRTPQDNQLAYVKCNGNDTLILAPTRVVASEMAEALKGMPIRYQTTATKSEHTGREIVDNINTPEGIIPSLFEPEREKSAAIDGEYRLRGEARKTFVDLMRRGDLPVWLAYRVAAEGINYADRRWCFDGERNNQILEENMEVEIWTKEGERKKLKPRWLDARIYSDPLALKEFKEFAAGRDELRPRWLDARTYSDPMALKDFKEFCNCCNDHPYKTWAYHGSYETKQTGSASSMVNGVVKLLTKPWDVVPMVTQMAMTDTTPFGQQRVFTNPTQHTDWDFVVTTDISEMGANFKADRVIDPRRCLKPVILTDGPERVILAGPMPVTHSSAAQRRGRHPGAGKTRRYLPAIVREAIKRGLRTLILAPTRVVAAEMEEALRGLPIRYQTPAIRAEHTGREIVDLMCHATFTMRLLSPVRVPNYNLIIMDEAHFTDPASIAARGYISTRVEMGEAAAIFMTAGPGHEEPIPMSTYGWNLVKLQSEEDDCVTQVLMMRTTWALCEALTLATGPISTLWEGSPGKFWNTTIAVSMANIFRGSYLAGAGLAFSIMKCIDDENCICVYNMMGKREKKLGEFGKAKGSRAIWYMWLGARFLEFEALGFLNEDHWFSRENSLSGVEGEGLHKLGYDDRARISQGAGWSLRETACLGKSYAQMWSLMYFHRRDLRLAANAICSAVPSHWVPTSRTTWSIHAHHQWMTTEDMLTVWSCQPPEDIDLRPASAWTLYAVATTFVTPMLRHTIENGSGATNFSLLKQAGDVEENPGPMLKRGVARVSPFGGLKRLPAGLLLGHGPIRMVLAILAFLRFTAIRDKEAMEIIKKFKKDLAAMLRIINARKEKKRRGADKGKVGLLLTTAMAAEVTRRGSAYYMYLDRNDAGKAISFATTLGMNKCYIQIMDLGHMCDATMSYECPMLLRPASAWAIYAALTTFITPAVQHAVTTSYNNYSLMAMATQAGVLFGMGKGMPFYAWDFGVPLLMMGCYSQLTPLTLIVAIILLVAHYMYLIPGLGPGLWEGSPNKYWNSSTATSLCNIFRGSYLAGASLIYTVTRNAGLVKRRGGGTGETLGEKWKARLNQMSALEFYSYKEPTNDDRGQWFFDENHPYRTWAYHGSYEAPTQGSASSLINGVVRLLSKPWDVVTGVTGIAMTDTTPYGQQRVFKEKVDTRVPDPQEGTRQVMNMVSSWLWKELGKRETACLAKSYAQMWQLLYFHRRDLRLMANAICSSVPVDWVPTGRTTWSIHGKGEWMTTEDMLVVWNRVWI

SEQ ID NO: 145 - DNA, 4074 bp, synthetic construct
  source 1..4074: mol_type = other DNA
atgctgaagaggggggtggccagggtgagccccttcggaggactgaagaggctgcccgccgggctgctgctgggacatggacctatcaggatggtgctggccatcctggccttcctgaggtttacagcaattagggataaggaggccatggagattattaaaaagttcaaaaaggacctggccgccatgctgagaattatcaacgcaaggaaggagaaaaagaggagaggcgccgacaagggcaaggtgggactgctgctgaccaccgccatggccgccgaggtgacaaggaggggctccgcttactacatgtacctggacaggaacgacgccggcaaagcaatcagcttcgccaccacactgggcatgaacaaatgctacatccagatcatggatctggggcacatgtgcgacgccaccatgtcctacgagtgcccaatgctgctgaggcccgccagtgcctgggccatctacgccgctctgaccaccttcatcacccccgccgtccagcacgccgtcaccacatcctacaacaactactccctgatggccatggccacccaggccggagtgctgttcggcatgggcaagggcatgcccttctacgcctgggactttggagtgcccctgctgatgatgggctgttactctcagctgacccccctgaccctgattgtggccattattctgctggtggctcactacatgtatctgatccccggcctgggccccggcctgtgggagggaagccctaacaaatactggaactcctccaccgccacctccctgtgcaacatcttcaggggctcctatctggccggcgcctccctgatctacactgtgacccgcaacgccggcctggtgaaaagacggggcggcggtaccggcgagacactgggagagaaatggaaggccaggctgaaccagatgtctgccctggagttctactcctacaaagagcccaccaacgatgatagggggcagtggttcttcgatgaaaaccacccctacaggacatgggcctaccacggaagctacgaagcccccacccagggctccgcctcctctctgatcaacggcgtggtgaggctgctgtccaagccctgggacgtggtgaccggcgtgaccggaatcgctatgaccgacacaaccccctacggccagcagcgggtgtttaaggagaaggtggacaccagggtgcccgacccccaggagggaacaaggcaggtgatgaacatggtgagcagttggctgtggaaggagctgggcaaaagggagacagcttgcctggctaagagctacgcccagatgtggcagctgctgtactttcacaggagggacctgaggctgatggccaatgccatctgttctagcgtgcccgtggattgggtgcccacaggaaggactacctggtcaatccacggcaagggcgagtggatgaccaccgaggacatgctggtggtgtggaacagagtgtggatcggatctggtgccaccaacttttccctgctgaaacaggccggcgatgtggaggaaaacccaggccccatggtgaccgacttccagggcaagaccgtgtggttcgtgccctccatcaaggccggcgagaagtccaggaagaccttcgacaccgagtaccagaagaccaagaagaccccccgcatgtgcaccagggaggagttcagagacggagacctgcccgtgtggctgtcttacaaggtggccagcgagggcatctcctatacagacagaaagtggtgcttcgatgagcagcagcctcactggatcgccgccagcatcatcctggagttctttctgatggtgctgctgatcccagagccagagaagcagaggaccccacaggataaccagctggcctacgtgaagtgtaacggcaatgacacactgatcctggcacctacccgggtggtggcaagcgagatggcagaggccctgaagggcatgccaatcaggtatcagaccacagccacaaagtccgagcacaccggccgcgagatcgtggataacatcaatacacctgagggcatcatcccaagcctgttcgagccagagagggagaagtccgccgcaatcgacggagagtacaggctgagaggagaggcacggaagacctttgtggacctgatgaggagaggcgatctgcccgtgtggctggcatacagagtggcagcagagggcatcaactatgccgacaggcgctggtgctttgatggcgagaggaacaatcagatcctggaggagaatatggaggtggagatctggaccaaggagggcgagagaaagaagctgaagccccggtggctggacgccagaatctacagcgatcctctggccctgaaggagttcaaggagtttgcagcaggaagggatgagctgaggccaagatggctggatgccagaacatactccgatcccatggccctgaaggacttcaaggagttttgtaactgctgtaatgaccacccctataagacctgggcctaccacggcagctatgagacaaagcagacaggctccgccagctccatggtgaatggcgtggtgaagctgctgaccaagccttgggatgtggtgccaatggtgacacagatggccatgaccgacaccacaccattcggccagcagagggtgtttaccaaccccacacagcacaccgactgggatttcgtggtgaccacagatatcagcgagatgggcgccaacttcaaggccgaccgcgtgatcgatccacggagatgcctgaagcccgtgatcctgacagacggacctgagagagtgatcctggcaggacctatgccagtgacccactctagcgccgcacagaggaggggccggcaccctggcgccggcaagacccggagatacctgccagccatcgtgagagaggccatcaagaggggcctgcgcacactgatcctggcccctaccagggtggtggcagcagagatggaggaggccctgaggggcctgcctatccgctatcagacaccagcaatcagggcagagcacaccggaagagagatcgtggatctgatgtgccacgccaccttcacaatgaggctgctgtccccagtgcgcgtgcccaactacaatctgatcatcatggatgaggcccactttacagacccagcctctatcgccgccagaggctatatcagcaccagagtggagatgggagaggcagcagcaatcttcatgacagcaggaccaggacacgaggagccaatccctatgtctacctacggctggaacctggtgaagctgcagagcgaggaggacgattgcgtgacacaggtgctgatgatgaggaccacatgggcactgtgcgaggccctgacactggcaaccggaccaatcagcaccctgtgggagggctcccctggcaagttctggaacaccacaatcgccgtgtccatggccaatatcttcagaggctcttacctggcaggagcaggcctggcctttagcatcatgaagtgcatcgacgatgagaactgcatctgcgtgtacaatatgatgggcaagagggagaagaagctgggcgagtttggcaaggccaagggctccagggccatctggtacatgtggctgggcgcccgcttcctggagtttgaggccctgggcttcctgaacgaggaccactggttttctcgcgagaattccctgtctggagtggagggagagggcctgcacaagctgggctatgacgatagggcaagaatctctcagggagcaggatggagcctgagggagacagcctgcctgggcaagtcctacgcccagatgtggagcctgatgtatttccacaggcgcgatctgagactggccgccaatgccatctgttctgccgtgcccagccactgggtgcctacatccaggaccacatggtctatccacgcccaccaccagtggatgaccacagaggacatgctgaccgtgtggtcctgtcagccccctgaggacatcgatctgaggcccgcctctgcctggaccctgtatgctgtcgccacaaccttcgtcacacctatgctgaggcacaccattgaaaactaa

SEQ ID NO: 146 - protein, 1357 aa, unidentified
MLKRGVARVSPFGGLKRLPAGLLLGHGPIRMVLAILAFLRFTAIRDKEAMEIIKKFKKDLAAMLRIINARKEKKRRGADKGKVGLLLTTAMAAEVTRRGSAYYMYLDRNDAGKAISFATTLGMNKCYIQIMDLGHMCDATMSYECPMLLRPASAWAIYAALTTFITPAVQHAVTTSYNNYSLMAMATQAGVLFGMGKGMPFYAWDFGVPLLMMGCYSQLTPLTLIVAIILLVAHYMYLIPGLGPGLWEGSPNKYWNSSTATSLCNIFRGSYLAGASLIYTVTRNAGLVKRRGGGTGETLGEKWKARLNQMSALEFYSYKEPTNDDRGQWFFDENHPYRTWAYHGSYEAPTQGSASSLINGVVRLLSKPWDVVTGVTGIAMTDTTPYGQQRVFKEKVDTRVPDPQEGTRQVMNMVSSWLWKELGKRETACLAKSYAQMWQLLYFHRRDLRLMANAICSSVPVDWVPTGRTTWSIHGKGEWMTTEDMLVVWNRVWIGSGATNFSLLKQAGDVEENPGPMVTDFQGKTVWFVPSIKAGEKSRKTFDTEYQKTKKTPRMCTREEFRDGDLPVWLSYKVASEGISYTDRKWCFDEQQPHWIAASIILEFFLMVLLIPEPEKQRTPQDNQLAYVKCNGNDTLILAPTRVVASEMAEALKGMPIRYQTTATKSEHTGREIVDNINTPEGIIPSLFEPEREKSAAIDGEYRLRGEARKTFVDLMRRGDLPVWLAYRVAAEGINYADRRWCFDGERNNQILEENMEVEIWTKEGERKKLKPRWLDARIYSDPLALKEFKEFAAGRDELRPRWLDARTYSDPMALKDFKEFCNCCNDHPYKTWAYHGSYETKQTGSASSMVNGVVKLLTKPWDVVPMVTQMAMTDTTPFGQQRVFTNPTQHTDWDFVVTTDISEMGANFKADRVIDPRRCLKPVILTDGPERVILAGPMPVTHSSAAQRRGRHPGAGKTRRYLPAIVREAIKRGLRTLILAPTRVVAAEMEEALRGLPIRYQTPAIRAEHTGREIVDLMCHATFTMRLLSPVRVPNYNLIIMDEAHFTDPASIAARGYISTRVEMGEAAAIFMTAGPGHEEPIPMSTYGWNLVKLQSEEDDCVTQVLMMRTTWALCEALTLATGPISTLWEGSPGKFWNTTIAVSMANIFRGSYLAGAGLAFSIMKCIDDENCICVYNMMGKREKKLGEFGKAKGSRAIWYMWLGARFLEFEALGFLNEDHWFSRENSLSGVEGEGLHKLGYDDRARISQGAGWSLRETACLGKSYAQMWSLMYFHRRDLRLAANAICSAVPSHWVPTSRTTWSIHAHHQWMTTEDMLTVWSCQPPEDIDLRPASAWTLYAVATTFVTPMLRHTIEN

SEQ ID NO: 147 - DNA, 3714 bp, synthetic construct
  source 1..3714: mol_type = other DNA
atggtgaccgacttccagggcaagaccgtgtggttcgtgccctccatcaaggccggcgagaagtccaggaagaccttcgacaccgagtaccagaagaccaagaagaccccccgcatgtgcaccagggaggagttcagagacggagacctgcccgtgtggctgtcttacaaggtggccagcgagggcatctcctatacagacagaaagtggtgcttcgatgagcagcagcctcactggatcgccgccagcatcatcctggagttctttctgatggtgctgctgatcccagagccagagaagcagaggaccccacaggataaccagctggcctacgtgaagtgtaacggcaatgacacactgatcctggcacctacccgggtggtggcaagcgagatggcagaggccctgaagggcatgccaatcaggtatcagaccacagccacaaagtccgagcacaccggccgcgagatcgtggataacatcaatacacctgagggcatcatcccaagcctgttcgagccagagagggagaagtccgccgcaatcgacggagagtacaggctgagaggagaggcacggaagacctttgtggacctgatgaggagaggcgatctgcccgtgtggctggcatacagagtggcagcagagggcatcaactatgccgacaggcgctggtgctttgatggcgagaggaacaatcagatcctggaggagaatatggaggtggagatctggaccaaggagggcgagagaaagaagctgaagccccggtggctggacgccagaatctacagcgatcctctggccctgaaggagttcaaggagtttgcagcaggaagggatgagctgaggccaagatggctggatgccagaacatactccgatcccatggccctgaaggacttcaaggagttttgtaactgctgtaatgaccacccctataagacctgggcctaccacggcagctatgagacaaagcagacaggctccgccagctccatggtgaatggcgtggtgaagctgctgaccaagccttgggatgtggtgccaatggtgacacagatggccatgaccgacaccacaccattcggccagcagagggtgtttaccaaccccacacagcacaccgactgggatttcgtggtgaccacagatatcagcgagatgggcgccaacttcaaggccgaccgcgtgatcgatccacggagatgcctgaagcccgtgatcctgacagacggacctgagagagtgatcctggcaggacctatgccagtgacccactctagcgccgcacagaggaggggccggcaccctggcgccggcaagacccggagatacctgccagccatcgtgagagaggccatcaagaggggcctgcgcacactgatcctggcccctaccagggtggtggcagcagagatggaggaggccctgaggggcctgcctatccgctatcagacaccagcaatcagggcagagcacaccggaagagagatcgtggatctgatgtgccacgccaccttcacaatgaggctgctgtccccagtgcgcgtgcccaactacaatctgatcatcatggatgaggcccactttacagacccagcctctatcgccgccagaggctatatcagcaccagagtggagatgggagaggcagcagcaatcttcatgacagcaggaccaggacacgaggagccaatccctatgtctacctacggctggaacctggtgaagctgcagagcgaggaggacgattgcgtgacacaggtgctgatgatgaggaccacatgggcactgtgcgaggccctgacactggcaaccggaccaatcagcaccctgtgggagggctcccctggcaagttctggaacaccacaatcgccgtgtccatggccaatatcttcagaggctcttacctggcaggagcaggcctggcctttagcatcatgaagtgcatcgacgatgagaactgcatctgcgtgtacaatatgatgggcaagagggagaagaagctgggcgagtttggcaaggccaagggctccagggccatctggtacatgtggctgggcgcccgcttcctggagtttgaggccctgggcttcctgaacgaggaccactggttttctcgcgagaattccctgtctggagtggagggagagggcctgcacaagctgggctatgacgatagggcaagaatctctcagggagcaggatggagcctgagggagacagcctgcctgggcaagtcctacgcccagatgtggagcctgatgtatttccacaggcgcgatctgagactggccgccaatgccatctgttctgccgtgcccagccactgggtgcctacatccaggaccacatggtctatccacgcccaccaccagtggatgaccacagaggacatgctgaccgtgtggtcctgtcagccccctgaggacatcgatctgaggcccgcctctgcctggaccctgtatgctgtcgccacaaccttcgtcacacctatgctgaggcacaccattgaaaacggatctggtgccaccaacttttccctgctgaaacaggccggcgatgtggaggaaaacccaggccccatggctctcgctctcggaggagtgctcatcttcctgtccaccgctgtgtccgctgatgtgggatgttccgtggatttctccaagaaggagacccgctgcggaaccggagtgttcgtctacaacgacgtggaggcatggagagacaggtacaagtaccaccctgactcccccaggcgcttggctgcagcagtcaagcaggcctgggaagatggaatctgtgggatctcctccgtgtcacggatggagaacatcatgtggcggtccgtggagggggaattgaacgccatccttgaggagaacggggtgcagcttaccgtcgtggtggggtctgtcaaaaaccctatgtggcggggcccacaaagactccctgtgcctgtgaacgaacttcctcacggctggaaggcctgggggaaatcttacttcgtccgggccgccaagacaaacaactccttcgtcgtggatggcgataccttgaaggagtgcccactgaagcaccgggcctggaattcttttctggtggaggatcacgggtttggggtctttcacaccagcgtctggctcaaggtgcgggaggactacagcctggagtgcgatccagccgtgatcgggacagccgtgaaagggaaggaagccgtccatagtgatctgggctactggatcgagagcgagaagaacgacacctggaggttgaagagggcccacctgatcgagatgaagacctgcgagtggccaaagagccacacactgtggacagacgggattgaggagagcgacctgattattcccaagagcctggccgggccactcagccaccacaacacacgggagggctacagaacacagatgaaggggccctggcatagcgaggagctggaaattaggtttgaggagtgccccggcactaaggtccatgtggaggagacatgcggcactcggggccccagtctgagaagtacaactgcctcaggcagagtgattgaggagtggtgctgcagggagtgcacaatgccccccctgtcatttagggccaaggacggctgttggtatggcatggagattcggcccaggaaagaacccgaaagcaacctggtcaggagcatggtcactgccggcagcacttaatga

SEQ ID NO: 148 - protein, 1236 aa, unidentified
MVTDFQGKTVWFVPSIKAGEKSRKTFDTEYQKTKKTPRMCTREEFRDGDLPVWLSYKVASEGISYTDRKWCFDEQQPHWIAASIILEFFLMVLLIPEPEKQRTPQDNQLAYVKCNGNDTLILAPTRVVASEMAEALKGMPIRYQTTATKSEHTGREIVDNINTPEGIIPSLFEPEREKSAAIDGEYRLRGEARKTFVDLMRRGDLPVWLAYRVAAEGINYADRRWCFDGERNNQILEENMEVEIWTKEGERKKLKPRWLDARIYSDPLALKEFKEFAAGRDELRPRWLDARTYSDPMALKDFKEFCNCCNDHPYKTWAYHGSYETKQTGSASSMVNGVVKLLTKPWDVVPMVTQMAMTDTTPFGQQRVFTNPTQHTDWDFVVTTDISEMGANFKADRVIDPRRCLKPVILTDGPERVILAGPMPVTHSSAAQRRGRHPGAGKTRRYLPAIVREAIKRGLRTLILAPTRVVAAEMEEALRGLPIRYQTPAIRAEHTGREIVDLMCHATFTMRLLSPVRVPNYNLIIMDEAHFTDPASIAARGYISTRVEMGEAAAIFMTAGPGHEEPIPMSTYGWNLVKLQSEEDDCVTQVLMMRTTWALCEALTLATGPISTLWEGSPGKFWNTTIAVSMANIFRGSYLAGAGLAFSIMKCIDDENCICVYNMMGKREKKLGEFGKAKGSRAIWYMWLGARFLEFEALGFLNEDHWFSRENSLSGVEGEGLHKLGYDDRARISQGAGWSLRETACLGKSYAQMWSLMYFHRRDLRLAANAICSAVPSHWVPTSRTTWSIHAHHQWMTTEDMLTVWSCQPPEDIDLRPASAWTLYAVATTFVTPMLRHTIENGSGATNFSLLKQAGDVEENPGPMALALGGVLIFLSTAVSADVGCSVDFSKKETRCGTGVFVYNDVEAWRDRYKYHPDSPRRLAAAVKQAWEDGICGISSVSRMENIMWRSVEGELNAILEENGVQLTVVVGSVKNPMWRGPQRLPVPVNELPHGWKAWGKSYFVRAAKTNNSFVVDGDTLKECPLKHRAWNSFLVEDHGFGVFHTSVWLKVREDYSLECDPAVIGTAVKGKEAVHSDLGYWIESEKNDTWRLKRAHLIEMKTCEWPKSHTLWTDGIEESDLIIPKSLAGPLSHHNTREGYRTQMKGPWHSEELEIRFEECPGTKVHVEETCGTRGPSLRSTTASGRVIEEWCCRECTMPPLSFRAKDGCWYGMEIRPRKEPESNLVRSMVTAGST

SEQ ID NO: 149 - DNA, 3699 bp, synthetic construct
  source 1..3699: mol_type = other DNA
atggctctcgctctcggaggagtgctcatcttcctgtccaccgctgtgtccgctgatgtgggatgttccgtggatttctccaagaaggagacccgctgcggaaccggagtgttcgtctacaacgacgtggaggcatggagagacaggtacaagtaccaccctgactcccccaggcgcttggctgcagcagtcaagcaggcctgggaagatggaatctgtgggatctcctccgtgtcacggatggagaacatcatgtggcggtccgtggagggggaattgaacgccatccttgaggagaacggggtgcagcttaccgtcgtggtggggtctgtcaaaaaccctatgtggcggggcccacaaagactccctgtgcctgtgaacgaacttcctcacggctggaaggcctgggggaaatcttacttcgtccgggccgccaagacaaacaactccttcgtcgtggatggcgataccttgaaggagtgcccactgaagcaccgggcctggaattcttttctggtggaggatcacgggtttggggtctttcacaccagcgtctggctcaaggtgcgggaggactacagcctggagtgcgatccagccgtgatcgggacagccgtgaaagggaaggaagccgtccatagtgatctgggctactggatcgagagcgagaagaacgacacctggaggttgaagagggcccacctgatcgagatgaagacctgcgagtggccaaagagccacacactgtggacagacgggattgaggagagcgacctgattattcccaagagcctggccgggccactcagccaccacaacacacgggagggctacagaacacagatgaaggggccctggcatagcgaggagctggaaattaggtttgaggagtgccccggcactaaggtccatgtggaggagacatgcggcactcggggccccagtctgagaagtacaactgcctcaggcagagtgattgaggagtggtgctgcagggagtgcacaatgccccccctgtcatttagggccaaggacggctgttggtatggcatggagattcggcccaggaaagaacccgaaagcaacctggtcaggagcatggtcactggatctggtgccaccaacttttccctgctgaaacaggccggcgatgtggaggaaaacccaggccccatggtgaccgacttccagggcaagaccgtgtggttcgtgccctccatcaaggccggcgagaagtccaggaagaccttcgacaccgagtaccagaagaccaagaagaccccccgcatgtgcaccagggaggagttcagagacggagacctgcccgtgtggctgtcttacaaggtggccagcgagggcatctcctatacagacagaaagtggtgcttcgatgagcagcagcctcactggatcgccgccagcatcatcctggagttctttctgatggtgctgctgatcccagagccagagaagcagaggaccccacaggataaccagctggcctacgtgaagtgtaacggcaatgacacactgatcctggcacctacccgggtggtggcaagcgagatggcagaggccctgaagggcatgccaatcaggtatcagaccacagccacaaagtccgagcacaccggccgcgagatcgtggataacatcaatacacctgagggcatcatcccaagcctgttcgagccagagagggagaagtccgccgcaatcgacggagagtacaggctgagaggagaggcacggaagacctttgtggacctgatgaggagaggcgatctgcccgtgtggctggcatacagagtggcagcagagggcatcaactatgccgacaggcgctggtgctttgatggcgagaggaacaatcagatcctggaggagaatatggaggtggagatctggaccaaggagggcgagagaaagaagctgaagccccggtggctggacgccagaatctacagcgatcctctggccctgaaggagttcaaggagtttgcagcaggaagggatgagctgaggccaagatggctggatgccagaacatactccgatcccatggccctgaaggacttcaaggagttttgtaactgctgtaatgaccacccctataagacctgggcctaccacggcagctatgagacaaagcagacaggctccgccagctccatggtgaatggcgtggtgaagctgctgaccaagccttgggatgtggtgccaatggtgacacagatggccatgaccgacaccacaccattcggccagcagagggtgtttaccaaccccacacagcacaccgactgggatttcgtggtgaccacagatatcagcgagatgggcgccaacttcaaggccgaccgcgtgatcgatccacggagatgcctgaagcccgtgatcctgacagacggacctgagagagtgatcctggcaggacctatgccagtgacccactctagcgccgcacagaggaggggccggcaccctggcgccggcaagacccggagatacctgccagccatcgtgagagaggccatcaagaggggcctgcgcacactgatcctggcccctaccagggtggtggcagcagagatggaggaggccctgaggggcctgcctatccgctatcagacaccagcaatcagggcagagcacaccggaagagagatcgtggatctgatgtgccacgccaccttcacaatgaggctgctgtccccagtgcgcgtgcccaactacaatctgatcatcatggatgaggcccactttacagacccagcctctatcgccgccagaggctatatcagcaccagagtggagatgggagaggcagcagcaatcttcatgacagcaggaccaggacacgaggagccaatccctatgtctacctacggctggaacctggtgaagctgcagagcgaggaggacgattgcgtgacacaggtgctgatgatgaggaccacatgggcactgtgcgaggccctgacactggcaaccggaccaatcagcaccctgtgggagggctcccctggcaagttctggaacaccacaatcgccgtgtccatggccaatatcttcagaggctcttacctggcaggagcaggcctggcctttagcatcatgaagtgcatcgacgatgagaactgcatctgcgtgtacaatatgatgggcaagagggagaagaagctgggcgagtttggcaaggccaagggctccagggccatctggtacatgtggctgggcgcccgcttcctggagtttgaggccctgggcttcctgaacgaggaccactggttttctcgcgagaattccctgtctggagtggagggagagggcctgcacaagctgggctatgacgatagggcaagaatctctcagggagcaggatggagcctgagggagacagcctgcctgggcaagtcctacgcccagatgtggagcctgatgtatttccacaggcgcgatctgagactggccgccaatgccatctgttctgccgtgcccagccactgggtgcctacatccaggaccacatggtctatccacgcccaccaccagtggatgaccacagaggacatgctgaccgtgtggtcctgtcagccccctgaggacatcgatctgaggcccgcctctgcctggaccctgtatgctgtcgccacaaccttcgtcacacctatgctgaggcacaccattgaaaactaa

SEQ ID NO: 150 - protein, 1232 aa, unidentified
MALALGGVLIFLSTAVSADVGCSVDFSKKETRCGTGVFVYNDVEAWRDRYKYHPDSPRRLAAAVKQAWEDGICGISSVSRMENIMWRSVEGELNAILEENGVQLTVVVGSVKNPMWRGPQRLPVPVNELPHGWKAWGKSYFVRAAKTNNSFVVDGDTLKECPLKHRAWNSFLVEDHGFGVFHTSVWLKVREDYSLECDPAVIGTAVKGKEAVHSDLGYWIESEKNDTWRLKRAHLIEMKTCEWPKSHTLWTDGIEESDLIIPKSLAGPLSHHNTREGYRTQMKGPWHSEELEIRFEECPGTKVHVEETCGTRGPSLRSTTASGRVIEEWCCRECTMPPLSFRAKDGCWYGMEIRPRKEPESNLVRSMVTGSGATNFSLLKQAGDVEENPGPMVTDFQGKTVWFVPSIKAGEKSRKTFDTEYQKTKKTPRMCTREEFRDGDLPVWLSYKVASEGISYTDRKWCFDEQQPHWIAASIILEFFLMVLLIPEPEKQRTPQDNQLAYVKCNGNDTLILAPTRVVASEMAEALKGMPIRYQTTATKSEHTGREIVDNINTPEGIIPSLFEPEREKSAAIDGEYRLRGEARKTFVDLMRRGDLPVWLAYRVAAEGINYADRRWCFDGERNNQILEENMEVEIWTKEGERKKLKPRWLDARIYSDPLALKEFKEFAAGRDELRPRWLDARTYSDPMALKDFKEFCNCCNDHPYKTWAYHGSYETKQTGSASSMVNGVVKLLTKPWDVVPMVTQMAMTDTTPFGQQRVFTNPTQHTDWDFVVTTDISEMGANFKADRVIDPRRCLKPVILTDGPERVILAGPMPVTHSSAAQRRGRHPGAGKTRRYLPAIVREAIKRGLRTLILAPTRVVAAEMEEALRGLPIRYQTPAIRAEHTGREIVDLMCHATFTMRLLSPVRVPNYNLIIMDEAHFTDPASIAARGYISTRVEMGEAAAIFMTAGPGHEEPIPMSTYGWNLVKLQSEEDDCVTQVLMMRTTWALCEALTLATGPISTLWEGSPGKFWNTTIAVSMANIFRGSYLAGAGLAFSIMKCIDDENCICVYNMMGKREKKLGEFGKAKGSRAIWYMWLGARFLEFEALGFLNEDHWFSRENSLSGVEGEGLHKLGYDDRARISQGAGWSLRETACLGKSYAQMWSLMYFHRRDLRLAANAICSAVPSHWVPTSRTTWSIHAHHQWMTTEDMLTVWSCQPPEDIDLRPASAWTLYAVATTFVTPMLRHTIEN

SEQ ID NO: 151 - DNA, 8624 bp, synthetic construct
  source 1..8624: mol_type = other DNA
ggcgttgggagctttttgcaaaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcccgacattgattattgactagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccgaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggctgatacgcgccggaaaccctgcagggaattccccagctgtagttataaacagaagttctccttctgctaggtagcattcaaagatcttaatcttctgggtttccgttttctcgaatgaaaaatgcaggtccgagcagttaactggcgggggcaccattagcaagtcacttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagctgacagcattcgggccgagcgatcggatccgccaccatggacccttacctgggcaaacgggaggaccagtggtgtggaagcctgattgggctgactattgatgacgacagacggggagacctgcccgtgtggctgtcttacaaggtggccagcgagggcatctcctatacagacagaaagtggtgcttcgatgagcagcagcctcactggatcgccgccagcatcatcctggagttctttctgatggtgctgctgatcccagagccagagaagcagaggaccccacaggataaccagctggcctacgtgaagtgtaacggcaatgacaccctaatcctagcacccacccgggttgtggccagcgagatggcagaggccctgaagggcatgccaatcaggtatcagaccacagccacaaagtccgagcacaccggccgcgagatcgtggataacatcaatacacctgagggcatcatcccaagcctgttcgagccagagagggagaagtccgccgcaatcgacggagagtacaggctgagaggagaggcacggaagacctttgtggacctgatgaggagaggcgatctgcccgtgtggctggcatacagagtggcagcagagggcatcaactatgccgacaggcgctggtgctttgatggcgagaggaacaatcagatcctggaggagaatatggaggtggagatctggaccaaggagggcgagagaaagaagctgaagccccggtggctggacgccagaatctacagcgatcctctggctctgaaagagtttaaggagttcgcagcaggaagggatgagctgaggccaagatggctggatgccagaacatactccgatcccatggctctgaaggacttcaaggagttttgtaactgctgtaatgaccacccctataagacctgggcctaccacggcagctatgagacaaagcagacaggctccgccagctccatggtgaatggcgtggtgaagctgctgaccaagccttgggatgtggtgccaatggtgacacagatggccatgaccgacaccacaccattcggccagcagagggtgtttaccaaccccacacagcacaccgactgggatttcgtggtgaccacagatatcagcgagatgggcgccaacttcaaggccgaccgcgtgatcgatccacggagatgcctgaagcccgtgatcctgacagacggacctgagagagtgatcctggcaggacctatgccagtgacccactctagcgccgcacagaggaggggccggcaccctggcgccggcaagacccggagatacctgccagccatcgtgagagaggccatcaagaggggcctgcgcacactgatcctggcccctaccagggtggtggcagcagagatggaggaggccctgaggggcctgcctatccgctatcagacaccagcaatcagggcagagcacaccggaagagagatcgtggatctgatgtgccacgccaccttcacaatgaggctgctgtccccagtgcgcgtgcccaactacaatctgatcatcatggatgaggcccactttacagacccagcctctatcgccgccagaggctatatcagcaccagagtggagatgggagaggcagcagcaatcttcatgacagcaggaccaggacacgaggagccaatccctatgtctacctacggctggaacctggtgaagctgcagagcgaggaggacgattgcgtgacacaggtgctgatgatgaggaccacatgggcactgtgcgaggccctgacactggcaaccggaccaatcagcaccctgtgggagggctcccctggcaagttctggaacaccacaatcgccgtgtccatggccaatatcttcagaggctcttacctggcaggagcaggcctggcctttagcatcatgaagtgcatcgacgatgagaactgcatctgcgtgtacaatatgatgggcaagagggagaagaagctgggcgagtttggcaaggccaagggctccagggccatctggtacatgtggctgggcgcccgcttcctggagtttgaggccctgggcttcctgaacgaggaccactggttttctcgcgagaattccctgtctggagtggagggagagggcctgcacaagctgggctatgacgatagggcaagaatctctcagggagcaggatggagcctgagggagacagcctgcctgggcaagtcctacgcccagatgtggagcctgatgtatttccacaggcgcgatctgagactggccgccaatgccatctgttctgccgtgcccagccactgggtgcctacatccaggaccacatggtctatccacgcccaccaccagtggatgaccacagaggacatgctgaccgtgtggtcctgtcagccccctgaggacatcgatctgaggcccgcctctgcctggaccctgtatgctgtcgccacaaccttcgtcacacctatgctgaggcacaccattgaaaactaactcgagaattcccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccgcggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcggtacctttaagaccaatgacttacaaggcagctgtagatcttagccactttttaaaagaaaaggggggactggaagggctaattcactcccaacgaagacaaaatcgtcgagagatgctgcatataagcagctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagttctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttcttgctgcttcgcgatgtacgggccagatatacgc

SEQ ID NO: 152 - DNA, 8687 bp, synthetic construct
  source 1..8687: mol_type = other DNA
ggcgttgggagctttttgcaaaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcccgacattgattattgactagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccgaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggctgatacgcgccggaaaccctgcagggaattccccagctgtagttataaacagaagttctccttctgctaggtagcattcaaagatcttaatcttctgggtttccgttttctcgaatgaaaaatgcaggtccgagcagttaactggcgggggcaccattagcaagtcacttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagctgacagcattcgggccgagcgatcggatccgccaccatggtgaccgacttccagggcaagaccgtgtggttcgtgccctccatcaaggccggcgagaagtccaggaagaccttcgacaccgagtaccagaagaccaagaagaccccccgcatgtgcaccagggaggagttcagagacggagacctgcccgtgtggctgtcttacaaggtggccagcgagggcatctcctatacagacagaaagtggtgcttcgatgagcagcagcctcactggatcgccgccagcatcatcctggagttctttctgatggtgctgctgatcccagagccagagaagcagaggaccccacaggataaccagctggcctacgtgaagtgtaacggcaatgacacactgatcctggcacctacccgggtggtggcaagcgagatggcagaggccctgaagggcatgccaatcaggtatcagaccacagccacaaagtccgagcacaccggccgcgagatcgtggataacatcaatacacctgagggcatcatcccaagcctgttcgagccagagagggagaagtccgccgcaatcgacggagagtacaggctgagaggagaggcacggaagacctttgtggacctgatgaggagaggcgatctgcccgtgtggctggcatacagagtggcagcagagggcatcaactatgccgacaggcgctggtgctttgatggcgagaggaacaatcagatcctggaggagaatatggaggtggagatctggaccaaggagggcgagagaaagaagctgaagccccggtggctggacgccagaatctacagcgatcctctggccctgaaggagttcaaggagtttgcagcaggaagggatgagctgaggccaagatggctggatgccagaacatactccgatcccatggccctgaaggacttcaaggagttttgtaactgctgtaatgaccacccctataagacctgggcctaccacggcagctatgagacaaagcagacaggctccgccagctccatggtgaatggcgtggtgaagctgctgaccaagccttgggatgtggtgccaatggtgacacagatggccatgaccgacaccacaccattcggccagcagagggtgtttaccaaccccacacagcacaccgactgggatttcgtggtgaccacagatatcagcgagatgggcgccaacttcaaggccgaccgcgtgatcgatccacggagatgcctgaagcccgtgatcctgacagacggacctgagagagtgatcctggcaggacctatgccagtgacccactctagcgccgcacagaggaggggccggcaccctggcgccggcaagacccggagatacctgccagccatcgtgagagaggccatcaagaggggcctgcgcacactgatcctggcccctaccagggtggtggcagcagagatggaggaggccctgaggggcctgcctatccgctatcagacaccagcaatcagggcagagcacaccggaagagagatcgtggatctgatgtgccacgccaccttcacaatgaggctgctgtccccagtgcgcgtgcccaactacaatctgatcatcatggatgaggcccactttacagacccagcctctatcgccgccagaggctatatcagcaccagagtggagatgggagaggcagcagcaatcttcatgacagcaggaccaggacacgaggagccaatccctatgtctacctacggctggaacctggtgaagctgcagagcgaggaggacgattgcgtgacacaggtgctgatgatgaggaccacatgggcactgtgcgaggccctgacactggcaaccggaccaatcagcaccctgtgggagggctcccctggcaagttctggaacaccacaatcgccgtgtccatggccaatatcttcagaggctcttacctggcaggagcaggcctggcctttagcatcatgaagtgcatcgacgatgagaactgcatctgcgtgtacaatatgatgggcaagagggagaagaagctgggcgagtttggcaaggccaagggctccagggccatctggtacatgtggctgggcgcccgcttcctggagtttgaggccctgggcttcctgaacgaggaccactggttttctcgcgagaattccctgtctggagtggagggagagggcctgcacaagctgggctatgacgatagggcaagaatctctcagggagcaggatggagcctgagggagacagcctgcctgggcaagtcctacgcccagatgtggagcctgatgtatttccacaggcgcgatctgagactggccgccaatgccatctgttctgccgtgcccagccactgggtgcctacatccaggaccacatggtctatccacgcccaccaccagtggatgaccacagaggacatgctgaccgtgtggtcctgtcagccccctgaggacatcgatctgaggcccgcctctgcctggaccctgtatgctgtcgccacaaccttcgtcacacctatgctgaggcacaccattgaaaactaactcgagaattcccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccgcggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcggtacctttaagaccaatgacttacaaggcagctgtagatcttagccactttttaaaagaaaaggggggactggaagggctaattcactcccaacgaagacaaaatcgtcgagagatgctgcatataagcagctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagttctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttcttgctgcttcgcgatgtacgggccagatatacgc

SEQ ID NO: 153 - DNA, 7646 bp, synthetic construct
  source 1..7646: mol_type = other DNA
ggcgttgggagctttttgcaaaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcccgacattgattattgactagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccgaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggctgatacgcgccggaaaccctgcagggaattccccagctgtagttataaacagaagttctccttctgctaggtagcattcaaagatcttaatcttctgggtttccgttttctcgaatgaaaaatgcaggtccgagcagttaactggcgggggcaccattagcaagtcacttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagctgacagcattcgggccgagcgatcggatccgccaccatgctgaagaggggggtggccagggtgagccccttcggaggactgaagaggctgcccgccgggctgctgctgggacatggacctatcaggatggtgctggccatcctggccttcctgaggtttacagcaattagggataaggaggccatggagattattaaaaagttcaaaaaggacctggccgccatgctgagaattatcaacgcaaggaaggagaaaaagaggagaggcgccgacaagggcaaggtgggactgctgctgaccaccgccatggccgccgaggtgacaaggaggggctccgcttactacatgtacctggacaggaacgacgccggcaaagcaatcagcttcgccaccacactgggcatgaacaaatgctacatccagatcatggatctggggcacatgtgcgacgccaccatgtcctacgagtgcccaatgctgctgaggcccgccagtgcctgggccatctacgccgctctgaccaccttcatcacccccgccgtccagcacgccgtcaccacatcctacaacaactactccctgatggccatggccacccaggccggagtgctgttcggcatgggcaagggcatgcccttctacgcctgggactttggagtgcccctgctgatgatgggctgttactctcagctgacccccctgaccctgattgtggccattattctgctggtggctcactacatgtatctgatccccggcctgggccccggcctgtgggagggaagccctaacaaatactggaactcctccaccgccacctccctgtgcaacatcttcaggggctcctatctggccggcgcctccctgatctacactgtgacccgcaacgccggcctggtgaaaagacggggcggcggtaccggcgagacactgggagagaaatggaaggccaggctgaaccagatgtctgccctggagttctactcctacaaagagcccaccaacgatgatagggggcagtggttcttcgatgaaaaccacccctacaggacatgggcctaccacggaagctacgaagcccccacccagggctccgcctcctctctgatcaacggcgtggtgaggctgctgtccaagccctgggacgtggtgaccggcgtgaccggaatcgctatgaccgacacaaccccctacggccagcagcgggtgtttaaggagaaggtggacaccagggtgcccgacccccaggagggaacaaggcaggtgatgaacatggtgagcagttggctgtggaaggagctgggcaaaagggagacagcttgcctggctaagagctacgcccagatgtggcagctgctgtactttcacaggagggacctgaggctgatggccaatgccatctgttctagcgtgcccgtggattgggtgcccacaggaaggactacctggtcaatccacggcaagggcgagtggatgaccaccgaggacatgctggtggtgtggaacagagtgtggatctaactcgagaattcccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccgcggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcggtacctttaagaccaatgacttacaaggcagctgtagatcttagccactttttaaaagaaaaggggggactggaagggctaattcactcccaacgaagacaaaatcgtcgagagatgctgcatataagcagctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagttctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttcttgctgcttcgcgatgtacgggccagatatacgc

SEQ ID NO: 154 - DNA, 7286 bp, synthetic construct
  source 1..7286: mol_type = other DNA
ggcgttgggagctttttgcaaaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcccgacattgattattgactagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccgaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggctgatacgcgccggaaaccctgcagggaattccccagctgtagttataaacagaagttctccttctgctaggtagcattcaaagatcttaatcttctgggtttccgttttctcgaatgaaaaatgcaggtccgagcagttaactggcgggggcaccattagcaagtcacttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagctgacagcattcgggccgagcgatcggatccgccaccatggctctcgctctcggaggagtgctcatcttcctgtccaccgctgtgtccgctgatgtgggatgttccgtggatttctccaagaaggagacccgctgcggaaccggagtgttcgtctacaacgacgtggaggcatggagagacaggtacaagtaccaccctgactcccccaggcgcttggctgcagcagtcaagcaggcctgggaagatggaatctgtgggatctcctccgtgtcacggatggagaacatcatgtggcggtccgtggagggggaattgaacgccatccttgaggagaacggggtgcagcttaccgtcgtggtggggtctgtcaaaaaccctatgtggcggggcccacaaagactccctgtgcctgtgaacgaacttcctcacggctggaaggcctgggggaaatcttacttcgtccgggccgccaagacaaacaactccttcgtcgtggatggcgataccttgaaggagtgcccactgaagcaccgggcctggaattcttttctggtggaggatcacgggtttggggtctttcacaccagcgtctggctcaaggtgcgggaggactacagcctggagtgcgatccagccgtgatcgggacagccgtgaaagggaaggaagccgtccatagtgatctgggctactggatcgagagcgagaagaacgacacctggaggttgaagagggcccacctgatcgagatgaagacctgcgagtggccaaagagccacacactgtggacagacgggattgaggagagcgacctgattattcccaagagcctggccgggccactcagccaccacaacacacgggagggctacagaacacagatgaaggggccctggcatagcgaggagctggaaattaggtttgaggagtgccccggcactaaggtccatgtggaggagacatgcggcactcggggccccagtctgagaagtacaactgcctcaggcagagtgattgaggagtggtgctgcagggagtgcacaatgccccccctgtcatttagggccaaggacggctgttggtatggcatggagattcggcccaggaaagaacccgaaagcaacctggtcaggagcatggtcactgccggcagcacttaatgactcgagaattcccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccgcggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcggtacctttaagaccaatgacttacaaggcagctgtagatcttagccactttttaaaagaaaaggggggactggaagggctaattcactcccaacgaagacaaaatcgtcgagagatgctgcatataagcagctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagttctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttcttgctgcttcgcgatgtacgggccagatatacgc

SEQ ID NO: 155 - DNA, 7781 bp, synthetic construct
  source 1..7781: mol_type = other DNA
ggcgttgggagctttttgcaaaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcccgacattgattattgactagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccgaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggctgatacgcgccggaaaccctgcagggaattccccagctgtagttataaacagaagttctccttctgctaggtagcattcaaagatcttaatcttctgggtttccgttttctcgaatgaaaaatgcaggtccgagcagttaactggcgggggcaccattagcaagtcacttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagctgacagcattcgggccgagcgatcggatccgccaccatggccctggtgtccggctggaactccatcaccgtgatgcccctgctgtgcgggatcggctgcgccatgctgcactggtccctgatcctgaaaaaggaggagaggatggccatgaccgacaccacccccttcggccagcagcgcgtgttcaaggagaagagatcccacgccgccatcggtgcctacaaagacaagctgctgtggaacggacccatggcagtgtccatgaccggcgtgatgaggggcaaccattacgcattcgtgggcgtgatgtacaacctgtggaaaatgaagaccggaaggagggacgagtttggcaaggcaaagggcagcagggctatctggtacatgtggctgggcgccaggtacctggagttcgaggctctgggcttcctgaacgaggaccattggagggacctggccgccatggacggaggaggattctatgccgactgtgagaggaggaggctgaggacactggtgctggctcctactagggtggtgctgagcgaaatgaaggaggcctttcacggactggatgtgaagtttcatacccaggctttcaaggagaaagaaatcgacgcaatgtgccacgccaccctgacctacaggatgctgggacactgcaaaggacacgccgtgcccttcggactggtgagtatgaagctgaccgtggctgtgggactgcacttcgacgacatgtacatggccctgatcgccgccttcggagtgatgggaggcctgtggaaatacgaaccctggaacactggccatgactggatcttggcagataaaaggcctacagcctggttcctgccctctatccgtgccgccaacgtgatggctaagggcacaagtggctcccccatcgtgaacaggaacggcgaggtgattggcctgtacggaaatggaattctggtgggcgacaacaaacacagggacctggtgggcgcagcactgcaccccttcgccctgctgctggtcctggctggctggctgttccacgtgggccccggaagcgtgaaggaggacctggtggcttacggcggctcctggaagctgggacccgaggctatggacaccatctccgtctttctgcattccgaagaaggtagcagggcttataggaacgccctgagcatgatgcccgaagctatgaccatcgtgatgctgttcatcctggccggactgctgacctccggcatggtgattttcgagggcgaggatacactggtggccctgaccctgacctcctacctggggctgacccagccctttctgggcctgtgcgctttccgtaggcccattgacgaccgctttgggctgaaggcctatgctaacatgtggtccctgatgtaccgcaacaaggacggcggcgtgaaacccacccatatctcctacattatgctgatcttctttgtcctgagggaggagaggtacctgatcatcggcatcctgaccctgactagggacgagtgtatgcccgccctgtacgagaaaaagctggccctgtacctgctgctggccctgaagttcaaggacgacagcgagtacatgacctcatggttctacgacaacgacaacccctataggacctggcactattgcggcagctactaactcgagaattcccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccgcggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcggtacctttaagaccaatgacttacaaggcagctgtagatcttagccactttttaaaagaaaaggggggactggaagggctaattcactcccaacgaagacaaaatcgtcgagagatgctgcatataagcagctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagttctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttcttgctgcttcgcgatgtacgggccagatatacgc

SEQ ID NO: 156 - DNA, 7326 bp, synthetic construct
  source 1..7326: mol_type = other DNA
ggcgttgggagctttttgcaaaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcccgacattgattattgactagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccgaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggctgatacgcgccggaaaccctgcagggaattccccagctgtagttataaacagaagttctccttctgctaggtagcattcaaagatcttaatcttctgggtttccgttttctcgaatgaaaaatgcaggtccgagcagttaactggcgggggcaccattagcaagtcacttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagctgacagcattcgggccgagcgaatcggatccgccaccatggccctgctgatgaccggcggcgtgaccctggtgaaggacaaggtgcaggggttcatcttcttcttcctgttcaacatcctgaccgggaagaaaatcaccgcccacctgaagaggctgtggaaaatgctggaccctaggcagggcctggccgtgctgaggaaggtgaagagggtggtggcctctctgatgagggggctgtccagtaggaagcggtactggtgccccgactccatggagtacaggcaggagaagtggatgacaggcaggatgggagaaaggcagctgcagaagattgaaaggtggttcgtgaggaatccctttttcgccgtgactgctctgaccatcgcttacctggtgggcagtaacatgactcagagggtggagaccgtggccatcgacaggcccgcagaggtgaggaaggtctgttacaatgccgtgctgacacacgtgaaaatcaacttcagtaacagggtgtggaactccttcaaagacagagacaagtccgcccacggaagccccacctttaaggataagcggaagctggcctccatcgtgaaggcctcatttgagaagagggagaagatcccagtgatcgtggctgacgacctgaacaagggcatcctggtcaccgtgaacccaatcgcctccaccggcaggagggataagtccatgtccctgtttgaggtggaccagacaaagatccagtatgtgatccgggcccagctgcacgtccgcgaggtcgaagaaaccgccgtcgactttggcaacagctacatcgccgagatggagaccgagagctggattgtggacagacagtgggctcaggacctgacactggaggaatgcgatgacgtgctgattgaggtgaatccccccttcaaggactgcgagaaagtgagcagccacaaccacatccccggctataggagggaccacggaggactgaactggatcaccaaggtgatcatgggcgccgtgctgatctgggtcggcatcaacaccaggaacatgactatgtccatgtccatgatcctggtgggcgtgatcatgatgtttctgtccctgcccgacgacgacaagtgcaaggtgtacatggacgccgtgttcgaatacaccaggaagaacaagaccaacggcccctggatgcaggtgcctctgtaactcgagaattcccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccgcggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcggtacctttaagaccaatgacttacaaggcagctgtagatcttagccactttttaaaagaaaaggggggactggaagggctaattcactcccaacgaagacaaaatcgtcgagagatgctgcatataagcagctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagttctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttcttgctgcttcgcgatgtacgggccagatatacgc

SEQ ID NO: 157 - DNA, 10235 bp, synthetic construct
  source 1..10235: mol_type = other DNA
ggcgttgggagctttttgcaaaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcccgacattgattattgactagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccgaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggctgatacgcgccggaaaccctgcagggaattccccagctgtagttataaacagaagttctccttctgctaggtagcattcaaagatcttaatcttctgggtttccgttttctcgaatgaaaaatgcaggtccgagcagttaactggcgggggcaccattagcaagtcacttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagctgacagcattcgggccgagcgatcggatccgccaccatggtgaccgacttccagggcaagaccgtgtggttcgtgccctccatcaaggccggcgagaagtccaggaagaccttcgacaccgagtaccagaagaccaagaagaccccccgcatgtgcaccagggaggagttcagagacggagacctgcccgtgtggctgtcttacaaggtggccagcgagggcatctcctatacagacagaaagtggtgcttcgatgagcagcagcctcactggatcgccgccagcatcatcctggagttctttctgatggtgctgctgatcccagagccagagaagcagaggaccccacaggataaccagctggcctacgtgaagtgtaacggcaatgacacactgatcctggcacctacccgggtggtggcaagcgagatggcagaggccctgaagggcatgccaatcaggtatcagaccacagccacaaagtccgagcacaccggccgcgagatcgtggataacatcaatacacctgagggcatcatcccaagcctgttcgagccagagagggagaagtccgccgcaatcgacggagagtacaggctgagaggagaggcacggaagacctttgtggacctgatgaggagaggcgatctgcccgtgtggctggcatacagagtggcagcagagggcatcaactatgccgacaggcgctggtgctttgatggcgagaggaacaatcagatcctggaggagaatatggaggtggagatctggaccaaggagggcgagagaaagaagctgaagccccggtggctggacgccagaatctacagcgatcctctggccctgaaggagttcaaggagtttgcagcaggaagggatgagctgaggccaagatggctggatgccagaacatactccgatcccatggccctgaaggacttcaaggagttttgtaactgctgtaatgaccacccctataagacctgggcctaccacggcagctatgagacaaagcagacaggctccgccagctccatggtgaatggcgtggtgaagctgctgaccaagccttgggatgtggtgccaatggtgacacagatggccatgaccgacaccacaccattcggccagcagagggtgtttaccaaccccacacagcacaccgactgggatttcgtggtgaccacagatatcagcgagatgggcgccaacttcaaggccgaccgcgtgatcgatccacggagatgcctgaagcccgtgatcctgacagacggacctgagagagtgatcctggcaggacctatgccagtgacccactctagcgccgcacagaggaggggccggcaccctggcgccggcaagacccggagatacctgccagccatcgtgagagaggccatcaagaggggcctgcgcacactgatcctggcccctaccagggtggtggcagcagagatggaggaggccctgaggggcctgcctatccgctatcagacaccagcaatcagggcagagcacaccggaagagagatcgtggatctgatgtgccacgccaccttcacaatgaggctgctgtccccagtgcgcgtgcccaactacaatctgatcatcatggatgaggcccactttacagacccagcctctatcgccgccagaggctatatcagcaccagagtggagatgggagaggcagcagcaatcttcatgacagcaggaccaggacacgaggagccaatccctatgtctacctacggctggaacctggtgaagctgcagagcgaggaggacgattgcgtgacacaggtgctgatgatgaggaccacatgggcactgtgcgaggccctgacactggcaaccggaccaatcagcaccctgtgggagggctcccctggcaagttctggaacaccacaatcgccgtgtccatggccaatatcttcagaggctcttacctggcaggagcaggcctggcctttagcatcatgaagtgcatcgacgatgagaactgcatctgcgtgtacaatatgatgggcaagagggagaagaagctgggcgagtttggcaaggccaagggctccagggccatctggtacatgtggctgggcgcccgcttcctggagtttgaggccctgggcttcctgaacgaggaccactggttttctcgcgagaattccctgtctggagtggagggagagggcctgcacaagctgggctatgacgatagggcaagaatctctcagggagcaggatggagcctgagggagacagcctgcctgggcaagtcctacgcccagatgtggagcctgatgtatttccacaggcgcgatctgagactggccgccaatgccatctgttctgccgtgcccagccactgggtgcctacatccaggaccacatggtctatccacgcccaccaccagtggatgaccacagaggacatgctgaccgtgtggtcctgtcagccccctgaggacatcgatctgaggcccgcctctgcctggaccctgtatgctgtcgccacaaccttcgtcacacctatgctgaggcacaccattgaaaacggatctggtgccaccaacttttccctgctgaaacaggccggcgatgtggaggaaaacccaggccccatgctgaagaggggggtggccagggtgagccccttcggaggactgaagaggctgcccgccgggctgctgctgggacatggacctatcaggatggtgctggccatcctggccttcctgaggtttacagcaattagggataaggaggccatggagattattaaaaagttcaaaaaggacctggccgccatgctgagaattatcaacgcaaggaaggagaaaaagaggagaggcgccgacaagggcaaggtgggactgctgctgaccaccgccatggccgccgaggtgacaaggaggggctccgcttactacatgtacctggacaggaacgacgccggcaaagcaatcagcttcgccaccacactgggcatgaacaaatgctacatccagatcatggatctggggcacatgtgcgacgccaccatgtcctacgagtgcccaatgctgctgaggcccgccagtgcctgggccatctacgccgctctgaccaccttcatcacccccgccgtccagcacgccgtcaccacatcctacaacaactactccctgatggccatggccacccaggccggagtgctgttcggcatgggcaagggcatgcccttctacgcctgggactttggagtgcccctgctgatgatgggctgttactctcagctgacccccctgaccctgattgtggccattattctgctggtggctcactacatgtatctgatccccggcctgggccccggcctgtgggagggaagccctaacaaatactggaactcctccaccgccacctccctgtgcaacatcttcaggggctcctatctggccggcgcctccctgatctacactgtgacccgcaacgccggcctggtgaaaagacggggcggcggtaccggcgagacactgggagagaaatggaaggccaggctgaaccagatgtctgccctggagttctactcctacaaagagcccaccaacgatgatagggggcagtggttcttcgatgaaaaccacccctacaggacatgggcctaccacggaagctacgaagcccccacccagggctccgcctcctctctgatcaacggcgtggtgaggctgctgtccaagccctgggacgtggtgaccggcgtgaccggaatcgctatgaccgacacaaccccctacggccagcagcgggtgtttaaggagaaggtggacaccagggtgcccgacccccaggagggaacaaggcaggtgatgaacatggtgagcagttggctgtggaaggagctgggcaaaagggagacagcttgcctggctaagagctacgcccagatgtggcagctgctgtactttcacaggagggacctgaggctgatggccaatgccatctgttctagcgtgcccgtggattgggtgcccacaggaaggactacctggtcaatccacggcaagggcgagtggatgaccaccgaggacatgctggtggtgtggaacagagtgtggatctaactcgagaattcccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccgcggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcggtacctttaagaccaatgacttacaaggcagctgtagatcttagccactttttaaaagaaaaggggggactggaagggctaattcactcccaacgaagacaaaatcgtcgagagatgctgcatataagcagctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagttctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttcttgctgcttcgcgatgtacgggccagatatacgc

SEQ ID NO: 158 - DNA, 10235 bp, synthetic construct
  source 1..10235: mol_type = other DNA
ggcgttgggagctttttgcaaaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcccgacattgattattgactagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccgaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggctgatacgcgccggaaaccctgcagggaattccccagctgtagttataaacagaagttctccttctgctaggtagcattcaaagatcttaatcttctgggtttccgttttctcgaatgaaaaatgcaggtccgagcagttaactggcgggggcaccattagcaagtcacttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagctgacagcattcgggccgagcgatcggatccgccaccatgctgaagaggggggtggccagggtgagccccttcggaggactgaagaggctgcccgccgggctgctgctgggacatggacctatcaggatggtgctggccatcctggccttcctgaggtttacagcaattagggataaggaggccatggagattattaaaaagttcaaaaaggacctggccgccatgctgagaattatcaacgcaaggaaggagaaaaagaggagaggcgccgacaagggcaaggtgggactgctgctgaccaccgccatggccgccgaggtgacaaggaggggctccgcttactacatgtacctggacaggaacgacgccggcaaagcaatcagcttcgccaccacactgggcatgaacaaatgctacatccagatcatggatctggggcacatgtgcgacgccaccatgtcctacgagtgcccaatgctgctgaggcccgccagtgcctgggccatctacgccgctctgaccaccttcatcacccccgccgtccagcacgccgtcaccacatcctacaacaactactccctgatggccatggccacccaggccggagtgctgttcggcatgggcaagggcatgcccttctacgcctgggactttggagtgcccctgctgatgatgggctgttactctcagctgacccccctgaccctgattgtggccattattctgctggtggctcactacatgtatctgatccccggcctgggccccggcctgtgggagggaagccctaacaaatactggaactcctccaccgccacctccctgtgcaacatcttcaggggctcctatctggccggcgcctccctgatctacactgtgacccgcaacgccggcctggtgaaaagacggggcggcggtaccggcgagacactgggagagaaatggaaggccaggctgaaccagatgtctgccctggagttctactcctacaaagagcccaccaacgatgatagggggcagtggttcttcgatgaaaaccacccctacaggacatgggcctaccacggaagctacgaagcccccacccagggctccgcctcctctctgatcaacggcgtggtgaggctgctgtccaagccctgggacgtggtgaccggcgtgaccggaatcgctatgaccgacacaaccccctacggccagcagcgggtgtttaaggagaaggtggacaccagggtgcccgacccccaggagggaacaaggcaggtgatgaacatggtgagcagttggctgtggaaggagctgggcaaaagggagacagcttgcctggctaagagctacgcccagatgtggcagctgctgtactttcacaggagggacctgaggctgatggccaatgccatctgttctagcgtgcccgtggattgggtgcccacaggaaggactacctggtcaatccacggcaagggcgagtggatgaccaccgaggacatgctggtggtgtggaacagagtgtggatcggatctggtgccaccaacttttccctgctgaaacaggccggcgatgtggaggaaaacccaggccccatggtgaccgacttccagggcaagaccgtgtggttcgtgccctccatcaaggccggcgagaagtccaggaagaccttcgacaccgagtaccagaagaccaagaagaccccccgcatgtgcaccagggaggagttcagagacggagacctgcccgtgtggctgtcttacaaggtggccagcgagggcatctcctatacagacagaaagtggtgcttcgatgagcagcagcctcactggatcgccgccagcatcatcctggagttctttctgatggtgctgctgatcccagagccagagaagcagaggaccccacaggataaccagctggcctacgtgaagtgtaacggcaatgacacactgatcctggcacctacccgggtggtggcaagcgagatggcagaggccctgaagggcatgccaatcaggtatcagaccacagccacaaagtccgagcacaccggccgcgagatcgtggataacatcaatacacctgagggcatcatcccaagcctgttcgagccagagagggagaagtccgccgcaatcgacggagagtacaggctgagaggagaggcacggaagacctttgtggacctgatgaggagaggcgatctgcccgtgtggctggcatacagagtggcagcagagggcatcaactatgccgacaggcgctggtgctttgatggcgagaggaacaatcagatcctggaggagaatatggaggtggagatctggaccaaggagggcgagagaaagaagctgaagccccggtggctggacgccagaatctacagcgatcctctggccctgaaggagttcaaggagtttgcagcaggaagggatgagctgaggccaagatggctggatgccagaacatactccgatcccatggccctgaaggacttcaaggagttttgtaactgctgtaatgaccacccctataagacctgggcctaccacggcagctatgagacaaagcagacaggctccgccagctccatggtgaatggcgtggtgaagctgctgaccaagccttgggatgtggtgccaatggtgacacagatggccatgaccgacaccacaccattcggccagcagagggtgtttaccaaccccacacagcacaccgactgggatttcgtggtgaccacagatatcagcgagatgggcgccaacttcaaggccgaccgcgtgatcgatccacggagatgcctgaagcccgtgatcctgacagacggacctgagagagtgatcctggcaggacctatgccagtgacccactctagcgccgcacagaggaggggccggcaccctggcgccggcaagacccggagatacctgccagccatcgtgagagaggccatcaagaggggcctgcgcacactgatcctggcccctaccagggtggtggcagcagagatggaggaggccctgaggggcctgcctatccgctatcagacaccagcaatcagggcagagcacaccggaagagagatcgtggatctgatgtgccacgccaccttcacaatgaggctgctgtccccagtgcgcgtgcccaactacaatctgatcatcatggatgaggcccactttacagacccagcctctatcgccgccagaggctatatcagcaccagagtggagatgggagaggcagcagcaatcttcatgacagcaggaccaggacacgaggagccaatccctatgtctacctacggctggaacctggtgaagctgcagagcgaggaggacgattgcgtgacacaggtgctgatgatgaggaccacatgggcactgtgcgaggccctgacactggcaaccggaccaatcagcaccctgtgggagggctcccctggcaagttctggaacaccacaatcgccgtgtccatggccaatatcttcagaggctcttacctggcaggagcaggcctggcctttagcatcatgaagtgcatcgacgatgagaactgcatctgcgtgtacaatatgatgggcaagagggagaagaagctgggcgagtttggcaaggccaagggctccagggccatctggtacatgtggctgggcgcccgcttcctggagtttgaggccctgggcttcctgaacgaggaccactggttttctcgcgagaattccctgtctggagtggagggagagggcctgcacaagctgggctatgacgatagggcaagaatctctcagggagcaggatggagcctgagggagacagcctgcctgggcaagtcctacgcccagatgtggagcctgatgtatttccacaggcgcgatctgagactggccgccaatgccatctgttctgccgtgcccagccactgggtgcctacatccaggaccacatggtctatccacgcccaccaccagtggatgaccacagaggacatgctgaccgtgtggtcctgtcagccccctgaggacatcgatctgaggcccgcctctgcctggaccctgtatgctgtcgccacaaccttcgtcacacctatgctgaggcacaccattgaaaactaactcgagaattcccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccgcggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcggtacctttaagaccaatgacttacaaggcagctgtagatcttagccactttttaaaagaaaaggggggactggaagggctaattcactcccaacgaagacaaaatcgtcgagagatgctgcatataagcagctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagttctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttcttgctgcttcgcgatgtacgggccagatatacgc

SEQ ID NO: 159 - DNA, 9875 bp, synthetic construct
  source 1..9875: mol_type = other DNA
ggcgttgggagctttttgcaaaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcccgacattgattattgactagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccgaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggctgatacgcgccggaaaccctgcagggaattccccagctgtagttataaacagaagttctccttctgctaggtagcattcaaagatcttaatcttctgggtttccgttttctcgaatgaaaaatgcaggtccgagcagttaactggcgggggcaccattagcaagtcacttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagctgacagcattcgggccgagcgatcggatccgccaccatggtgaccgacttccagggcaagaccgtgtggttcgtgccctccatcaaggccggcgagaagtccaggaagaccttcgacaccgagtaccagaagaccaagaagaccccccgcatgtgcaccagggaggagttcagagacggagacctgcccgtgtggctgtcttacaaggtggccagcgagggcatctcctatacagacagaaagtggtgcttcgatgagcagcagcctcactggatcgccgccagcatcatcctggagttctttctgatggtgctgctgatcccagagccagagaagcagaggaccccacaggataaccagctggcctacgtgaagtgtaacggcaatgacacactgatcctggcacctacccgggtggtggcaagcgagatggcagaggccctgaagggcatgccaatcaggtatcagaccacagccacaaagtccgagcacaccggccgcgagatcgtggataacatcaatacacctgagggcatcatcccaagcctgttcgagccagagagggagaagtccgccgcaatcgacggagagtacaggctgagaggagaggcacggaagacctttgtggacctgatgaggagaggcgatctgcccgtgtggctggcatacagagtggcagcagagggcatcaactatgccgacaggcgctggtgctttgatggcgagaggaacaatcagatcctggaggagaatatggaggtggagatctggaccaaggagggcgagagaaagaagctgaagccccggtggctggacgccagaatctacagcgatcctctggccctgaaggagttcaaggagtttgcagcaggaagggatgagctgaggccaagatggctggatgccagaacatactccgatcccatggccctgaaggacttcaaggagttttgtaactgctgtaatgaccacccctataagacctgggcctaccacggcagctatgagacaaagcagacaggctccgccagctccatggtgaatggcgtggtgaagctgctgaccaagccttgggatgtggtgccaatggtgacacagatggccatgaccgacaccacaccattcggccagcagagggtgtttaccaaccccacacagcacaccgactgggatttcgtggtgaccacagatatcagcgagatgggcgccaacttcaaggccgaccgcgtgatcgatccacggagatgcctgaagcccgtgatcctgacagacggacctgagagagtgatcctggcaggacctatgccagtgacccactctagcgccgcacagaggaggggccggcaccctggcgccggcaagacccggagatacctgccagccatcgtgagagaggccatcaagaggggcctgcgcacactgatcctggcccctaccagggtggtggcagcagagatggaggaggccctgaggggcctgcctatccgctatcagacaccagcaatcagggcagagcacaccggaagagagatcgtggatctgatgtgccacgccaccttcacaatgaggctgctgtccccagtgcgcgtgcccaactacaatctgatcatcatggatgaggcccactttacagacccagcctctatcgccgccagaggctatatcagcaccagagtggagatgggagaggcagcagcaatcttcatgacagcaggaccaggacacgaggagccaatccctatgtctacctacggctggaacctggtgaagctgcagagcgaggaggacgattgcgtgacacaggtgctgatgatgaggaccacatgggcactgtgcgaggccctgacactggcaaccggaccaatcagcaccctgtgggagggctcccctggcaagttctggaacaccacaatcgccgtgtccatggccaatatcttcagaggctcttacctggcaggagcaggcctggcctttagcatcatgaagtgcatcgacgatgagaactgcatctgcgtgtacaatatgatgggcaagagggagaagaagctgggcgagtttggcaaggccaagggctccagggccatctggtacatgtggctgggcgcccgcttcctggagtttgaggccctgggcttcctgaacgaggaccactggttttctcgcgagaattccctgtctggagtggagggagagggcctgcacaagctgggctatgacgatagggcaagaatctctcagggagcaggatggagcctgagggagacagcctgcctgggcaagtcctacgcccagatgtggagcctgatgtatttccacaggcgcgatctgagactggccgccaatgccatctgttctgccgtgcccagccactgggtgcctacatccaggaccacatggtctatccacgcccaccaccagtggatgaccacagaggacatgctgaccgtgtggtcctgtcagccccctgaggacatcgatctgaggcccgcctctgcctggaccctgtatgctgtcgccacaaccttcgtcacacctatgctgaggcacaccattgaaaacggatctggtgccaccaacttttccctgctgaaacaggccggcgatgtggaggaaaacccaggccccatggctctcgctctcggaggagtgctcatcttcctgtccaccgctgtgtccgctgatgtgggatgttccgtggatttctccaagaaggagacccgctgcggaaccggagtgttcgtctacaacgacgtggaggcatggagagacaggtacaagtaccaccctgactcccccaggcgcttggctgcagcagtcaagcaggcctgggaagatggaatctgtgggatctcctccgtgtcacggatggagaacatcatgtggcggtccgtggagggggaattgaacgccatccttgaggagaacggggtgcagcttaccgtcgtggtggggtctgtcaaaaaccctatgtggcggggcccacaaagactccctgtgcctgtgaacgaacttcctcacggctggaaggcctgggggaaatcttacttcgtccgggccgccaagacaaacaactccttcgtcgtggatggcgataccttgaaggagtgcccactgaagcaccgggcctggaattcttttctggtggaggatcacgggtttggggtctttcacaccagcgtctggctcaaggtgcgggaggactacagcctggagtgcgatccagccgtgatcgggacagccgtgaaagggaaggaagccgtccatagtgatctgggctactggatcgagagcgagaagaacgacacctggaggttgaagagggcccacctgatcgagatgaagacctgcgagtggccaaagagccacacactgtggacagacgggattgaggagagcgacctgattattcccaagagcctggccgggccactcagccaccacaacacacgggagggctacagaacacagatgaaggggccctggcatagcgaggagctggaaattaggtttgaggagtgccccggcactaaggtccatgtggaggagacatgcggcactcggggccccagtctgagaagtacaactgcctcaggcagagtgattgaggagtggtgctgcagggagtgcacaatgccccccctgtcatttagggccaaggacggctgttggtatggcatggagattcggcccaggaaagaacccgaaagcaacctggtcaggagcatggtcactgccggcagcacttaatgactcgagaattcccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccgcggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcggtacctttaagaccaatgacttacaaggcagctgtagatcttagccactttttaaaagaaaaggggggactggaagggctaattcactcccaacgaagacaaaatcgtcgagagatgctgcatataagcagctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagttctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttcttgctgcttcgcgatgtacgggccagatatacgc

SEQ ID NO: 160 - DNA, 9860 bp, synthetic construct
  source 1..9860: mol_type = other DNA
ggcgttgggagctttttgcaaaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcccgacattgattattgactagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccgaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggctgatacgcgccggaaaccctgcagggaattccccagctgtagttataaacagaagttctccttctgctaggtagcattcaaagatcttaatcttctgggtttccgttttctcgaatgaaaaatgcaggtccgagcagttaactggcgggggcaccattagcaagtcacttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagctgacagcattcgggccgagcgatcggatccgccaccatggctctcgctctcggaggagtgctcatcttcctgtccaccgctgtgtccgctgatgtgggatgttccgtggatttctccaagaaggagacccgctgcggaaccggagtgttcgtctacaacgacgtggaggcatggagagacaggtacaagtaccaccctgactcccccaggcgcttggctgcagcagtcaagcaggcctgggaagatggaatctgtgggatctcctccgtgtcacggatggagaacatcatgtggcggtccgtggagggggaattgaacgccatccttgaggagaacggggtgcagcttaccgtcgtggtggggtctgtcaaaaaccctatgtggcggggcccacaaagactccctgtgcctgtgaacgaacttcctcacggctggaaggcctgggggaaatcttacttcgtccgggccgccaagacaaacaactccttcgtcgtggatggcgataccttgaaggagtgcccactgaagcaccgggcctggaattcttttctggtggaggatcacgggtttggggtctttcacaccagcgtctggctcaaggtgcgggaggactacagcctggagtgcgatccagccgtgatcgggacagccgtgaaagggaaggaagccgtccatagtgatctgggctactggatcgagagcgagaagaacgacacctggaggttgaagagggcccacctgatcgagatgaagacctgcgagtggccaaagagccacacactgtggacagacgggattgaggagagcgacctgattattcccaagagcctggccgggccactcagccaccacaacacacgggagggctacagaacacagatgaaggggccctggcatagcgaggagctggaaattaggtttgaggagtgccccggcactaaggtccatgtggaggagacatgcggcactcggggccccagtctgagaagtacaactgcctcaggcagagtgattgaggagtggtgctgcagggagtgcacaatgccccccctgtcatttagggccaaggacggctgttggtatggcatggagattcggcccaggaaagaacccgaaagcaacctggtcaggagcatggtcactggatctggtgccaccaacttttccctgctgaaacaggccggcgatgtggaggaaaacccaggccccatggtgaccgacttccagggcaagaccgtgtggttcgtgccctccatcaaggccggcgagaagtccaggaagaccttcgacaccgagtaccagaagaccaagaagaccccccgcatgtgcaccagggaggagttcagagacggagacctgcccgtgtggctgtcttacaaggtggccagcgagggcatctcctatacagacagaaagtggtgcttcgatgagcagcagcctcactggatcgccgccagcatcatcctggagttctttctgatggtgctgctgatcccagagccagagaagcagaggaccccacaggataaccagctggcctacgtgaagtgtaacggcaatgacacactgatcctggcacctacccgggtggtggcaagcgagatggcagaggccctgaagggcatgccaatcaggtatcagaccacagccacaaagtccgagcacaccggccgcgagatcgtggataacatcaatacacctgagggcatcatcccaagcctgttcgagccagagagggagaagtccgccgcaatcgacggagagtacaggctgagaggagaggcacggaagacctttgtggacctgatgaggagaggcgatctgcccgtgtggctggcatacagagtggcagcagagggcatcaactatgccgacaggcgctggtgctttgatggcgagaggaacaatcagatcctggaggagaatatggaggtggagatctggaccaaggagggcgagagaaagaagctgaagccccggtggctggacgccagaatctacagcgatcctctggccctgaaggagttcaaggagtttgcagcaggaagggatgagctgaggccaagatggctggatgccagaacatactccgatcccatggccctgaaggacttcaaggagttttgtaactgctgtaatgaccacccctataagacctgggcctaccacggcagctatgagacaaagcagacaggctccgccagctccatggtgaatggcgtggtgaagctgctgaccaagccttgggatgtggtgccaatggtgacacagatggccatgaccgacaccacaccattcggccagcagagggtgtttaccaaccccacacagcacaccgactgggatttcgtggtgaccacagatatcagcgagatgggcgccaacttcaaggccgaccgcgtgatcgatccacggagatgcctgaagcccgtgatcctgacagacggacctgagagagtgatcctggcaggacctatgccagtgacccactctagcgccgcacagaggaggggccggcaccctggcgccggcaagacccggagatacctgccagccatcgtgagagaggccatcaagaggggcctgcgcacactgatcctggcccctaccagggtggtggcagcagagatggaggaggccctgaggggcctgcctatccgctatcagacaccagcaatcagggcagagcacaccggaagagagatcgtggatctgatgtgccacgccaccttcacaatgaggctgctgtccccagtgcgcgtgcccaactacaatctgatcatcatggatgaggcccactttacagacccagcctctatcgccgccagaggctatatcagcaccagagtggagatgggagaggcagcagcaatcttcatgacagcaggaccaggacacgaggagccaatccctatgtctacctacggctggaacctggtgaagctgcagagcgaggaggacgattgcgtgacacaggtgctgatgatgaggaccacatgggcactgtgcgaggccctgacactggcaaccggaccaatcagcaccctgtgggagggctcccctggcaagttctggaacaccacaatcgccgtgtccatggccaatatcttcagaggctcttacctggcaggagcaggcctggcctttagcatcatgaagtgcatcgacgatgagaactgcatctgcgtgtacaatatgatgggcaagagggagaagaagctgggcgagtttggcaaggccaagggctccagggccatctggtacatgtggctgggcgcccgcttcctggagtttgaggccctgggcttcctgaacgaggaccactggttttctcgcgagaattccctgtctggagtggagggagagggcctgcacaagctgggctatgacgatagggcaagaatctctcagggagcaggatggagcctgagggagacagcctgcctgggcaagtcctacgcccagatgtggagcctgatgtatttccacaggcgcgatctgagactggccgccaatgccatctgttctgccgtgcccagccactgggtgcctacatccaggaccacatggtctatccacgcccaccaccagtggatgaccacagaggacatgctgaccgtgtggtcctgtcagccccctgaggacatcgatctgaggcccgcctctgcctggaccctgtatgctgtcgccacaaccttcgtcacacctatgctgaggcacaccattgaaaactaactcgagaattcccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccgcggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcggtacctttaagaccaatgacttacaaggcagctgtagatcttagccactttttaaaagaaaaggggggactggaagggctaattcactcccaacgaagacaaaatcgtcgagagatgctgcatataagcagctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagttctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttcttgctgcttcgcgatgtacgggccagatatacgc

SEQ ID NO: 161 - DNA, 6155 bp, synthetic construct
  source 1..6155: mol_type = other DNA
ggcgttgggagctttttgcaaaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcccgacattgattattgactagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccgaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggctgatacgcgccggaaaccctgcagggaattccccagctgtagttataaacagaagttctccttctgctaggtagcattcaaagatcttaatcttctgggtttccgttttctcgaatgaaaaatgcaggtccgagcagttaactggcgggggcaccattagcaagtcacttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagctgacagcattcgggccgagcgatcggatccctcgagaattcccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccgcggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcggtacctttaagaccaatgacttacaaggcagctgtagatcttagccactttttaaaagaaaaggggggactggaagggctaattcactcccaacgaagacaaaatcgtcgagagatgctgcatataagcagctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagttctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttcttgctgcttcgcgatgtacgggccagatatacgc

SEQ ID NO: 162 - protein, 3392 aa, unidentified
MNNQRKKTGRPSFNMLKRARNRVSTVSQLAKRFSKGLLSGQGPMKLVMAFIAFLRFLAIPPTAGILARWGSFKKNGAIKVLRGFKKEISNMLNIMNRRKRSVTMLLMLLPTALAFHLTTRGGEPHMIVSKQERGKSLLFKTSAGVNMCTLIAMDLGELCEDTMTYKCPRITEAEPDDVDCWCNATDTWVTYGTCSQTGEHRRDKRSVALAPHVGLGLETRTETWMSSEGAWKQIQKVETWALRHPGFTVIALFLAHAIGTSITQKGIIFILLMLVTPSMAMRCVGIGNRDFVEGLSGATWVDVVLEHGSCVTTMAKDKPTLDIELLKTEVTNPAVLRKLCIEAKISNTTTDSRCPTQGEATLVEEQDQNFVCRRTFVDRGWGNGCGLFGKGSLITCAKFKCVTKLEGKIVQYENLKYSVIVTVHTGDQHQVGNETTEHGTTATITPQAPTSEIQLTDYGALTLDCSPRTGLDFNEMVLLTMKEKSWLVHKQWFLDLPLPWTSGASTSQETWNRQDLLVTFKTAHAKKQEVVVLGSQEGAMHTALTGATEIQTSGTTTIFAGHLKCRLKMDKLTLKGMSYVMCTGSFKLEKEVAETQHGTVLVQVKYEGTDAPCKIPFSIQDEKGVTQNGRLITANPIVTDKEKPVNIEAEPPFGESYIVVGAGEKALKLSWFKKGSSIGKMFEATARGARRMAILGDTAWDFGSIGGVFTSVGKLVHQIFGTAYGVLFSGVSWTMKIGIGILLTWLGLNSRSTSLSMTCIAVGMVTLYLGVMVQADSGCVINWKGRELKCGSGIFVTNEVHTWTEQYKFQADSPKRLSAAIGKAWEEGVCGIRSATRLENIMWKQISNELNHILLENDMKFTVVVGDVKGILAQGKKMIRPQPMEHKYSWKSWGKAKIIGADVQNSTFIIDGPNTPECPDDQRAWNIWEVEDYGFGIFTTNIWLKLRDSYTQVCDHRLMSAAIKDSKAVHADMGYWIESEKNETWKLARASFIEVKTCVWPKSHTLWSNGVLESEMIIPKIYGGPISQHNYRPGYFTQTAGPWHLGKLELDFDLCEGTTVVVDEHCGNRGPSLRTTTVTGKIIHEWCCRSCTLPPLRFKGEDGCWYGMEIRPVKEKEENLVKSMVSAGSGEVDSFSLGLLCISIMIEEVMRSRWSRKMLMTGTLAVFLLLIMGQLTWNDLIRLCIMVGANASDRMGMGTTYLALMATFKMRPMFAVGLLFRRLTSREVLLLTIGLSLVASVELPNSLEELGDGLAMGIMILKLLTDFQSHQLWATLLSLTFIKTTXSLHYAWKTMAMVLSIVSLFPLCLSTTSQKTTWLPVLLGSLGCKPLTMFLITENKIWGRKSWPLNEGIMAVGIVSILLSSLLKNDVPLAGPLIAGGMLIACYVISGSSADLSLEKAAEVSWEEEAEHSGASHNILVEVQDDGTMKIKDEERDDTLTILLKATLLAVSGVYPMSIPATLFVWYFWQKKKQRSGVLWDTPSPPEVERAVLDDGIYRILQRGLLGRSQVGVGVFQEGVFHTMWHVTRGAVLMYQGKRLEPSWASVKKDLISYGGGWRFQGSWNTGEEVQVIAVEPGKNPKNVQTAPGTFKTPEGEVGAIALDFKPGTSGSPIVNREGKIVGLYGNGVVTTSGTYVSAIAQAKASQEGPLPEIEDEVFRKRNLTIMDLHPGSGKTRRYLPAIVREAIKRKLRTLILAPTRVVASEMAEALKGMPIRYQTTAVKSEHTGKEIVDLMCHATFTMRLLSPVRVPNYNMIIMDEAHFTDPASIAARGYISTRVGMGEAAAIFMTATPPGSVEAFPQSNAVIQDEERDIPERSWNSGYDWITDFPGKTVWFVPSIKSGNDIANCLRKNGKRVIQLSRKTFDTEYQKTKNNDWDYVVTTDISEMGANFRADRVIDPRRCLKPVILKDGPERVILAGPMPVTVASAAQRRGRIGRNQNKEGDQYIYMGQPLNNDEDHAHWTEAKMLLDNINTPEGIIPALFEPEREKSAAIDGEYRLRGEARKTFVELMRRGDLPVWLSYKVASEGFQYSDRRWCFDGERNNQVLEENMDVEIWTKEGERKKLRPRWLDARTYSDPLALREFKEFAAGRRSVSGDLILEIGKLPQHLTQRAQNALDNLVMLHNSEQGGKAYRHAMEELPDTIETLMLLALIAVLTGGVTLFFLSGRGLGKTSIGLLCVMASSALLWMASVEPHWIAASIILEFFLMVLLIPEPDRQRTPQDNQLAYVVIGLLFMILTVAANEMGLLETTKKDLGIGHVAAENHHHAAMLDVDLHPASAWTLYAVATTIITPMMRHTIENTTANISLTAIANQAAILMGLDKGWPISKMDIGVPLLALGCYSQVNPLTLTAAVLMLVAHYAIIGPGLQAKATREAQKRTAAGIMKNPTVDGIVAIDLDPVVYDAKFEKQLGQIMLLILCTSQILLMRTTWALCESITLATGPLTTLWEGSPGKFWNTTIAVSMANIFRGSYLAGAGLAFSLMKSLGGGRRGTGAQGETLGEKWKRQLNQLSKSEFNTYKRSGIIEVDRSEAKEGLKRGETTKHAVSRGTAKLRWFVERNLVKPEGKVIDLGCGRGGWSYYCAGLKKVTEVKGYTKGGPGHEEPIPMATYGWNLVKLHSGKDVFFIPPEKCDTLLCDIGESSPNPTIEEGRTLRVLKMVEPWLRGNQFCIKILNPYMPSVVETLEQMQRKHGGMLVRNPLSRNSTHEMYWVSCGTGNIVSAVNMTSRMLLNRFTMAHRKPTYERDVDLGAGTRHVAVEPEVANLDIIGQRIENIKNEHKSTWHYDEDNPYKTWAYHGSYEVKPSGSASSMVNGVVRLLTKPWDVIPMVTQIAMTDTTPFGQQRVFKEKVDTRTPKAKRGTAQIMEVTARWLWGFLSRNKKPRICTREEFTRKVRSNAAIGAVFVDENQWNSAKEAVEDERFWDLVHRERELHKQGKCATCVYNMMGKREKKLGEFGKAKGSRAIWYMWLGARFLEFEALGFMNEDHWFSRENSLSGVEGEGLHKLGYILRDISKIPGGNMYADDTAGWDTRITEDDLQNEAKITDIMEPEHALLATSIFKLTYQNKVVRVQRPAKNGTVMDVISRRDQRGSGQVGTYGLNTFTNMEAQLIRQMESEGIFSPSELETPNLAERVLDWLEKYGVERLKRMAISGDDCVVKPIDDRFATALTALNDMGKVRKDIPQWEPSKGWNDWQQVPFCSHHFHQLIMKDGREIVVPCRNQDELVGRARVSQGAGWSLRETACLGKSYAQMWQLMYFHRRDLRLAANAICSAVPVDWVPTSRTTWSIHAHHQWMTTEDMLSVWNRVWIEENPWMEDKTHVSSWEDVPYLGKREDQWCGSLIGLTARATWATNIQVAINQVRRLIGNENYLDYMTSMKRFKNESDPEGALW

SEQ ID NO: 163 - protein, 3391 aa, unidentified
MNNQRKKARNTPFNMLKRERNRVSTVQQLTKRFSLGMLQGRGPLKLFMALVAFLRFLTIPPTAGILKRWGTIKKSKAINVLRGFRKEIGRMLNILNRRRRTAGXIIMLIPTVMAFHLTTRNGEPHMIVSRQEKGKSLLFKTEDGVNMCTLMAMDLGELCEDTITYKCPLLRQNEPEDIDCWCNSTSTWVTYGTCTTTGEHRREKRSVALVPHVGMGLETRTETWMSSEGAWKHAQRIETWILRHPGFTIMAAILAYTIGTTHFQRVLIFILLTAVAPSMTMRCIGISNRDFVEGVSGGSWVDIVLEHGSCVTTMAKNKPTLDFELIKTEAKQPATLRKYCIEAKLTNTTTDSRCPTQGEPSLNEEQDKRFVCKHSMVDRGWGNGCGLFGKGGIVTCAMFTCKKNMEGKIVQPENLEYTIVITPHSGEEHAVGNDTGKHGKEIKVTPQSSITEAELTGYGTVTMECSPRTGLDFNEMVLLQMENKAWLVHRQWFLDLPLPWLPGADTQGSNWIQKETLVTFKNPHAKKQDVVVLGSQEGAMHTALTGATEIQMSSGNLLFTGHLKCRLRMDKLQLKGMSYSMCTGKFKVVKEIAETQHGTIVIRVQYEGDGSPCKIPFEIMDLEKRHVLGRLITVNPIVTEKDSPVNIEAEPPFGDSYIIIGVEPGQLKLNWFKKGSSIGQMFETTMRGAKRMAILGDTAWDFGSLGGVFTSIGKALHQVFGAIYGAAFSGVSWTMKILIGVIITWIGMNSRSTSLSVSLVLVGIVTLYLGVMVQADSGCVVSWKNKELKCGSGIFITDNVHTWTEQYKFQPESPSKLASAIQKAHEEGICGIRSVTRLENLMWKQITPELNHILSENEVKLTIMTGDIKGIMQAGKRSLRPQPTELKYSWKTWGKAKMLSTEXHNQTFLIDGPETAECPNTNRAWNSLEVEDYGFGVFTTNIWLKLKEKQDVFCDSKLMSAAIKDNRAVHADMGYWIESALNDTWKIEKASFIEVKSCHWPKSHTLWSNGVLESEMIIPKNFAGPVSQHNYRPGYHTQTAGPWHLGKLEMDFDFCEGTTVVVTEDCGNRGPSLRTTTASGKLITEWCCRSCTLPPLRYRGEDGCWYGMEIRPLKEKEENLVNSLVTAGHGQIDNFSLGVLGMALFLEEMLRTRVGTKHAILLVAVSFVTLITGNMSFRDLGRVMVMVGATMTDDIGMGVTYLALLAAFKVRPTFAAGLLLRKLTSKELMMTTIGIVLLSQSTIPETILELTDALALGMMVLKIVRNMEKYQLAVTIMAILCVPNAVILQNAWKVSCTILAVVSVSPLLLTSSQQKADWIPLALTIKGLNPTAIFLTTLSRTSKKRSWPLNEAIMAVGMVSILASSLLKNDIPMTGPLVAGGLLTVCYVLTGRSADLELERAADVKWEDQAEISGSSPILSITISEDGSMSIKNEEEEQTLTILIRTGLLVISGLFPVSIPITAAAWYLWEVKKQRAGVLWDVPSPPPVGKAELEDGAYRIKQKGILGYSQIGAGVYKEGTFHTMWHVTRGAVLMHKGKRIEPSWADVKKDLISYGGGWKLEGEWKEGEEVQVLALEPGKNPRAVQTKPGLFKTNTGTIGAVSLDFSPGTSGSPIVDKKGKVVGLYGNGVVTRSGAYVSAIAQTEKSIEDNPEIEDDIFRKKRLTIMDLHPGAGKTKRYLPAIVREAIKRGLRTLILAPTRVVAAEMEEALRGLPIRYQTPAIRAEHTGREIVDLMCHATFTMRLLSPVRVPNYNLIIMDEAHFTDPASIAARGYISTRVEMGEAAGIFMTATPPGSRDPFPQSNAPIMDEEREIPERSWNSGHEWVTDFKGKTVWFVPSIKAGNDIAACLRKNGKKVIQLSRKTFDSEYIKTRTNDWDFVVTTDISEMGANFKAERVIDPRRCMKPVILTDGEERVILAGPMPVTHSSAAQRRGRIGRNPKNENDQYIYMGEPLENDEDCAHWKEAKMLLDNINTPEGIIPSMFEPEREKVDAIDGEYRLRGEARKTFVDLMRRGDLPVWLAYRVAAEGINYADRRWCFDGEKNNQILEENVEVEIWTKEGERKKLKPRWLDARIYSDPLALKEFKEFAAGRKSLTLNLITEMGRLPTFMTQKARDALDNLAVLHTAEAGGRAYNHALSELPETLETLLLLTLLATVTGGIFLFLMSGKGIGKMTLGMCCIITASILLWYAQIQPHWIAASIILEFFLIVLLIPEPEKQRTPQDNQLTYVVIAILTVVAATMANEMGFLEKTKKDLGLGSIXTQQPESNILDIDLRPASAWTLYAVATTFITPMLRHSIENSSVNVSLTAIANQATVLMGLGKGWPLSKMDIGVPLLAIGCYSQVNPITLTAALLLLVAHYAIIGPGLQAKATREAQKRAAAGIMKNPTVDGITVIDLDPIPYDPKFEKQLGQVMLLVLCVTQVLMMRTTWALCEALTLATGPISTLWEGNPGRFWNTTIAVSMANIFRGSYLAGAGLLFSIMKNTTNTRRGTGNIGETLGEKWKSRLNALGKSEFQIYKKSGIQEVDRTLAKEGIKRGETDHHAVSRGSAKLRWFVERNLVTPEGKVVDLGCGRGGWSYYCGGLKNVREVKGLTKGGPGHEEPIPMSTYGWNLVRLQSGVDVFFTPPEKCDTLLCDIGESSPNPTVEAGRTLRVLNLVENWLNNNTQFCIKVLNPYMPSVIEKMEALQRKYGGALVRNPLSRNSTHEMYWVSNASGNIVSSVNMISRMLINRFTMRHKKATYEPDVDLGSGTRNIGIESEIPNLDIIGKRIEKIKQEHETSWHYDQDHPYKTWAYHGSYETKQTGSASSMVNGVVRLLTKPWDVXPMVTQMAMTDTTPFGQQRVFKEKVDTRTQEPKEGTKKLMKITAEWLWKELGKKKTPRMCTREEFTRKVRSNAALGAIFTDENKWKSAREAVEDSRFWELVDKERNLHLEGKCETCVYNMMGKREKKLGEFGKAKGSRAIWYMWLGARFLEFEALGFLNEDHWFSRENSLSGVEGEGLHKLGYILRDVSKKEGGAMYADDTAGWDTRITLEDLKNEEMVTNHMEGEHKKLAEAIFKLTYQNKVVRVQRPTPRGTVMDIISRRDQRGSGQVGTYGLNTFTNMEAQLIRQMEGEGVFKSIQHLTVTEEIAVQNWLARVGRERLSRMAISGDDCVVKPLDDRFASALTALNDMGKVRKDIQQWEPSRGWNDWTQVPFCSHHFHELIMKDGRVLVVPCRNQDELIGRARISQGAGWSLRETACLGKSYAQMWSLMYFHRRDLRLAANAICSAVPSHWVPTSRTTWSIHAKHEWMTTEDMLTVWNRVWIQENPWMEDKTPVESWEEIPYLGKREDQWCGSLIGLTSRATWAKNIQTAINQVRSLIGNEEYTDYMPSMKRFRREEEEAGVLW

SEQ ID NO: 164 - protein, 3390 aa, unidentified
MNNQRKKTGKPSINMLKRVRNRVSTGSQLAKRFSRGLLNGQGPMKLVMAFIAFLRFLAIPPTAGVLARWGTFKKSGAIKVLKGFKKEISNMLSIINKRKKTSLCLMMMLPATLAFHLTSRDGEPRMIVGKNERGKSLLFKTASGINMCTLIAMDLGEMCDDTVTYKCPHITEVEPEDIDCWCNLTSTWVTYGTCNQAGEHRRDKRSVALAPHVGMGLDTRTQTWMSAEGAWRQVEKVETWALRHPGFTILALFLAHYIGTSLTQKVVIFILLMLVTPSMTMRCVGVGNRDFVEGLSGATWVDVVLEHGGCVTTMAKNKPTLDIELQKTEATQLATLRKLCIEGKITNITTDSRCPTQGEAILPEEQDQNYVCKHTYVDRGWGNGCGLFGKGSLVTCAKFQCLESIEGKVVQHENLKYTVIITVHTGDQHQVGNETQGVTAEITPQASTTEAILPEYGTLGLECSPRTGLDFNEMILLTMKNKAWMVHRQWFFDLPLPWTSGATTETPTWNRKELLVTFKNAHAKKQEVVVLGSQEGAMHTALTGATEIQNSGGTSIFAGHLKCRLKMDKLELKGMSYAMCTNTFVLKKEVSETQHGTILIKVEYKGEDAPCKIPFSTEDGQGKAHNGRLITANPVVTKKEEPVNIEAEPPFGESNIVIGIGDKALKINWYKKGSSIGKMFEATARGARRMAILGDTAWDFGSVGGVLNSLGKMVHQIFGSAYTALFSGVSWIMKIGIGVLLTWIGLNSKNTSMSFSCIAIGIITLYLGAVVQADMGCVINWKGKELKCGSGIFVTNEVHTWTEQYKFQADSPKRLATAIAGAWENGVCGIRSTTRMENLLWKQIANELNYILWENNIKLTVVVGDIIGVLEQGKRTLTPQPMELKYSWKTWGKAKIVTAETQNSSFIIDGPNTPECPSASRAWNVWEVEDYGFGVFTTNIWLKLREVYTQLCDHRLMSAAVKDERAVHADMGYWIESQKNGSWKLEKASLIEVKTCTWPKSHTLWSNGVLESDMIIPKSLAGPISQHNHRPGYHTQTAGPWHLGKLELDFNYCEGTTVVITENCGTRGPSLRTTTVSGKLIHEWCCRSCTLPPLRYMGEDGCWYGMEIRPINEKEENMVKSLVSAGSGKVDNFTMGVLCLAILFEEVMRGKFGKKHMIAGVLFTFVLLLSGQITWRDMAHTLIMIGSNASDRMGMGVTYLALIATFKIQPFLALGFFLRKLTSRENLLLGVGLAMATTLQLPEDIEQMANGIALGLMALKLITQFETYQLWTALVSLTCSNTIFTLTVAWRTATLILAGVSLLPVCQSSSMRKTDWLPMTVAAMGVPPLPLFIFSLKDTLKRRSWPLNEGVMAVGLVSILASSLLRNDVPMAGPLVAGGLLIACYVITGTSADLTVEKAADVTWEEEAEQTGVSHNLMITVDDDGTMRIKDDETENILTVLLKTALLIVSGIFPYSIPATLLVWHTWQKQTQRSGVLWDVPSPPETQKAELEEGVYRIKQQGIFGKTQVGVGVQKEGVFHTMWHVTRGAVLTHNGKRLEPNWASVKKDLISYGGGWRLSAQWQKGEEVQVIAVEPGKNPKNFQTMPGIFQTTTGEIGAIALDFKPGTSGSPIINREGKVVGLYGNGVVTKNGGYVSGIAQTNAEPDGPTPELEEEMFKKRNLTIMDLHPGSGKTRKYLPAIVREAIKRRLRTLILAPTRVVAAEMEEALKGLPIRYQTTATKSEHTGREIVDLMCHATFTMRLLSPVRVPNYNLIIMDEAHFTDPASIAARGYISTRVGMGEAAAIFMTATPPGTADAFPQSNAPIQDEERDIPERSWNSGNEWITDFAGKTVWFVPSIKAGNDIANCLRKNGKKVIQLSRKTFDTEYQKTKLNDWDFVVTTDISEMGANFKADRVIDPRRCLKPVILTDGPERVILAGPMPVTAASAAQRRGRVGRNPQKENDQYIFTGQPLNNDEDHAHWTEAKMLLDNINTPEGIIPALFEPEREKSAAIDGEYRLKGESRKTFVELMRRGDLPVWLAHKVASEGIKYTDRKWCFDGERNNQILEENMDVEIWTKEGEKKKLRPRWLDARTYSDPLALKEFKDFAAGRKSIALDLVTEIGRVPSHLAHRTRNALDNLVMLHTSEHGGRAYRHAVEELPETMETLLLLGLMILLTGGAMLFLISGKGIGKTSIGLICVIASSGMLWMADVPLQWIASAIVLEFFMMVLLIPEPEKQRTPQDNQLAYVVIGILTLAAIVAANEMGLLETTKRDLGMSKEPGVVSPTSYLDVDLHPASAWTLYAVATTVITPMLRHTIENSTANVSLAAIANQAVVLMGLDKGWPISKMDLGVPLLALGCYSQVNPLTLTAAVLLLVTHYAIIGPGLQAKATREAQKRTAAGIMKNPTVDGIMTIDLDPVIYDSKFEKQLGQVMLLVLCAVQLLLMRTSWALCEALTLATGPITTLWEGSPGKFWNTTIAVSMANIFRGSYLAGAGLAFSIMKSVGTGKRGTGSQGETLGEKWKKKLNQLSRKEFDLYKKSGITEVDRTEAKEGLKRGETTHHAVSRGSAKLQWFVERNMVIPEGRVIDLGCGRGGWSYYCAGLKKVTEVRGYTKGGPGHEEPVPMSTYGWNIVKLMSGKDVFYLPPEKCDTLLCDIGESSPSPTVEESRTIRVLKMVEPWLKNNQFCIKVLNPYMPTVIEHLERLQRKHGGMLVRNPLSRNSTHEMYWISNGTGNIVSSVNMVSRLLLNRFTMTHRRPTIEKDVDLGAGTRHVNAEPETPNMDVIGERIKRIKEEHSSTWHYDDENPYKTWAYHGSYEVKATGSASSMINGVVKLLTKPWDVVPMVTQMAMTDTTPFGQQRVFKEKVDTRTPRPMPGTRKVMEITAEWLWRTLGRNKRPRLCTREEFTKKVRTNAAMGAVFTEENQWDSARAAVEDEEFWKLVDRERELHKLGKCGSCVYNMMGKREKKLGEFGKAKGSRAIWYMWLGARYLEFEALGFLNEDHWFSRENSYSGVEGEGLHKLGYILRDISKIPGGAMYADDTAGWDTRITEDDLHNEEKITQQMDPEHRQLANAIFKLTYQNKVVKVQRPTPKGTVMDIISRKDQRGSGQVGTYGLNTFTNMEAQLIRQMEGEGVLSKADLENPHLLEKKITQWLETKGVERLKRMAISGDDCVVKPIDDRFANALLALNDMGKVRKDIPQWQPSKGWHDWQQVPFCSHHFHELIMKDGRKLVVPCRPQDELIGRARISQGAGWSLRETACLGKAYAQMWSLMYFHRRDLRLASNAICSAVPVHWVPTSRTTWSIHAHHQWMTTEDMLTVWNRVWIEDNPWMEDKTPVTTWEDVPYLGKREDQWCGSLIGLTSRATWAQNILTAIQQVRSLIGNEEFLDYMPSMKRFRKEEESEGAIW

SEQ ID NO: 165 - protein, 3387 aa, unidentified
MNQRKKVVRPPFNMLKRERNRVSTPQGLVKRFSTGLFSGKGPLRMVLAFITFLRVLSIPPTAGILKRWGQLKKNKAIKILTGFRKEIGRMLNILNGRKRSTXTLLCLIPTVMAFHLSTRDGEPLMIVAKHERGRPLLFKTTEGINKCTLIAMDLGEMCEDTVTYKCPLLVNTEPEDIDCWCNLTSAWVMYGTCTQSGERRREKRSVALTPHSGMGLETRAETWMSSEGAWKHAQRVESWILRNPGFALLAGFMAYMIGQTGIQRTVFFILMMLVAPSYGMRCVGVGNRDFVEGVSGGAWVDLVLEHGGCVTTMAQGKPTLDFELIKTTAKEVALLRTYCIEASISNITTATRCPTQGEPYLKEEQDQQYICRRDVVDRGWGNGCGLFGKGGVVTCAKFSCSGKITGNLVQIENLEYTVVVTVHNGDTHAVGNDTSNHGVTATITPRSPSVEVKLPDYGELTLDCEPRSGIDFNEMILMKMKKKTWLVHKQWFLDLPLPWTAGADTSEVHWNRKERMVTFKVPHAKRQDVTVLGSQEGAMHSALTGATEVDSGDGNHMFAGHLKCKVRMEKLRIKGMSYTMCSGKFSIDKEMAETQHGTTVVKVKYEGAGAPCKVPIEIRDVNKEKVVGRIISSTPFAENTNSVTNIELEPPFGDSYIVIGVGDSALTLHWFRKGSSIGKMFESTYRGAKRMAILGETAWDFGSVGGLLTSLGKAVHQVFGSVYTTMFGGVSWMVRILIGFLVLWIGTNSRNTSMAMSCIAVGGITLFLGFTVHADMGCAVSWSGKELKCGSGIFVIDNVHTWTEQYKFQPESPARLASAILNAHKDGVCGIRSTTRLENVMWKQITNELNYVLWEGGHDLTVVAGDVKGVLSKGKRALAPPVNDLKYSWKTWGKAKIFTPEXRNSTFLIDGPDTSECPNERRAWNFWEVEDYGFGMFTTNIWMKFREGSSEVCDHRLMSAAIKDQKAVHADMGYWIESSKNQTWQIEKASLIEVKTCLWPKTHTLWSNGVLESQMLIPKAYAGPFSQHNYRQGYATQTVGPWHLGKLEIDFGECPGTTVTIQEDCDHRGPSLRTTTASGKLVTQWCCRSCTMPPLRFLGEDGCWYGMEIRPLSEKEENMVKSQVSAGQGTSETFSMGLLCLTLFVEECLRRRVTRKHMILVVVTTLCAIILGGLTWMDLLRALIMLGDTMSGRMGGQIHLAIMAVFKMSPGYVLGIFLRKLTSRETALMVIGMAMTTVLSIPHDLMEFIDGISLGLILLKMVTHFDNTQVGTLALSLTFIKSTMPLVMAWRTIMAVLFVVTLIPLCRTSCLQKQSHWVEITALILGAQALPVYLMTLMKGASKRSWPLNEGIMAVGLVSLLGSALLKNDVPLAGPMVAGGLLLAAYVMSGSSADLSLEKAANVQWDEMADITGSSPIIEVKQDEDGSFSIRDVEETNMITLLVKLALITVSGLYPLAIPVTMTLWYMWQVKTQRSGALWDVPSPAAAQKATLTEGVYRIMQRGLFGKTQVGVGIHMEGVFHTMWHVTRGSVICHEGGRLEPSWADVRNDMISYGGGWRLGDKWDKEEDVQVLAIEPGKNPKHVQTKPGLFKTLTGEIGAVTLDFKPGTSGSPIINRKGKVIGLYGNGVVTKSGDYVSAITQAERIGEPDYEVDEDIFRKKRLTIMDLHPGAGKTKRILPSIVREALKRRLRTLILAPTRVVAAEMEEALRGLPIRYQTPAVKSEHTGREIVDLMCHATFTTRLLSSTRVPNYNLIVMDEAHFTDPSSVAARGYISTRVEMGEAAAIFMTATPPGTTDPFPQSNSPIEDIEREIPERSWNTGFDWITDYQGKTVWFVPSIKAGNDIANCLRKSGKKVIQLSRKTFDTEYPKTKLTDWDFVVTTDISEMGANFRAGRVIDPRRCLKPVILTDGPERVILAGPIPVTPASAAQRRGRIGRNPAQEDDQYVFSGDPLNNDEDHAHWTEAKMLLDNIYTPEGIIPTLFGPEREKTQAIDGEFRLRGEQRKTFVELMRRGDLPVWLSYKVASAGISYKDREWCFTGERNNQILEENMEVEIWTREGEKKKLRPKWLDARVYADPMALKDFKEFASGRKSITLDILTEIASLPTYLSSRAKLALDNIVMLHTTERGGKAYQHALNELPESLETLMLVALLGAMTAGIFLFFMQGKGIGKLSMGLIAIAVASGLLWVAEIQPQWIAASIILEFFLMVLLIPEPEKQRTPQDNQLIYVILTILTIIGLVAANEMGLIEKTKTDFGFYQVKTETTILDVDLRPASAWTLYAVATTILTPMLRHTIENTSANLSLAAIANQAAVLMGLGKGWPLHRMDLGVPLLAMGCYSQVNPTTLTASLVMLLVHYAIIGPGLQAKATREAQKRTAAGIMKNPTVDGITVIDLEPISYDPKFEKQLGQVMLLVLCAGQLLLMRTTWAFCEVLTLATGPILTLWEGNPGRFWNTTIAVSTANIFRGSYLAGAGLAFSLIKNAQTPRRGTGTTGETLGEKWKRQLNSLDRKEFEEYKRSGILEVDRTEAKSALKDGSKIKHAVSRGSSKIRWIVERGMVKPKGKVVDLGCGRGGWSYYMATLKNVTEVKGYTKGGPGHEEPIPMATYGWNLVKLHSGVDVFYKPTEQVDTLLCDIGESSSNPTIEEGRTLRVLKMVEPWLSSKPEFCIKVLNPYMPTVIEELEKLQRKHGGSLVRCPLSRNSTHEMYWVSGVSGNIVSSVNTTSKMLLNRFTTRHRKPTYEKDVDLGAGTRSVSTETEKPDMTIIGRRLQRLQEEHKETWHYDQENPYRTWAYHGSYEAPSTGSASSMVNGVVKLLTKPWDVXPMVTQLAMTDTTPFGQQRVFKEKVDTRTPQPKPGTRMVMTTTANWLWALLGKKKNPRLCTREEFISKVRSNAAIGAVFQEEQGWTSASEAVNDSRFWELVDKERALHQEGKCESCVYNMMGKREKKLGEFGRAKGSRAIWYMWLGARFLEFEALGFLNEDHWFGRENSWSGVEGEGLHRLGYILEDIDKKDGDLIYADDTAGWDTRITEDDLLNEELITEQMAPHHKILAKAIFKLTYQNKVVKVLRPTPKGAVMDIISRKDQRGSGQVGTYGLNTFTNMEVQLIRQMEAEGVITQDDMHNPKGLKERVEKWLKECGVDRLKRMAISGDDCVVKPLDERFSTSLLFLNDMGKVRKDIPQWEPSKGWKNWQEVPFCSHHFHKIFMKDGRSLVVPCRNQDELIGRARISQGAGWSLRETACLGKAYAQMWSLMYFHRRDLRLASMAICSAVPTEWFPTSRTTWSIHAHHQWMTTEDMLKVWNRVWIEDNPNMTDKTPVHSWEDIPYLGKREDLWCGSLIGLSSRATWAKNIHTAITQVRNLIGKEEYVDYMPVMKRYSAPFESEGVL

SEQ ID NO: 166 - protein, 3392 aa, unidentified
MNNQRKKTGRPSFNMLKRERNRVSTVSQLAKRFSXGLLSGQGPMKLVMAFIAFLRFLAIPPTAGILARWGTFKKSGAIKVLRGFKKEISXMLNILNRRKRSXXXLLMLXPTVLAFHLTTRDGEPHMIVXKQERGKSLLFKTXXGINMCTLIAMDLGELCEDTVTYKCPLITEXEPEDIDCWCNLTSTWVTYGTCTQTGEHRRDKRSVALAPHVGMGLETRTETWMSSEGAWKQAQRVETWALRHPGFTILALFLAHXIGTSXTQKVVIFILLMLVTPSMTMRCVGVGNRDFVEGLSGATWVDVVLEHGSCVTTMAKNKPTLDIELIKTEATQPATLRKLCIEAKITNTTTDSRCPTQGEAXLXEEQDQNFVCKHTXVDRGWGNGCGLFGKGSLVTCAKFXCXXKIEGKIVQXENLKYTVXVTVHTGDQHQVGNXTXXHGVTATITPQASTTEAXLTDYGTLTLECSPRTGLDFNEMVLLTMKNKAWLVHRQWFLDLPLPWTSGADTSXXTWNRKELLVTFKNAHAKKQEVVVLGSQEGAMHTALTGATEIQXSGGTXIFAGHLKCRLKMDKLXLKGMSYXMCTGKFKLXKEVAETQHGTILVKVKYEGXDAPCKIPFEIXDXXGXXVNGRLITANPIVTXKESPVNIEAEPPFGESYIVIGVGDKALKLNWFKKGSSIGKMFEATARGARRMAILGDTAWDFGSVGGVFTSLGKAVHQVFGSAYGALFSGVSWTMKIGIGVLLTWIGLNSRSTSLSMSCIAVGIITLYLGVMVQADSGCVINWKGKELKCGSGIFVTNEVHTWTEQYKFQADSPKRLASAIXKAWEEGVCGIRSTTRLENLMWKQITNELNHILWENXXKLTVVVGDXKGILXQGKRXLRPQPMELKYSWKTWGKAKIXTAEXQNSTFIIDGPNTPECPNXXRAWNXWEVEDYGFGVFTTNIWLKLREXYTQVCDHRLMSAAIKDXRAVHADMGYWIESXKNXTWKLEKASFIEVKTCXWPKSHTLWSNGVLESEMIIPKXYAGPISQHNYRPGYHTQTAGPWHLGKLELDFDXCEGTTVVVTEDCGNRGPSLRTTTVSGKLIHEWCCRSCTLPPLRYXGEDGCWYGMEIRPLKEKEENLVKSLVSAGSGXVDNFSLGXLCLAIFXEEVMRXRVGRKHMILGVLXTFXLLIXGQLTWRDLXRXLIMVGANASDRMGMGVTYLALMATFKMRPXFALGLLLRKLTSREXLLLTIGLALXTXLXLPEDXEELXDGLALGLMXLKLVTXFEXYQLWTTLLSLTCXKTTXXLXXAWRTXXXILAVVSLXPLCLSSSXQKTDWLPXXLXILGXXPLPXFLXTLXKTXKRRSWPLNEGIMAVGLVSILASSLLKNDVPMAGPLVAGGLLIACYVITGSSADLSLEKAADVXWEEEAEISGSSHNLXITVDDDGTMSIKDEEEENTLTILLKTALLXVSGLFPXSIPATLXVWYXWQKKKQRSGVLWDVPSPPEVQKAELEDGVYRIKQRGILGKSQVGVGVXKEGVFHTMWHVTRGAVLMHXGKRLEPSWASVKKDLISYGGGWRLXGXWXKGEEVQVIAVEPGKNPKNVQTKPGLFKTXTGEIGAIALDFKPGTSGSPIINREGKVVGLYGNGVVTKSGXYVSAIAQAEASIEGPXPEIEEEIFRKRNLTIMDLHPGSGKTRRYLPAIVREAIKRRLRTLILAPTRVVAAEMEEALKGLPIRYQTTAVKSEHTGREIVDLMCHATFTMRLLSPVRVPNYNLIIMDEAHFTDPASIAARGYISTRVGMGEAAAIFMTATPPGSXDAFPQSNAPIQDEERDIPERSWNSGXEWITDFXGKTVWFVPSIKAGNDIANCLRKNGKKVIQLSRKTFDTEYQKTKLNDWDFVVTTDISEMGANFKADRVIDPRRCLKPVILTDGPERVILAGPMPVTXASAAQRRGRIGRNPXKENDQYIYMGQPLNNDEDHAHWTEAKMLLDNINTPEGIIPALFEPEREKSAAIDGEYRLRGEARKTFVELMRRGDLPVWLAYKVASEGIXYXDRRWCFDGERNNQILEENMDVEIWTKEGERKKLRPRWLDARTYSDPLALKEFKEFAAGRKSITLDLITEIGRLPTHLTQRARNALDNLVMLHTSEXGGRAYRHALEELPETLETLLLLALLAXLTGGIFLFLMSGKGIGKTSIGLICVIASSGLLWMAXIQPHWIAASIILEFFLMVLLIPEPEKQRTPQDNQLAYVVIGILTXIAXVAANEMGLLETTKKDLGXGXVXTXXXXTXILDVDLRPASAWTLYAVATTIITPMLRHTIENSTANVSLTAIANQAAVLMGLDKGWPISKMDIGVPLLALGCYSQVNPLTLTAAVLLLVAHYAIIGPGLQAKATREAQKRTAAGIMKNPTVDGITVIDLDPVXYDPKFEKQLGQVMLLVLCAXQLLLMRTTWALCEALTLATGPITTLWEGSPGKFWNTTIAVSMANIFRGSYLAGAGLAFSIMKSXGTGRRGTGXQGETLGEKWKRQLNQLSKSEFXXYKRSGIXEVDRTEAKEGLKRGETTHHAVSRGSAKLRWFVERNMVKPEGKVIDLGCGRGGWSYYCAGLKKVTEVKGYTKGGPGHEEPIPMSTYGWNLVKLXSGKDVFFXPPEKCDTLLCDIGESSPNPTIEEGRTLRVLKMVEPWLXNNQFCIKVLNPYMPSVIEXLEXLQRKHGGMLVRNPLSRNSTHEMYWVSNGTGNIVSSVNMTSRMLLNRFTMRHRKPTYEKDVDLGAGTRHVXXEPEXPNLDIIGXRIERIKEEHKSTWHYDQDNPYKTWAYHGSYEVKXTGSASSMVNGVVKLLTKPWDVXPMVTQMAMTDTTPFGQQRVFKEKVDTRTPXPKPGTRKVMEITAEWLWXXLGRNKXPRLCTREEFTRKVRSNAAIGAVFTDENQWXSAREAVEDERFWELVDRERELHKEGKCETCVYNMMGKREKKLGEFGKAKGSRAIWYMWLGARFLEFEALGFLNEDHWFSRENSLSGVEGEGLHKLGYILRDISKIPGGAMYADDTAGWDTRITEDDLXNEEKITXQMEPEHKXLAXAIFKLTYQNKVVRVQRPTPXGTVMDIISRRDQRGSGQVGTYGLNTFTNMEAQLIRQMEGEGVFXXXDLETPXLXEXXVXXWLEXXGVERLKRMAISGDDCVVKPXDDRFATALTALNDMGKVRKDIPQWEPSKGWNDWQQVPFCSHHFHELIMKDGRXLVVPCRNQDELIGRARISQGAGWSLRETACLGKSYAQMWSLMYFHRRDLRLAANAICSAVPVHWVPTSRTTWSIHAHHQWMTTEDMLTVWNRVWIEENPWMEDKTPVXSWEDVPYLGKREDQWCGSLIGLTSRATWAKNIQTAINQVRSLIGNEEYLDYMPSMKRFRXEEESEGALW

SEQ ID NO: 167 - protein, 3423 aa, unidentified
MKNPKKKSGGFRIVNMLKRGVARVSPFGGLKRLPAGLLLGHGPIRMVLAILAFLRFTAIKPSLGLINRWGSVGKKEAMEIIKKFKKDLAAMLRIINARKEKKRRGADTSVGIVGLLLTTAMAAEVTRRGSAYYMYLDRNDAGEAISFPTTLGMNKCYIQIMDLGHMCDATMSYECPMLDEGVEPDDVDCWCNTTSTWVVYGTCHHKKGEARRSRRAVTLPSHSTRKLQTRSQTWLESREYTKHLIRVENWIFRNPGFALAAAAIAWLLGSSTSQKVIYLVMILLIAPAYSIRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGSTIGKAFEATVRGAKRMAVLGDTAWDFGSVGGALNSLGKGIHQIFGAAFKSLFGGMSWFSQILIGTLLMWLGLNTKNGSISLMCLALGGVLIFLSTAVSADVGCSVDFSKKETRCGTGVFVYNDVEAWRDRYKYHPDSPRRLAAAVKQAWEDGICGISSVSRMENIMWRSVEGELNAILEENGVQLTVVVGSVKNPMWRGPQRLPVPVNELPHGWKAWGKSYFVRAAKTNNSFVVDGDTLKECPLKHRAWNSFLVEDHGFGVFHTSVWLKVREDYSLECDPAVIGTAVKGKEAVHSDLGYWIESEKNDTWRLKRAHLIEMKTCEWPKSHTLWTDGIEESDLIIPKSLAGPLSHHNTREGYRTQMKGPWHSEELEIRFEECPGTKVHVEETCGTRGPSLRSTTASGRVIEEWCCRECTMPPLSFRAKDGCWYGMEIRPRKEPESNLVRSMVTAGSTDHMDHFSLGVLVILLMVQEGLKKRMTTKIIISTSMAVLVAMILGGFSMSDLAKLAILMGATFAEMNTGGDVAHLALIAAFKVRPALLVSFIFRANWTPRESMLLALASCLLQTAISALEGDLMVLINGFALAWLAIRAMVVPRTDNITLAILAALTPLARGTLLVAWRAGLATCGGFMLLSLKGKGSVKKNLPFVMALGLTAVRLVDPINVVGLLLLTRSGKRSWPPSEVLTAVGLICALAGGFAKADIEMAGPMAAVGLLIVSYVVSGKSVDMYIERAGDITWEKDAEVTGNSPRLDVALDESGDFSLVEDDGPPMREIILKVVLMTICGMNPIAIPFAAGAWYVYVKTGKRSGALWDVPAPKEVKKGETTDGVYRVMTRRLLGSTQVGVGVMQEGVFHTMWHVTKGSALRSGEGRLDPYWGDVKQDLVSYCGPWKLDAAWDGHSEVQLLAVPPGERARNIQTLPGIFKTKDGDIGAVALDYPAGTSGSPILDKCGRVIGLYGNGVVIKNGSYVSAITQGRREEETPVECFEPSMLKKKQLTVLDLHPGAGKTRRVLPEIVREAIKTRLRTVILAPTRVVAAEMEEALRGLPVRYMTTAVNVTHSGTEIVDLMCHATFTSRLLQPIRVPNYNLYIMDEAHFTDPSSIAARGYISTRVEMGEAAAIFMTATPPGTRDAFPDSNSPIMDTEVEVPERAWSSGFDWVTDHSGKTVWFVPSVRNGNEIAACLTKAGKRVIQLSRKTFETEFQKTKHQEWDFVVTTDISEMGANFKADRVIDSRRCLKPVILDGERVILAGPMPVTHASAAQRRGRIGRNPNKPGDEYLYGGGCAETDEDHAHWLEARMLLDNIYLQDGLIASLYRPEADKVAAIEGEFKLRTEQRKTFVELMKRGDLPVWLAYQVASAGITYTDRRWCFDGTTNNTIMEDSVPAEVWTRHGEKRVLKPRWMDARVCSDHAALKSFKEFAAGKRGAAFGVMEALGTLPGHMTERFQEAIDNLAVLMRAETGSRPYKAAAAQLPETLETIMLLGLLGTVSLGIFFVLMRNKGIGKMGFGMVTLGASAWLMWLSEIEPARIACVLIVVFLLLVVLIPEPEKQRSPQDNQMAIIIMVAVGLLGLITANELGWLERTKSDLSHLMGRREEGATIGFSMDIDLRPASAWAIYAALTTFITPAVQHAVTTSYNNYSLMAMATQAGVLFGMGKGMPFYAWDFGVPLLMMGCYSQLTPLTLIVAIILLVAHYMYLIPGLQAAAARAAQKRTAAGIMKNPVVDGIVVTDIDTMTIDPQVEKKMGQVLLIAVAVSSAILSRTAWGWGEAGALITAATSTLWEGSPNKYWNSSTATSLCNIFRGSYLAGASLIYTVTRNAGLVKRRGGGTGETLGEKWKARLNQMSALEFYSYKKSGITEVCREEARRALKDGVATGGHAVSRGSAKLRWLVERGYLQPYGKVIDLGCGRGGWSYYAATIRKVQEVKGYTKGGPGHEEPVLVQSYGWNIVRLKSGVDVFHMAAEPCDTLLCDIGESSSSPEVEEARTLRVLSMVGDWLEKRPGAFCIKVLCPYTSTMMETLERLQRRYGGGLVRVPLSRNSTHEMYWVSGAKSNTIKSVSTTSQLLLGRMDGPRRPVKYEEDVNLGSGTRAVVSCAEAPNMKIIGNRIERIRSEHAETWFFDENHPYRTWAYHGSYEAPTQGSASSLINGVVRLLSKPWDVVTGVTGIAMTDTTPYGQQRVFKEKVDTRVPDPQEGTRQVMSMVSSWLWKELGKHKRPRVCTKEEFINKVRSNAALGAIFEEEKEWKTAVEAVNDPRFWALVDKEREHHLRGECQSCVYNMMGKREKKQGEFGKAKGSRAIWYMWLGARFLEFEALGFLNEDHWMGRENSGGGVEGLGLQRLGYVLEEMSRIPGGRMYADDTAGWDTRISRFDLENEALITNQMEKGHRALALAIIKYTYQNKVVKVLRPAEKGKTVMDIISRQDQRGSGQVVTYALNTFTNLVVQLIRNMEAEEVLEMQDLWLLRRSEKVTNWLQSNGWDRLKRMAVSGDDCVVKPIDDRFAHALRFLNDMGKVRKDTQEWKPSTGWDNWEEVPFCSHHFNKLHLKDGRSIVVPCRHQDELIGRARVSPGAGWSIRETACLAKSYAQMWQLLYFHRRDLRLMANAICSSVPVDWVPTGRTTWSIHGKGEWMTTEDMLVVWNRVWIEENDHMEDKTLVTKWTDIPYLGKREDLWCGSLIGHRPRTTWAENIKNTVNMVRRIIGDEEKYMDYLSTQVRYLGEEGSTPGVL

SEQ ID NO: 168 - protein, 3423 aa, unidentified
MKNPKKKSGGFRIVNMLKRGVARVNPLGGLKRLPAGLLLGHGPIRMVLAILAFLRFTAIKPSLGLINRWGSVGKKEAMEIIKKFKKDLAAMLRIINARKERKRRGADTSIGIVGLLLTTAMAAEITRRGSAYYMYLDRSDAGKAISFATTLGVNKCHVQIMDLGHMCDATMSYECPMLDEGVEPDDVDCWCNTTSTWVVYGTCHHKKGEARRSRRAVTLPSHSTRKLQTRSQTWLESREYTKHLIKVENWIFRNPGFALVAVAIAWLLGSSTSQKVIYLVMILLIAPAYSIRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFTCSKKMTGKSIQPENLEYRIMLSVHGSQHSGMIVNDIGYETDENRAKVEVTPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGKLFSGHLKCRLKMDKLRLKGVSYSLCTAAFTFTKVPAETLHGTVTVEVQYAGTDGPCKIPAQMAVDMQTLTPVGRLITANPVITESTENSKMMLELDPPFGDSYIVIGVGDKKITHHWHRSGSTIGKAFEATVRGAKRMAVLGDTAWDFGSVGGVFNSLGKGIHQIFGAAFKSLFGGMSWFSQILIGTLLVWLGLNTKNGSISLTCLALGGVMIFLSTAVSADVGCSVDFSKKETRCGTGVFIYNDVEAWRDRYKYHPDSPRRLAAAVKQAWEEGICGISSVSRMENIMWKSVEGELNAILEENGVQLTVVVGSVKNPMWRGPQRLPVPVNELPHGWKAWGKSYFVRAAKTNNSFVVDGDTLKECPLKHRAWNSFLVEDHGFGIFHTSVWLKVREDYSLECDPAVIGTAVKGKEAAHSDLGYWIESEKNDTWRLKRAHLIEMKTCEWPKSHTLWTDGVEESDLIIPKSLAGPLSHHNTREGYRTQVKGPWHSEELEIRFEECPGTKVYVEETCGTRGPSLRSTTASGRVIEEWCCRECTMPPLSFRAKDGCWYGMEIRPRKEPESNLVRSMVTAGSTDHMDHFSLGVLVILLMVQEGLKKRMTTKIIMSTSMAVLVAMILGGFSMSDLAKLVILMGATFAEMNTGGDVAHLALVAAFKVRPALLVSFIFRANWTPRESMLLALASCLLQTAISALEGELMVLINGFALAWLAIRAMAVPRTDNIALAILAALTPLARGTLLVAWRAGLATCGGIMLLSLKGKGSVKKNLPFVMALGLTAVRIVDPINVVGLLLLTRSGKRSWPPSEVLTAVGLICALAGGFAKADIEMAGPMAAVGLLIVSYVVSGKSVDMYIERAGDITWEKDAEVTGNSPRLDVALDESGDFSLVEEDGPPMREIILKVVLMAICGMNPIAIPFAAGAWYVYVKTGKRSGALWDVPAPKEVKKGETTDGVYRVMTRRLLGSTQVGVGVMQEGVFHTMWHVTKGAALRSGEGRLDPYWGDVKQDLVSYCGPWKLDAAWDGLSEVQLLAVPPGERARNIQTLPGIFKTKDGDIGAVALDYPAGTSGSPILDKCGRVIGLYGNGVVIKNGSYVSAITQGKREEEAPVECFEPSMLKKKQLTVLDLHPGAGKTRRVLPEIVREAIKKRLRTVILAPTRVVAAEMEEALRGLPVRYMTTAVNVTHSGTEIVDLMCHATFTSRLLQPIRVPNYNLYIMDEAHFTDPSSIAARGYISTRVEMGEAAAIFMTATPPGTRDAFPDSNSPIMDTEVEVPERAWSSGFDWVTDHSGKTIWFVPSVRNGNEIAACLTKAGKRVIQLSRKTFETEFQKTKNQEWDFVITTDISEMGANFKADRVIDSRRCLKPVILDGERVILAGPMPVTHASAAQRRGRIGRNPNKPGDEYMYGGGCAETDEGHAHWLEARMLLDNIYLQDGLIASLYRPEADKVAAIEGEFKLRTEQRKTFVELMKRGDLPVWLAYQVASAGITYTDRRWCFDGTTNNTIMEDSVPAEVWTKYGEKRVLKPRWMDARVCSDHAALKSFKEFAAGKRGAALGVMEALGTLPGHMTERFQEAIDNLAVLMRAETGSRPYKAAAAQLPETLETIMLLGLLGTVSLGIFFVLMRNKGIGKMGFGMVTLGASAWLMWLSEIEPARIACVLIVVFLLLVVLIPEPEKQRSPQDNQMAIIIMVAVGLLGLITANELGWLERTKSDIAHLMGRKEEGATMGFSMDIDLRPASAWAIYAALTTLITPAVQHAVTTSYNNYSLMAMATQAGVLFGMGKGMPFYAWDFGVPLLMMGCYSQLTPLTLIVAIILLVAHYMYLIPGLQAAAARAAQKRTAAGIMKNPVVDGIVVTDIDTMTIDPQVEKKMGQVLLIAVAISSAVLLRTAWGWGEAGALITAATSTLWEGSPNKYWNSSTATSLCNIFRGSYLAGASLIYTVTRNAGLVKRRGGGTGETLGEKWKARLNQMSALEFYSYKKSGITEVCREEARRALKDGVATGGHAVSRGSAKLRWLVERGYLQPYGKVVDLGCGRGGWSYYAATIRKVQEVRGYTKGGPGHEEPMLVQSYGWNIVRLKSGVDVFHMAAEPCDTLLCDIGESSSSPEVEETRTLRVLSMVGDWLEKRPGAFCIKVLCPYTSTMMETMERLQRRHGGGLVRVPLSRNSTHEMYWVSGAKSNIIKSVSTTSQLLLGRMEGPRRPVKYEEDVNLGSGTRAVASCAEAPNMKIIGRRIERIRNEHAETWFFDENHPYRTWAYHGSYEAPTQGSASSLVNGVVRLLSKPWDVVTGVTGIAMTDTTPYGQQRVFKEKVDTRVPDPQEGTRQVMNMVSSWLWKELGKRKRPRVCTKEEFINKVRSNAALGAIFEEEKEWKTAVEAVNDPRFWALVDKEREHHLRGECHSCVYNMMGKREKKQGEFGKAKGSRAIWYMWLGARFLEFEALGFLNEDHWMGRENSGGGVEGLGLQRLGYILEEMNRAPGGKMYADDTAGWDTRISKFDLENEALITNQMEEGHRALALAVIKYTYQNKVVKVLRPAEGGKTVMDIISRQDQRGSGQVVTYALNTFTNLVVQLIRNMEAEEVLEMQDLWLLRKPEKVTRWLQSNGWDRLKRMAVSGDDCVVKPIDDRFAHALRFLNDMGKVRKDTQEWKPSTGWSNWEEVPFCSHHFNKLYLKDGRSIVVPCRHQDELIGRARVSPGAGWSIRETACLAKSYAQMWQLLYFHRRDLRLMANAICSAVPVDWVPTGRTTWSIHGKGEWMTTEDMLMVWNRVWIEENDHMEDKTPVTKWTDIPYLGKREDLWCGSLIGHRPRTTWAENIKDTVNMVRRIIGDEEKYMDYLSTQVRYLGEEGSTPGVL

SEQ ID NO: 169 - protein, 3423 aa, unidentified
MKNPKKKSGGFRIVNMLKRGVARVXPXGGLKRLPAGLLLGHGPIRMVLAILAFLRFTAIKPSLGLINRWGSVGKKEAMEIIKKFKKDLAAMLRIINARKEXKRRGADTSIGIVGLLLTTAMAAEXTRRGSAYYMYLDRXDAGXAISFXTTLGXNKCXXQIMDLGHMCDATMSYECPMLDEGVEPDDVDCWCNTTSTWVVYGTCHHKKGEARRSRRAVTLPSHSTRKLQTRSQTWLESREYTKHLIKVENWIFRNPGFALAAAAIAWLLGSSTSQKVIYLVMILLIAPAYSIRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFACSKKMTGKSIQPENLEYRIMLSVHGSQHSGMIVNDIGYETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGKLSSGHLKCRLKMDKLRLKGVSYSLCTAAFTFTKIPAETLHGTVTVEVQYAGTDGPCKIPAQMAVDMQTLTPVGRLITANPVITESTENSKMMLELDPPFGDSYIVIGVGEKKITHHWHRSGSTIGKAFEATVRGAKRMAVLGDTAWDFGSVGGALNSLGKGIHQIFGAAFKSLFGGMSWFSQILIGTLLVWLGLNTKNGSISLTCLALGGVLIFLSTAVSADVGCSVDFSKKETRCGTGVFIYNDVEAWRDRYKYHPDSPRRLAAAVKQAWEEGICGISSVSRMENIMWKSVEGELNAILEENGVQLTVVVGSVKNPMWRGPQRLPVPVNELPHGWKAWGKSYFVRAAKTNNSFVVDGDTLKECPLKHRAWNSFLVEDHGFGIFHTSVWLKVREDYSLECDPAVIGTAVKGKEAAHSDLGYWIESEKNDTWRLKRAHLIEMKTCEWPKSHTLWTDGIEESDLIIPKSLAGPLSHHNTREGYRTQVKGPWHSEELEIRFEECPGTKVYVEETCGTRGPSLRSTTASGRVIEEWCCRECTMPPLSFRAKDGCWYGMEIRPRKEPESNLVRSMVTAGSTDHMDHFSLGVLVILLMVQEGLKKRMTTKIIISTSMAVLVAMILGGFSMSDLAKLAILMGATFAEMNTGGDVAHLALIAAFKVRPALLVSFIFRANWTPRESMLLALASCLLQTAISALEGELMVLINGFALAWLAIRAMAVPRTDNIALAILAALTPLARGTLLVAWRAGLATCGGIMLLSLKGKGSVKKNLPFVMALGLTAVRLVDPINVVGLLLLTRSGKRSWPPSEVLTAVGLICALAGGFAKADIEMAGPMAAVGLLIVSYVVSGKSVDMYIERAGDITWEKDAEVTGNSPRLDVALDESGDFSLVEEDGPPMREIILKVVLMAICGMNPIAIPFAAGAWYVYVKTGKRSGALWDVPAPKEVKKGETTDGVYRVMTRRLLGSTQVGVGVMQEGVFHTMWHVTKGAALRSGEGRLDPYWGDVKQDLVSYCGPWKLDAAWDGLSEVQLLAVPPGERARNIQTLPGIFKTKDGDIGAVALDYPAGTSGSPILDKCGRVIGLYGNGVVIKNGSYVSAITQGKREEEAPVECFEPSMLKKKQLTVLDLHPGAGKTRRVLPEIVREAIKKRLRTVILAPTRVVAAEMEEALRGLPVRYMTTAVNVTHSGTEIVDLMCHATFTSRLLQPIRVPNYNLYIMDEAHFTDPSSIAARGYISTRVEMGEAAAIFMTATPPGTRDAFPDSNSPIMDTEVEVPERAWSSGFDWVTDHSGKTIWFVPSVRNGNEIAACLTKAGKRVIQLSRKTFETEFQKTKNQEWDFVITTDISEMGANFKADRVIDSRRCLKPVILDGERVILAGPMPVTHASAAQRRGRIGRNPNKPGDEYLYGGGCAETDEGHAHWLEARMLLDNIYLQDGLIASLYRPEADKVAAIEGEFKLRTEQRKTFVELMKRGDLPVWLAYQVASAGITYTDRRWCFDGTTNNTIMEDSVPAEVWTKYGEKRVLKPRWMDARVCSDHAALKSFKEFAAGKRGAALGVMEALGTLPGHMTERFQEAIDNLAVLMRAETGSRPYKAAAAQLPETLETIMLLGLLGTVSLGIFFVLMRNKGIGKMGFGMVTLGASAWLMWLSEIEPARIACVLIVVFLLLVVLIPEPEKQRSPQDNQMAIIIMVAVGLLGLITANELGWLERTKSDLAHLMGRKEEGATIGFSMDIDLRPASAWAIYAALTTXITPAVQHAVTTSYNNYSLMAMATQAGVLFGMGKGMPFYAWDFGVPLLMMGCYSQLTPLTLIVAIILLVAHYMYLIPGLQAAAARAAQKRTAAGIMKNPVVDGIVVTDIDTMTIDPQVEKKMGQVLLIAVAISSAILLRTAWGWGEAGALITAATSTLWEGSPNKYWNSSTATSLCNIFRGSYLAGASLIYTVTRNAGLVKRRGGGTGETLGEKWKARLNQMSALEFYSYKKSGITEVCREEARRALKDGVATGGHAVSRGSAKLRWLVERGYLQPYGKVIDLGCGRGGWSYYAATIRKVQEVKGYTKGGPGHEEPVLVQSYGWNIVRLKSGVDVFHMAAEPCDTLLCDIGESSSSPEVEEARTLRVLSMVGDWLEKRPGAFCIKVLCPYTSTMMETLERLQRRYGGGLVRVPLSRNSTHEMYWVSGAKSNIIKSVSTTSQLLLGRMEGPRRPVKYEEDVNLGSGTRAVASCAEAPNMKIIGRRIERIRSEHAETWFFDENHPYRTWAYHGSYEAPTQGSASSLXNGVVRLLSKPWDVVTGVTGIAMTDTTPYGQQRVFKEKVDTRVPDPQEGTRQVMXMVSSWLWKELGKXKRPRVCTKEEFINKVRSNAALGAIFEEEKEWKTAVEAVNDPRFWALVDKEREHHLRGECQSCVYNMMGKREKKQGEFGKAKGSRAIWYMWLGARFLEFEALGFLNEDHWMGRENSGGGVEGLGLQRLGYILEEMSRIPGGKMYADDTAGWDTRISKFDLENEALITNQMEKGHRALALAIIKYTYQNKVVKVLRPAEGGKTVMDIISRQDQRGSGQVVTYALNTFTNLVVQLIRNMEAEEVLEMQDLWLLRKSEKVTRWLQSNGWDRLKRMAVSGDDCVVKPIDDRFAHALRFLNDMGKVRKDTQEWKPSTGWSNWEEVPFCSHHFNKLYLKDGRSIVVPCRHQDELIGRARVSPGAGWSIRETACLAKSYAQMWQLLYFHRRDLRLMANAICSXVPVDWVPTGRTTWSIHGKGEWMTTEDMLXVWNRVWIEENDHMEDKTLVTKWTDIPYLGKREDLWCGSLIGHRPRTTWAENIKNTVNMVRRIIGDEEKYMDYLSTQVRYLGEEGSTPGVL

SEQ ID NO: 170 - DNA, 516 bp, synthetic construct
  source 1..516: mol_type = other DNA
gaaaccctgcagggaattccccagctgtagttataaacagaagttctccttctgctaggtagcattcaaagatcttaatcttctgggtttccgttttctcgaatgaaaaatgcaggtccgagcagttaactggcgggggcaccattagcaagtcacttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagctgacagcattcgggccgagc

SEQ ID NO: 171 - DNA, 715 bp, synthetic construct
  source 1..715: mol_type = other DNA
cttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacggaaaccctgcagggaattccccagctgtagttataaacagaagttctccttctgctaggtagcattcaaagatcttaatcttctgggtttccgttttctcgaatgaaaaatgcaggtccgagcagttaactggcgggggcaccattagcaagtcacttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgg

SEQ ID NO: 172 - DNA, 926 bp, synthetic construct
  source 1..926: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaagatctgttaacttccccgaagtagcaatgtatttcccagaaaaggaataatccttctgggaattctggcgaggtttccgggaaagcagcaccgcccttggccgtcctgccaatttcactttctagtttcactttcccttttgtaactaaaatgtaaatgacataggaaaactgaaagggagaagtgaaagtgggaaattcctctgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagctgacagcattcgggccgagtaacaactccgccccattgacgcaaatgggcggtaggcgtgtacggtgggaggtctatataagcagagctcgtttagtgaaccg

SEQ ID NO: 173 - DNA, 597 bp, synthetic construct
  source 1..597: mol_type = other DNA
ttcccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccgcggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 174 - DNA, 592 bp, synthetic construct
  source 1..592: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctg

SEQ ID NO: 175 - DNA, 20 bp, synthetic construct
  source 1..20: mol_type = other DNA
ctgctgcactataccagaca

SEQ ID NO: 176 - DNA, 20 bp, synthetic construct
  source 1..20: mol_type = other DNA
ctgctgcactataccagaca

SEQ ID NO: 177 - DNA, 20 bp, synthetic construct
  source 1..20: mol_type = other DNA
tctcctctgacttcaacagc

SEQ ID NO: 178 - DNA, 20 bp, synthetic construct
  source 1..20: mol_type = other DNA
ccctgcactttttaagagcc

SEQ ID NO: 179 - DNA, 24 bp, unidentified
  source 1..24: mol_type = other DNA
garagaccagagatcctgctgtct

SEQ ID NO: 180 - DNA, 21 bp, synthetic construct
  source 1..21: mol_type = other DNA
accattccattttctggcgtt

SEQ ID NO: 181 - DNA, 17 bp, synthetic construct
  source 1..17: mol_type = other DNA
agcatcattccaggcac

SEQ ID NO: 182 - DNA, 21 bp, synthetic construct
  source 1..21: mol_type = other DNA
accaatccatctcgcggcgct

SEQ ID NO: 183 - DNA, 17 bp, synthetic construct
  source 1..17: mol_type = other DNA
aacatcaatccaggcac

SEQ ID NO: 184 - protein, 15 aa, unidentified
VARVSPFGGLKRLPA

SEQ ID NO: 185 - protein, 12 aa, unidentified
AMLRIINARKEK

SEQ ID NO: 186 - protein, 9 aa, unidentified
VGLLLTTAM

SEQ ID NO: 187 - protein, 12 aa, unidentified
EVTRRGSAYYMY

SEQ ID NO: 188 - protein, 16 aa, unidentified
KAISFATTLGMNKCYI

SEQ ID NO: 189 - protein, 14 aa, unidentified
HMCDATMSYECPML

SEQ ID NO: 190 - protein, 15 aa, unidentified
RPASAWAIYAALTTF

SEQ ID NO: 191 - protein, 15 aa, unidentified
VQHAVTTSYNNYSLM

SEQ ID NO: 192 - protein, 11 aa, unidentified
SPNKYWNSSTA

SEQ ID NO: 193 - protein, 9 aa, unidentified
YLAGASLIY

SEQ ID NO: 194 - protein, 10 aa, unidentified
MSALEFYSYK

SEQ ID NO: 195 - protein, 15 aa, unidentified
DENHPYRTWAYHGSY

SEQ ID NO: 196 - protein, 10 aa, unidentified
SSLINGVVRL

SEQ ID NO: 197 - protein, 15 aa, unidentified
DTTPYGQQRVFKEKV

SEQ ID NO: 198 - protein, 15 aa, unidentified
RQVMNMVSSWLWKEL

SEQ ID NO: 199 - protein, 11 aa, unidentified
KSYAQMWQLLY

SEQ ID NO: 200 - protein, 9 aa, unidentified
NAICSSVPV

SEQ ID NO: 201 - protein, 8 aa, unidentified
VPTGRTTW

SEQ ID NO: 202 - protein, 9 aa, unidentified
LVVWNRVWI

SEQ ID NO: 203 - protein, 10 aa, unidentified
FIFFFLFNIL

SEQ ID NO: 204 - protein, 15 aa, unidentified
NPFFAVTALTIAYLV

SEQ ID NO: 205 - protein, 10 aa, unidentified
TNGPWMQVPL

SEQ ID NO: 206 - protein, 10 aa, unidentified
AALHPFALLL

SEQ ID NO: 207 - protein, 9 aa, unidentified
IVNRNGEVI

SEQ ID NO: 208 - protein, 8 aa, unidentified
ATLTYRML

SEQ ID NO: 209 - protein, 10 aa, unidentified
VSGWNSITVM

SEQ ID NO: 210 - protein, 9 aa, unidentified
LALYLLLAL

SEQ ID NO: 211 - protein, 9 aa, unidentified
VMRGNHYAF

SEQ ID NO: 212 - protein, 10 aa, unidentified
VMYNLWKMKT

SEQ ID NO: 213 - DNA, 31 bp, synthetic construct
  source 1..31: mol_type = other DNA
cgggatcccgagccaaaaagtcatatacttg

SEQ ID NO: 214 - DNA, 33 bp, synthetic construct
  source 1..33: mol_type = other DNA
accgctcgagtcagtttcatgtcctgtgtcatt

SEQ ID NO: 215 - DNA, 20 bp, synthetic construct
  source 1..20: mol_type = other DNA
aacccacacatgcaggacaa

SEQ ID NO: 216 - DNA, 20 bp, synthetic construct
  source 1..20: mol_type = other DNA
gttgcaggtcagcatccaca

SEQ ID NO: 217 - protein, 19 aa, unidentified
LLNFDLLKLAGDVESNPGP

SEQ ID NO: 218 - protein, 22 aa, unidentified
GSGATNFSLLKQAGDVEENPGP

(19) United States
(12) Patent Application Publication
(10) Pub. No.: US 2026/0097108 A1
(43) Pub. Date: Apr. 9, 2026

(54) POLYNUCLEOTIDES AND LENTIVIRAL VECTORS EXPRESSING NON-STRUCTURAL ANTIGENS OF A FLAVIVIRUS SELECTED FROM THE GROUP OF DENV, ZIKV AND YFV, INDUCING PROTECTIVE CD8+ T-CELL IMMUNITY IN A HOST

(71) Applicants: INSTITUT PASTEUR, PARIS CEDEX 15 (FR); THERAVECTYS, PARIS CEDEX 15 (FR)

(72) Inventors: Pierre CHARNEAU, PARIS (FR); Kirill NEMIROV, PARIS (FR); Pierre AUTHIE, PARIS (FR); Philippe SOUQUE, PLAISIR (FR); Amandine NOIRAT, PARIS (FR); Fanny MONCOQ, PARIS (FR)

(21) Appl. No.: 19/120,869

(22) PCT Filed: Oct. 20, 2023
(86) PCT No.: PCT/EP2023/079275

§ 371 Date: Apr. 14, 2025

(30) Foreign Application Priority Data
Oct. 21, 2022 (EP) 22306605.1

(51) Int. Cl. A61K 39/12; A61K 39/00; C07K 14/74; C12N 5/071; C12N 7/04; C12N 15/62; C12N 15/86

(52) U.S. Cl. CPC A61K 39/12; C07K 14/70539; C12N 5/0602; C12N 7/04; C12N 15/625; C12N 15/86; A61K 2039/53; A61K 2039/70; C07K 2319/00; C12N 2740/15043; C12N 2770/24134

(57) ABSTRACT

The invention relates to recombinant polynucleotides encoding at least a recombinant polynucleotide expressing at least a first fusion polypeptide that comprises MHC class I T-cell epitopes suitable to elicit a T cell immune response in a host in need thereof, wherein the MHC class I T-cell epitopes originate from a plurality of antigens wherein the antigens comprise at least non-structural antigens and are from at least one flavivirus selected from the group of 10 Dengue virus (DENV), ZIKA virus (ZIKV) and Yellow Fever virus (YFV). The invention also relates to the polypeptides comprising polyepitopes of said antigens encoded by the recombinant polynucleotides.

### FIELD OF THE INVENTION

The invention relates to recombinant polynucleotides encoding at least a recombinant polynucleotide expressing at least a first fusion polypeptide that comprises MHC class I T-cell epitopes suitable to elicit a T cell immune response in a host in need thereof, wherein the MHC class I T-cell epitopes originate from a plurality of antigens wherein the antigens comprise at least non-structural antigens and are from at least one flavivirus selected from the group of Dengue virus (DENV), ZIKA virus (ZIKV) and Yellow Fever virus (YFV). The invention also relates to the polypeptides comprising polyepitopes of said antigens encoded by the recombinant polynucleotides.

In another aspect, the invention relates to lentiviral vectors designed to provide an immune response against an infection or against the onset or the development of a condition or disease related to infection by a flavivirus selected from the group of Dengue virus (DENV), ZIKA virus (ZIKV) and Yellow Fever virus (YFV), especially by the induction of CD8⁺ T-cell responses.

In particular, the invention relates to such lentiviral vectors expressing fusion polypeptide(s) selected for their capability to elicit an immunological response in a host, in particular a mammalian host, especially a human host in need thereof wherein the immunological response encompasses a specific CD8+ T-cell response. The fusion polypeptide(s) may be expressed as new antigen(s) from an insert or a plurality of inserts in the lentiviral backbone of the vector wherein the insert(s) contain(s) or consist(s) of at least one polynucleotide encoding a fusion polypeptide or a plurality of fusion polypeptides each comprising the selected MHC class I T-cell epitopes originating from multiple antigens of a determined virus selected from the Dengue virus (DENV), the ZIKA virus (ZIKV) and the Yellow Fever virus (YFV). The new antigen results from the fusion in a polypeptide of the expression products of polynucleotide regions recombined or assembled from distinct genes of the virus.

The recombinant polynucleotides(s), the lentiviral vector(s) or the fusion polypeptide(s) of the invention is provided or expressed for use in the design of immunological compositions, preferably of a vaccine candidate, in particular a vaccine, especially a prophylactic vaccine, suitable for a mammalian host, especially a human host.

### BACKGROUND OF THE INVENTION

Dengue virus (DENV), ZIKA virus (ZIKV) and Yellow Fever virus (YFV) are prevalent viral pathogens transmitted by mosquitoes. DENV is responsible for annual incidence of 390 million infections, mainly in tropical and subtropical regions. While majority of cases are asymptomatic, 96 million cases per year result in clinical manifestations, including 2 million cases of severe forms of the disease: dengue hemorrhagic fever and dengue shock syndrome, leading to 12,500 deaths annually (1, 2).

Development of an efficient vaccine against DENV has been a particularly challenging task despite several decades of research and vaccine development. The failure to develop such a vaccine could be explained by particular epidemiology of DENV: the disease is caused by at least four different serotypes (DENV-1 to DENV-4) that co-circulate in the endemic regions. Primary infection with one DENV serotype usually results in a long-lasting immunity against that serotype but only a short-time immunity against other serotypes that may last for several months. Re-infection with a different serotype after that period often results in a more severe disease, because of the antigenic differences between DENV serotypes. More specifically, antibodies generated in response to a primary infection are unable to efficiently neutralize DENV of a different serotype but bind to viral particles increasing their uptake by antigen-presenting cells—the main target cells for DENV in human organism. This phenomenon, known as antibody-dependent enhancement (ADE) greatly increases replication of DENV in the body, leading to a severe disease. Understanding of the role that antibody response may play in dengue pathogenesis has focused DENV vaccine R&D on attempts to develop a vaccine that induces simultaneous immune response against 4 serotypes of DENV. Only one such vaccine has been developed so far (CYD-TDV, Sanofi Pasteur), however its widespread use has been hampered by several important shortcomings: it has provided lower protection against some DENV serotypes and also led to enhanced infections in individuals that were not previously infected with DENV (3). Other candidate vaccines that are currently undergoing most advanced clinical trials are all live-attenuated vaccines (LAV), and thus present known problems with production and safety associated with that approach. Another approach aiming to circumvent the problem presented by antibody response in DENV pathogenesis is to focus on other elements of immune response, i.e. T cell response. Although initially T cell immune responses were suspected to play a detrimental role in DENV disease via the mechanism called “original antigenic sin”, recent studies have indicated that T cell responses are largely beneficial and could induce cross-serotype specific protection against different serotypes of DENV (2, 4, 5). Responses of CD8+ T cells appeared to be particularly important for the control of DENV infection in mice and humans and were shown to mainly target epitopes located in conserved non-structural proteins of DENV: NS3, NS5 and NS4B (5-9). T cell responses (in particular responses of cytotoxic CD8+ cells) were shown to protect mice against ADE and reduce the severity of heterotypic DENV infections (10, 11). Such results suggested a possibility to create a purely “T cell” vaccine, i.e. a vaccine that avoids generating humoral response against structural proteins of DENV that may result in ADE, instead relying on T cell response for simultaneous protection against different serotypes of DENV (12).

Zika virus (ZIKV) has been responsible for a large outbreak of the congenital syndrome in new borne children and Guillain-Barre syndrome in adults. Between 2007 and 2016, this virus has spread across the Pacific islands and into South America and South-East Asia, culminating in the large outbreak in Brazil in 2016, involving at least 100,000 human cases. That situation has prompted WHO to declare Zika virus epidemics a global health emergency in 2016. Although in the following years the number of Zika virus cases have diminished, the potential for its re-emergence remains high and no licensed vaccine or effective treatment against this virus has been developed so far. Recent spread of ZIKV over the world has initiated intensive effort of vaccine development, and a number of vaccine candidates using a variety of vaccine platforms have been tested in pre-clinical studies. These include nucleic acid-based vaccines (DNA and mRNA), virus-like particles (VLP), inactivated viral vaccines, live-attenuated vaccines, and viral vector vaccines (using the adenoviral-, measles-, and vaccinia-vectored platforms) (49, 50). Many of those vaccine candidates demonstrated protection against ZIKV in mouse and/or Non-Human Primate (NHP) models and some have entered clinical trials. Most of the candidate vaccines currently undergo phase I trials, two are at the stage of phase I/II trials (reviewed in (50)). With ≈55-56% of amino acid identity ZIKV is more closely related to DENV than other flaviviruses (2). The level of antigenic similarity between ZIKV and DENV rose concerns that antibody responses against ZIKV could predispose individuals to a more severe DENV disease via ADE mechanism. Such concerns have been substantiated by the number of studies (51-56). In spite of these concerns, the vast majority of ZIKV vaccines that currently undergo preclinical or clinical development are formulated to induce neutralizing antibody response and are directed against surface proteins. Since DENV and ZIKV co-circulate in the same geographic regions (2) there is a need for protective vaccine against the two viruses that would not induce a cross-specific antibody response that could enhance the severity of DENV and ZIKV diseases.

Yellow Fever virus (YFV) is endemic to tropical and subtropical regions of South America and Africa. Although a majority of human YFV infections are asymptomatic, severe YF occurs in about 12% of infected individuals and may manifest with jaundice, hemorrhage, and multisystem organ failure (57). The disease that has been controlled via vaccination and the mosquito control measures has re-emerged in South America since the 1970s, when the mosquito eradication program was relaxed. There are an estimated 200,000 cases of infection, and 30,000 deaths annually, and 400-500 million unvaccinated people are living in at-risk areas (58). Although an efficient vaccine against YFV (YF-VAX, Sanofi Pasteur) has been used since 1930s and provides high level of protection against the disease, it is based on live attenuated stain of YFV (17D-204) and thus its safety profile is not optimal. In addition, in rare cases it can produce neurological complications. Several analyses of vaccinated travelers have estimated the incidence of serious adverse events, i.e. death, life-threatening illness, hospitalization, or permanent disability, at 1.1-4.7 per 100,000 doses (59). Additionally, production of YF-VAX vaccine is limited by technical issues and currently some endemic regions experience shortage of the vaccine (60). Thus, an easy-to-produce vaccine with the improved safety profile is needed for the continuous protection against YFV. A cell-passaged inactivated viral vaccine candidate XRX-001, developed as a potential alternative, was evaluated in phase I clinical trial. Although vaccination with this candidate induced 100% seroconversion in 24 human subjects without severe adverse events, its safety profile could not be compared with the live-attenuated vaccine due to a limited number of subjects enrolled in this study. (59, 61).

Lentiviral Vectors (LV) provide one of the most efficient vaccine platforms, relied on their outstanding potential of gene transfer to the nuclei of the host cells, including notably Antigen Presenting Cells (APC). These vectors are widely used in gene therapy due to their ability to integrate in the genome of target cells and induce sustained persistent antigen presentation by the APCs (13) and strong induction of T cell immunity (13). So far, application of these vectors to vaccine development was limited due to safety concerns because this technology implied insertion of LV-derived genetic material into the genome of target cells. However, recent studies using the integration-deficient LV demonstrated that efficient antigen presentation can be achieved without integration of LVs in the genome, greatly improving safety of these vectors (14, 15). Nuclear transfer of genes by lentiviral vectors initiates expression of antigens which readily access the Major Histocompatibility Complex Class-I (MHC-1) presentation machinery, i.e., proteasome, for further triggering of CD8⁺ T cells. In net contrast with their substantial ability at routing the endogenously produced antigens into the MHC-1 pathway, viral vectors, including LV, are not very effective in triggering CD4⁺ T cell response. The inventors have demonstrated that CD8⁺ T cells contribute largely to the immune control of infectious diseases caused by infection by a flavivirus selected from the group of Dengue virus (DENV), ZIKA virus (ZIKV) and Yellow Fever virus (YFV). Therefore, leveraging the potential of LV to induce CD8⁺ T cells against polyepitopes originating from a plurality of non-structural antigens wherein the antigens are from at least one flavivirus selected from the group of Dengue virus (DENV), ZIKA virus (ZIKV) and Yellow Fever virus (YFV) will offer new vaccine strategies, especially prophylactic strategy.

### SUMMARY OF THE INVENTION

The Inventors disclose the development of several candidate vaccines, in particular candidate LV vaccines that were shown to induce simultaneous protection of IFNAR-KO mice against infection with 4 serotypes of DENV and/or candidate LV vaccines that induced protection of said mice against infections by Zika virus (ZIKV) and yellow fever virus (YFV). The protective effect is primarily attributed to the induction of CD8+ T cell response directed against conserved regions of non-structural DENV proteins or respectively against conserved regions of ZIKV or YFV located in non-structural and structural proteins.

Although the tested vaccines demonstrate only partial protection in the mouse model, it should be emphasized that this model does not recapitulate well human disease and that it features only some indicators of infection (e.g. viremia). Even though the mouse model does not well recapitulate human flavivirus-mediated diseases, it is the most used small preclinical animal model that allows to measure flavivirus viremia. Besides, testing of a vaccine designed for optimized human T cell response in mice is likely to induce only limited protection in mice, because localization of epitopes targeted by T cell responses is different in humans and mice. However, in the present study, this preclinical model allowed to establish the robust and statistically significant protective potential of the three LV-based vaccine candidates.

Thus, the inventors demonstrated for the first time that the recombinant polynucleotides of the invention, in particular when vectorized by LVs to take advantage of the capability of the vector to elicit a T-cell response, could be used to develop polyvalent vaccines that induce simultaneous protection against different pathogens of the same group, such as DENV of different serotypes or different pathogens of the same genus i.e., flavivirus. Proposed antigenic design approach initiated with DENV antigens allowed further modifications of the immunogenic fusion polypeptide, such as addition of antigenic modules designed for protection against other flaviviruses, e.g. Zika virus (ZIKV) and yellow fever virus (YFV), thus creating a polyvalent vaccine that could simultaneously protect against several flaviviruses at the same time.

In one aspect, the present invention relates to a recombinant polynucleotide comprising at least one polynucleotide encoding a fusion polypeptide, which comprises MHC class I T-cell epitopes suitable to elicit a T cell response, wherein the MHC class I T-cell epitopes originate from a plurality of conserved proteins, in particular non-structural proteins wherein the antigens are from at least one flavivirus selected from the group of Dengue virus (DENV), ZIKA virus (ZIKV) and Yellow Fever virus (YFV).

In a particular aspect of the invention, a first recombinant polynucleotide encodes a first fusion polypeptide which comprises MHC class I T-cell epitopes originating from more than one non-structural DENV proteins and forming an assembled DENV-based antigen exhibiting a consensus amino acid sequence of DENV-1, DENV-2, DENV-3 and DENV-4 strains.

In another aspect of the invention, another, second, recombinant polynucleotide is provided that encodes a, second, fusion polypeptide which comprises MHC class I T-cell epitopes originating from more than one conserved ZIKV protein, in particular from more than one non-structural ZIKV protein and accordingly forming an assembled ZIKV-based antigen.

In another aspect of the invention, another, third, polynucleotide is provided that encodes a, third, fusion polypeptide which comprises MHC class I T-cell epitopes originating from more than one conserved YFV protein in particular from more than one non-structural YFV protein and accordingly forming an assembled YFV-based antigen.

The invention also relates to a recombinant lentiviral vector genome comprising at least one of the plurality of the herein disclosed recombinant polynucleotides encoding fusion polypeptide(s) wherein each fusion polypeptide comprises MHC class I T-cell epitopes originating from more than one conserved DENV, ZIKV and/or YFV proteins, in particular from more than one non-structural DENV, ZIKV and/or YFV proteins.

The present invention further relates to a DNA plasmid comprising the recombinant lentiviral vector genome according to the invention.

The present invention also relates to a recombinant lentiviral vector i.e., a recombinant lentiviral vector particle which comprises the recombinant lentiviral vector genome according to the invention.

The present invention also relates to a fusion polypeptide encoded by the recombinant polynucleotide and to a fusion polypeptide expressed by the recombinant lentiviral vector.

The invention further relates to a host cell, preferably a mammalian host cell, in particular a human host cell, transfected with a DNA plasmid according to the invention, in particular wherein said host cell is a HEK-293T cell line or a K562 cell line.

In another aspect, the invention relates to a pharmaceutical composition, in particular a vaccine composition, suitable for administration to a mammalian host, in particular a human host, comprising a recombinant polynucleotide or a recombinant lentiviral vector of the invention, together with one or more pharmaceutically acceptable excipient(s) suitable for administration to a host in need thereof, in particular a mammalian host, especially a human host.

In particular, the invention relates to the pharmaceutical composition for use in the elicitation of a protective, preferentially prophylactic, immune response by the elicitation of T-cell responses, especially CD8+ T-cell responses, directed against epitopes contained in the antigenic fusion polypeptide(s) or immunogenic fragments thereof in a host in need thereof, in particular a mammalian host, especially a human host.

Another aspect of the invention relates to a method for the preparation of recombinant lentiviral vector particles suitable for the preparation of a pharmaceutical composition, in particular a vaccine composition, comprising the following steps:
a) transfecting the recombinant lentiviral transfer vector carrying the lentiviral vector genome according to the invention, or the DNA plasmid according to the invention in a host cell, for example a HEK-293T cell line or a K562 cell line;
b) co-transfecting the cell of step a) with: (i) a plasmid vector encoding the lentiviral GAG and POL or mutated POL protein as packaging construct; and (ii) a plasmid encoding an envelope protein of a virus that is not a HIV virus and advantageously not a lentivirus, such as a VSV-G Indiana or New Jersey envelope;
c) culturing the host cell under conditions suitable for the production of recombinant lentiviral vector particles expressing the fusion polypeptide(s) of the invention;
d) recovering the recombinant lentiviral particles expressing the fusion polypeptide(s) of the invention.

### DETAILED DESCRIPTION OF THE INVENTION

The inventors have designed and prepared a recombinant polynucleotide comprising at least a recombinant polynucleotide encoding a fusion polypeptide which comprises MHC class I T-cell epitopes suitable to elicit a T cell response, wherein the MHC class I T-cell epitopes originate from a plurality of antigens, in particular non-structural antigens, wherein the antigens are from at least one flavivirus selected from the group of Dengue virus (DENV), ZIKA virus (ZIKV) and Yellow Fever virus (YFV).

In an embodiment, a recombinant polynucleotide of the invention enables the expression of a fusion polypeptide comprising epitopes for the elicitation of a multivalent immune response against multiple serotypes of the Dengue virus (DENV), especially against the 4 known serotypes of the Dengue virus. The invention accordingly concerns a recombinant polynucleotide comprising a first polynucleotide encoding a first fusion polypeptide which comprises MHC class I T-cell epitopes originating from more than one non-structural DENV proteins and forming an assembled DENV-based consensus antigen of DENV-1, DENV-2, DENV-3 and DENV-4 strains. DENV-1, DENV-2, DENV-3 and DENV-4 strains are respectively virus strains of the DENV serotype 1 (DENV-1), DENV serotype 2 (DENV-2), DENV serotype 3 (DENV-3) and DENV serotype 4 (DENV-4).

In another embodiment, a recombinant polynucleotide of the invention enables the expression of a second fusion polypeptide comprising epitopes for the elicitation of an immune response against the ZIKA virus. The invention accordingly concerns a recombinant polynucleotide comprising a polynucleotide encoding a fusion polypeptide which comprises MHC class I T-cell epitopes originating from more than one ZIKV proteins and forming an assembled ZIKV-based antigen, in particular an assembled non-structural antigen.

In another embodiment, a recombinant polynucleotide of the invention enables the expression of a third fusion polypeptide comprising epitopes for the elicitation of an immune response against the Yellow Fever virus. The invention accordingly concerns a recombinant polynucleotide comprising a polynucleotide encoding a fusion polypeptide which comprises MHC class I T-cell epitopes originating from more than one YFV proteins and forming an assembled YFV-based antigen, in particular an assembled non-structural antigen.

In an embodiment the first polynucleotide contains or consists of a single Open Reading Frame (ORF), in particular an ORF encoding a first fusion polypeptide which is an assembled DENV-based consensus antigen. In another embodiment the recombinant polynucleotide comprises or consists of 2 or 3 ORFs wherein each ORF encodes a fusion polypeptide which is an assembled DENV-based consensus antigen, an assembled ZIKV-based antigen or an assembled YFV-based antigen as disclosed herein. In such a case the fusion polypeptide encompasses the first, the second and the third fusion polypeptides.

The expression “T-cell epitope” refers to antigenic determinants that are involved in the adaptive immune response driven by T cells. In particular said T-cell epitopes elicit T cells, when delivered to the host in suitable conditions. According to the invention the fusion polypeptides comprise epitope(s) mediating CD8⁺ T-cell response. In particular the T-cell epitopes of the fusion polypeptide of the invention are MHC Class-I (MHC-I) epitopes suitable for immune response through MHC Class-I presentation machinery, i.e., proteasome, for further triggering of CD8⁺ T cells in a host, especially specific CD8⁺ T cells against the virus targeted with the fusion polypeptide.

The expressions “originating” or “originates” in plural or singular used in the present description by reference to the MHC class I T-cell epitopes, refers to the fact that the expressed epitopes are characteristic of a viral antigen in that they have immunogenic properties that allow a targeted immune response against this determined virus antigen. For this reason, the MHC class I T-cell epitopes that “originate” from a viral antigen may also be designated herein as MHC class I T-cell epitopes “from” such viral antigen thereby expressing that the MHC class I T-cell epitopes is a region separated from or contained in the whole viral antigen or that such region in the viral antigen sequence is a model for the design of the MHC class I T-cell epitopes.

The epitopes are also said to originate from a virus antigen when the fusion polypeptide according to the invention that contains them elicits a targeted immune response against the virus when administered to a host, in particular when the fusion polypeptide is expressed from a LV vector administered to the host. Known methods may be used to assess the immunogenic properties of a T-cell epitope that encompass intracytoplasmic cytokine staining, ELISpot, in vitro stimulation, or proliferation of immune cells. In particular, the epitopes originating from a determined virus antigen have been selected starting from the known available sequences (amino acid sequences and/or nucleotide sequences) of virus antigens. Epitopes for use in the invention are characterized by an amino acid sequence that reflects the native sequence of a determined antigen in the virus or are derived from such sequence containing known or predicted T-cell epitopes for the virus by amino acid mutations. The T-cell epitope may accordingly be identical to a sequence in a native epitope-containing region of a determined antigen of the virus or may be designed as a mutated sequence with respect to such native sequence, e.g. to define a consensus sequence (such as SEQ ID No. 166 for DENV1-4 serotypes or SEQ ID No. 169 for ZIKV_all), or an optimized consensus sequence. Accordingly a mutated sequence or a consensus sequence may be designed using tools available to determine epitopes and tested for presentation by HLA allele, in particular by human MHC class I (MHC class I T-cell epitopes). Prediction tools for the identification of epitopes are known from the person skilled in the art and include T-cell epitope prediction tools available at IEDB database and analysis resource and at the website of Technical University of Denmark (DTU)/Department of Health Technology (Health Tech), such as TepiTool, Proteasomal cleavage/TAP transport/MHC class I combined predictor, and netCTLpan (21, 25). In particular the obtained MHC class I T-cell epitopes expressed by the polynucleotide(s) of the invention are 8-, 9-, 10- and/or 11-mer peptides. The Examples disclosed herein provide a detailed description of the identification of epitopes suitable for the Dengue virus and such description may be adapted for other viruses of interest in the present invention such as ZIKV or YFV to enable preparation of a fusion polypeptide encompassing such epitopes. The viral antigens selected to identify the T-cell epitopes encompass non-structural proteins (for the DENV, for the ZIKV and for the YFV) and may optionally encompass structural proteins (especially for the ZIKV and for the YFV).

As mentioned above, in an embodiment, the invention concerns a recombinant polynucleotide comprising a first polynucleotide encoding a first fusion polypeptide which comprises MHC class I T-cell epitopes originating from more than one non-structural DENV proteins and forming an assembled DENV-based consensus antigen of DENV-1, DENV-2, DENV-3 and DENV-4 strains, wherein the non-structural proteins or antigens are selected from the group of NS1, NS2A, NS2B, NS3, NS4A, NS4B or NS5. The epitopes are said to originate from a viral antigen when the fusion polypeptide that contains them elicits a targeted immune response against a Dengue virus, especially against one or more strains, preferably against all strains, selected from the group of the DENV-1, DENV-2, DENV-3 and DENV-4 serotypes and more preferably against strains in the 4 DENV serotypes. Such epitopes are characterized by an amino acid sequence that reflects the native sequence of an antigen of a determined DENV strain and/or are based on or derived from such sequence containing known or predicted T cell epitopes, by mutations, especially point mutation with respect to the sequence of the native antigen of one or more DENV serotypes. The T cell epitope may accordingly be identical to a sequence in a native epitope-containing region of a DENV antigen selected from the group of non-structural proteins of DENV serotypes or may be a defined consensus sequence, or an optimized consensus sequence designed using tools available to determine epitopes and tested for presentation by HLA alleles, in particular by human HLA alleles class I (MHC class I T cell epitopes). The Examples herein provide a detailed description of the identification of DENV epitopes and preparation of fusion polypeptides encompassing such epitopes. The design of the polyepitopes is accordingly based on the preparation of a consensus sequence (primary consensus) of the antigens of interest for each of the 4 DENV serotypes and the alignment of these 4 consensus sequences for the preparation of a further level of consensus sequence (master consensus sequence) of these 4 consensus sequences. The resulting master consensus sequence is either used to provide the epitopes of the fusion polypeptide or is modified by point mutations or by addition of further epitopes considered suitable to reflect specific variability among the 4 serotypes. Point mutations at a few positions may allow to switch for an amino acid residue that is most represented in the dataset of the epitope containing region(s) in the antigen used to identify the primary 4 consensus sequences of the DENV serotypes. Optionally, in particular when amino acid variation in an epitope-containing region of the original antigen is substantial among the considered dataset of the sequences to design the primary consensus sequences, an additional short sequence of the antigen may be advantageously included in the fusion polypeptide to represent the consensus of the remaining genotypes (with respect to the genotypes reflected in the primary or the master consensus sequence) in the final polyepitope(s). The prediction tools to start with the identification of epitopes are known from the person skilled in the art and include T cell epitope prediction tools available at IEDB database and analysis resource and at the website of Technical University of Denmark (DTU)/Department of Health Technology (Health Tech), such as TepiTool, Proteasomal cleavage/TAP transport/MHC class I combined predictor, and netCTLpan (21, 25).

In a particular embodiment, the recombinant polynucleotide encodes a first fusion polypeptide which comprises MHC class I T-cell epitopes that originate from at least 2 antigens of DENV selected from the group of the NS3, NS4A, NS4B and NS5 antigens and preferably originate from each of the NS3, NS4A, NS4B and NS5 antigens. The wild-type antigens of the various serotypes of DENV are well known in the art and are available from the public databases such as NCBI and IEDB.

In a particular embodiment, the recombinant polynucleotide encodes a second fusion polypeptide which comprises MHC class I T-cell epitopes that originate from at least 2 non-structural antigens of ZIKV that are the NS4B and NS5 antigens and further comprises MHC class I T-cell epitopes that originate from C and PrM antigens of the ZIKV. The wild-type antigens of ZIKV are well known in the art and are available from the public databases such as NCBI and IEDB.

In a particular embodiment, the recombinant polynucleotide encodes a third fusion polypeptide which comprises MHC class I T-cell epitopes that originate from at least 2 antigens of YFV selected from the group of the NS1, NS2A, NS2B, NS3, NS4A, NS4B and NS5 antigens and preferably originate from each of the NS2A, NS2B, NS3, NS4A, NS4B and NS5 antigens. The wild-type antigens of YFV are well known in the art and are available from the public databases such as NCBI and IEDB.

The recombinant polynucleotides of the ZIKV and YFV constructs may be designed in accordance with the description provided herein in relation to the DENV serotypes, especially involving a step of preparation of a consensus sequence for each of ZIKV and YFV.

In an embodiment, the recombinant polynucleotide encodes a fusion polypeptide(s) as disclosed above and in further embodiments hereafter which comprise(s) MHC class I T-cell epitopes that are selected (for example using NetCTLpan tool) for their properties to contain human epitopes suitable for presentation by 12 human HLA supertypes alleles (HLA-A*01:01, HLA-A*02:01, HLA-A*03:01, HLA-A*24:02, HLA-A*26:01, HLA-B*07:02, HLA-B*08:01, HLA-B*27:05, HLA-B*39:01, HLA-B*40:01, HLA-B*58:01, HLA-B*15:01) or advantageously by 27 human HLA alleles most commonly distributed worldwide, including HLA-A*01:01, HLA-A*02:01, HLA-A*02:03, HLA-A*02:06, HLA-A*03:01, HLA-A*11:01, HLA-A*23:01, HLA-A*24:02, HLA-A*26:01, HLA-A*30:01, HLA-A*30:02, HLA-A*31:01, HLA-A*32:01, HLA-A*33:01, HLA-A*68:01, HLA-A*68:02, HLA-B*07:02, HLA-B*08:01, HLA-B*15:01, HLA-B*35:01, HLA-B*40:01, HLA-B*44:02, HLA-B*44:03, HLA-B*51:01, HLA-B*53:01, HLA-B*57:01, and HLA-B*58:01, in particular when they originate from non-structural DENV antigens as disclosed herein. The above listed 12 HLA supertypes are chosen to best represent the global variety of the HLA molecules (i.e. predictions based on that set should approximate representation by ALL possible HLA-A and HLA-B alleles) (36). The above set of 27 most prevalent human HLA-A and HLA-B alleles includes alleles that should by expressed by 97% of human population (37), i.e., includes alleles that are most common in global human population, but not necessarily most divergent. These 2 sets accordingly do not overlap completely.

The recombinant polynucleotide(s) of the invention are provided as fusion polynucleotide(s) wherein the fragments originating from the distinct viral antigens or the MHC class I T-cell epitopes of such antigens are assembled or fused together through junction regions. In a preferred embodiment of the invention the formed junction regions are devoid of non-specific epitopes or neoepitopes that could elicit a non-specific immune response in a host.

The expression «junction region» relates to each region in the assembled recombinant polynucleotide, in particular in the recombinant polynucleotide that encodes the DENV-based consensus antigen, the ZIKV-based antigen or the YFV-based antigen, that links successive protein domains originating from the virus, in particular from the non-structural proteins of the virus, when such domains are not naturally consecutive in the native or in the consensus sequence originating from the considered viral protein(s). In an embodiment, the junction region merely consists of a nucleic acid region of the recombinant polynucleotide encoding the amino acid residues that belong to two different domains or proteins that are fused and that are adjacent to the site where the fusion of the two domains or protein fragments takes place, in particular the junction region encodes a region of 2 to 10 amino acid residues displayed around the fusion site of the two domains. In such embodiment the junction region does not add nucleotide or amino acid residues to those constituting the antigenic domains. In another embodiment, the junction region consists of a nucleic acid region encoding the amino acid residues that belong to two different domains or protein fragments that are fused and that are adjacent to the site where the fusion of the two domains or protein fragments takes place with the provision that the fusion site further includes nucleotides encoding a determined linker. According to this embodiment, the junction region may encode a region added to the antigenic domains, as an addition of 2 to 10 amino acid residues, in particular of 2 to 9 amino acid residues that is functionally a linker. Linkers are determined by the person skilled in the art in the context of the adjacent antigenic domains and usually contain hydrophobic amino acid residues. Examples of linkers are provided in the constructs disclosed herein and should be used or adapted to avoid neoepitope formation within the junction region formed with adjacent viral antigenic domains.

In a preferred embodiment, the junction regions include hydrophobic amino acid linkers and are devoid of sequences encoding non-specific immunodominant epitopes. The present disclosure describes specific linkers suitable for use according to the invention, by their sequence. Such specific examples should not be considered limiting as the person skilled in the art should be able to design alternative linkers especially taking into account the following conditions illustrated by steps taken for assembly of complete antigens of DENV (DENV-Ag1) and YFV (YFV-Ag1 and YFV-Ag2):
1. Chosen antigenic regions were assembled pairwise and used to predict human MHC class I epitopes (using NetCTLpan tool) presentable by 12 HLA Class I supertypes (HLA-A*01:01, HLA-A*02:01, HLA-A*03:01, HLA-A*24:02, HLA-A*26:01, HLA-B*07:02, HLA-B*08:01, HLA-B*27:05, HLA-B*39:01, HLA-B*40:01, HLA-B*58:01, HLA-B*15:01) to assure that the epitopes were predicted to form correctly and no immunodominant neoepitopes were created due to joining of regions together. In the case if a neoepitope was not detected by epitope prediction algorithm, the assembly of regions was retained and the next region was joined either to the N-terminus or the C-terminus of the assembly, after which the prediction was repeated again. If such stepwise assembly process resulted in the detection of predictable neoepitope after addition of a particular region, another region was tried in its place until all combinations of regions that could be assembled without creating a neoepitope were identified.
2. At the second step (for the regions that could not be added to the antigen without creating neoepitopes), each adjacent antigenic region was extended by adding 3-4 amino acid residues (preferably 3-4 amino acid-long sequences that were bordering (adjacent to) each antigenic regions in the “native” (e.g. consensus) viral sequence). If such extension did not eliminate neoepitope, certain amino acids in the 6-8 aa-long “perspective” junction region that were found to be (empirical observation) frequently present in MHC class I epitopes (mostly non-polar amino acids: M, L, F, V, I, A, T, Y) were replaced one by one by the amino acids that are detected less often (certain non-polar amino acids: G, C, N, P, W, and polar amino acids: D, E, K, R, Q, H, S).
3. If the approach outlined above resulted in elimination of a predicted neoepitope, the junction was further optimized by reducing the number of “extra” amino acids between the regions. Alternatively, if the outlined approach was not successful, the junction region was extended (again, by adding amino acids that are less often detected inside MHC class I epitopes) until the neo-epitope at the junction site was no longer detectable.

Same strategy was also followed to design ZIKV-Ag, except that in that case no rearrangement of antigenic regions was performed and they were assembled in the same order in which they appear in viral genome.

In an embodiment, the invention relates to a recombinant polynucleotide wherein the assembled DENV-based consensus antigen comprises the polynucleotides encoding the MHC class I T-cell epitopes: SEQ ID No. 2, SEQ ID No. 4, SEQ ID No. 6, SEQ ID No. 8, SEQ ID No. 10, SEQ ID No. 12, SEQ ID No. 14, SEQ ID No. 16, SEQ ID No. 18, SEQ ID No. 20, SEQ ID No. 22, SEQ ID No. 24, SEQ ID No. 26, SEQ ID No. 28 or a variant thereof which is devoid of SEQ ID No. 2 and comprises starting in the 5′ end, SEQ ID No. 32, SEQ ID No. 34 and SEQ ID No. 36, wherein optionally the polynucleotides encoding the above listed polypeptides are provided from 5′ to 3′ in the recombinant polynucleotide in accordance with the order of the amino acid sequences in the above list.

In a particular embodiment of this aspect of the invention, the recombinant polynucleotide is selected among:
a. the recombinant polynucleotide which comprises the polynucleotides encoding the MHC class I T-cell epitopes of the following sequences, arranged from 5′ to 3′ in the recombinant polynucleotide in accordance with the following order: SEQ ID No. 2, SEQ ID No. 4, SEQ ID No. 6, SEQ ID No. 8, SEQ ID No. 10, SEQ ID No. 12, SEQ ID No. 14, SEQ ID No. 16, SEQ ID No. 18, SEQ ID No. 20, SEQ ID No. 22, SEQ ID No. 24, SEQ ID No. 26 and SEQ ID No. 28 and wherein the junction regions between said polynucleotides encoding the MHC class I T-cell epitopes are devoid of sequences encoding non-specific immunodominant epitopes, in particular the recombinant polynucleotide wherein a hydrophobic amino acid linker sequence is inserted as a junction region between all consecutive above sequences except between SEQ ID No. 8 and SEQ ID No. 10, between SEQ ID No. 16 and SEQ ID No. 18 and between SEQ ID No. 18 and SEQ ID No. 20 or,
b. the recombinant polynucleotide which comprises the polynucleotides encoding the MHC class I T-cell epitopes of the following sequences, arranged from 5′ to 3′ in the recombinant polynucleotide in accordance with the following order: SEQ ID No. 32, SEQ ID No. 34 and SEQ ID No. 36, SEQ ID No. 4, SEQ ID No. 6, SEQ ID No. 8, SEQ ID No. 10, SEQ ID No. 12, SEQ ID No. 14, SEQ ID No. 16, SEQ ID No. 18, SEQ ID No. 20, SEQ ID No. 22, SEQ ID No. 24, SEQ ID No. 26, SEQ ID No. 28, and wherein the junction regions between said polynucleotides encoding the MHC class I T-cell epitopes are devoid of sequences encoding non-specific immunodominant epitopes, in particular the recombinant polynucleotide wherein a hydrophobic amino acid linker sequence is inserted as a junction region between all consecutive above sequences except between SEQ ID No. 34 and SEQ ID No. 36, between SEQ ID No. 8 and SEQ ID No. 10, between SEQ ID No. 16 and SEQ ID No. 18 and between SEQ ID No. 18 and SEQ ID No. 20.

In an embodiment, the invention relates to a recombinant polynucleotide wherein the assembled ZIKV-based antigen comprises the polynucleotides encoding the MHC class I T-cell epitopes: SEQ ID No. 40, SEQ ID No. 42, SEQ ID No. 44, SEQ ID No. 46, SEQ ID No. 48, SEQ ID No. 50, SEQ ID No. 52, wherein optionally the polynucleotides encoding the above listed polypeptides are provided from 5′ to 3′ in the recombinant polynucleotide in accordance with the order of the amino acid sequences in the above list.

In a particular embodiment of this aspect of the invention, the recombinant polynucleotide comprises the polynucleotides encoding the MHC class I T-cell epitopes arranged from 5′ to 3′ in the recombinant polynucleotide in accordance with the following order: SEQ ID No. 40, SEQ ID No. 42, SEQ ID No. 44, SEQ ID No. 46, SEQ ID No. 48, SEQ ID No. 50 and SEQ ID No. 52 and wherein the junction regions between said polynucleotides encoding the MHC class I T-cell epitopes are devoid of sequences encoding non-specific immunodominant epitopes, in particular the recombinant polynucleotide wherein a hydrophobic amino acid linker sequence is inserted as a junction region between all consecutive above sequences except between SEQ ID No. 44 and SEQ ID No. 46 and between SEQ ID No. 50 and SEQ ID No. 52;

In an embodiment, the invention relates to a recombinant polynucleotide wherein the assembled antigen based on non-structural proteins of YFV (YFV-Ag1) comprises the polynucleotides encoding the MHC class I T-cell epitopes: SEQ ID No. 58, SEQ ID No. 60, SEQ ID No. 62, SEQ ID No. 64, SEQ ID No. 66, SEQ ID No. 68, SEQ ID No. 70, SEQ ID No. 72, SEQ ID No. 74, SEQ ID No. 76, SEQ ID No. 78, SEQ ID No. 80, SEQ ID No. 82, SEQ ID No. 84, SEQ ID No. 86, SEQ ID No. 88, SEQ ID No. 90, SEQ ID No. 92, SEQ ID No. 94, SEQ ID No. 96, SEQ ID No. 98, SEQ ID No. 100, SEQ ID No. 102, SEQ ID No. 104 wherein optionally the polynucleotides encoding the above listed polypeptides are provided from 5′ to 3′ in the recombinant polynucleotide in accordance with the order of the amino acid sequences in the above list.

In an embodiment, the invention relates to a recombinant polynucleotide wherein the assembled antigen based on structural proteins of YFV and non-structural protein NS1 (YFV-Ag2) comprises the polynucleotides encoding the MHC class I T-cell epitopes: SEQ ID No. 108, SEQ ID No. 110, SEQ ID No. 112, SEQ ID No. 114, SEQ ID No. 116, SEQ ID No. 118, SEQ ID No. 120, SEQ ID No. 122, SEQ ID No. 124, SEQ ID No. 126, SEQ ID No. 128, SEQ ID No. 130, SEQ ID No. 132, SEQ ID No. 134, SEQ ID No. 136, SEQ ID No. 138, SEQ ID No. 140, wherein optionally the polynucleotides encoding the above listed polypeptides are provided from 5′ to 3′ in the recombinant polynucleotide in accordance with the order of the amino acid sequences in the above list.

In a particular embodiment of this aspect of the invention, the recombinant polynucleotide is selected among:
a. the polynucleotide which comprises polynucleotides encoding the MHC class I T-cell epitopes arranged from 5′ to 3′ in the recombinant polynucleotide in accordance with the following order: SEQ ID No. 58, SEQ ID No. 60, SEQ ID No. 62, SEQ ID No. 64, SEQ ID No. 66, SEQ ID No. 68, SEQ ID No. 70, SEQ ID No. 72, SEQ ID No. 74, SEQ ID No. 76, SEQ ID No. 78, SEQ ID No. 80, SEQ ID No. 82, SEQ ID No. 84, SEQ ID No 86, SEQ ID No. 88, SEQ ID No. 90, SEQ ID No. 92, SEQ ID No. 94, SEQ ID No. 96, SEQ ID No. 98, SEQ ID No. 100, SEQ ID No. 102 and SEQ ID No. 104 and wherein the junction regions between said polynucleotides encoding the MHC class I T-cell epitopes are devoid of sequences encoding non-specific immunodominant epitopes, in particular the recombinant polynucleotide wherein a hydrophobic amino acid linker sequence is inserted as a junction region between all consecutive above sequences except between SEQ ID No. 60 and SEQ ID No. 62, between SEQ ID No. 66 and SEQ ID No. 68, between SEQ ID No. 74 and SEQ ID No. 76, between SEQ ID No. 78 and SEQ ID No. 80 and SEQ ID No. 82 and between SEQ ID No. 94 and SEQ ID No. 96, or
b. the polynucleotide which comprises polynucleotides encoding the MHC class I T-cell epitopes arranged from 5′ to 3′ in the recombinant polynucleotide in accordance with the following order: SEQ ID No. 108, SEQ ID No. 110, SEQ ID No. 112, SEQ ID No. 114, SEQ ID No. 116, SEQ ID No. 118, SEQ ID No. 120, SEQ ID No. 122, SEQ ID No. 124, SEQ ID No. 126, SEQ ID No. 128, SEQ ID No. 130, SEQ ID No. 132, SEQ ID No. 134, SEQ ID No. 136, SEQ ID No. 138 and SEQ ID No. 140 and wherein the junction regions between said polynucleotides encoding the MHC class I T-cell epitopes are devoid of sequences encoding non-specific immunodominant epitopes, in particular the recombinant polynucleotide wherein a hydrophobic amino acid linker sequence is inserted as a junction region between all consecutive above sequences except between SEQ ID No. 110, SEQ ID No. 112, SEQ ID No. 114, SEQ ID No. 116 and SEQ ID No. 118 and between SEQ ID No. 124 and SEQ ID No 126.

In another embodiment, the recombinant polynucleotide is a nucleic acid molecule whose sequence is modified with respect to at least one of the sequences of SEQ ID No. disclosed above for DENV fusion polynucleotides or with respect to at least one of the sequences of SEQ ID No. disclosed above for ZIKV polynucleotide or with respect to at least one of the sequences of SEQ ID No. disclosed above for YFV polynucleotide, wherein the modification consists of point mutation of one or more nucleotides, in particular of substitution or deletion of nucleotides, and the modified sequence encodes a fusion polypeptide that has at least 90% sequence identity, at least 94%, or at least 95% sequence identity or has from 94% to 99% sequence identity with the sequence of the original fusion polypeptide. A modified sequence as herein defined is regarded as a variant sequence with respect to the sequence of reference.

In an embodiment, the recombinant fusion polynucleotide, including one which would have a variant sequence as defined herein encodes an antigenic domain of fusion polypeptide of the sequences or a variant thereof as disclosed above, wherein the polynucleotide comprises the following operably linked nucleotides sequences:
(i) SEQ ID No. 1, SEQ ID No. 3, SEQ ID No. 5, SEQ ID No. 7, SEQ ID No. 9, SEQ ID No. 11, SEQ ID No. 13, SEQ ID No. 15, SEQ ID No. 17, SEQ ID No. 19, SEQ ID No. 21, SEQ ID No. 23, SEQ ID No. 25, SEQ ID No. 27, or a variant thereof wherein SEQ ID No. 1 is deleted and which comprises in the 5′ end, SEQ ID No. 31 and/or SEQ ID No. 33 and/or SEQ ID No. 35, wherein optionally these polynucleotide fragments are provided from 5′- to 3′- in the fusion polynucleotide in accordance with the order in the above list (from SEQ ID No. 1 to SEQ ID No. 27 or from SEQ ID No. 31 to SEQ ID No. 27 (excluding SEQ ID No. 1) according to the above disclosure) or
(ii) SEQ ID No. 39, SEQ ID No. 41, SEQ ID No. 43, SEQ ID No. 45, SEQ ID No. 47, SEQ ID No. 49, SEQ ID No. 51, wherein optionally these polynucleotide fragments are provided from 5′- to 3′- in the fusion polynucleotide in accordance with the order in the above list (from SEQ ID No. 39 to SEQ ID No. 51 according to the above disclosure), or
(iii) SEQ ID No. 57, SEQ ID No. 59, SEQ ID No. 61, SEQ ID No. 63, SEQ ID No. 65, SEQ ID No. 67, SEQ ID No. 69, SEQ ID No. 71, SEQ ID No. 73, SEQ ID No. 75, SEQ ID No. 77, SEQ ID No. 79, SEQ ID No. 81, SEQ ID No. 83, SEQ ID No 85, SEQ ID No. 87, SEQ ID No. 89, SEQ ID No. 91, SEQ ID No. 93, SEQ ID No. 95, SEQ ID No. 97, SEQ ID No. 99, SEQ ID No. 101, SEQ ID No. 103, wherein optionally these polynucleotide fragments are provided from 5′- to 3′- in the fusion polynucleotide in accordance with the order in the above list (from SEQ ID No. 57 to SEQ ID No. 103 according to the above disclosure), or
(iv) SEQ ID No. 107, SEQ ID No. 109, SEQ ID No. 111, SEQ ID No. 113, SEQ ID No. 115, SEQ ID No. 117, SEQ ID No. 119, SEQ ID No. 121, SEQ ID No. 123, SEQ ID No. 125, SEQ ID No. 127, SEQ ID No. 129, SEQ ID No. 131, SEQ ID No. 133, SEQ ID No. 135, SEQ ID No. 137, SEQ ID No. 139, wherein optionally these polynucleotides are provided from 5′ to 3′ in the recombinant polynucleotide in accordance with the order in the above list (from SEQ ID No. 107 to SEQ ID No. 139 according to the above disclosure).

In a particular embodiment, the recombinant polynucleotide further contains a sequence encoding a signal peptide at its 5′-end.

In a particular embodiment, the recombinant polynucleotide is a nucleic acid molecule whose sequence consists of SEQ ID No. 29 or SEQ ID No. 37.

In a particular embodiment, the recombinant polynucleotide is a nucleic acid molecule whose sequence consists of SEQ ID No. 53 or SEQ ID No. 55.

In a particular embodiment, the recombinant polynucleotide is a nucleic acid molecule whose sequence consists of SEQ ID No. 105 or SEQ ID No. 141.

In a particular embodiment, the recombinant polynucleotide is a fusion of one polynucleotide selected from the group of SEQ ID No. 29 and SEQ ID No. 37 with one polynucleotide selected from the group of SEQ ID No. 53 and SEQ ID No. 55 and/or one polynucleotide selected from the group of SEQ ID No. 105 and SEQ ID No. 141. In a particular embodiment, the recombinant polynucleotide is a fusion of the polynucleotide of SEQ ID No. 29 or SEQ ID No. 37 with a polynucleotide of SEQ ID No. 53 and with a polynucleotide of SEQ ID No. 105 or SEQ ID No. 141. The order of appearance of the above polynucleotides defined by their SEQ ID No. within the sequence of the recombinant polynucleotide is chosen by the person skilled in the art. In a particular embodiment the order is such that the polynucleotide encoding MHC Class I T-cell epitopes originating from DENV appears first in the 5′ to 3′ sense of the recombinant polynucleotide.

In a particular embodiment, the recombinant polynucleotide is selected from the group of SEQ ID No. 143, SEQ ID No. 145, SEQ ID No. 147 and SEQ ID No. 149.

In another embodiment, the recombinant polynucleotide is a nucleic acid molecule whose sequence is modified with respect to the sequence of SEQ ID No. 29 or SEQ ID No. 37 or with respect to the sequence of SEQ ID No. 53 or SEQ ID No. 55 or with respect to the sequence of SEQ ID No. 105 or SEQ ID No. 141, or with respect to one of the sequences of SEQ ID No. 143, SEQ ID No. 145, SEQ ID No. 147 and SEQ ID No. 149 wherein the modification consists of point mutation of one or more nucleotides, in particular of substitution or deletion of nucleotides, and the modified sequence encodes a fusion polypeptide that has at least 90% sequence identity, at least 94%, or at least 95% sequence identity or has from 94% to 99% sequence identity with the sequence of the original fusion polypeptide.

In an embodiment, the recombinant fusion polynucleotide, including one which would have a variant sequence as defined herein encodes a fusion polypeptide of SEQ ID No. 30 or SEQ ID No 38 or of SEQ ID No. 54 or SEQ ID No. 56 or of SEQ ID No. 106 or SEQ ID No. 142 or a variant thereof as disclosed above.

In a particular embodiment, the sequence of the polynucleotide is modified with respect to the sequence of SEQ ID No. 29 or SEQ ID No. 37 wherein the modification consists of substitution of antigenic domains in the NH₂-terminal sequence of the fusion polypeptide, by supplementary antigenic regions representative of a antigenic domains of a selected subgroup of DENV serotypes.

In an embodiment the point mutation(s) consists in changing amino acid residues for residues present in DENV-1, DENV-2, DENV-3 and/or DENV-4 genotype(s), especially to select amino acid residues shared by at least two of these genotypes.

In an embodiment the recombinant fusion polynucleotide is a fusion ORF encoding MHC class I T-cell epitopes as disclosed herein wherein the coding sequence is under the control of transcription and translation control elements, especially within a single transcriptional regulation unit under the control of a single promoter for at least the MHC class I T-cell epitopes originating from the same virus group. Transcription and translation control elements may be such as disclosed hereafter in relation for the transfer vector of the vector genome.

In an embodiment the recombinant polynucleotide may be nucleic acid molecule encoding MHC class I T-cell epitopes originating from DENV proteins and from at least one of the ZIKV proteins and YFV proteins as disclosed herein wherein the nucleic acid sequences are operably linked. According to such embodiment, the recombinant polynucleotide may comprise one or more expression cassettes for MHC class I T-cell epitopes originating from different viruses including DENV and at least one of ZIKV and YFV. In a particular embodiment, the recombinant polynucleotide comprises one expression cassette for MHC class I T-cell epitopes originating from Dengue virus and ZIKA virus wherein the nucleic acid sequence encoding the MHC class I T-cell epitopes originating from Dengue virus and the nucleic acid sequence encoding the MHC class I T-cell epitopes originating from Zika virus are separated by a sequence encoding a self-cleavage peptide, such as a 2A self-cleavage peptide, optionally associated with a spacer sequence (such as one encoding GSG located N-terminal to the 2A self-cleavage peptide). 2A peptides are well known in the art and encompass peptides of 19 to 22 amino acid residues such as the peptide of sequence LLNFDLLKLAGDVESNPGP (SEQ ID No. 217) or 2A-like such as P2A (GSGATNFSLLKQAGDVEENPGP SEQ ID No. 218), T2A, E2A, F2A disclosed in the art, suitable to mediate the simultaneous expression and cleavage of the fusion ORF and cause secretion of the expressed polypeptides.

In a particular embodiment, the recombinant polynucleotide according to the invention comprises (i) a first polynucleotide encoding a first fusion polypeptide comprising MHC class I T-cell epitopes originating from non-structural DENV proteins and forming an assembled DENV-based antigen and further comprises (ii) a second polynucleotide encoding either a second fusion polypeptide comprising MHC class I T-cell epitopes originating from structural and from non-structural ZIKV proteins and forming an assembled ZIKV-based antigen, or an ORF coding for NS1 protein of ZIKV preceded by a signal peptide originating from E protein (SEQ ID No. 56); and further comprises (iii) a third polynucleotide encoding a third fusion polypeptide comprising MHC class I T-cell epitopes originating from structural and/or from non-structural YFV proteins and forming an assembled YFV-based antigen, wherein the first and, when present, the second and third polynucleotides are operably linked in an expression cassette and are separated by a sequence encoding a self-cleavage peptide such as a 2A self-cleavage peptide, optionally associated with a spacer sequence (such as one encoding GSG located N-terminal to the 2A self-cleavage peptide) as disclosed herein.

In a particular embodiment, the first and second polynucleotides encoding respectively MHC class I T-cell epitopes originating from Dengue virus and either MHC class I T-cell epitopes originating from ZIKA virus or an ORF (SEQ ID No. 55) encoding NS1 protein of ZIKV preceded by a signal peptide originating from E protein are assembled from 5′-end to 3′-end as the first polynucleotide followed by the second polynucleotide (such as in exemplified Flavi-2 or Flavi-4 construct) or as the second polynucleotide followed by the first polynucleotide (such as in exemplified Flavi-3 or Flavi-5 construct).

In any of the disclosed embodiments, the recombinant polynucleotide construct may additionally comprise in its 5′-terminal end a nucleic acid sequence encoding a signal peptide and/or additional nucleotides or codons necessary or advantageous for translation of the polynucleotide as a fusion polypeptide disclosed herein, such as nucleotides (as shown in the sequence of the transgenes disclosed herein) encoding the MD amino acid residues in the DENV-Ag1, or MA amino acid residues in the YFV-Ag1 and YFV-Ag2. The added M (Met) codon enables to initiate the translation when such codon is missing in the selected region for assembly. The additional codon is chosen to favor having a stronger Kozak sequence (GCCACCATGG—SEQ ID No. 172) at the 5′ end of the coding region that could lead to a more efficient translation. The last 4 nucleotides of stronger Kozak sequence are part of the coding region, and the last nucleotide (G) is the first position of the codon for the second amino acid. Thus, the preferred amino acid at that position is either A, V, D, E, or G. To choose an amino acid from that list, the inventors performed MHC class I epitope prediction for sequences containing each amino acid in turns, to select an amino acid that would least interfere with correct processing of the first MHC class I epitope located at the N-terminal part of polyepitope.

The nucleic acid of the recombinant polynucleotide disclosed herein may be DNA, in particular cDNA or may be RNA, in particular stabilized RNA. The RNA sequences are deducted from the DNA sequences wherein the Thymine (T) nucleobase is replaced by an Uracile (U) nucleobase. RNA polynucleotides may be obtained by transcription of DNA or cDNA or may be synthesized.

The nucleic acid molecule may further comprise control nucleotide sequences for the transcription or for the expression of the fusion polypeptides. It may also be modified, in order to be operably ligated to a distinct polynucleotide such as a plasmid or a vector genome, in particular a transfer plasmid, in particular a lentiviral vector genome, especially a HIV-1 vector genome as disclosed hereafter. It may also be modified, in particular to be rendered more stable such as for use as RNA. In a further embodiment, the nucleic acid is a mammalian codon-optimized, in particular a human codon-optimized sequence for expression in mammalian, respectively human cells. Examples of codon-optimized nucleic acids are provided in the exemplified constructs of the transgenes.

The invention hence discloses a recombinant lentiviral vector genome comprising at least one recombinant polynucleotide of the invention encoding a fusion polypeptide of the invention wherein the fusion polypeptide is expressed as a multi-domain recombinant protein comprising several antigenic domains comprising MHC class I T-cell epitopes of one or more viruses selected from the group of DENV, ZIKV and YFV.

The fusion polypeptide is encoded by a recombinant polynucleotide as defined herein that is inserted in the backbone of the lentiviral transfer vector to provide a vector genome comprising the recombinant polynucleotide of the invention in order to enable preparing lentiviral vector particles expressing the fusion polypeptide(s) harboring the MHC class I T-cell epitopes for elicitation of an immunological response, in particular a protective immunogenic response or advantageously a sterile protection against the virus(es) from which the epitopes originate.

The “vector genome” of the herein disclosed vector particles is a recombinant nucleic acid which also comprises as an inserted sequence the polynucleotide or transgene of interest encoding the fusion polypeptide according to the invention comprising one or more antigenic polypeptide(s) or immunogenic fragment(s) thereof originating from viruses as disclosed herein, especially wherein the virus is a flavivirus selected from the group of Dengue virus, especially one of the known serotypes DENV-1, DENV-2, DENV-3 and DENV-4, or the Zika virus (ZIKV) or the Yellow Fever virus (YFV). The lentiviral-based sequence and polynucleotide/transgene of the vector genome are borne by a plasmid vector thus giving rise to the “transfer vector” also referred to as “sequence vector” to prepare the lentiviral vector by transfection of producing cells. Accordingly, these expressions are used interchangeably in the present description.

The vector genome as defined herein accordingly contains, apart from the so-called recombinant polynucleotide(s) of the invention encoding the fusion polypeptide of the invention comprising the antigenic polypeptide(s) placed under control of proper regulatory sequences for its expression, the sequences of the original lentiviral genome which are non-coding regions of said genome and are necessary to provide recognition signals for DNA or RNA synthesis and processing (mini-viral genome). These sequences are especially cis-acting sequences necessary for packaging (ψ), reverse transcription (LTRs possibly mutated with respect to the original ones) and transcription and optionally integration (RRE) and furthermore for the particular purpose of the invention, they contain a functional sequence favouring nuclear import in cells and accordingly transgene transfer efficiency in said cells, which element is described as a DNA Flap element that contains or consists of the so-called central cPPT-CTS nucleotidic domain present in lentiviral genome sequences especially in HIV-1 or in some retroelements such as those of yeasts.

The structure and composition of the vector genome used to prepare the lentiviral vectors of the invention are based on the principles described in the art and on examples of such lentiviral vectors primarily disclosed in Zennou et al, 2000; Firat H. et al, 2002; VandenDriessche T. et al., 2002. Constructs of this type have been deposited at the CNCM (Institut Pasteur, France) as will be referred to herein. In this respect reference is also made to the disclosure, including to the deposited biological material, in patent applications WO 99/55892, WO 01/27300 and WO 01/27304.

According to a particular embodiment of the invention, a vector genome may be a replacement vector in which all the viral protein coding sequences between the 2 long terminal repeats (LTRs) have been replaced by the recombinant polynucleotide encoding the fusion polypeptide of the invention comprising the antigenic polypeptide(s) as disclosed herein, and wherein the DNA-Flap element has been re-inserted in association with the required cis-acting sequences described herein. Further features relating to the composition of the vector genome are disclosed in relation to the preparation of the particles.

In a particular embodiment, a lentiviral vector of the invention may comprise in its genome one or more than one recombinant polynucleotide encoding at least one fusion polypeptide according to the invention by way of the vector genome. In particular, said vector genome comprises two polynucleotides which are consecutive or separated on the genome and which encode different fusion polypeptides of distinct antigens of the same virus pathogen or of distinct viruses.

In a particular embodiment, the invention thus relates to a recombinant lentiviral vector genome comprising at least one recombinant polynucleotide (in particular 1, 2 or 3 recombinant polynucleotides) as disclosed in the various embodiments herein and encoding a fusion polypeptide or multiple fusion polypeptides, wherein said fusion polypeptides are as disclosed herein.

In a more particular embodiment, the recombinant lentiviral vector genome encodes a fusion polypeptide that comprises:
a polypeptide comprising MHC class I T-cell epitopes of SEQ ID No. 2, SEQ ID No. 4, SEQ ID No. 6, SEQ ID No. 8, SEQ ID No. 10, SEQ ID No. 12, SEQ ID No. 14, SEQ ID No. 16, SEQ ID No. 18, SEQ ID No. 20, SEQ ID No. 22, SEQ ID No. 24, SEQ ID No. 26, SEQ ID No. 28 or a variant thereof which is devoid of SEQ ID No. 2 and comprises in the 5′ end, SEQ ID No. 32, SEQ ID No. 33 and SEQ ID No. 34 wherein optionally the sequences coding for the epitopes in the above SEQ ID No. are arranged from N-terminal to C-terminal ends according to the order in the above recitation (from SEQ ID No. 2 to SEQ ID No. 28 or from SEQ ID No. 32 to SEQ ID No. 28 (excluding SEQ ID No. 2) according to the above disclosure) or comprising variants thereof as disclosed herein and/or
a polypeptide comprising the MHC class I T-cell epitopes: SEQ ID No. 40, SEQ ID No. 42, SEQ ID No. 44, SEQ ID No. 46, SEQ ID No. 48, SEQ ID No. 50, SEQ ID No. 52, wherein optionally the sequences coding for the epitopes in the above SEQ ID No. are arranged from N-terminal to C-terminal ends according to the order in the above recitation (from SEQ ID No. 40 to SEQ ID No. 52 according to the above disclosure) or comprising variants thereof as disclosed herein and/or
a polypeptide comprising MHC class I T-cell epitopes: SEQ ID No. 58, SEQ ID No. 60, SEQ ID No. 62, SEQ ID No. 64, SEQ ID No. 66, SEQ ID No. 68, SEQ ID No. 70, SEQ ID No. 72, SEQ ID No. 74, SEQ ID No. 76, SEQ ID No. 78, SEQ ID No. 80, SEQ ID No. 82, SEQ ID No. 84, SEQ ID No 86, SEQ ID No. 88, SEQ ID No. 90, SEQ ID No. 92, SEQ ID No. 94, SEQ ID No. 96, SEQ ID No. 98, SEQ ID No. 100, SEQ ID No. 102, SEQ ID No. 104, wherein optionally the sequences coding for the epitopes in the above SEQ ID No. are arranged from N-terminal to C-terminal ends according to the order in the above recitation (from SEQ ID No. 58 to SEQ ID No. 104 according to the above disclosure) or comprising variants thereof as disclosed herein and/or
a polypeptide comprising MHC class I T-cell epitopes: SEQ ID No. 108, SEQ ID No. 110, SEQ ID No. 112, SEQ ID No. 114, SEQ ID No. 116, SEQ ID No. 118, SEQ ID No. 120, SEQ ID No. 122, SEQ ID No. 124, SEQ ID No. 126, SEQ ID No. 128, SEQ ID No. 130, SEQ ID No. 132, SEQ ID No. 134, SEQ ID No. 136, SEQ ID No. 138, SEQ ID No. 140, wherein optionally the sequences coding for the epitopes in the above SEQ ID No. are arranged from N-terminal to C-terminal ends according to the order in the above recitation (from SEQ ID No. 108 to SEQ ID No. 140 according to the above disclosure) or comprising variants thereof as disclosed herein.

In an embodiment of this aspect of the invention, a recombinant lentiviral vector genome comprises at least one recombinant polynucleotide (in particular 1, 2 or 3 recombinant polynucleotides) encoding a fusion polypeptide wherein the polynucleotide comprises the following operably linked nucleotides sequences:
(i) SEQ ID No. 1, SEQ ID No. 3, SEQ ID No. 5, SEQ ID No. 7, SEQ ID No. 9, SEQ ID No. 11, SEQ ID No. 13, SEQ ID No. 15, SEQ ID No. 17, SEQ ID No. 19, SEQ ID No. 21, SEQ ID No. 23, SEQ ID No. 25, SEQ ID No. 27, or a variant thereof wherein SEQ ID No. 1 is deleted and which comprises in the 5′ end, SEQ ID No. 31 and/or SEQ ID No. 33 and/or SEQ ID No. 35, wherein optionally these polynucleotide fragments are provided from 5′- to 3′- in the fusion polynucleotide in accordance with the order in the above list (from SEQ ID No. 1 to SEQ ID No. 27 or from SEQ ID No. 31 to SEQ ID No. 27 (excluding SEQ ID No. 1) according to the above disclosure) and/or
(ii) SEQ ID No. 39, SEQ ID No. 41, SEQ ID No. 43, SEQ ID No. 45, SEQ ID No. 47, SEQ ID No. 49, SEQ ID No. 51, wherein optionally these polynucleotide fragments are provided from 5′- to 3′- in the fusion polynucleotide in accordance with the order in the above list (from SEQ ID No. 39 to SEQ ID No. 51 according to the above disclosure), and/or
(iii) SEQ ID No. 57, SEQ ID No. 59, SEQ ID No. 61, SEQ ID No. 63, SEQ ID No. 65, SEQ ID No. 67, SEQ ID No. 69, SEQ ID No. 71, SEQ ID No. 73, SEQ ID No. 75, SEQ ID No. 77, SEQ ID No. 79, SEQ ID No. 81, SEQ ID No. 83, SEQ ID No 85, SEQ ID No. 87, SEQ ID No. 89, SEQ ID No. 91, SEQ ID No. 93, SEQ ID No. 95, SEQ ID No. 97, SEQ ID No. 99, SEQ ID No. 101, SEQ ID No. 103, wherein optionally these polynucleotide fragments are provided from 5′- to 3′- in the fusion polynucleotide in accordance with the order in the above list (from SEQ ID No. 57 to SEQ ID No. 103 according to the above disclosure), or
(iv) SEQ ID No. 107, SEQ ID No. 109, SEQ ID No. 111, SEQ ID No. 113, SEQ ID No. 115, SEQ ID No. 117, SEQ ID No. 119, SEQ ID No. 121, SEQ ID No. 123, SEQ ID No. 125, SEQ ID No. 127, SEQ ID No. 129, SEQ ID No. 131, SEQ ID No. 133, SEQ ID No. 135, SEQ ID No. 137, SEQ ID No. 139, wherein optionally these polynucleotides are provided from 5′ to 3′ in the recombinant polynucleotide in accordance with the order in the above list (from SEQ ID No. 107 to SEQ ID No. 139 according to the above disclosure).

In a particular embodiment, a recombinant polynucleotide of the invention, in particular a recombinant lentiviral vector genome comprises at least one polynucleotide (in particular 1, 2 or 3 distinct polynucleotides) encoding a fusion polypeptide selected in the group of:
a fusion polypeptide of sequence SEQ ID No. 30,
a fusion polypeptide of sequence SEQ ID No. 38,
a fusion polypeptide of sequence SEQ ID No. 54,
a fusion polypeptide of sequence SEQ ID No. 56,
a fusion polypeptide of sequence SEQ ID No. 106 and
a fusion polypeptide of sequence SEQ ID No. 142.

In a particular embodiment, a recombinant polynucleotide of the invention, in particular a recombinant lentiviral vector genome comprises at least one polynucleotide (in particular 1, 2 or 3 distinct polynucleotides) selected in the group of:
a recombinant polynucleotide of sequence SEQ ID No. 29,
a recombinant polynucleotide of sequence SEQ ID No. 37,
a recombinant polynucleotide of sequence SEQ ID No. 53,
a recombinant polynucleotide of sequence SEQ ID No. 55,
a recombinant polynucleotide of sequence SEQ ID No. 105 and
a recombinant polynucleotide of sequence SEQ ID No. 141.

In another embodiment a recombinant lentiviral vector genome is provided as the insert in the plasmid pFlap-beta2m-DENV-Ag1-WPREm of SEQ ID No. 151 (CNCM I-5883) or in pFlap-beta2m-DENV-Ag2-WPREm of SEQ ID No. 152 (CNCM I-5885) or in pFlap-beta2m-ZIKV-Ag-WPREm of SEQ ID No. 153 (CNCM I-5882) or in pFlap-beta2m-ZIKV-NS1-WPREm of SEQ ID No. 154 (CNCM I-5887) or in pFlap-beta2m-YFV-Ag1-WPREm of SEQ ID No. 155 (CNCM I-5884) or in pFlap-beta2m-YFV-Ag2-WPREm of SEQ ID No. 156 (CNCM I-5886) or in pFlap-beta2m-DENV-Ag2_ZIKV-Ag-WPREm_(Flavi-2) of SEQ ID No. 157 (CNCM I-5888) or in pFlap-beta2m-ZIKV-Ag_DENV-Ag2-WPREm_(Flavi-3) of SEQ ID No. 158 (CNCM I-5889) or in pFlap-beta2m-DENV-Ag2_ZIKV-NS1-WPREm_(Flavi-4) of SEQ ID No. 159 (CNCM I-5890) or in pFlap-beta2m-ZIKV-NS1-DENV-Ag2-WPREm_(Flavi-5) of SEQ ID No. 160 (CNCM I-5891). Plasmid pFlap-beta2m-WPRE used for the insertion of the polynucleotide of the invention may alternatively be designated pFlap-deltaU3-beta2m-WPRE.

In another embodiment a recombinant lentiviral vector genome is provided as the insert in the plasmids deposited at the CNCM (Collection Nationale de Cultures de Microorganismes, Institut Pasteur 25 rue du Dr Roux—75724 Paris Cedex 15—France) on Sep. 13, 2022 as pFlap-beta2m-DENV-Ag1-WPREm with N^{o} CNCM 1-5883 or pFlap-beta2m-DENV-Ag2-WPREm with N^{o} CNCM 1-5885 or pFlap-beta2m-ZIKV-Ag-WPREm with N^{o} CNCM 1-5882 or pFlap-beta2m-ZIKV-NS1-WPREm with N^{o} CNCM 1-5887 or pFlap-beta2m-YFV-Ag1-WPREm with N^{o} CNCM 1-5884 or pFlap-beta2m-YFV-Ag2-WPREm with N^{o} CNCM 1-5886 or pFlap-beta2m-DENV-Ag2_ZIKV-Ag-WPREm_(Flavi-2) with N^{o} CNCM 1-5888 or pFlap-beta2m-ZIKV-Ag_DENV-Ag2-WPREm_(Flavi-3) with N^{o} CNCM 1-5889 or pFlap-beta2m-DENV-Ag2_ZIKV-NS1-WPREm_(Flavi-4) with N^{o} CNCM 1-5890 or pFlap-beta2m-ZIKV-NS1-DENV-Ag2-WPREm_(Flavi-5) with N^{o} CNCM 1-5891.

According to an embodiment, the lentiviral vector genome comprises a recombinant polynucleotide as disclosed herein which is cloned under control of a promoter functional in mammalian cells, in particular the CMV promoter, the human beta-2 microglobulin promoter, the SP1-human beta-2 microglobulin promoter of SEQ ID No. 170 or the composite BCUAG promoter of SEQ ID No. 172 and wherein the vector optionally comprises post-transcriptional regulatory element of the woodchuck hepatitis virus (WPRE), in particular a mutant WPRE as set forth in SEQ ID No. 173 and/or a KOZAK sequence.

The invention also relates to a plasmid vector recombined with a nucleic acid molecule of the recombinant polynucleotide encoding the fusion polypeptide(s) comprising MHC class I T-cell epitopes selected for the elicitation of an immune response in a host as disclosed herein. In an embodiment, the plasmid vector is accordingly an expression vector.

In an embodiment, the plasmid vector is a transfer vector in particular a lentiviral transfer vector, especially a HIV-1 transfer vector suitable to provide the genome of a lentiviral vector of the invention. The lentiviral vector expresses the fusion polypeptide(s) when expressed in vivo in a host.

In a particular embodiment, the nucleic acid molecule containing the genome of the transfer vector is provided as a plasmid comprising the lentiviral backbone vector (especially the HIV-1 backbone vector) recombined with a polynucleotide encoding the selected antigen(s) of the pathogen, for their expression as a fusion polypeptide when said vector genome is provided in a lentiviral vector particle that is used for administration to a host.

Additionally, the recombinant polynucleotide or the vector containing it, in particular the nucleic acid molecule containing the genome of the transfer vector may contain sequences for the control of transcription and/or for the control of expression, and/or may contain sequences for ligation to a distinct nucleic acid such as for ligation to a plasmid or a vector genome. Hence the nucleic acid may contain one or more sequences for restriction site(s) (such as BamHI (GGATCC) XhoI (CTCGAG)), Kozak sequence (GCCACC), stop codon (TAA or TAATGA) sequence, promoter or other sequences as disclosed herein and illustrated in the examples.

The invention accordingly relates to a DNA plasmid comprising the recombinant lentiviral vector genome according to the definitions provided herein, in particular wherein said genome is inserted within the vector plasmid, preferably the vector plasmid of nucleotide sequence SEQ ID No. 161, wherein the fusion polypeptide according to the invention is inserted between restriction sites BamHI and XhoI.

In an embodiment, the plasmid vector of the invention is selected from the group of: the plasmid pFlap-beta2m-DENV-Ag1-WPREm of SEQ ID No. 151 or in pFlap-beta2m-DENV-Ag2-WPREm of SEQ ID No. 152 or in pFlap-beta2m-ZIKV-Ag-WPREm of SEQ ID No. 153 or pFlap-beta2m-ZIKV-NS1-WPREm of SEQ ID No. 154 or pFlap-beta2m-YFV-Ag1-WPREm of SEQ ID No. 155 or pFlap-beta2m-YFV-Ag2-WPREm of SEQ ID No. 156 or pFlap-beta2m-DENV-Ag2_ZIKV-Ag-WPREm_(Flavi-2) of SEQ ID No. 157 or pFlap-beta2m-ZIKV-Ag_DENV-Ag2-WPREm_(Flavi-3) of SEQ ID No. 158 or pFlap-beta2m-DENV-Ag2_ZIKV-NS1-WPREm_(Flavi-4) of SEQ ID No. 159 or pFlap-beta2m-ZIKV-NS1-DENV-Ag2-WPREm_(Flavi-5) of SEQ ID No. 160.

In another embodiment, the invention relates to the plasmids deposited at the CNCM as pFlap-beta2m-DENV-Ag1-WPREm with N^{o} CNCM I-5883 or pFlap-beta2m-DENV-Ag2-WPREm with N^{o} CNCM I-5885 or pFlap-beta2m-ZIKV-Ag-WPREm with N^{o} CNCM I-5882 or pFlap-beta2m-ZIKV-NS1-WPREm with N^{o} CNCM I-5887 or pFlap-beta2m-YFV-Ag1-WPREm with N^{o} CNCM I-5884 or pFlap-beta2m-YFV-Ag2-WPREm with N^{o} CNCM I-5886 or pFlap-beta2m-DENV-Ag2_ZIKV-Ag-WPREm_(Flavi-2) with N^{o} CNCM I-5888 or pFlap-beta2m-ZIKV-Ag_DENV-Ag2-WPREm_(Flavi-3) with N^{o} CNCM I-5889 or pFlap-beta2m-DENV-Ag2_ZIKV-NS1-WPREm_(Flavi-4) with N^{o} CNCM I-5890 or pFlap-beta2m-ZIKV-NS1-DENV-Ag2-WPREm_(Flavi-5) with N^{o} CNCM I-5891.

The invention also concerns a fusion polypeptide as disclosed herein, encoded by a recombinant polynucleotide of the invention, in particular a fusion polypeptide encoded by a nucleic acid molecule disclosed herein by reference to its SEQ ID No. In an embodiment, the fusion polypeptide is selected from the group of:
a polypeptide comprising MHC class I T-cell epitopes of SEQ ID No. 2, SEQ ID No. 4, SEQ ID No. 6, SEQ ID No. 8, SEQ ID No. 10, SEQ ID No. 12, SEQ ID No. 14, SEQ ID No. 16, SEQ ID No. 18, SEQ ID No. 20, SEQ ID No. 22, SEQ ID No. 24, SEQ ID No. 26, SEQ ID No. 28 or a variant thereof which is devoid of SEQ ID No. 2 and comprises in the N-terminal end, SEQ ID No. 32, SEQ ID No. 33 and SEQ ID No. 34, wherein optionally the sequences coding for the epitopes in the above SEQ ID No. are arranged from N-terminal to C-terminal ends according to the order in the above recitation or comprising variants thereof as disclosed herein and/or
a polypeptide comprising the MHC class I T-cell epitopes: SEQ ID No. 40, SEQ ID No. 42, SEQ ID No. 44, SEQ ID No. 46, SEQ ID No. 48, SEQ ID No. 50, SEQ ID No. 52, wherein optionally the sequences coding for the epitopes in the above SEQ ID No. are arranged from N-terminal to C-terminal ends according to the order in the above recitation or comprising variants thereof as disclosed herein and/or
a polypeptide comprising MHC class I T-cell epitopes: SEQ ID No. 58, SEQ ID No. 60, SEQ ID No. 62, SEQ ID No. 64, SEQ ID No. 66, SEQ ID No. 68, SEQ ID No. 70, SEQ ID No. 72, SEQ ID No. 74, SEQ ID No. 76, SEQ ID No. 78, SEQ ID No. 80, SEQ ID No. 82, SEQ ID No. 84, SEQ ID No 86, SEQ ID No. 88, SEQ ID No. 90, SEQ ID No. 92, SEQ ID No. 94, SEQ ID No. 96, SEQ ID No. 98, SEQ ID No. 100, SEQ ID No. 102, SEQ ID No. 104, wherein optionally the sequences coding for the epitopes in the above SEQ ID No. are arranged from N-terminal to C-terminal ends according to the order in the above recitation or comprising variants thereof as disclosed herein and/or
a polypeptide comprising MHC class I T-cell epitopes: SEQ ID No. 108, SEQ ID No. 110, SEQ ID No. 112, SEQ ID No. 114, SEQ ID No. 116, SEQ ID No. 118, SEQ ID No. 120, SEQ ID No. 122, SEQ ID No. 124, SEQ ID No. 126, SEQ ID No. 128, SEQ ID No. 130, SEQ ID No. 132, SEQ ID No. 134, SEQ ID No. 136, SEQ ID No. 138, SEQ ID No. 140, wherein optionally the sequences coding for the epitopes in the above SEQ ID No. are arranged from N-terminal to C-terminal ends according to the order in the above recitation or comprising variants thereof as disclosed herein.

In an embodiment, the invention relates to a fusion polypeptide selected in the group of:
a fusion polypeptide of sequence SEQ ID No. 30,
a fusion polypeptide of sequence SEQ ID No. 38,
a fusion polypeptide of sequence SEQ ID No. 54,
a fusion polypeptide of sequence SEQ ID No. 56,
a fusion polypeptide of sequence SEQ ID No. 106 and
a fusion polypeptide of sequence SEQ ID No. 142.

According to the invention, two antigens, epitopes, antigenic domains polypeptides or antigenic polypeptides are fused to each other when the nucleotide sequences encoding the two antigens, epitopes, antigenic domains polypeptides or antigenic polypeptides are joined to each other in-frame to create a recombinant polynucleotide or gene encoding a fusion polypeptide. The fusion between two polypeptide sequences may be direct or indirect. Two polypeptides are fused directly when the C-terminus of the first polypeptide chain is covalently bonded to the N-terminus of the second polypeptide chain. In such a case the junction region of the fused polypeptides consists of the terminal amino acid residues of the polypeptides that are adjacent to the ligated residues. Alternatively, the polypeptides are fused indirectly, i.e. a linker or spacer peptide or a further polypeptide is present between the two fused polypeptides to create a junction region the amino acid residues of which are not originally contained in the polypeptides to be fused. Junction regions using linkers have been disclosed herein.

The polypeptide chain of each peptide or antigenic domain providing the MHC class I T-cell epitopes comprises, in particular consists of a sequence selected in the group of SEQ ID No. 30 SEQ ID No. 38, SEQ ID No. 54, SEQ ID No. 56, SEQ ID No. 106 and SEQ ID No. 142, as disclosed herein, or is a variant thereof that comprises an amino acid sequence with at least 85% amino acid sequence identity, preferably 90%, 94%, 95% still preferably 98 or 99% or from 94% to 99% sequence identity with the sequence of the original fusion polypeptide such as the DENV-Ag1, DENV-Ag2, ZIKV-Ag, ZIKV-NS1, or YFV-Ag1 and YFV-Ag2 used as sequence of reference. In one embodiment, the polypeptide chain has 1 to 10, in particular 1 to 5, more particularly 1 to 3 amino acid changes with respect to the corresponding sequence of reference. As used herein, an amino acid change may consist in an amino acid substitution, addition or deletion. Preferably, the amino acid substitution is a conservative amino acid substitution.

In an embodiment of the variant of the fusion polypeptide providing the MHC class I T-cell epitopes disclosed above the polypeptide chain of the variant is obtained by substitution of amino acid residues. Preferably, the amino acid substitution is a conservative amino acid substitution.

According to the invention, the fusion polypeptide carries several polypeptides that comprise or are the MHC class I T-cell epitopes or antigenic domains containing the same of distinct non-structural antigens of the same virus or carries several polypeptides that comprise or are the MHC class I T-cell epitopes or of distinct antigens of the different viruses among DENV, ZIKV and YFV, in particular of DENV and ZIKV or DENV and YFV. In a particular embodiment, in addition to the MHC class I T-cell epitopes or antigenic domains originating from non-structural proteins of the virus, the fusion polypeptide also comprises MHC class I T-cell epitopes or antigenic domains originating from structural proteins of the virus.

Accordingly, the fusion polypeptides of the invention are poly-antigenic polypeptides.

An “antigen” or an “antigenic polypeptide” is defined herein as a wild type or native antigen of a virus among the DENV, ZIKV and YFV or as a fragment of such wild type a native antigen or as a mutated polypeptide or as a synthetic antigen derived from the alignment of available amino acid sequences of the native antigens or of a consensus sequence as disclosed herein. A fragment of the wild type or the native antigen or a synthetic antigen advantageously keeps the immunogenic properties of the polypeptide from which it derives or shows improved immunogenic properties when it is expressed by the lentiviral vector of the invention and advantageously shows immune protective properties when expressed in a host. Such a fragment or synthetic antigen is accordingly an immunogenic fragment of an antigen or an immunogenic antigen. A antigen used to provide the fusion polypeptide of the invention has an amino acid sequence which is sufficient to provide one or advantageously several epitope(s) in particular T-cell epitopes and more particularly CD8+ T-cell epitopes and which keeps the immunogenic, especially the protective properties leading to the protective activity of the antigenic polypeptide from which it derives and/or exhibits such protective properties in particular when expressed by the lentiviral vector of the invention.

In an embodiment, the association of the antigenic domains in the fusion polypeptide is an arrangement of the antigenic domains from N- to C-terminal in the same order as they appear in the antigen from which they originate. In an embodiment, the association of the antigenic domains in the fusion polypeptide is an arrangement of the antigenic domains from N- to C-terminal in a modified order with respect to the order in which they appear in the antigen from which they originate. Examples of such modified arrangements are illustrated in the disclosed fusion polypeptides.

In a particular embodiment more than one recombinant fusion protein is expressed by the lentiviral particles of the lentiviral vector of the invention. In a particular embodiment the fusion polypeptides of the DENV, ZIKV and YFV, in particular of DENV and ZIKV or DENV and YFV are expressed by the same lentiviral particles of the lentiviral vector of the invention or by a mixture of particles.

In a particular embodiment the fusion polypeptide provides at least 2, in particular at least 3 or at least 4 or at least 5 and in particular are especially 2, 3, 4 or 5, and accordingly encompass at least 2, at least 3 or at least 4 antigens and/or antigenic fragments (antigenic domains) or mutated antigens and/or fragments thereof with respect to a native or wild type determined antigen of a pathogen. In a particular embodiment the antigenic polypeptide contained in the fusion polypeptide comprises or consists of a fusion of up to 10 antigens, advantageously up to 25 antigenic fragments (such as the epitopes encoded by the recombinant polynucleotides of the invention expressed by the lentiviral vectors disclosed herein), in particular from 7 to 25 antigenic fragments or mutated fragments thereof. The inventors have demonstrated that the fusion polypeptide of the invention is capable of driving the expression of large antigenic polypeptides, as one or more than one fusion polypeptide expressed by the lentiviral vectors disclosed herein. In one embodiment, the fusion polypeptide comprises at least 300 amino acids, in particular at least 400 amino acids, more particularly at least 400 or 500 amino acids. In one embodiment, the fusion polypeptide comprises from 300 to 1400 amino acids, in particular from 300 to 850 amino acids. In one embodiment, the fusion polypeptide(s), expressed by the lentiviral vector, comprise(s) at least 300 amino acids, more particularly at least 400 or 500 amino acids. In one embodiment, the antigenic polypeptide comprises from 300 to 1400 amino acids, in particular from 300 to 850 amino acids.

According to an embodiment, the antigenic polypeptide(s) may be fused to give rise to the fusion polypeptide via a linker.

Linker sequences are used accordingly to avoid the formation of neo-epitopes that would interfere with the specific immune response against the epitopes of the antigen(s) of the pathogen(s) in the host. Suitable linkers are selected by the person skilled in the art according to well-known techniques and are shown in the Examples.

In an embodiment, the one or more antigenic polypeptides are selected and arranged within the fusion polypeptide with or without added linkers to reduce the occurrence of neo-epitopes between the epitopic regions.

In another aspect of the invention, the inventors have designed and prepared a lentiviral vector i.e., lentiviral vector particles, encoding a fusion polypeptide of the invention, in which MHC class I T-cell epitopes originating from more than one non-structural proteins of DENV, ZIKV and/or YFV are fused and accordingly may be expressed in recombinant lentiviral particles.

The invention accordingly provides new lentiviral vectors expressing recombinant fusion polypeptide(s) as recited in any of the embodiment disclosed herein, eliciting T-cell immunogenicity encompassing a CD8⁺ T-cell immune response against the fusion polypeptide(s) in a host, or against a DENV, ZIKV or YFV virus responsive to the immune response elicited by the administration of the lentiviral vector of the invention, especially in a mammalian host, in particular a human host.

The expression “lentiviral vector” or “lentiviral vector particles” relates to biological or chemical entities suitable for the delivery of the recombinant polynucleotides encoding the fusion polypeptides of the invention to the cells of the host administered with such vectors. Viral vectors as those described herein such as lentiviral vectors capable of inducing human immune response. The invention relates in particular to the use of HIV vectors, especially HIV-1 vectors which are illustrated in the Examples. Details for the construction for HIV-1 vectors are known in the art and provided hereafter and in the Examples.

In accordance with the invention, lentiviral vectors expressing fusion polypeptide(s) of the invention are provided wherein the vectors have or comprise in their genome (vector genome) a recombinant polynucleotide which encodes a fusion polypeptide according to the invention. In an embodiment the vectors have or comprise in their genome (vector genome) a recombinant polynucleotide which encodes a plurality of fusion polypeptides according to the invention, wherein collectively the fusion polypeptide(s) originate from more than one virus, in particular originate from 2 or 3 different viruses of the Flavivirus genus that are selected from Dengue virus, Zika virus (ZIKV) and/or Yellow fever virus (YFV). As far as Dengue virus is concerned, the 4 known serotypes of the virus may be used to derive the antigenic domains used in the fusion polypeptide. Specific embodiments of the vector genome of the lentiviral vector of the invention have been disclosed above and in the Examples.

The lentiviral vectors of the invention, especially the preferred HIV-1 based vectors, may be replication-incompetent pseudotyped lentiviral vectors, in particular a replication-incompetent pseudotyped HIV-1 lentiviral vector, wherein said vector contains a genome comprising a mammal codon-optimized synthetic nucleic acid, in particular a human-codon optimized synthetic nucleic acid, wherein said synthetic nucleic acid encodes at least one fusion polypeptide according to the invention, comprising (an) antigenic polypeptide(s), in particular the antigenic polypeptide(s) of a determined virus as disclosed herein infecting a mammal, in particular a human host. The lentiviral vector may be advantageously pseudotyped with a viral envelope protein that is not a lentiviral, in particular not a HIV-1 retroviral, envelope protein or glycoprotein. The lentiviral vector may be pseudotyped with the glycoprotein G from a Vesicular Stomatitis Virus (V-SVG) of Indiana or of New Jersey serotype.

Use of codon-optimized sequences in the genome of the vector particles allows in particular strong expression of the antigenic polypeptide in the cells of the host administered with the vector, especially by improving mRNA stability or reducing secondary structures. In addition, the expressed antigenic polypeptide undergoes post translational modifications which are suitable for processing of the antigenic polypeptide in the cells of the host, in particular by modifying translation modification sites (such as glycosylation sites) in the encoded polypeptide. Codon optimization tools are well known in the art, including algorithms and services such as those made available by GeneArt (Life technologies-USA) and DNA2.0 (Menlo Park, California—USA). In a particular embodiment codon-optimization is carried out on the open reading frame (ORF) sequence encoding the antigenic polypeptide and the optimization is carried out prior to the introduction of the sequence encoding the ORF into the plasmid intended for the preparation of the vector genome. In another embodiment, additional sequences of the vector genome are also codon-optimized. Codon-optimized nucleic acids for the recombinant polynucleotides of the invention are provided as examples.

The active ingredients consisting of the viral vectors may be integrative pseudotyped lentiviral vectors, especially replication-incompetent integrative pseudotyped lentiviral vectors, in particular a HIV-1 vector. Such lentiviral vectors may in addition contain a genome comprising a mammal-codon optimized synthetic nucleic acid, in particular a human-codon optimized nucleic acid, such as the insert contained in recombinant pFLAP of SEQ ID No. 151, SEQ ID No. 152, SEQ ID No. 153, SEQ ID No. 154, SEQ ID No. 155, SEQ ID No. 156, SEQ ID No. 157, SEQ ID No. 158, SEQ ID No. 159, or SEQ ID No. 160 wherein said nucleic acid encodes a fusion polypeptide according to the invention.

Alternatively, the lentiviral vector and in particular the HIV-1 based vector may be a non-integrative replication-incompetent pseudotyped lentiviral vector.

A particular embodiment of a lentiviral vector suitable to achieve the invention relates to a lentiviral vector whose genome is obtained from the pTRIP vector plasmid or the the pFLAPdeltaU3 plasmid known in the art wherein the nucleic acid encoding the fusion polypeptide has been cloned under control of a promoter functional in mammalian cells, in particular the CMV promoter, the human β2-microglobulin promoter (SEQ ID No. 170), the SP1-β2m promoter of SEQ ID No. 171 or the composite “BCUAG” promoter of SEQ ID No. 172, preferably the SP1-β2m promoter, and wherein the vector optionally comprises post-transcriptional regulatory element of the woodchuck hepatitis virus (WPRE—SEQ ID No. 174), wild type or mutated. In particular, the WPRE is a mutant WPRE as set forth in SEQ ID No. 173.

The pFLAP-beta2m-WPREm (SEQ ID No. 161) is a lentiviral plasmid vector derived from pFLAPdeltaU3 plasmid or pFLAP plasmid, which is a lentiviral plasmid vector derived from the pTRIP plasmid. Examples of pFLAP plasmids of the invention are pFlap-beta2m-DENV-Ag1-WPREm of SEQ ID No. 151 or in pFlap-beta2m-DENV-Ag2-WPREm of SEQ ID No. 152 or in pFlap-beta2m-ZIKV-Ag-WPREm of SEQ ID No. 153 or pFlap-beta2m-ZIKV-NS1-WPREm of SEQ ID No. 154 or pFlap-beta2m-YFV-Ag1-WPREm of SEQ ID No. 155 or pFlap-beta2m-YFV-Ag2-WPREm of SEQ ID No. 156 or pFlap-beta2m-DENV-Ag2_ZIKV-Ag-WPREm_(Flavi-2) of SEQ ID No. 157 or pFlap-beta2m-ZIKV-Ag_DENV-Ag2-WPREm_(Flavi-3) of SEQ ID No. 158 or pFlap-beta2m-DENV-Ag2_ZIKV-NS1-WPREm_(Flavi-4) of SEQ ID No. 159 or pFlap-beta2m-ZIKV-NS1-DENV-Ag2-WPREm_(Flavi-5) of SEQ ID No. 160.

In a further embodiment of the invention, the lentiviral vector particle expressing the fusion polypeptide according to the features herein described is pseudotyped with the glycoprotein G from a Vesicular Stomatitis Virus (VSV-G) of Indiana or of New Jersey serotype.

The particular features of such lentiviral vectors will be further discussed in detail below.

The invention further relates to a host cell, preferably a mammalian host cell, comprising the lentiviral vector genome of the invention, or transfected with a DNA plasmid according to the invention. In particular, said host cell is a HEK-293T cell line or a K562 cell line. The invention further relates to a culture of said host cells.

The invention also relates to a formulation or pharmaceutical composition, in particular a vaccine composition, suitable for administration to a mammalian host, comprising a recombinant lentiviral vector of the invention together with one or more pharmaceutically acceptable excipient(s) suitable for administration to a host in need thereof, in particular a mammalian host, especially a human host.

The invention also relates to a formulation suitable for administration to a mammalian host, in particular a human host comprising as an active ingredient lentiviral vector particles as defined herein for protection against a viral infection or against the viral-induced condition or disease, wherein the virus is a flavivirus as disclosed herein or a plurality of flaviviruses selected from the group of Dengue virus, especially one or at least one of the known serotypes DENV-1, DENV-2, DENV-3 and DENV-4, in particular a viral infection by any or all of the DENV-1, DENV-2, DENV-3 or DENV-4, or the Zika virus (ZIKV) or the Yellow Fever virus (YFV), together with excipient(s) suitable for administration to a host in need thereof, in particular a human host.

The pharmaceutical composition, in particular the vaccine composition, or the formulation according to the invention may also comprise an adjuvant component and/or an immunostimulatory component.

In particular, the composition or formulation may comprise a pro-Th1 adjuvant such as polyinosinic-polycytidylic acid (polyI:C) or a derivative thereof. A derivative of poly (1:C) refers to a mismatched dsRNA obtained by modifying the specific configuration of poly (1:C) through the introduction of unpaired bases thereinto, and includes poly (I:CxU), poly (IxU:C) (where x is on average a number from 3 to 40) and the like. Preferably, a derivative of poly (1:C) is poly (I:C12U) or poly (C:I12U), which is commercially available under the trade name Ampligen™ The composition or formulation may also comprise a pro-Th1/Th17 adjuvant such as a cyclic dinucleotide adjuvant. Cyclic nucleotide adjuvants are also referred to as STING-activating cyclic dinucleotide adjuvant. The term “cyclic dinucleotides” (“CDNs”) as used herein refers to a class of molecules comprising 2′-5′ and/or 3′-5′ phosphodiester linkages between two purine nucleotides. This includes 2′-5-2′,5′, 2′-5′-3′5′, and 3′,5′-3′,5′ linkages. CDNs are ubiquitous small molecule second messengers synthesized by bacteria that regulate diverse processes and are a relatively new class of adjuvants that have been shown to increase vaccine potency. CDNs activate innate immunity by directly binding the endoplasmic reticulum-resident receptor STING (stimulator of interferon genes), activating a signaling pathway that induces the expression of interferon-β (IFN-β) and also nuclear factor-κB (NF-κB) dependent inflammatory cytokines. Preferably, the CDN is cyclic Guanine-Adenine dinucleotide (cGAMP).

The use of adjuvants, in particular pro-Th1 and/or pro Th17 adjuvants, together with the lentiviral vector of the invention may elicit the generation of Th1 CD8⁺ T cells.

In another aspect of the invention, the active ingredient, in particular the lentiviral vector particles, or the composition or the formulation comprising the same is for use in the protective immunization against a viral infection or against viral-induced condition or disease wherein the virus is a flavivirus as disclosed herein or a plurality of flaviviruses selected from the group of Dengue virus, especially one or at least one of the known serotypes DENV-1, DENV-2, DENV-3 and DENV-4 in particular a viral infection by any or all of the DENV-1, DENV-2, DENV-3 or DENV-4, or the Zika virus (ZIKV) or the Yellow Fever virus (YFV), in a mammalian host, especially a human host, optionally in association with an appropriate delivery vehicle and optionally with an adjuvant component and/or with an immunostimulant component, e.g. an adjuvant component and/or immunostimulant component as defined in the present specification.

Accordingly, the active ingredient, or the composition, in particular the lentiviral vector particles of the invention, when administered to a host in need thereof, especially to a mammalian, in particular to a human host, elicits an immune response that encompasses a CD8+ T-cell response directed against the antigenic polypeptide or immunogenic fragments thereof expressed by the fusion polypeptide(s). Said immune response may encompass activation of naïve lymphocytes and generation of effector T-cell response and generation of immune memory antigen-specific T-cell response against antigen(s) of the pathogen.

One aspect of the invention relates to the active ingredient, in particular the lentiviral vector particles, the pharmaceutical composition and/or formulation of the invention, for use in preventing and/or treating an infection by a virus wherein the virus is a flavivirus as disclosed herein or a plurality of flaviviruses selected from the group of Dengue virus, especially one or at least one of the known serotypes DENV-1, DENV-2, DENV-3 and DENV-4 in particular a viral infection by any or all of the DENV-1, DENV-2, DENV-3 or DENV-4, or the Zika virus (ZIKV) or the Yellow Fever virus (YFV) in a mammalian host in need thereof. The invention also relates to a method of preventing and/or treating an infection by a virus wherein the virus is a flavivirus as disclosed herein or a plurality of flaviviruses selected from the group of Dengue virus, especially one of the known serotypes DENV-1, DENV-2, DENV-3 and DENV-4, or the Zika virus (ZIKV) or the Yellow Fever virus (YFV) in a mammalian host in need thereof.

In a particular embodiment the active ingredient, in particular the lentiviral vector particles, the pharmaceutical composition and/or formulation of the invention, is for use in preventing and/or treating an infection by any or all virus of the DENV-1, DENV-2, DENV-3 or DENV-4. Accordingly the invention enables protection of the host administered with the active ingredient, in particular the lentiviral vector particles, the pharmaceutical composition and/or formulation of the invention against all the serotypes of the DENV.

The immune response involves the induction of MHC-I restricted presentation of the antigenic polypeptide or immunogenic fragments thereof contained in the fusion polypeptide of the invention, by an antigen-presenting cell, in particular a dendritic cell, and the induction of a CD8-mediated immune response.

The lentiviral vector of the invention is particularly capable of eliciting the generation of polypotent T cells, including CD8+ T cells secreting one or more of IFN-γ, TNF-α, IL-2 and lymphocyte degranulation marker CD107a.

The immune response may either prevent the infection by the virus or may prevent (protect against) the onset or the development of a pathological state resulting from infection by a Dengue virus, especially one of the known serotypes DENV-1, DENV-2, DENV-3 and DENV-4 or in particular any or all of the known serotypes DENV-1, DENV-2, DENV-3 and DENV-4, or the Zika virus (ZIKV) or the Yellow Fever virus (YFV).

Physiologically acceptable vehicles may be chosen with respect to the administration route of the immunization composition. In a preferred embodiment, administration may be carried out by injection, in particular intramuscularly, intradermally, subcutaneously, or, by intranasal administration or topical skin application.

Recombinant lentiviral vector particles of the invention are used for elicitation in a host, in particular a mammalian host, especially a human host, of an immune response against the virus wherein the virus is a flavivirus as disclosed herein or a plurality of flaviviruses selected from the group of Dengue virus, especially one of the known serotypes DENV-1, DENV-2, DENV-3 and DENV-4 or in particular any or all of the known serotypes DENV-1, DENV-2, DENV-3 and DENV-4, or the Zika virus (ZIKV) or the Yellow Fever virus (YFV), said use involving an immunization pattern comprising administering an effective amount of an active ingredient. In an embodiment, the lentiviral particles that elicit the cellular immune response of the host are administered as a single dose. In an embodiment, the lentiviral particles that elicit the cellular immune response of the host are administered as a prime, and later in time administering an effective amount of the same active ingredient or another active ingredient, e.g. the lentiviral particles, is performed to boost the cellular immune response of the host, and optionally repeating (once or several times) said administration step for boosting.

For each step of administration of the lentiviral vector particles, in particular in a regimen that encompasses multiple administration steps, it may be advantageous that the pseudotyping envelope protein(s) of the vector particles is(are) different from the one used in the other step(s), especially originate from different viruses, in particular different serotypes of VSV. In the prime-boost regimen, the administered combination of compounds of each step comprises lentiviral vectors as defined herein.

Priming and boosting steps are separated in time by at least 2 weeks, in particular 6 weeks, in particular by at least 8 weeks.

In a particular embodiment, the recombinant lentiviral vector particles of the invention are used for elicitation in a host, in particular a mammalian host, especially a human host, of an immune response against the virus providing the antigens expressed by the particles, said use involving an immunization pattern comprising a heterologous prime-boost regimen wherein the recombinant lentiviral vector particles of the invention are used for a prime or for a boost. Details on the administration regimen will be discussed further below.

The LV particles provide a cellular immune response (T-cell immune response), particularly a CD8+ T-cell immune response, i.e., an adaptive immune response which is mediated by activated T cells harbouring CD8 receptors.

In a particularly advantageous embodiment, the immune response conferred by the LV particles, is a long-lasting immune response i.e., said immune response encompasses memory cells response and in particular central memory cells response; in a particular embodiment it can be still detected at least several months after the last administration step.

In accordance with the invention when the lentiviral particles are used in a prime-boost regimen or a multiple steps administration regimen, lentiviral vector particles are provided which are pseudotyped with a first determined pseudotyping envelope G protein obtained from the VSV, strain Indiana or New Jersey, and later administered lentiviral vector particles are provided which are pseudotyped with a second determined pseudotyping envelope G protein obtained from a VSV, strain New Jersey or Indiana. The order of use in the prime-boost regimen of the first and second compounds thus described may alternatively be inversed. Thus, the lentiviral vector particles contained in the separate active ingredients/compounds of the combinations or compositions of the invention when intended for use in a prime-boost regimen are distinct from each other, at least due to the particular pseudotyping envelope protein(s) used for pseudotyping the vector particles.

Doses of lentiviral vectors intended for elicitation of the cellular immune response which are used in the administration pattern, may comprise from 105 TU to 10¹⁰ TU of recombinant lentiviral particles especially from 105 to 10⁸ TU, when integrative vectors are used. The dose intended for administration to the host may comprise from 10⁸ to 10¹⁰ of each type of recombinant lentiviral vector particles when integrative-incompetent vectors are used.

The invention also concerns a method of providing immunization in a mammalian host, especially in a human host, comprising the step of administering, as a prime or as a boost, the recombinant lentiviral vector particles of the invention to elicit the immune response, and optionally repeating the administration steps one or several times, in particular to boost said response, in accordance with the present disclosure.

Optionally, the recombinant lentiviral vector particles may be used in association with an adjuvant compound suitable for administration to a mammalian, especially a human host, and/or with an immunostimulant compound, together with an appropriate delivery vehicle. Suitable adjuvants and immunostimulant compounds are described in the present specification.

The recombinant lentiviral vector particles can be administered to the host via injection through different routes including subcutaneous (s.c.), intradermal (i.d.), intramuscular (i.m.) or intravenous (i.v.) injection or may be administered orally to topically trough mucosal or skin administration, especially intranasal (i.n.) administration or inhalation. The quantity to be administered (dosage) depends on the subject to be treated, including considering the condition of the patient, the state of the individual's immune system, the route of administration and the size of the host. Suitable dosages range may be determined with respect to the content in equivalent transducing units of HIV-1-derived lentiviral vector particles.

Other examples and features of the invention will be apparent when reading the examples and the figures which illustrate the preparation and application of the lentiviral vector particles with features that may be individually combined with the definitions given in the present description.

### Detailed Description of the Lentiviral Vectors for Use According to the Invention

The invention accordingly involves lentiviral vectors which are recombinant lentiviral particles (i.e. recombinant vector particles), and which may be replication-incompetent lentiviral vectors, especially replication-incompetent HIV-1 based vectors characterized in that: (i) they are pseudotyped with a determined heterologous viral envelope protein or viral envelope proteins originating from a RNA virus which is not HIV, and (ii) they comprise in their genome at least one recombinant polynucleotide encoding a fusion polypeptide of the invention, comprising at least one antigenic polypeptide (or polypeptide derivative thereof such as immunogenic fragment(s) thereof) carrying epitope(s) of an antigen of a virus wherein the virus is a flavivirus as disclosed herein or a plurality of flaviviruses selected from the group of Dengue virus, especially one of the known serotypes DENV-1, DENV-2, DENV-3 and DENV-4, or in particular any or all of the known serotypes DENV-1, DENV-2, DENV-3 and DENV-4, or the Zika virus (ZIKV) or the Yellow Fever virus (YFV) wherein said epitopes encompass T-cell epitope(s), in particular CD8+ T-cell epitopes.

According to a particular embodiment of the invention, the lentiviral vectors are either designed to express proficient (i.e., integrative-competent) or deficient (i.e., integrative-incompetent) particles. According to a particular embodiment of the invention, the recombinant lentiviral vector particles are both integration-incompetent and replication-incompetent.

The preparation of the lentiviral vectors is well known from the skilled person and has been extensively disclosed in the literature (confer for review Sakuma T. et al (Biochem. J. (2012) 443, 603-618). The preparation of such vectors is also illustrated herein in the Examples.

In a particular embodiment of the invention, the polynucleotide(s) encoding the antigenic polypeptides (ORF) of the lentiviral vector has(have) been mammal-codon optimized (CO) in particular human-codon optimized. Optionally the lentiviral sequences of the genome of said particles have also a mammal-codon optimized nucleotide sequence. In a particular aspect of the invention the codon optimization has been carried out for expression in mouse cells. In another embodiment the sequence of the polynucleotide(s) encoding the antigenic polypeptides of the lentiviral vector has(have) been human-codon optimized (CO).

It has been observed that codon optimized nucleotide sequences, especially when optimized for expression in mammalian and in particular in human cells, enable the production of higher yield of particles in such mammalian or human cells. Production cells are illustrated in the examples. Accordingly, when lentiviral vector particles of the invention are administered to a mammalian, especially to a human host, higher amounts of particles are produced in said host which favour the elicitation of a strong immune response.

The recombinant lentiviral vector (i.e., lentiviral vectors particles or lentiviral-based vector particles) defined in the present invention are pseudotyped lentiviral vectors consisting of vector particles bearing envelope protein or envelope proteins which originate from a virus different from the particular lentivirus (especially a virus different from HIV, in particular HIV-1), which provides the vector genome of the lentiviral vector particles. Accordingly, said envelope protein or envelope proteins, are “heterologous” viral envelope protein or viral envelope proteins with respect to the vector genome of the particles. In the following pages, reference will also be made to “envelope protein(s)” to encompass any type of envelope protein or envelope proteins suitable to perform the invention.

When reference is made to “lentiviral” vectors (lentiviral-based vectors) in the present disclosure, it relates in particular, to HIV-based vectors and especially HIV-1-based vectors.

The lentiviral vectors suitable to perform the invention are so-called replacement vectors, meaning that the sequences of the original lentivirus encoding the lentiviral proteins are essentially deleted in the genome of the vector or, when present, are modified, and especially mutated, especially truncated, to prevent expression of biologically active lentiviral proteins, in particular, in the case of HIV, to prevent the expression by said transfer vector providing the genome of the recombinant lentiviral vector particles, of functional ENV, GAG, and POL proteins and optionally of further structural and/or accessory and/or regulatory proteins of the lentivirus, especially of HIV.

In a particular embodiment, the lentiviral vector is built from a first-generation vector, in particular a first-generation of a HIV-based vector which is characterized in that it is obtained using separate plasmids to provide (i) the packaging construct, (ii) the envelope and (iii) the transfer vector genome. Alternatively, it may be built from a second-generation vector, in particular a second-generation of a HIV-based vector which in addition, is devoid of viral accessory proteins (such as in the case of HIV-1, Vif, Vpu, Vpr or Nef) and therefore includes only four out of nine HIV full genes: gag, pol, tat and rev. In another embodiment, the vector is built from a third-generation vector, in particular a third-generation of a HIV-based vector which is furthermore devoid of said viral accessory proteins and also is Tat-independent; these third-generation vectors may be obtained using 4 plasmids to provide the functional elements of the vector, including one plasmid encoding the Rev protein of HIV when the vector is based on HIV-1. Such vector system comprises only three of the nine genes of HIV-1. The structure and design of such generations of HIV-based vectors is well known in the art.

In any of these generations of the vector, modifications are additionally provided according to the invention by insertion in the vector backbone of the polynucleotide encoding the fusion polypeptide as described herein, to provide a LV vector leveraged to target and activate APC, in particular dendritic to induce a cellular immune response, in particular a CD8+ T-cell response.

Particular features of the lentiviral vectors used in accordance with the various embodiments of the invention are also disclosed in the Examples, such features being either taken alone or in combination to produce the vectors.

According to an embodiment of the invention, the lentiviral vector particles are pseudotyped with a heterologous viral envelope protein or viral polyprotein of envelope originating from an RNA virus which is not the lentivirus providing the lentiviral sequences of the genome of the lentiviral particles.

As examples of typing envelope proteins for the preparation of the lentiviral vector, the invention relates to viral transmembrane glycosylated (so-called G proteins) envelope protein(s) of a Vesicular Stomatitis Virus (VSV), which is(are) for example chosen in the group of VSV-G protein(s) of the Indiana strain and VSV-G protein(s) of the New Jersey strain.

Other examples of VSV-G proteins that may be used to pseudotype the lentiviral vectors of the invention encompass VSV-G glycoprotein may especially be chosen among species classified in the vesiculovirus genus: Carajas virus (CJSV), Chandipura virus (CHPV), Cocal virus (COCV), Isfahan virus (ISFV), Maraba virus (MARAV), Piry virus (PIRYV), Vesicular stomatitis Alagoas virus (VSAV), Vesicular stomatitis Indiana virus (VSIV) and Vesicular stomatitis New Jersey virus (VSNJV) and/or stains provisionally classified in the vesiculovirus genus as Grass carp rhabdovirus, BeAn 157575 virus (BeAn 157575), Boteke virus (BTKV), Calchaqui virus (CQIV), Eel virus American (EVA), Gray Lodge virus (GLOV), Jurona virus (JURV), Klamath virus (KLAV), Kwatta virus (KWAV), La Joya virus (LJV), Malpais Spring virus (MSPV), Mount Elgon bat virus (MEBV), Perinet virus (PERV), Pike fry rhabdovirus (PFRV), Porton virus (PORV), Radi virus (RADIV), Spring viremia of carp virus (SVCV), Tupaia virus (TUPV), Ulcerative disease rhabdovirus (UDRV) and Yug Bogdanovac virus (YBV).

The envelope glycoprotein of the vesicular stomatitis virus (VSV-G) is a transmembrane protein that functions as the surface coat of the wild type viral particles. It is also a suitable coat protein for engineered lentiviral vectors. Presently, nine virus species are definitively classified in the VSV gender, and nineteen rhabdoviruses are provisionally classified in this gender, all showing various degrees of cross-neutralisation. When sequenced, the protein G genes indicate sequence similarities. The VSV-G protein presents an N-terminal ectodomain, a transmembrane region and a C-terminal cytoplasmic tail. It is exported to the cell surface via the trans-Golgi network (endoplasmic reticulum and Golgi apparatus).

Vesicular stomatitis Indiana virus (VSIV) and Vesicular stomatitis New Jersey virus (VSNJV) are preferred strains to pseudotype the lentiviral vectors of the invention, or to design recombinant envelope protein(s) to pseudotype the lentiviral vectors. Their VSV-G proteins are disclosed in GenBank, where several strains are presented. For VSV-G New Jersey strain reference is especially made to the sequence having accession number V01214. For VSV-G of the Indiana strain, reference is made to the sequence having accession number AAA48370.1 in Genbank corresponding to strain JO2428.

Said viral envelope protein(s) are capable of uptake by antigen presenting cells and especially by dendritic cells including by liver dendritic cells by mean of fusion and/or of endocytosis. In a particular embodiment, the efficiency of the uptake may be used as a feature to choose the envelope of a VSV for pseudotyping. In this respect the relative titer of transduction (Titer DC/Titer of other transduced cells e.g., 293T cells) may be considered as a test and envelope having a relatively good ability to fuse with DC would be preferred.

Antigen Presenting Cells (APC) and especially Dentritic cells (DC) are proper target cells for pseudotyped lentiviral vectors which are used as immune compositions accordingly.

The VSV-G envelope protein(s) are expressed from a polynucleotide containing the coding sequence for said protein(s), which polynucleotide is inserted in a plasmid (designated envelope expression plasmid or pseudotyping env plasmid) used for the preparation of the lentiviral vector particles of the invention. The polynucleotide encoding the envelope protein(s) is under the control of regulatory sequences for the transcription and/or expression of the coding sequence including optionally post-transcriptional regulatory elements (PRE) especially a polynucleotide such as the element of the Woodchuck hepatitis virus, i.e. the WPRE sequence, obtainable from Invitrogen or a mutant sequence of WPRE as set forth in SEQ ID No. 174.

Accordingly, a nucleic acid construct is provided which comprises an internal promoter suitable for the use in mammalian cells, especially in human cells in vivo and the nucleic acid encoding the envelope protein under the control of said promoter. A plasmid containing this construct is used for transfection of cells suitable for the preparation of vector particles. Promoters may in particular be selected for their properties as constitutive promoters, tissue-specific promoters, or inducible promoters. Examples of suitable promoters encompass the promoters of the following genes: MHC Class-I promoters, human beta-2 microglobulin gene (β2M promoter), EF1α, human PGK, PPI (preproinsulin), thiodextrin, HLA DR invariant chain (P33), HLA DR alpha chain, Ferritin L chain or Ferritin H chain, Chymosin beta 4, Chymosin beta 10, Cystatin Ribosomal Protein L41, CMVie or chimeric promoters such as GAG(CMV early enhancer/chicken β actin) disclosed in Jones S. et al (Jones S. et al Human Gene Therapy, 20:630-640(June 2009)) or beta-2m-CMV (BCUAG) as disclosed herein.

These promoters may also be used in regulatory expression sequences involved in the expression of gag-pol derived proteins from the encapsidation plasmids, and/or to express the antigenic polypeptides from the transfer vector.

Alternatively, when the envelope expression plasmid is intended for expression in stable packaging cell lines, especially for stable expression as continuously expressed viral particles, the internal promoter to express the envelope protein(s) is advantageously an inducible promoter such as one disclosed in Cockrell A. S. et al. (Mol. Biotechnol. (2007) 36:184-204). As examples of such promoters, reference is made to tetracycline and ecdysone inducible promoters. The packaging cell line may be the STAR packaging cell line (ref Cockrell A. S. et al (2007), Ikedia Y. et al (2003) Nature Biotechnol. 21: 569-572) or a SODk packaging cell line, such as SODk0 derived cell lines, including SODk1 and SODk3 (ref Cockrell A. S. et al (2007), Cockrell A. S. et al (2006) Molecular Therapy, 14: 276-284, Xu K. et al. (2001), Kafri T. et al (1999) Journal of Virol. 73:576-584).

According to the invention, the lentiviral vectors are the product recovered from co-transfection of mammalian cells, with:
a vector plasmid comprising (i) lentiviral, especially HIV-1, cis-active sequences necessary for packaging, reverse transcription, and transcription and further comprising a functional lentiviral, especially derived from HIV-1, DNA flap element and (ii) at least one polynucleotide encoding the fusion polypeptide of the invention, itself comprising one or more antigenic polypeptide(s) or immunogenic fragment(s) thereof of one or more viruses against which an immune response is sought, wherein the virus is a flavivirus as disclosed herein or a plurality of flaviviruses selected from the group of Dengue virus, especially one of the known serotypes DENV-1, DENV-2, DENV-3 and DENV-4, or the Zika virus (ZIKV) or the Yellow Fever virus (YFV) under the control of regulatory expression sequences, preferably a human β2 microglobulin promoter or a modified human β2-microglobulin promoter such as the SP1-β2m promoter, and optionally comprising sequences for integration into the genome of the host cell;
an expression plasmid encoding a pseudotyping envelope derived from an RNA virus, said expression plasmid comprising a polynucleotide encoding an envelope protein or proteins for pseudotyping, wherein said envelope pseudotyping protein is advantageously from a VSV and is in particular a VSV-G of the Indiana strain or of the New Jersey strain and,
an encapsidation plasmid, which either comprises lentiviral, especially HIV-1, gag-pol packaging sequences suitable for the production of integration-competent vector particles or modified gag-pol packaging sequences suitable for the production of integration-deficient vector particles.

The invention thus also concerns lentiviral vector particles as described above, which are the product recovered from a stable cell line transfected with:
a vector plasmid comprising (i) lentiviral, especially HIV-1, cis-active sequences necessary for packaging, reverse transcription, and transcription and further comprising a functional lentiviral, especially HIV-1, DNA flap element and optionally comprising cis-active sequences necessary for integration, said vector plasmid further comprising, (ii) a recombinant polynucleotide, especially a recombinant polynucleotide of codon-optimized sequence for murine or for human, encoding the fusion polypeptide(s) of the invention, comprising one or more antigenic polypeptide(s) or immunogenic fragment(s) thereof of one or more viruses as disclosed herein, under the control of regulatory expression sequences, especially a promoter;
a VSV-G envelope expression plasmid comprising a polynucleotide encoding a VSV-G envelope protein in particular VSV-G of the Indiana strain or of the New Jersey strain, wherein said polynucleotide is under the control of regulating expression sequences, in particular regulatory expression sequences comprising a promoter, and;
an encapsidation plasmid, wherein the encapsidation plasmid either comprises lentiviral, especially HIV-1, gag-pol coding sequences suitable for the production of integration-competent vector particles or modified gag-pol coding sequences suitable for the production of integration-deficient vector particles, wherein said gag-pol sequences are from the same lentivirus sub-family as the DNA flap element, wherein said lentiviral gag-pol or modified gag-pol sequence is under the control of regulating expression sequences.

The stable cell lines expressing the vector particles of the invention are in particular obtained by transfection of the plasmids.

Accordingly, the vector plasmid may comprise one or several expression cassettes for the expression of the various fusion polypeptides or may comprise bi-cistronic or multi-cistronic expression cassettes where the recombinant polynucleotides encoding the fusion polypeptide(s) comprising the antigenic polypeptide(s) are optionally separated by an IRES sequence of viral origin (Internal Ribosome Entry Site), or by the sequence encoding a 2A peptide as disclosed herein.

The internal promoter contained in the vector genome and controlling the expression of the recombinant polynucleotide encoding a fusion polypeptide of the virus (as a transgene or in an expression cassette) may be selected from the promoters of the following genes: MHC Class I promoters, such as human β2-microglobulin promoter (β2M promoter), the SP1-β2m promoter, or EF1α, human PGK, PPI (preproinsulin), thiodextrin, HLA DR invariant chain (P33), HLA DR alpha chain, Ferritin L chain or Ferritin H chain, Chymosin beta 4, Chimosin beta 10, or Cystatin Ribosomal Protein L41 CMVie or chimeric promoters such as GAG(CMV early enhancer/chicken β actin) disclosed in Jones S. et al (2009) or BCUAG.

A promoter among the above-cited internal promoters may also be selected for the expression of the envelope protein(s) and packaging (gag-pol derived) proteins.

The following particular embodiments may be carried out when preparing the lentiviral vector based on human lentivirus, and especially based on HIV-1 virus.

According to the invention, the genome of the lentiviral vector is derived from a human lentivirus, especially from the HIV lentivirus. In particular, the pseudotyped lentiviral vector is an HIV-based vector, such as an HIV-1, or HIV-2 based vector, in particular is derived from HIV-1M, for example from the BRU or LAI isolates. Alternatively, the lentiviral vector providing the necessary sequences for the vector genome may be originating from lentiviruses such as EIAV, CAEV, VISNA, FIV, BIV, SIV, HIV-2, HIV-O which are capable of transducing mammalian cells.

As stated above, when considering it apart from the recombinant polynucleotide that it finally contains, the vector genome is a replacement vector in which the nucleic acid between the 2 long terminal repeats (LTRs) in the original lentivirus genome has been restricted to cis-acting sequences for DNA or RNA synthesis and processing, including for the efficient delivery of the transgene to the nuclear of cells in the host, or at least is deleted or mutated for essential nucleic acid segments that would enable the expression of lentiviral structure proteins including biological functional GAG polyprotein and possibly POL and ENV proteins.

In a particular embodiment, the 5′ LTR and 3′ LTR sequences of the lentivirus are used in the vector genome, but the 3′ LTR at least is modified with respect to the 3′ LTR of the original lentivirus at least in the U3 region which for example can be deleted or partially deleted for the enhancer (delta U3). The 5′ LTR may also be modified, especially in its promoter region where for example a Tat-independent promoter may be substituted for the U3 endogenous promoter.

In a particular embodiment the vector genome comprises one or several of the coding sequences for Vif-, Vpr, Vpu- and Nef-accessory genes (for HIV-1 lentiviral vectors). Alternatively, these sequences can be deleted independently or each other or can be non-functional (second-generation lentiviral vector).

The vector genome of the lentiviral vector particles comprises, as an inserted cis-acting fragment, at least one polynucleotide consisting in the DNA flap element or containing such DNA flap element. In a particular embodiment, the DNA flap is inserted upstream of the polynucleotide encoding the fusion polypeptide of the invention carrying the antigenic polypeptide(s) and is advantageously—although not necessarily—located in an approximate central position in the vector genome. A DNA flap suitable for the invention may be obtained from a retrovirus, especially from a lentivirus, in particular a human lentivirus especially a HIV-1 retrovirus, or from a retrovirus-like organism such as retrotransposon. It may be alternatively obtained from the CAEV (Caprine Arthritis Encephalitis Virus) virus, the EIAV (Equine Infectious Anaemia Virus) virus, the VISNA virus, the SIV (Simian Immunodeficiency Virus) virus or the FIV (Feline Immunodeficiency Virus) virus. The DNA flap may be either prepared synthetically (chemical synthesis) or by amplification of the DNA providing the DNA Flap from the appropriate source as defined above such as by Polymerase chain reaction (PCR). In a more preferred embodiment, the DNA flap is obtained from an HIV retrovirus, for example HIV-1 or HIV-2 virus including any isolate of these two types.

The DNA flap (also designated cPPT/CTS) (defined in Zennou V. et al. ref 27, 2000, Cell vol 101, 173-185 or in WO 99/55892 and WO 01/27304), is a structure which is central in the genome of some lentiviruses especially in HIV, where it gives rise to a 3-stranded DNA structure normally synthesized during especially HIV reverse transcription and which acts as a cis-determinant of HIV genome nuclear import. The DNA flap enables a central strand displacement event controlled in cis by the central polypurine tract (cPPT) and the central termination sequence (CTS) during reverse transcription. When inserted in lentiviral-derived vectors, the polynucleotide enabling the DNA flap to be produced during reverse-transcription, stimulates gene transfer efficiency and complements the level of nuclear import to wild-type levels (Zennou et al., Cell, 2000 Cell vol 101, 173-185 or in WO 99/55892 and WO 01/27304). Sequences of DNA flaps have been disclosed in the prior art, especially in the above cited patent applications. These sequences are also disclosed in the sequence of the pTRIP vector herein described. They are preferably inserted as a fragment, optionally with additional flanking sequences, in the vector genome, in a position which is preferably near the centre of said vector genome. Alternatively, they may be inserted immediately upstream from the promoter controlling the expression of the polynucleotide(s) encoding the fusion polypeptide of the invention. Said fragments comprising the DNA flap, inserted in the vector genome may have a sequence of about 80 to about 200 bp, depending on its origin and preparation.

According to a particular embodiment, a DNA flap has a nucleotide sequence of about 90 to about 140 nucleotides.

In HIV-1, the DNA flap is a stable 99-nucleotide-long plus strand overlap. When used in the genome vector of the lentiviral vector of the invention, it may be inserted as a longer sequence, especially when it is prepared as a PCR fragment. A particular appropriate polynucleotide comprising the structure providing the DNA flap is a 124-base pair polymerase chain reaction (PCR) fragment encompassing the cPPT and CTS regions of the HIV-1 DNA.

It is specified that the DNA flap used in the genome vector and the polynucleotides of the encapsidation plasmid encoding the GAG and POL polyproteins should originate from the same lentivirus sub-family or from the same retrovirus-like organism.

Preferably, the other cis-activating sequences of the genome vector also originate from the same lentivirus or retrovirus-like organism, as the one providing the DNA flap.

The vector genome may further comprise one or several unique restriction site(s) for cloning the recombinant polynucleotide.

In a preferred embodiment, in said vector genome, the 3′ LTR sequence of the lentiviral vector genome is devoid of at least the activator (enhancer) and possibly the promoter of the U3 region. In another particular embodiment, the 3′ LTR region is devoid of the U3 region (delta U3). In this respect, reference is made to the description in WO 01/27300 and WO 01/27304.

In a particular embodiment, in the vector genome, the U3 region of the LTR 5′ is replaced by a non lentiviral U3 or by a promoter suitable to drive tat-independent primary transcription. In such a case, the vector is independent of tat transactivator (third generation vector).

The vector genome also comprises the psi (T) packaging signal. The packaging signal is derived from the N-terminal fragment of the gag ORF. In a particular embodiment, its sequence could be modified by frameshift mutation(s) in order to prevent any interference of a possible transcription/translation of gag peptide, with that of the transgene.

The vector genome may optionally also comprise elements selected among a splice donor site (SD), a splice acceptor site (SA) and/or a Rev-responsive element (RRE).

According to a particular embodiment, the vector plasmid (or added genome vector) comprises the following cis-acting sequences for a transgenic expression cassette:
1. The LTR sequence (Long-Terminal Repeat), required for reverse transcription, the sequences required for transcription and including optionally sequences for viral DNA integration. The 3′ LTR is deleted in the U3 region at least for the promoter to provide SIN vectors (Self-inactivating), without perturbing the functions necessary for gene transfer, for two major reasons: first, to avoid trans-activation of a host gene, once the DNA is integrated in the genome and secondly to allow self-inactivation of the viral cis-sequences after retrotranscription. Optionally, the tat-dependent U3 sequence from the 5′-LTR which drives transcription of the genome is replaced by a non endogenous promoter sequence. Thus, in target cells only sequences from the internal promoter will be transcribed (transgene).
2. The T region, necessary for viral RNA encapsidation.
3. The RRE sequence (REV Responsive Element) allowing export of viral messenger RNA from the nucleus to the cytosol after binding of the Rev protein.
4. The DNA flap element (cPPT/CTS) to facilitate nuclear import.
5. Optionally post-transcriptional regulatory elements, especially elements that improve the expression of fusion polypeptide and/or antigenic polypeptide in dendritic cells, such as the WPRE cis-active sequence (Woodchuck hepatitis B virus Post-Responsive Element) also added to optimize stability of mRNA (Zufferey et al., 1999), the matrix or scaffold attachment regions (SAR and MAR sequences) such as those of the immunoglobulin-kappa gene (Park F. et al Mol Ther 2001; 4: 164-173).

The lentiviral vector of the invention is non replicative (replication-incompetent) i.e., the vector and lentiviral vector genome are regarded as suitable to alleviate concerns regarding replication competent lentiviruses and especially are not able to form new particles budding from the infected host cell after administration. This may be achieved in well-known ways as the result of the absence in the lentiviral genome of the gag, pol or env genes, or their absence as “functional genes”. The gag and pol genes are thus, only provided in trans. This can also be achieved by deleting other viral coding sequence(s) and/or cis-acting genetic elements needed for particles formation.

By “functional” it is meant a gene that is correctly transcribed, and/or correctly expressed. Thus, if present in the lentiviral vector genome of the invention in this embodiment contains sequences of the gag, pol, or env are individually either not transcribed or incompletely transcribed; the expression “incompletely transcribed” refers to the alteration in the transcripts gag, gag-pro or gag-pro-pol, one of these or several of these being not transcribed. Other sequences involved in lentiviral replication may also be mutated in the vector genome, in order to achieve this status. The absence of replication of the lentiviral vector should be distinguished from the replication of the lentiviral genome. Indeed, as described before, the lentiviral genome may contain an origin of replication ensuring the replication of the lentiviral vector genome without ensuring necessarily the replication of the vector particles.

In order to obtain lentiviral vectors according to the invention, the vector genome (as a vector plasmid) must be encapsidated in particles or pseudo-particles. Accordingly, lentiviral proteins, except the envelope proteins, have to be provided in trans to the vector genome in the producing system, especially in producing cells, together with the vector genome, having recourse to at least one encapsidation plasmid carrying the gag gene and either the pol lentiviral gene or an integrative-incompetent pol gene, and preferably lacking some or all of the coding sequences for Vif-, Vpr, Vpu- and Nef-accessory genes and optionally lacking Tat (for HIV-1 lentiviral vectors).

A further plasmid is used, which carries a polynucleotide encoding the envelope pseudotyping protein(s) selected for pseudotyping lentiviral vector particles.

In a preferred embodiment, the packaging plasmid encodes only the lentiviral proteins essential for viral particle synthesis. Accessory genes whose presence in the plasmid could raise safety concerns are accordingly removed. Accordingly, viral proteins brought in trans for packaging are respectively as illustrated for those originating from HIV-1:
1. GAG proteins for building of the matrix (MA, with apparent Molecular Weight p17), the capsid (CA, p24) and nucleocapsid (NC, p6).
2. POL encoded enzymes: integrase, protease and reverse transcriptase.
3. TAT and REV regulatory proteins, when TAT is necessary for the initiation of LTR-mediated transcription; TAT expression may be omitted if the U3 region of 5′LTR is substituted for a promoter driving tat-independent transcription. REV may be modified and accordingly used for example in a recombinant protein which would enable recognition of a domain replacing the RRE sequence in the vector genome or used as a fragment enabling binding to the RRE sequence through its RBD (RNA Binding Domain).

In order to avoid any packaging of the mRNA generated from the genes contained in the packaging plasmid in the viral particles, the T region is removed from the packaging plasmid. A heterologous promoter is inserted in the plasmid to avoid recombination issues and a poly-A tail is added 3′ from the sequences encoding the proteins. Appropriate promoters have been disclosed above.

The envelope plasmid encodes the envelope protein(s) for pseudotyping which are disclosed herein, under the control of an internal promoter, as disclosed herein.

Any or all the described plasmids for the preparation of the lentiviral vector particles of the invention may be codon optimized (CO) in the segment encoding proteins. Codon optimization according to the invention is preferably performed to improve translation of the coding sequences contained in the plasmids, in mammalian cells, murine or especially human cells. According to the invention, codon optimization is especially suited to directly or indirectly improve the preparation of the vector particles or to improve their uptake by the cells of the host to whom they are administered, or to improve the efficiency of the transfer of the polynucleotide encoding the fusion polypeptide comprising the antigenic polypeptide (transgene) in the genome of the transduced cells of the host. Codon optimization is illustrated for the coding sequences used in the examples.

In a particular embodiment of the invention, the pseudotyped lentiviral vector is also, or alternatively, integrative-competent, thus enabling the integration of the vector genome and of the recombinant polynucleotide which it contains into the genome of the transduced cells or in the cells of the host to whom it has been administered.

In another particular embodiment of the invention, the pseudotyped lentiviral vector is also, or alternatively, integrative-incompetent. In such a case, the vector genome and thus the recombinant polynucleotide which it contains do not integrate into the genome of the transduced cells or in the cells of the host to whom it has been administered.

The recombinant lentiviral vector particle of the invention may thus be a recombinant integration-deficient lentiviral vector particle, in particular wherein the recombinant integration-deficient lentiviral vector particle is a HIV-1 based vector particle and is integrase deficient as a result of a mutation of the integrase gene encoded in the genome of the lentivirus in such a way that the integrase is not expressed or not functionally expressed, in particular the mutation in the integrase gene leads to the expression of an integrase substituted on its amino acid residue 64, in particular the substitution is D64V in the catalytic domain of the HIV-1 integrase encoded by Pol.

The present invention relates to the use of a lentiviral vector wherein the expressed integrase protein is defective and which further comprises at least one polynucleotide especially encoding at least one fusion polypeptide of the invention, in particular comprising at least one antigenic polypeptide carrying epitope(s) of a virus as disclosed herein, in an immunogenic composition.

By “integration-incompetent”, it is meant that the integrase, preferably of lentiviral origin, is devoid of the capacity of integration of the lentiviral genome into the genome of the host cells i.e., an integrase protein mutated to specifically alter its integrase activity.

Integration-incompetent lentiviral vectors are obtained by modifying the pol gene encoding the Integrase, resulting in a mutated pol gene encoding an integrative deficient integrase, said modified pol gene being contained in the encapsidation plasmid. Such integration-incompetent lentiviral vectors have been described in patent application WO 2006/010834. Accordingly, the integrase capacity of the protein is altered whereas the correct expression from the encapsidation plasmid of the GAG, PRO and POL proteins and/or the formation of the capsid and hence of the vector particles, as well as other steps of the viral cycle, preceding or subsequent to the integration step, such as the reverse transcription, the nuclear import, stay intact. An integrase is said defective when the integration that it should enable is altered in a way that an integration step takes place less than 1 over 1000, preferably less than 1 over 10000, when compared to a lentiviral vector containing a corresponding wild-type integrase.

In a particular embodiment of the invention, the defective integrase results from a mutation of class 1, preferably amino acid substitutions (one-amino acid substitution) or short deletions fulfilling the requirements of the expression of a defective integrase. The mutation is carried out within the pol gene. These vectors may carry a defective integrase with the mutation D64V in the catalytic domain of the enzyme, which specifically blocks the DNA cleaving and joining reactions of the integration step. The D64V mutation decreases integration of pseudotyped HIV-1 up to 1/10,000 of wild type, but keep their ability to transduce non dividing cells, allowing efficient transgene expression.

Other mutations in the pol gene which are suitable to affect the integrase capacity of the integrase of HIV-1 are the following: H12N, H12C, H16C, H16V, S81 R, D41A, K42A, H51A, Q53C, D55V, D64E, D64V, E69A, K71A, E85A, E87A, D116N, D116I, D116A, N120G, N120I, N120E, E152G, E152A, D-35-E, K156E, K156A, E157A, K159E, K159A, K160A, R166A, D167A, E170A, H171A, K173A, K186Q, K186T, K188T, E198A, R199C, R199T, R199A, D202A, K211A, Q214L, Q216L, Q221 L, W235F, W235E, K236S, K236A, K246A, G247W, D253A, R262A, R263A and K264H.

In a particular embodiment, mutation in the pol gene is performed at either of the following positions D64, D116 or E152, or at several of these positions which are in the catalytic site of the protein. Any substitution at these positions is suitable, including those described above.

Another proposed substitution is the replacement of the amino acid residues RRK (positions 262 to 264) by the amino acid residues AAH.

In a particular embodiment of the invention, when the lentiviral vector is integration-incompetent, the lentiviral genome further comprises an origin of replication (ori), whose sequence is dependent on the nature of cells where the lentiviral genome has to be expressed. Said origin of replication may be from eukaryotic origin, preferably of mammalian origin, most preferably of human origin. It may alternatively be of viral origin, especially coming from circular episomic DNA, as in SV40 or RPS. It is an advantageous embodiment of the invention to have an origin or replication inserted in the lentiviral genome of the lentiviral vector of the invention. Indeed, when the lentiviral genome does not integrate into the cell host genome (because of the defective integrase), the lentiviral genome is lost in cells that undergo frequent cell divisions; this is particularly the case in immune cells, such as B or T cells. The presence of an origin of replication ensures that at least one lentiviral genome is present in each cell, even after cell division, accordingly maximizing the efficiency of the immune response.

The lentiviral vector genome of said lentiviral vectors of the invention may especially be derived from HIV-1 plasmid pFlap-beta2m-WPREm (6155 bp) (SEQ ID No. 161) which comprises restriction sites BamHI and XhoI for the insertion of the transgene(s) or the expression cassette(s).

Vector particles may be produced after transfection of appropriate cells (such as mammalian cells or human cells, such as Human Embryonic Kidney cells illustrated by 293 T cells) by said plasmids, or by other processes. In the cells used for the expression of the lentiviral particles, all or some of the plasmids may be used to stably express their coding polynucleotides, or to transiently or semi-stably express their coding polynucleotides.

The concentration of particles produced can be determined by measuring the P24 (capsid protein for HIV-1) content of cell supernatants.

The lentiviral vector of the invention, once administered into the host, infects cells of the host, possibly specific cells, depending on the envelope proteins it was pseudotyped with. The infection leads to the release of the lentiviral vector genome into the cytoplasm of the host cell where the retro-transcription takes place. Once under a triplex form (via the DNA flap), the lentiviral vector genome is imported into the nucleus, where the polynucleotide(s) encoding polypeptide(s) of antigen(s) of the pathogen is (are) expressed via the cellular machinery. When non-dividing cells are transduced (such as DC), the expression may be stable. When dividing cells are transduced, such as B cells, the expression is temporary in absence of origin of replication in the lentiviral genome, because of nucleic acid dilution and cell division. The expression may be longer by providing an origin of replication ensuring a proper diffusion of the lentiviral vector genome into daughter cells after cell division. The stability and/or expression may also be increased by insertion of MAR (Matrix Associated Region) or SAR (Scaffold Associated Region) elements in the vector genome.

Indeed, these SAR or MAR regions are AT-rich sequences and enable to anchor the lentiviral genome to the matrix of the cell chromosome, thus regulating the transcription of the polynucleotide encoding the fusion polypeptide of the invention comprising at least one antigenic polypeptide, and particularly stimulating gene expression of the transgene and improving chromatin accessibility.

If the lentiviral genome is non integrative, it does not integrate into the host cell genome. Nevertheless, the at least one polypeptide encoded by the transgene is sufficiently expressed and longer enough to be processed, associated with MHC molecules and finally directed towards the cell surface. Depending on the nature of the polynucleotide(s) encoding antigenic polypeptide(s) of a pathogen, the at least one polypeptide epitope associated with the MHC molecule triggers a cellular immune response.

Unless otherwise stated, or unless technically not relevant, the characteristics disclosed in the present application with respect to any of the various features, embodiments or examples of the structure or use of the lentiviral particles, especially regarding their envelope protein(s), or the recombinant polynucleotide, may be combined according to any possible combinations.

The invention further relates to a combination of compounds for separate administration to a mammalian host, which comprises at least:
(i) lentiviral vector particles of the invention which are pseudotyped with a first determined heterologous viral envelope pseudotyping protein or viral envelope pseudotyping proteins; such first pseudotyping protein may be from the NewJersey strain of VSV;
(ii) provided separately from lentiviral vector particles in (i), lentiviral vector particles of the invention which are pseudotyped with a second determined heterologous viral envelope pseudotyping protein or viral envelope pseudotyping proteins distinct from said first heterologous viral envelope pseudotyping protein(s); such second pseudotyping protein may be from the Indiana strain of VSV.

In another embodiment of the invention, possibly in combination with the above disclosed alternative forms of the nucleic acid, the recombinant polynucleotide encoding the fusion polypeptide of the invention, comprising at least one antigenic polypeptide is structurally modified and/or chemically modified. Illustrative thereof a polynucleotide comprises a Kozak consensus sequence in its 5′ region. Other nucleic acid sequences that are not of lentiviral origin may be present in the vector genome are IRES sequence(s) (Internal Ribosome entry site) suitable to initiate polypeptide synthesis, WPRE sequence or modified WPRE sequence as post-transcriptional regulatory element to stabilize the produced RNA, sequences of linkers or of 2A peptides.

Further features and properties of the present invention, including features to be used in the embodiments described above will be described in the examples and figures which follow and may accordingly be used to characterise the invention.

### List of SEQ ID No.

SEQ ID No. 1 and 2 DENV-NS5-5
SEQ ID No. 3 and 4 DENV-NS3-3A
SEQ ID No. 5 and 6 DENV-NS4A-2K
SEQ ID No. 7 and 8 DENV-NS3-1A
SEQ ID No. 9 and 10 DENV-NS3-3
SEQ ID No. 11 and 12 DENV-NS3-3B
SEQ ID No. 13 and 14 DENV-NS5-2
SEQ ID No. 15 and 16 DENV-NS3-2
SEQ ID No. 17 and 18 DENV-NS3-1
SEQ ID No. 19 and 20 DENV-NS5-1
SEQ ID No. 21 and 22 DENV-NS4B-2
SEQ ID No. 23 and 24 DENV-NS5-3
SEQ ID No. 25 and 26 DENV-NS5-4
SEQ ID No. 27 and 28 DENV-NS4B-1
SEQ ID No. 29 and 30 DENV-Ag1-transgene DENV-Ag1 (codon optimized)”
SEQ ID No. 31 and 32 DENV-NS3-4
SEQ ID No. 33 and 34 DENV-NS3-5
SEQ ID No. 35 and 36 DENV-NS3-6
SEQ ID No. 37 and 38 DENV-Ag2-transgene DENV-Ag2 (codon optimized)
SEQ ID No. 39 and 40 ZIKV-C-1
SEQ ID No. 41 and 42 ZIKV-C-2
SEQ ID No. 43 and 44 ZIKV-C-3/PrM
SEQ ID No. 45 and 46 ZIKV-NS4B-1
SEQ ID No. 47 and 48 ZIKV-NS4B-2/NS5-1
SEQ ID No. 49 and 50 ZIKV-NS5-2
SEQ ID No. 51 and 52 ZIKV-NS5-3
SEQ ID No. 53 and 54 ZIKV-Ag-transgene ZIKV-Ag (codon optimized)
SEQ ID No. 55 and 56 ZIKV-NS1-transgene ZIKV-NS1 (codon optimized)
SEQ ID No. 57 and 58 YFV-NS4B-1
SEQ ID No. 59 and 60 YFV-NS5-2
SEQ ID No. 61 and 62 YFV-NS5-3
SEQ ID No. 63 and 64 YFV-NS4B-3
SEQ ID No. 65 and 66 YFV-NS5-4
SEQ ID No. 67 and 68 YFV-NS5-5
SEQ ID No. 69 and 70 YFV-NS3-3
SEQ ID No. 71 and 72 YFV-NS3-4
SEQ ID No. 73 and 74 YFV-NS2A-1
SEQ ID No. 75 and 76 YFV-NS2A-2
SEQ ID No. 77 and 78 YFV-NS2A-3
SEQ ID No. 79 and 80 YFV-NS2A-4
SEQ ID No. 81 and 82 YFV-NS3-5/NS4A-1
SEQ ID No. 83 and 84 YFV-NS3-2
SEQ ID No. 85 and 86 YFV-NS2B-1
SEQ ID No. 87 and 88 YFV-NS3-1
SEQ ID No. 89 and 90 YFV-NS4A-2
SEQ ID No. 91 and 92 YFV-NS2A-5
SEQ ID No. 93 and 94 YFV-NS5-6
SEQ ID No. 95 and 96 YFV-NS5-7
SEQ ID No. 97 and 98 YFV-NS4A-3”
SEQ ID No. 99 and 100 YFV-2K-1
SEQ ID No. 101 and 102 YFV-NS4B-2
SEQ ID No. 103 and 104 YFV-NS5-1
SEQ ID No. 105 and 106 YFV-Ag1
SEQ ID No. 107 and 108 YFV-C-2
SEQ ID No. 109 and 110 YFV-C-1
SEQ ID No. 111 and 112 YFV-PrM-1
SEQ ID No. 113 and 114 YFV-M-1
SEQ ID No. 115 and 116 YFV-E-1
SEQ ID No. 117 and 118 YFV-NS1-2
SEQ ID No. 119 and 120 YFV-NS1-4
SEQ ID No. 121 and 122 YFV-NS1-1
SEQ ID No. 123 and 124 YFV-E-4
SEQ ID No. 125 and 126 YFV-E-5
SEQ ID No. 127 and 128 YFV-E-2
SEQ ID No. 129 and 130 YFV-E-3
SEQ ID No. 131 and 132 YFV-E-6
SEQ ID No. 133 and 134 YFV-NS1-5
SEQ ID No. 135 and 136 YFV-E-7
SEQ ID No. 137 and 138 YFV-NS1-3
SEQ ID No. 139 and 140 YFV-NS1-6
SEQ ID No. 141 and 142 YFV-Ag2-transgene YFV-Ag2 (codon optimized)
SEQ ID No. 143 and 144 DENV-Ag2_ZIKV-Ag-WPREm_(Flavi-2)-transgene DENV-Ag2_ZIKV-Ag-WPREm_(Flavi-2) (codon optimized)
SEQ ID No. 145 and 146 ZIKV-Ag_DENV-Ag2-WPREm_(Flavi-3)-transgene ZIKV-Ag_DENV-Ag2-WPREm_(Flavi-3) (codon optimized)
SEQ ID No. 147 and 148 DENV-Ag2_ZIKV-NS1-WPREm_(Flavi-4)-transgene DENV-Ag2_ZIKV-NS1-WPREm_(Flavi-4) (codon optimized)
SEQ ID No. 149 and 150 ZIKV-NS1-DENV-Ag2-WPREm_(Flavi-5)-transgene ZIKV-NS1-DENV-Ag2-WPREm_(Flavi-5) (codon optimized)
SEQ ID No. 151 pFlap-beta2m-DENV-Ag1-WPREm
SEQ ID No. 152 pFlap-beta2m-DENV-Ag2-WPREm
SEQ ID No. 153 pFlap-beta2m-ZIKV-Ag-WPREm
SEQ ID No. 154 pFlap-beta2m-ZIKV-NS1-WPREm
SEQ ID No. 155 pFlap-beta2m-YFV-Ag1-WPREm
SEQ ID No. 156 pFlap-beta2m-YFV-Ag2-WPREm
SEQ ID No. 157 pFlap-beta2m-DENV-Ag2_ZIKV-Ag-WPREm_(Flavi-2)
SEQ ID No. 158 pFlap-beta2m-ZIKV-Ag_DENV-Ag2-WPREm_(Flavi-3)
SEQ ID No. 159 pFlap-beta2m-DENV-Ag2_ZIKV-NS1-WPREm_(Flavi-4)
SEQ ID No. 160 pFlap-beta2m-ZIKV-NS1-DENV-Ag2-WPREm_(Flavi-5)
SEQ ID No. 161 empty vector pFlap-beta2m-WPREm
SEQ ID No. 162 Consensus sequence of DENV-1 serotype (DENV1_cons) based on sequences representing different phylogenetic lineages (GenBank accession N^{o}s: ADC92350.1, AJQ21317.2, ABG75761.1, AIG59667.1, AAQ19665.2)
SEQ ID No. 163 Consensus sequence of DENV-2 serotype (DENV2_cons) based on sequences representing different phylogenetic lineages (GenBank accession N^{o}s: AL116136.1, AAD18036.1, AUZ41807.1, AHA42535.1, ANT47239.1)
SEQ ID No. 164 Consensus sequence of DENV-3 serotype (DENV3_cons) based on sequences representing different phylogenetic lineages (GenBank accession N^{o}s: ACV04798.1, BAE48725.1, AIH13925.1, ALS05358.1, A1011765.1)
SEQ ID No. 165 Consensus sequence of DENV-4 serotype (DENV4_cons) based on sequences representing different phylogenetic lineages (GenBank accession N^{o}s: AVA30162.1, A L116138.1, AEJ33672.1, ARN79589.1)”
SEQ ID No. 166 Consensus sequence of all Dengue virus serotypes (DENV1-4_cons) based on consensus sequences of each serotype (DENV1_cons, DENV2_cons, DENV3_cons, and DENV4_cons)
SEQ ID No. 167 Consensus sequence of Asian phylogenetic lineage of ZIKV (ZIKV-Asian_cons) based on sequences of Pf13/251013-18 strain (GenBank accession N^{o} ARB08102.1) and BR/AM/16800005 strain (GenBank accession N^{o} AQU12485.1)
SEQ ID No. 168 Consensus sequence of African phylogenetic lineage of ZIKV (ZIKV-African_cons) based on sequences of SEN/1984/41671-DAK strain (GenBank accession N^{o} AMR39836.1) and MR766-NIID strain (GenBank accession N^{o} BAP47441.1)
SEQ ID No. 169 Consensus sequence of both phylogenetic lineages of ZIKV (ZIKV_ALL_cons) based on sequences of ZIKV-Asian_cons and ZIKV-African_cons
SEQ ID No. 170 human b2m
SEQ ID No. 171SP1-humain b2m promoter
SEQ ID No. 172 BCUAG composite promoter
SEQ ID No. 173 WPREm
SEQ ID No. 174 WPRE
SEQ ID No. 175 FLAP forward primer
SEQ ID No. 176 FLAP reverse primer
SEQ ID No. 177 GAPDH forward primer
SEQ ID No. 178 GAPDH reverse primer
SEQ ID No. 179 DENV forward primer
SEQ ID No. 180 DENV reverse primer
SEQ ID No. 181 DENV Taqman probe
SEQ ID No. 182 DENV4 reverse primer
SEQ ID No. 183 DENV4 Taqman probe

### LEGENDS OF THE FIGURES

FIG. 1 Genetic diversity of DENV. Phylogenetic tree based on the complete polyprotein sequences of DENV-1 (84 sequences), DENV-2 (71 sequences), DENV-3 (46 sequences), and DENV-4 (39 sequences) constructed with MEGA 7 software. Strains representing distinct phylogenic lineages of each genotype, that were selected to identify and predict MHC class I epitopes are shown on the right. Challenge strain origin specifies countries where DENV 35 strains used for experimental infection were originally isolated.

FIG. 2. Selection of epitope-containing regions for polyvalent DENV-Ag (DENV-Ag1). (A) Schematic representation of DENV polyprotein. (B) Amino acid identity plot demonstrating distribution of identical amino acids in the consensus sequences of four DENV serotypes. The consensus sequence for each genotype is SEQ ID No. 162 for DENV1 serotype, SEQ ID No. 163 for DENV2 serotype, SEQ ID No. 164 for DENV3 serotype, SEQ ID No. 165 for DENV4 serotype. Black line shows the regions with the identity score above 80%. (C) Distribution of human MHC class I (black) and class II (grey) epitopes that were referenced as positive in various T cell assays in IEDB database. (D and E) Distribution of human MHC class I epitopes predicted for 4 serotypes of DENV by IEDB and netCTLpan prediction servers, respectively. Each dot corresponds to the center of an epitope and shows its position along the sequence of DENV polyprotein (on the x axis). The y axis indicates the number of times that each epitope could be matched to alignment of DENV sequences used for prediction. For instance, y=5 for each epitope predicted in all used sequences of DENV-1, DENV-2, and DENV-3, and y=4 for such an epitope of DENV-4. Higher values on the y axis correspond to either multiallelic epitopes (presented by >1 allele) or clustered epitopes (those that have identical position on the x axis). Grey boxes outline the regions that were selected to create DENV antigen.

FIG. 3. Alignment of the amino acid sequences included in DENV antigen. Antigenic regions were selected from NS3 (A), NS4A, 2K, and NS4B (B) and NS5 (C) proteins. The first sequence in the alignment (DENV1-4_cons (SEQ ID No. 166)) shows 75% majority consensus sequence of 4 DENV serotypes, created based on the individual consensus of each serotype (DENV1_cons (SEQ ID No. 162), DENV2_cons (SEQ ID No. 163), DENV3_cons (SEQ ID No. 164), and DENV4_cons (SEQ ID No. 165)).

FIG. 4. Structure of DENV-based polyvalent antigens DENV-Ag1 and DENV-Ag2. (A) Arrangement of the individual protein fragments originating from non-structural proteins of DENV in DENV-Ag1. Amino acid linkers connecting different regions and designed to eliminate non-specific MHC class I epitopes at the junction sites are labeled L1 to L10. (B) Modified version of polyvalent DENV antigen (DENV-Ag2) has been developed by replacing the N-terminal 26 aa-long fragment of DENV-Ag1 (that included NS5-5 region and L1 linker) with a 47 aa-long sequence including 3 additional antigenic regions of NS-3 protein (NS3-4, NS3-5, and NS3-6). (C) Protein sequence of DENV-Ag1.

FIG. 5. Immunogenicity of integrative (A) or non-integrative (B) lentiviral vectors expressing DENV-Ag1 in A129 mice. (A) T cell response induced by the integrative vector iLV-DENV-Ag1 pseudotyped either with VSV-G of Indiana (IND) or New Jersey (NJ) serotypes 14 days after a single immunization. (B) T cell response induced by non-integrative vector LV-DENV-Ag1 after either a single immunization protocol (analyzed 14 days post-immunization) or a prime-boost protocol (analyzed 6 days after the second immunization). Statistical significance of the total responses was determined by one-way ANOVA test with Tukey corrections for multiple comparisons (*p<0.05, **p<0.01, ***p<0.001, ****p<0.0001).

FIG. 6. T cell response in IFNAR-BL6 mice after a single immunization with either LV-DENV-Ag1 or LV-GFP analyzed by the intracellular cytokine staining. (start) Gating strategy to identify live CD8+T lymphocytes among the splenocytes extracted from IFNAR-BL6 mice fourteen days post-immunization with a single dose of either LV-DENV-Ag1 or LV-GFP. (cont.) Detection of CD8+ cells expressing cytokines IFNγ, TNFα, and IL-2. (end) Detection of CD8+ cells expressing IFNγ and lymphocyte degranulation marker (CD107a) in response to stimulation with DENV-specific peptides. Last line shows CD8+ cells double positive for expression of IFNγ⁺/TNFα⁺, IFNγ⁺/IL-2⁺, or triple positive for expression of IFNγ⁺/TNFα⁺/IL-2⁺. Left panel: splenocytes of mice immunized with LV-DENV-Ag1 and stimulated with non-specific peptide (YF-C) that is not included in DENV-Ag1 (negative control). Middle panel: splenocytes of mice immunized with LV-GFP and stimulated with DENV-specific peptides (negative control). Right panel: splenocytes of mice immunized with LV-DENV-Ag1 and stimulated with DENV-specific peptides.

FIG. 7. Protection of A129 mice against DENV-1 and DENV-2 infections by single immunization with LV-DENV-Ag1. (A and B) Mean weight of A129 mice (n=10/group) that were immunized intra-muscularly (i.m.) with a single dose of 7.5×10⁶ TU/mouse of either LV-DENV-Ag1(IND) or LV-GFP(IND) vector, and one month post-immunization infected intra-venously (i.v.) with either 1×10⁷ FFU/mouse of DENV-1 or 5×10⁵ FFU/mouse of DENV-2, respectively. (C and D) Viremia in plasma of infected A129 mice measured by RT-qPCR and expressed as genome equivalents (G.E.)/ml. LOD=limit of detection. Statistical significance of the differences between groups was evaluated by unpaired non-parametric Mann-Whitney test (* p<0.05, ** p<0.01, *** p<0.001).

FIG. 8. Protection of IFNAR-BL6 mice against DENV-1 and DENV-2 infections by single immunization with LV-DENV-Ag1. (A and B) Mean weight of IFNAR-BL6 mice (n=5-6/group) that were immunized i.m. with a single dose of 3×10⁸ TU/mouse of either LV-DENV-Ag1(IND) or LV-GFP(IND) vector and one month post-immunization infected i.v. with either 1×10⁷ FFU/mouse of DENV-1 or 2×10⁶ FFU/mouse of DENV-2, respectively. (C and D) Viremia in plasma of infected IFNAR-BL6 mice measured by RT-qPCR and expressed as genome equivalents (G.E.)/ml. (E and F) Viral load in the organs of infected mice collected at day 4 post-infection. LOD=limit of detection. Statistical significance of the differences between groups was evaluated by unpaired non-parametric Mann-Whitney test (* p<0.05, ** p<0.01).

FIG. 9. Protection of IFNAR-BL6 mice against DENV-3 and DENV-4 infections by a single immunization with LV-DENV-Ag1. (A and B) Mean weight of mice (n=6/group) that were immunized i.m. with a single dose of 3×10⁸ TU/mouse of either LV-DENV-Ag1(IND) or LV-GFP(IND) vector and infected i.v. with either 8×10⁶ FFU/mouse of DENV-3 (2 months post-immunization) or with 1×10⁷ FFU/mouse of DENV-4 (1 month post-immunization), respectively. (C and D) Viremia in plasma of infected IFNAR-BL6 mice measured by RT-qPCR and expressed as G.E./ml (E) Viral load in the spleen of mice infected with DENV-3, and (F) viral load in organs of mice infected with DENV-4, expressed in G.E./1 μg of total RNA. Statistical significance of the differences between groups was evaluated by unpaired non-parametric Mann-Whitney test (* p<0.05, ** p<0.01, *** p<0.001, **** p<0.001).

FIG. 10. Protection of A129 mice against DENV-2 infection by prime-boost immunization with LV-DENV-Ag1. (A) Mean weights of A129 mice (n=12/group) that were immunized with 1×10⁷ TU/mouse of either LV-DENV-Ag1(IND) or LV-GFP(IND) and 2 months post-immunization boosted with a second i.m. injection of 2×10⁸ TU/mouse of either LV-DENV-Ag1(NJ) or LV-GFP(NJ), respectively. One month after boost both groups were infected i.v. with 1×10⁷ FFU/mouse of DENV-2. (B) Viremia in plasma of mice measured by RT-qPCR and expressed as G.E./ml. (C) Viral load in the spleen expressed in G.E./1 μg of total RNA. Statistical significance of the differences between groups was evaluated by unpaired non-parametric Mann-Whitney test (* p<0.05).

FIG. 11. Role of the CD8+ cells in the LV-DENV-Ag1-induced protection of IFNAR-BL6 mice against DENV-2 infection. Mean weight of mice after a prime-boost immunization with either LV-DENV-Ag1 or LV-GFP followed by infection with DENV-2 one month later. Before infection groups of 6 mice were pre-injected with either anti-Isotype control antibody (A), anti-CD4+(B) or anti-CD8+(C) antibodies to selectively deplete them from CD4+ or CD8+ cells, respectively. (D) Survival of mice immunized with LV-DENV-Ag1 or LV-GFP and either non-depleted of T cells (isotype control Ab), or depleted from CD4+ or CD8+ T cells (anti-CD4+ or anti-CD8+Ab, respectively). (E) Viremia in the blood of mice immunized with LV-DENV-Ag1 or LV-GFP and infected with DENV-2 that prior to infection were injected with anti-isotope antibodies (transparent violin), anti-CD4+ antibodies (light-grey violin), or anti-CD8+ antibodies (dark-grey violin).

FIG. 12. Selection of the T-cell epitope-containing regions of ZIKV and YFV. (A) Schematic representation of ZIKV and YFV polyproteins showing structural proteins capsid (C), matrix (M) and envelope (E), and non-structural proteins NS1, NS2A, NS2B, NS3, NS4A, NS4B and NS5. (B and C, top) Distribution of human MHC class I (blue/dark grey) and MHC class II (orange/light grey) ZIKV- and YFV-specific epitopes that are referenced as positive in T-cell assays in IEDB database, respectively. (B and C, bottom) Distribution of human MHC class I epitopes predicted from consensus sequences of ZIKV and YFV by IEDB (blue/dark grey) and netCTLpan (orange/light grey) prediction tools. Each dot shows localization of the center of the epitopes in polyprotein sequences of ZIKV or YFV (on x axis) and the number of epitopes that are located at each position of the sequence (on y axis). Regions selected to be included in each antigen are shaded: ZIKV-Ag (yellow/light grey), YFV-Ag1 (also designated YFV-NS) (green/left), and YFV-Ag2 (also designated YFV-S) (purple/middle and right). The y axis indicates the number of times that each epitope could be matched to the alignment of 2 ZIKV sequences representing 2 phylogenetic lineages of ZIKV (African and Asian). For instance, y=2 for each epitope predicted in all used sequences of ZIKV. Higher values on the y axis correspond to either multiallelic epitopes (presented by >1 allele) or clustered epitopes (those that have identical position on the x axis). Boxes outline the regions that were selected to compose ZIKV antigen. (C) Alignment of the amino acid sequences included in ZIKV antigen that were selected from C, PrM, NS4A, NS4B and NS5 proteins. The first sequence in the alignment (ZIKV_ALL_cons (SEQ ID No. 169)) shows consensus sequence of 2 ZIKV lineages i.e., Asian phylogenetic lineage of ZIKV (SEQ ID No. 167: ZIKV-Asian_cons) based on sequences of Pf13/251013-18 strain (GenBank accession N^{o} ARB08102.1) and BR/AM/16800005 strain (GenBank accession N^{o} AQU12485.1) and African phylogenetic lineage of ZIKV (SEQ ID No. 168:ZIKV-African_cons) based on sequences of SEN/1984/41671-DAK strain (GenBank accession N^{o} AMR39836.1) and MR766-NIID strain (GenBank accession N^{o} BAP47441.1), created based on the individual consensus of each lineage. (D) Arrangement of protein regions in ZIKV-Ag.

FIG. 13. Protein sequence of ZIKV-Ag. Sequence representation created with SnapGene 6.1.1, showing antigenic regions (grey boxes) and the amino acid linkers designed to eliminate non-specific MHC class I epitopes (black boxes). Empty colored boxes show peptide sequences containing predicted MHC class I epitopes of H-2^{b} mice that were used to assess immunogenicity of LV-ZIKV-Ag: peptide pool C (black), PrM (green), NS4B-A (orange), NS4B-B (blue), NS5-A (red), and NS5-B (purple).

FIG. 14. Immunogenicity of non-integrative lentiviral vector expressing ZIKV-Ag in A129 mice. A129 mice (n=5/group) were immunized with a single dose of 1×10⁸ TU/mouse of either LV-ZIKV-Ag or LV-GFP (control) vector and T cell response was evaluated by the Elispot test 14 days post-immunization. Splenocytes of immunized mice were re-stimulated with the pools of region-specific peptides predicted to be immunogenic in A129 mice (FIG. 13). The number of cells secreting IFNγ in response to such stimulation (per 10⁶ total splenocytes) is indicated on the y axis. Different shading indicates reaction to epitopes originating from different antigenic regions.

FIG. 15. T cell response in IFNAR-BL6 mice after a single immunization with either LV-ZIKV-Ag or LV-GFP, analyzed by the intracellular cytokine staining. (A) Gating strategy to identify CD8+T lymphocytes among the splenocytes extracted from IFNAR-BL6 mice 14 days post-immunization with 3×10⁸ TU of either LV-ZIKV-Ag or LV-GFP. (B) Detection of CD8+ cells expressing cytokines IFNγ, TNFα, IL-2, and lymphocyte degranulation marker (CD107a) in response to stimulation with ZIKV-specific peptides. Last line shows CD8+ cells double positive for expression of IFNγ⁺/TNFα⁺, IFNγ⁺/IL-2⁺, or triple positive for expression of IFNγ⁺/TNFα⁺/IL-2⁺. Left panel: splenocytes of mice immunized with LV-ZIKV-Ag and stimulated with non-specific peptide (YF-C) that is not included in LV-ZIKV-Ag (negative control). Middle panel: splenocytes of mice immunized with LV-GFP and stimulated with ZIKV-specific peptides (negative control). Right panel: splenocytes of mice immunized with LV-ZIKV-Ag and stimulated with ZIKV-specific peptides.

FIG. 16. T cell response in IFNAR-BL6 mice after a single immunization with either LV-ZIKV-NS1 or LV-GFP, analyzed by the intracellular cytokine staining. (A) Gating strategy to identify CD8+ cytotoxic T lymphocytes among the splenocytes extracted from IFNAR-BL6 mice 14 days post-immunization with 3×10⁸ TU of either LV-ZIKV-NS1 or LV-GFP. (B) Detection of CD8+ cells expressing cytokines IFNγ, TNFα, IL-2, and lymphocyte degranulation marker (CD107a) in response to stimulation with a single pool of 166 overlapping peptides covering NS1 protein of ZIKV. Last line shows CD8+ cells double positive for expression of IFNγ⁺/TNFα⁺, IFNγ⁺/IL-2⁺, or triple positive for expression of IFNγ⁺/TNFα⁺/IL-2⁺. Left panel: splenocytes of mice immunized with LV-ZIKV-NS1 and stimulated with non-specific peptide (YF-C) that is not included in LV-ZIKV-NS1 (negative control). Middle panel: splenocytes of mice immunized with LV-GFP and stimulated with ZIKV-NS1-specific peptides (negative control). Right panel: splenocytes of mice immunized with LV-ZIKV-NS1 and stimulated with ZIKV-specific peptides.

FIG. 17. Protection of A129 mice against ZIKV (strain PF-13) infection by single immunization with either LV-ZIKV-Ag or LV-ZIKV-NS1. (A) Mean weight of A129 mice following the infection with ZIKV. (B) Viremia in plasma of mice immunized by LV-ZIKV-Ag, LV-ZIKV-NS1 or LV-GFP and infected with the ZIKV measured by RT-qPCR and expressed in genome equivalents (G.E.). (C and D) Viral load measured in the organs of infected mice at the end of infection (day 12 post-infection) in the brain and spleen, respectively. Statistical significance of the differences between groups was evaluated by unpaired non-parametric Mann-Whitney test (* p<0.05).

FIG. 18. Protection of IFNAR-BL6 mice against ZIKV (strain PF-13) infection by single immunization with LV-ZIKV-NS1. (A) Mean weight of IFNAR-BL6 mice following the infection with ZIKV. (B) Survival of immunized mice following ZIKV infection. (C) Viremia in the blood of mice immunized by LV-ZIKV-NS1 or LV-GFP and infected with the ZIKV measured by PCR and expressed in genome equivalents (G.E.). (D) Viral load measured in the brain and spleen of mice that survived the infection (day 15 post-infection). Statistical significance of the differences between groups was evaluated by unpaired non-parametric Mann-Whitney test (* p<0.05, ** p<0.01).

FIG. 19. Selection of epitope-containing regions for YFV-Ag1 and YFV-Ag2. (A) Schematic representation of YFV polyprotein (upper), distribution of human MHC class I epitopes that were referenced as positive in various T cell assays in IEDB database (middle) and the epitopes predicted by IEDB and netCTLpan prediction servers (bottom). Each dot corresponds to the center of an epitope and shows its position along the sequence of YFV polyprotein (on the x axis). The y axis indicates the number of times that each epitope could be matched to the alignment of 3 YFV strains representing different phylogenetic lineages of YFV. Higher values on the y axis correspond to either multiallelic epitopes (presented by >1 allele) or clustered epitopes (those that have identical position on the x axis). (B) Arrangement of protein regions in included in YFV-Ag1 (upper) and YFV-Ag2 (lower) antigens.

FIG. 20. Protein sequences of YFV-Ag1 (also designated YFV-NS) and YFV-Ag2 (also designated YFV-S). The sequence of both antigens is identical to the sequence of corresponding regions (grey boxes) of YFV live-attenuated vaccine strain (17D-204). Non-YFV specific amino acids (linkers) connecting different regions and included to eliminate non-specific MHC class I epitopes (black boxes) are shown.

Orange (LV-YF-S) and red (LV-YF-NS) boxes show peptide sequences containing MHC class I epitopes of humans and H-2^{b} mice that were used to assess immunogenicity.

FIG. 21. Immunogenicity of non-integrative lentiviral vector expressing YFV-Ag1 and YFV-Ag2 in A129 mice. T cell response induced by a single immunization with either LV-YFV-Ag1, LV-YFV-Ag2 or LV-GFP (control) was evaluated by the Elispot test 14 days post-immunization. Splenocytes of immunized mice were extracted and stimulated with the pools of antigen-specific peptides either reported or predicted to be immunogenic in A129 mice. Pool-1 was comprised of 7 peptides specific for the non-structural protein regions included in YFV-Ag1 and pool-2 of 3 peptides specific for the structural protein regions included in YFV-Ag2. The number of cells secreting IFNγ in response to such stimulation (per 10⁶ total splenocytes) is indicated on the y axis. Each combination of vector immunization/peptide pool stimulation analyzed in the assay is marked by a different sign.

FIG. 22. T cell response in IFNAR-BL6 mice after a single immunization with either LV-YFV-Ag1 or LV-GFP, analyzed by the intracellular cytokine staining. (A) Gating strategy to identify CD8+ cytotoxic T lymphocytes among the splenocytes extracted from IFNAR-BL6 mice fourteen days post-immunization with a single dose of each vector. (B) Detection of CD8+ cells expressing cytokines IFNγ, TNFα, IL-2, and lymphocyte degranulation marker (CD107a) in response to stimulation with a single pool of 7 peptides derived from non-structural regions of YFV (pool 1). Last line shows CD8+ cells double positive for expression of IFNγ⁺/TNFα⁺, IFNγ⁺/IL-2⁺, or triple positive for expression of IFNγ⁺/TNFα⁺/IL-2⁺. Left panel: splenocytes of mice immunized with LV-YFV-Ag1 and stimulated with a non-specific peptide (YF-C) that was not included in LV-YFV-Ag1 (negative control). Middle panel: splenocytes of mice immunized with LV-GFP and stimulated with YFV-specific peptides (negative control). Right panel: splenocytes of mice immunized with YFV-Ag and stimulated with YFV-specific peptides.

FIG. 23. Protection of A129 mice against YFV (strain 17D-204) infection by a single immunization with either LV-YFV-Ag1, LV-YFV-Ag2 or LV-GFP (control). (A) Mean weight of A129 mice following the infection with YFV. (B) Viremia in the blood of mice immunized by LV-ZIKV-Aq, LV-ZIKV-NS1 or LV-GFP and infected with the YFV measured by RT-qPCR and expressed in genome equivalents (G.E.). (C) weight of spleen and (D) viral load in the spleen in mice immunized with different vectors and infected with YFV.

FIG. 24. Schematic representation of the strategy to modify DENV-Ag1 antigen (to produce DENV-Ag2), and use of DENV-Ag2, ZIKV-Ag, and ZIKV-NS1 to create a set of bivalent DENV/ZIKV antigens.

FIG. 25. Evaluation of T cell response induced by lentiviral vectors expressing DENV-specific antigens DENV-Ag1 and DENV-Ag2 and bivalent antigens Flavi-2, Flavi-3, Flavi-4 and Flavi-5. (A) T cell response induced by a single immunization of IFNAR-BL6 mice with individual vectors was evaluated by the Elispot test 14 days post-immunization. Splenocytes of immunized mice were extracted and stimulated with a combined single pool of 35 DENV-specific peptides that included all previously tested peptide pools that were positive in Elispot tests of LV-DENV-Ag1 in A129 mice. The number of cells secreting IFNγ (per 10⁶ total splenocytes) in response to each vector after stimulation with DENV-specific peptide pool is indicated on the y axis. (B) T cell response induced by a single immunization of C57BL/6 (wt) mice with each vector was evaluated by the Elispot test 14 days post-immunization. Reactivity against DENV was evaluated by extracting splenocytes of immunized animals and stimulating them with a single pool of 12 DENV-specific peptides that we tested previously (pools NS4B-1 and NS5-2 combined) and showed highest reactivity against LV-DENV-Ag1 in A129 mice. Reactivity against ZIKV-NS1 was evaluated by stimulating extracted splenocytes with a single pool of 166 overlapping 15-mer peptides covering the complete NS1 protein of ZIKV. Candidate vectors that were pre-selected for further analysis based on the Elispot results are encircled.

FIG. 26. Protection of IFNAR-BL6 mice against DENV-4 infection by the immunization with either LV-DENV-Ag1, LV-DENV-Ag2, LV-Flavi-3, LV-Flavi-4, or LV-Flavi-5 vectors. IFNAR-BL6 mice (A) Viremia in the blood of mice (n=3) immunized by a single dose of each vector (3×10⁸ TU/mouse) and one month later infected with 1×10⁷ FFU/mouse DENV-4, measured by RT-qPCR and expressed in genome equivalents (G.E.)/ml. (B) Viral load in the spleen of infected mice at d7 post-infection, measured by RT-qPCR and expressed as a number of viral genome equivalents per 1 μg of total RNA.

FIG. 27. Protection of IFNAR-BL6 male mice against ZIKV infection by immunization with either LV-Flavi-3, LV-Flavi-4 or LV-Flavi-5 bivalent vector. (A) Viremia in the plasma of mice immunized by a single dose of each vector (3×10⁸ TU/mouse) and one month later infected with 1×10³ FFU/mouse of ZIKV (PF-13), measured by RT-qPCR and expressed in genome equivalents (G.E.)/ml. (B) Viral load in the organs (brains and testes) of infected male mice at d9 post-infection, measured by RT-qPCR and expressed as a number of viral genome equivalents per 1 μg of total RNA.

FIG. 28. Protection of IFNAR-BL6 mice against DENV-1, DENV-2, DENV-3 and DENV-4 infections by a single-dose immunization with LV-Flavi-5 vector. (A, B, C, D: left panel) Mean weight of IFNAR-BL6 mice immunized either with LV-Flavi-5 or LV-GFP (control) and one-month post-immunization infected with DENV-1, DENV-2, DENV-3 and DENV-4, respectively. (A, B, C, D: right panel) Viremia in the blood of mice immunized with LV-Flavi-5 and one month later infected with DENV-1, DENV-2, DENV-3 and DENV-4, respectively, measured by RT-qPCR and expressed in genome equivalents (G.E.)/ml.

FIG. 29. Comparison of protective effect of LV-Flavi-5 immunization against ZIKV infection in male and female IFNAR-BL6 mice. Mean weight of IFNAR-BL6 female mice (A, left panel) and male mice (B, left panel) immunized either with LV-Flavi-5 or LV-GFP (control) and one-month post-immunization infected with 1×10³ FFU/mouse of ZIKV (PF-13). Survival of IFNAR-BL6 female mice (A, right panel) and male mice (B, right panel) immunized with LV-Flavi-5 and infected with ZIKV.

FIG. 30. Protection of IFNAR-BL6 mice against either DENV-2 or ZIKV infection by a heterologous prime-boost immunization with LV-Flavi-5 and LV-Flavi-3 vectors. (A) Mean weight (left panel) and viremia (right panel) of IFNAR-BL6 mice immunized consecutively with either LV-Flavi-5 and LV-Flavi-3 or twice with LV-GFP (control) and one-month post-immunization infected with DENV-2. (B) Mean weight (upper left panel), survival (upper right panel), viremia measured by RT-qPCR (bottom left panel), or viremia measured by viral titration assay (bottom right panel) of male IFNAR-BL6 mice immunized consecutively with either LV-Flavi-5 and LV-Flavi-3 or twice with LV-GFP (control) and one-month post-immunization infected with ZIKV (PF-13).

FIG. 31. Principle of antigenic design for ZIKV and YFV antigens. A phylogenetic tree representing major genetic lineages of ZIKV and YFV was created based on 17 and 19 complete sequences of each virus, respectively. Consensus sequences representing each lineage were inferred from the sequences and used to identify regions containing known human MHC class I and class II epitopes, as well as predicted MHC class I epitopes. Epitope-containing regions were assembled together and optimized as outlined in the Material and Methods.

FIG. 32. Histological analysis of organs from mice inoculated with ZIKV. Hematoxylin-eosin (H&E) staining of brain (A) and spleen (B) from IFNAR-BL6 mice that were either non-immunized and non-infected (left column), immunized with LV-ZIK and inoculated with ZIKV (central column), or immunized with LV-GFP and inoculated with ZIKV (right column). Representative pictures of 3 mice from each experimental group are shown. Red and white arrows indicate location of red and white pulp zones, respectively. Black arrows indicate vascular cuffing observed in the brain of mice immunized with LV-GFP vector and infected with ZIKV.

FIG. 33. Histological analysis of organs from mice inoculated with YFV. H&E staining of brain (A) and spleen (B) from IFNAR-BL6 mice that were either non-immunized and non-infected (left column), immunized with LV-YF-NS and inoculated with YFV (central column), or immunized with LV-GFP and inoculated with YFV (right column). Representative pictures of 3 animals from each group are shown. Red and white arrows indicate location of red and white pulp zones, respectively. Black arrows indicate vascular cuffing observed in the brain of mice immunized with LV-GFP vector and infected with YFV.

### EXAMPLES

### Polynucleotides and Lentiviral Vectors Expressing Non-Structural Antigens of Dengue Virus as Fusion Polypeptides

The following examples relate to the preparation of recombinant polynucleotides and lentiviral vectors expressing non-structural antigens of the Dengue virus as fusion proteins. Similar protocols have been applied to prepare recombinant polynucleotides and lentiviral vectors expressing non-structural antigens of the Zika virus and of the Yellow Fever virus, as fusion proteins. The design of the YFV fusion polypeptide however did not require the design of consensus sequences because it was based of the sequence of 17-204D yellow fever vaccine strain.

The experimental results and illustrative figures for constructs and lentiviral vectors expressing them for the Zika virus and the Yellow Fever virus are accordingly provided on this basis.

### Materials and Methods

### Design of Dengue Antigen (DENV-Ag1)

The complete polyprotein sequences of DENV were retrieved from nucleotide sequence database (NCBI) (16), aligned with ClustalX (17) and used to construct phylogenetic tree with Mega 7 software (18). A smaller set of sequences chosen to represent genetic variability of DENV included 5 sequences of each DENV-1 (GenBank accession N^{o}s: ADC92350.1, AJQ21317.2, ABG75761.1, AIG59667.1, AAQ19665.2), DENV-2 (GenBank accession N^{o}s: ALl16136.1, AAD18036.1, AUZ41807.1, AHA42535.1, ANT47239.1), DENV-3 (GenBank accession N^{o}s: ACV04798.1, BAE48725.1, AIH13925.1, ALS05358.1, AI011765.1), and 4 sequences of DENV-4 (GenBank accession N^{o}s: AVA30162.1, AL116138.1, AEJ33672.1, ARN79589.1). MAFFT software (19) was used to align sequences of known and predicted T cell epitopes to DENV polyprotein sequences. Alignments were visualized with BioEdit sequence editor to further facilitate selection of epitope-containing regions (20). Blast search algorithm (NCBI website) was used to match epitope sequences to the alignment of DENV polyproteins and determine localization of each epitope in the alignment (16). That data was used to construct XY-plots where each epitope was represented by a single dot showing its position in the alignment (x axis) and the number of times that it was matched to different DENV sequences (y axis). Conserved and/or multiallelic epitopes were identified by a higher matching score and the regions containing such epitopes were preferentially included in DENV antigen. MHC class I epitope predictions on the IEDB server (21) were performed independently for each of the four DENV serotypes using the Proteasomal cleavage/TAP transport/MHC class I binding combined predictor for the set of 27 most prevalent human alleles (22-24). All 8-, 9-, 10-, and 11-mer peptides with the total positive score were retained and combined in a single peptide pool. Predictions on the DTU Bioinformatics server were done using netCTLpan tool (25) for 9-mer peptides predicted to bind 20 most prevalent human alleles and retaining those with the consensus rank of less or equal to 1.0. Distributions of known and predicted T cell epitopes were compared and conserved regions containing maximal number of epitopes were selected. A 75% majority consensus sequence of each DENV genotype as well as a master consensus sequence (SEQ ID No. 166) representing all 4 genotypes (that served as a base for DENV-Ag1) were created using Consensus Maker software tool available at the Los Alamos HIV database website (26). Consensus sequences corresponding to the chosen polyprotein fragments were assembled together as a linear polyprotein and then epitope predictions were repeated to verify that all the epitopes located close to the junction sites were predicted to form correctly, and that no non-specific immunodominant epitopes were artificially created by joining of different regions together. In the case if such epitopes were identified, a de-optimization strategy was applied where hydrophobic amino acid linkers were inserted at the junction site, followed by additional rounds of epitope prediction, until such non-specific epitopes were no longer predicted.

### Production and Titration of Lentiviral Vectors (LV)

A DNA sequence encoding for an assembly of DENV genomic regions (DENV-Ag1), codon-optimized for the expression in mammalian cells, was synthesized commercially (Genescript) and inserted into pUC57 subcloning vector. The insert was excised on BamHI and XhoI restriction sites and re-cloned into pFLAPΔU3-β2m-mWPRE vector between β2m promoter and a mWPRE (mutated Woodchuck Posttranscriptional Regulatory Element) sequence in which the atg starting codon was mutated to avoid transcription of the downstream truncated “X” protein of Woodchuck Hepatitis Virus, in order to improve the vector safety. After recloning the sequence of the insert was verified by sequencing of the regions flanking the restriction sites (Eurofins, Ebersberg, Germany). Plasmids used for vector production were purified using the NucleoBond Xtra Maxi EF Kit (Macherey Nagel), resuspended in Tris-EDTA Endotoxin-Free buffer, quantified with a NanoDrop 2000c spectrophotometer (Thermo Fisher Scientific), aliquoted and stored at −20° C. LV were produced in Human Embryonic Kidney HEK293T cells, as previously detailed (27). Briefly, lentiviral particles were produced by transient calcium phosphate tri-transfection of HEK293T cells with the transfer vector plasmid (pFLAP-β2m-mWPRE, where specific antigen is inserted between β2m and mWPRE elements), an envelope plasmid expressing G protein of VSV (either Indiana (IND) or New Jersey (NJ) serotype), and a packaging plasmid (NDK or NDK-pD64V for the production of integration-proficient or integration-deficient vectors, respectively). Supernatants were harvested 48 h post-transfection, clarified by centrifugation at 2500 rpm at 4° C., and ultracentrifuged at 22 000 rpm during 1 h at 4° C. to concentrate lentiviral particles. Pellets were resuspended in sterile 20 mM PIPES buffer pH 7.2, supplemented with 2.5% glucose and 75 Mm NaCl, aliquoted, and stocked at −80° C. The titer of the lentiviral vector was determined by qPCR on vector-transduced HEK293T cells that were treated with aphidicolin to prevent cell division. In parallel, HEK-293T cells were transduced with a heat-inactivated vector (30 min at 70° C.) to control for plasmid contamination in vector preparation. After 48-72 h of transduction, cells were lysed, genomic DNA was isolated and viral titers were determined by qPCR. To determine the titers a fragment of lentiviral Flap region was amplified (forward primer: 5′-TGG AGG AGG AGA TAT GAG GG-3′ (SEQ ID No. 175); reverse primer: 5′-CTG CTG CAC TAT ACC AGA CA-3′ (SEQ ID No. 176)) as well as a fragment of a cellular GAPDH gene (forward primer: 5′-TCT CCT CTG ACT TCA ACA GC-3′ (SEQ ID No. 177); reverse primer: 5′-CCC TGC ACT TTT TAA GAG CC-3′ (SEQ ID No. 178)). The number of lentiviral vector copies per cell was determined as a ratio of the number of Flap copies to the number of GAPDH copies, which corresponded to the total number of HEK293T cells. Prior to immunization of mice lentiviral vectors were diluted to appropriate concentration in PBS.

### Mice

Ifnar1−/− mice (also called IFNAR-KO) carry Ifnar1tm1Agt allele on either 129 (A129) or C57BL/6J (IFNAR-BL6) genetic background were bred and maintained as colonies under specific pathogen-free conditions at Institute Pasteur. For immunization experiments mice at least 6 weeks-old were used. Immunization was performed by intra-muscular injection in the posterior muscle in a 50 μL volume. Infections by dengue viruses were performed intravenously (i.v.) in the caudal vein in a total volume of 150 μL. Infections by Zika and YFV were performed intra-peritoneally in 200 μl total volume. Mice were monitored for signs of illness (DENV: lethargy, ruffled fur, hunched posture; ZIKV and YFV: lethargy, ruffled fur, hunched posture, neurological symptoms (abnormal movements, paralysis of limbs) and weights were recorded daily during the period when the weight changes were observed (in some experiments excluding weekends). Mice were considered moribund if they lost more than 20% of their initial weight or if 10% weight loss was accompanied by neurological symptoms (i.e. limb paralysis). Blood samples were collected into Microvette 500 K3E EDTA-containing tubes (Starstedt) and centrifuged at 5000 g for 10 min in order to separate plasma from blood cells. Clarified plasma samples were kept at −80° C. before the RNA extraction followed by RT-qPCR analysis with DENV-specific primers. All the experiments were performed in the A3 isolator unit of Institute Pasteur animal facility. Experiments on animals were performed in accordance with the European and French guidelines, subsequent to approval by the Institute Pasteur Safety, Animal Care and Use Committee (protocol agreement delivered by local ethical committee: CETEA no. DAP1800077) and Ministry of High Education and Research (APAFIS #18428-2019010717408411_v2).

### Propagation and Titration of Viral Stocks

Dengue virus serotype 1 (DENV-1) strain KDH0026A was kindly provided by Dr. Lambrechts (Institute Pasteur, Paris, France). Mouse-adapted strain S221 of Dengue serotype 2 virus (DENV-2) was kindly provided by Dr. Shresta (La Jolla Institute for Allergy and Immunology, La Jolla, CA, USA). Dengue virus serotype 3 (DENV-3) strain PaH881/88 and DENV serotype 4 (DENV-4) strain ThD4_0087_77 were both isolated in Thailand in 1988 and 1977, respectively. Zika virus strain H/PF/2013 (also called PF13, GenBank: KJ776791) that belongs to Asian genetic lineage of ZIKV was obtained through the DENFREE (FP7/2007-2013) consortium. The vaccine strain of YFV (17D-204, Stamaryl) was obtained from the commercial lot of vaccine purchased from the Institute Pasteur vaccination center. All virus stocks were produced in Vero E6 cells grown in T-175 tissue flasks with filter cups. Titration were performed on Vero E6 cells grown on 24 well plates. Cells were infected with 300 μl of serial stock dilutions during 1 hour with periodic shaking, and, after removal of inoculation medium, overlayed with DMEM containing 1.6% carboxymethyl cellulose, 2% of FBS and antibiotics. After 5 days of incubation overlaying medium was removed, cells were fixed with 4% PFA for 30 min, and viral foci were revealed by staining with mouse-anti-DENV antibody (4G2) at 0.5 μg/ml (produced by the recombinant protein production facility of Institute Pasteur), followed by the second staining with Goat Anti-mouse IgG HRP conjugate (BioRad, France). The HPR signals were revealed with Vector VIP peroxidase substrate kit (Vector Laboratories, USA) following manufacturer's recommendations.

### Immunogenicity of Antigenic Constructs in Mice (Elispot).

Elispot plates pre-coated with the anti-mouse IFNγ antibodies (Mabtech AB, Nacka Strand, Sweden) were used according to the manufacturer's instructions. Splenocytes from immunized mice were added in triplicates at 1×10⁵ cells/well and stimulated with the peptides pools containing 2 μg/ml of each peptide. Unstimulated splenocytes and splenocytes stimulated by 2.5 μg/ml of Concanavalin A were used as negative and positive controls, respectively. After 24 h of incubation spots were revealed according to the manufacturers' protocol and counted with AID ELISpot Reader System ELR04 (Autoimmune Diagnostika GmbH, Strassberg, Germany). Background signals originating from the wells containing unstimulated cells were subtracted and results were expressed as a number of spot-forming cells per million of splenocytes.

### Analysis of Viremia and the Viral Load in Organs (RT-qPCR)

For the analysis of viremia plasma samples were collected from the individual animals by bleeding from sub-mandibular vein and processed as described above. RNA was extracted from 35 μl of plasma with QIAamp viral RNA mini kit (QIAGEN, Hilden, Germany). For the analysis of viral load in the peripheral organs, a whole organ was collected, weighted, and frozen at −80° C. until the moment of RNA extraction. During the extraction a frozen tissue samples were suspended in 1 ml of TRIzol and homogenized in the FastPrep-24 homogenizer (VWR, France) at 6.0 m/s for 30 sec. Total RNA was purified following the extraction protocol of TRIzol manufacturer. Concentration of RNA was measured by the Nanodrop spectrophotometer and total RNA concentration was adjusted to 0.1 μg/μl in all samples. Ten microliters of each RNA preparation (1 μg of total RNA) was used in the RT-qPCR reaction. For the analysis of DENV, 2-step RT-qPCR reaction (adapted from 28) was performed to measure viral load in plasma and peripheral organs. The RT was performed with Moloney murine leukemia virus (M-MLV) reverse transcriptase and then the resulting product was used to set up two identical qPCR reactions per sample (duplicates) that were ran on a QuantStudio 12K Flex real-time PCR system (Applied Biosystems, Carlsbad, CA, USA). Analysis of samples originating from mice infected with DENV-1, DENV-2 and DENV-3 was done with the following primers and probes described in (28) (forward primer: 5′-GARAGACCAGAGATCCTGCTGTCT-3′ (SEQ ID No. 179); reverse primer: 5′-ACCATTCCATTTTCTGGCGTT-3′ (SEQ ID No. 180); Taqman probe: [5′-FAM]AGCATCATTCCAGGCAC[MGBEQ]-3′) (SEQ ID No. 181). Samples originating from mice infected with DENV-4 were analyzed using the same RT-PCR protocol, but the reverse primer (5′-ACCAATCCATCTCGCGGCGCT-3′) (SEQ ID No. 182) and TaqMan probe (5′-[FAM]AACATCAATCCAGGCAC[MGBEQ]-3′) (SEQ ID No. 183) were modified to match the sequence of DENV-4 strain used for the challenge. Analysis of viremia and organ load of ZIKV was performed using RT-qPCR protocol similar to the one described for DENV, adopted from Lanciotti et al. (29), and analysis of YFV was done with the protocol adopted from Bae et al. (30).

### Antibodies and Other Reagents

Anti-mouse CD8α (clone 2,43), anti-mouse CD4 (clone GK1.5), and IgG2b isotype control (LTF-2) rat antibody (all from InVivoMab) were used in the T cell depletion experiments.

### Statistical Analysis

Statistical analysis was performed with the statistical tests implemented in GraphPad Prism 9 software. For pairwise comparisons either unpaired parametric t-test with or without Welch's corrections (depending on standard deviation (SD), determined from the dataset) or unpaired non-parametric Mann-Whitney test (for small groups of samples) were used. Multiple comparisons were performed either using 1-way ANOVA test (ordinary or Welch, depending on SD) or Kruskal-Wallis test. Data were considered significant when p values were less than 0.05.

### Results

### Design and Refinement of DENV T Cell Antigen

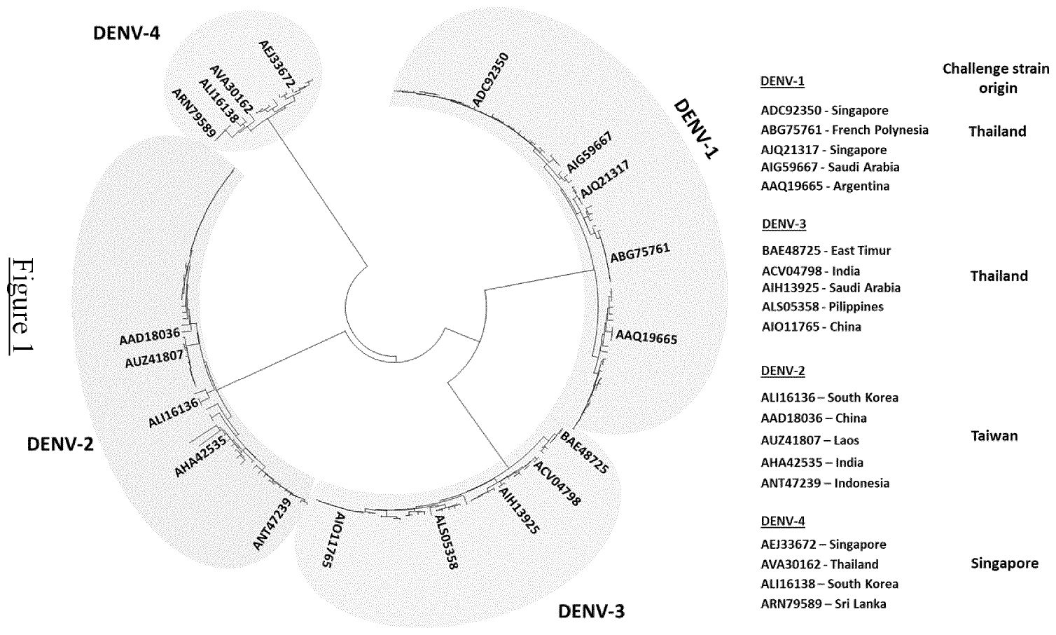

To design the antigen for a polyvalent DENV vaccine a phylogenetic tree was first constructed using 240 complete polyprotein sequences of four DENV serotypes. Based on that tree a smaller set of DENV sequences was selected representing each phylogenetic sub-lineages of each serotype by a single sequence (FIG. 1).

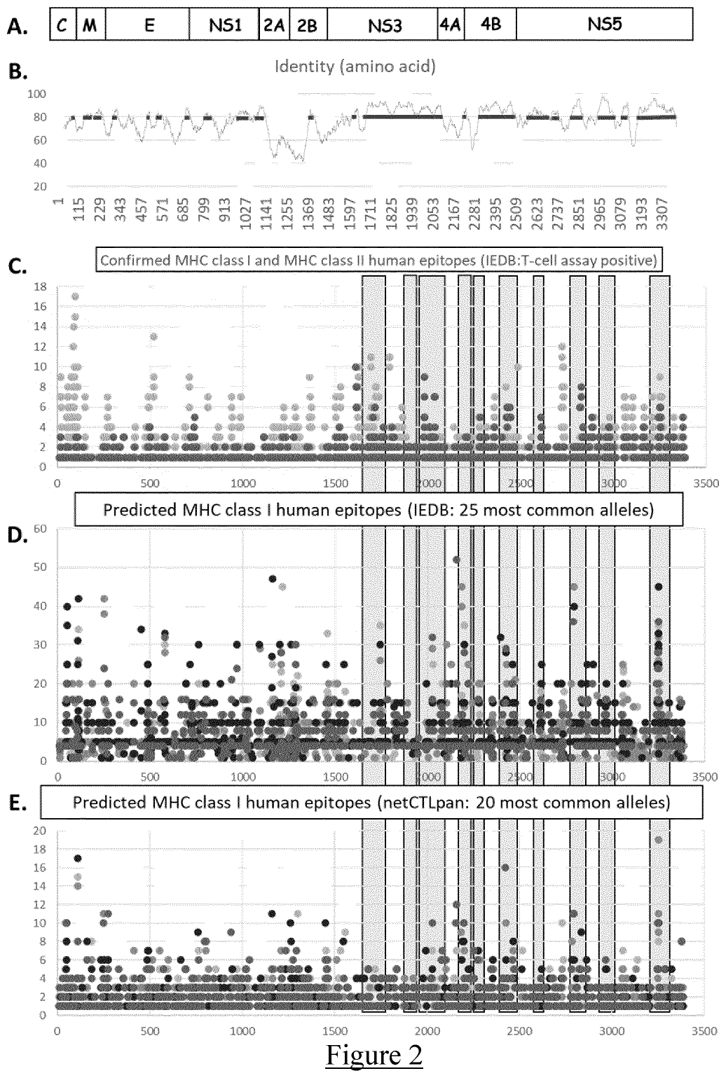

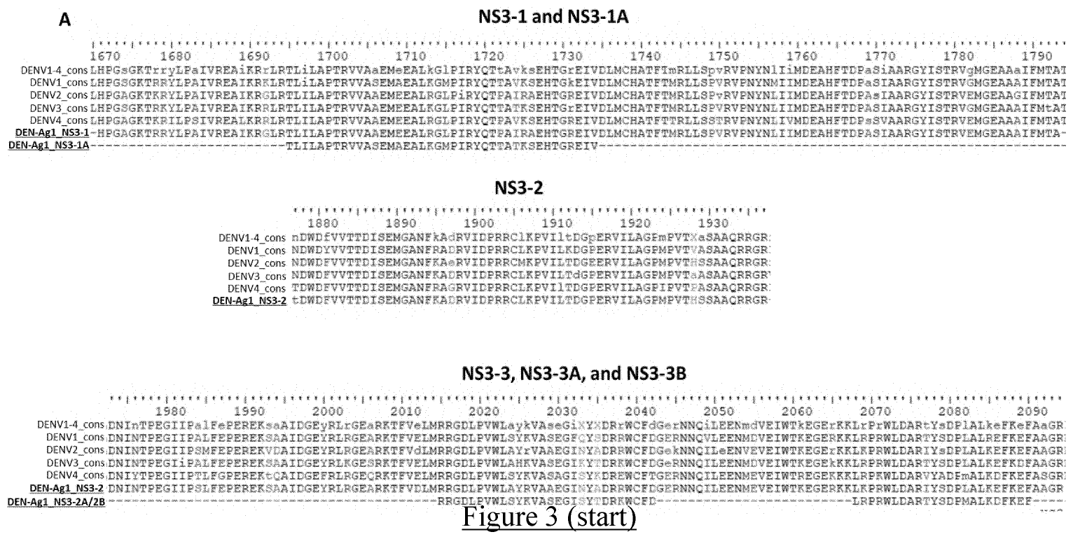

Mapping of the regions cross-conserved between different serotypes of DENV, performed by plotting the alignment score along the polyprotein length, demonstrated that most of such regions are located in the non-structural proteins NS3, NS4B and NS5 (FIGS. 2A and 2B). Several studies have reported previously that these proteins are targeted by cytotoxic T cell responses that could have cross-protective effect against different serotypes of DENV (5-7). To identify T cell epitope-containing regions in DENV sequences, all human MHC class I and MHC class II epitopes annotated as positive in various T cell assays (secretion of IFNγ and/or TNFα and IL2, cytotoxicity) were retrieved from the Immune Epitope Database and Analysis Resource (IEDB) and aligned to the representative set of DENV sequences (21).

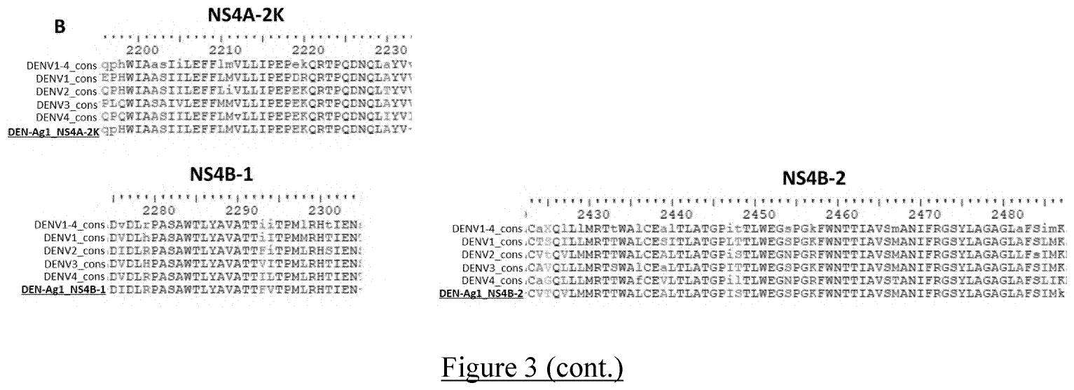

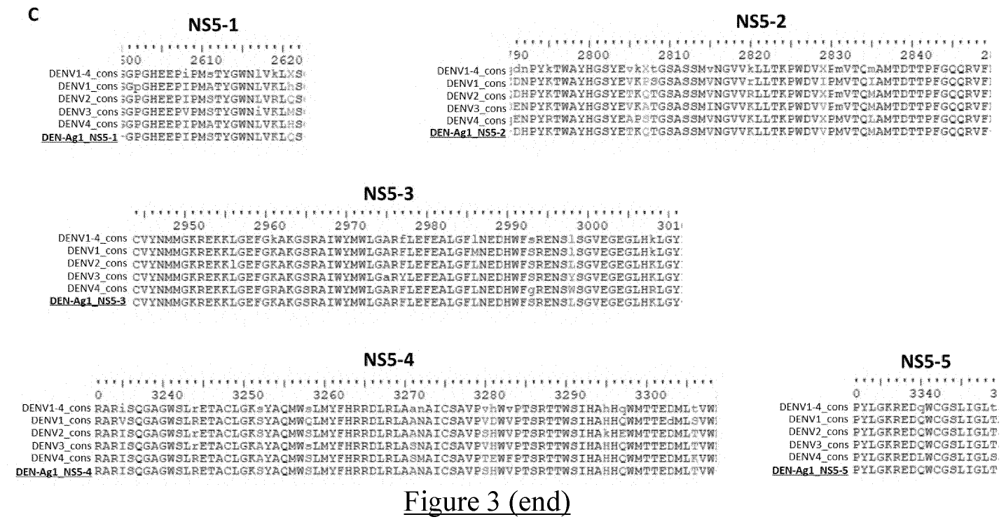

Distribution of known epitopes along DENV sequence alignment was also visualized by an XY-plot (FIG. 2C) that allows to identify epitope-containing regions (epitope clusters) as well as individual epitopes that are either highly similar in different sequences (conserved epitopes) and/or could be presented by multiple HLA alleles (multiallelic epitopes). In accordance with published studies, most of known functionally characterized human MHC class I epitopes were located in the non-structural proteins NS3, NS4B, and NS5, while MHC class II epitopes were more evenly distributed between structural and non-structural proteins. Although several thousands of DENV-specific MHC class I epitopes are referenced as positive in the IEDB, many of them were identified in studies performed in specific geographic regions where the distribution of MHC class I alleles does not necessarily reflect that of global human population. Additionally, many epitopes were identified and characterized in experimental studies performed in transgenic mice bearing human MHC class I alleles associated with strong and protective CTL response to DENV (e.g. HLA-B*07:02) (31). Thus, epitopes presented by such alleles could be over-represented in the database compared to epitopes presented by less protective but globally more prevalent human alleles (e.g. HLA-A*01:01 and HLA-A*24:02). Furthermore, since several serotypes of DENV usually co-circulate in many endemic regions, it is possible that T cell response induced by a particular DENV serotype may not provide optimal protection against another serotype. The abovementioned factors may lead to a biased representation of known human T cell epitopes in the database. In order to compensate for such potential bias and achieve a more balanced representation of epitopes in DENV-based antigen, the chosen dataset of DENV sequences was subjected to a computer prediction of MHC class I epitopes using prediction tools located at immune-epitope database (IEDB) and Technical University of Denmark (DTU Bioinformatics) websites (21, 25). Predicted epitopes were mapped to the alignment of DENV sequences and visualized by XY-plots (FIGS. 2D and 2E). Comparing the distribution of epitopes predicted by two different methods with the distribution of known epitopes allowed more precise selection of regions for DENV-Ag1. Prediction of MHC class II epitopes has not been performed, because algorithms used for prediction of such epitopes were reported to lack the efficiency and predictive power compare to those used for the prediction of MHC class I epitopes (32). Besides, studies of DENV in animal models suggested that cytotoxic T cell response targeting MHC class I epitopes plays more important role in protecting mice against DENV infection. To incorporate genetic variability presented by 4 DENV genotypes in a single sequence, a 75% majority consensus was inferred for each DENV genotype, and then a master consensus sequence was created based on 4 individual consensus sequences (FIG. 3).

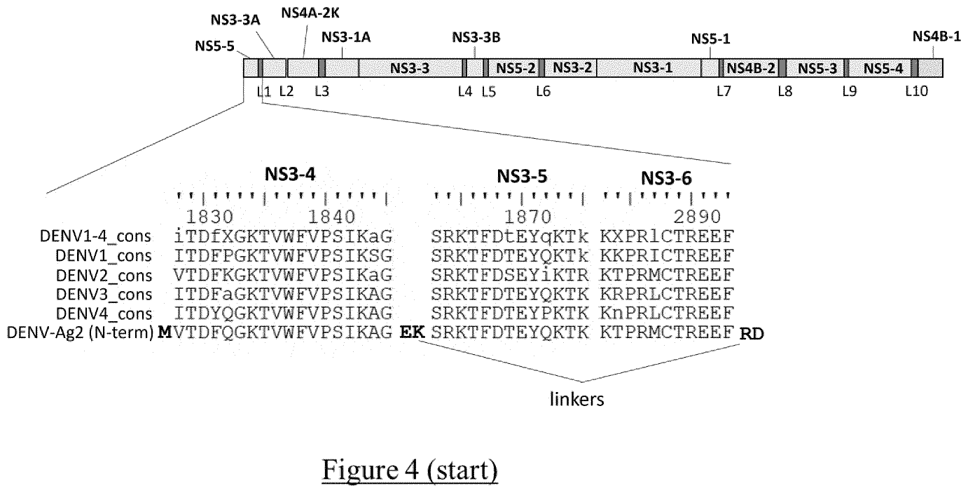

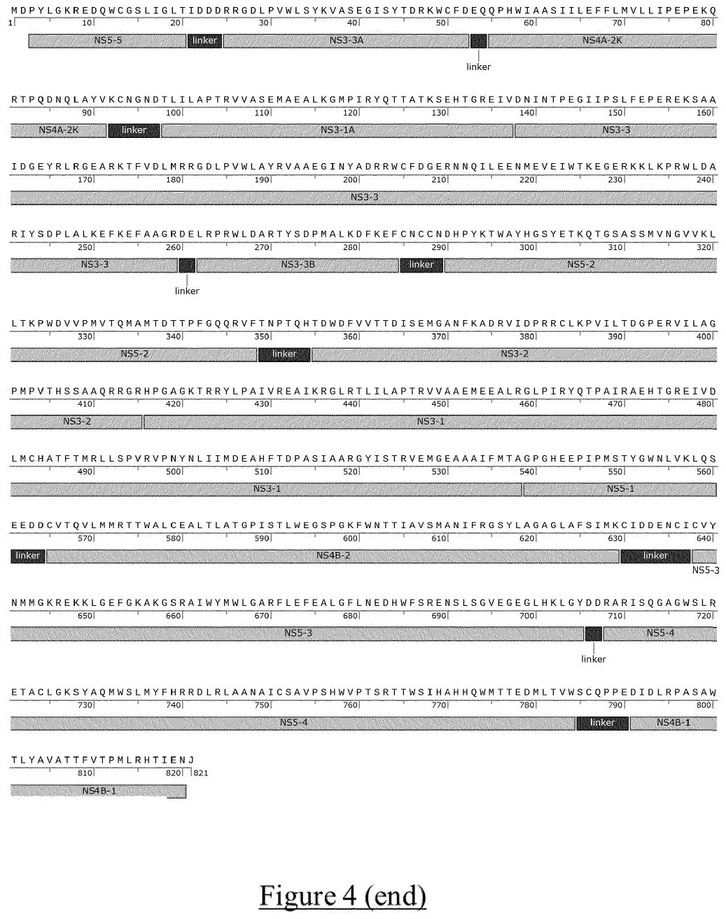

In general, sequence of DENV-Ag1 was identical to the master consensus sequence, except for a number of positions where variability was equally split between different genotypes (e.g. position 1674 of NS3-1 region where serine (S) is encoded by DENV-1 and DENV-3 genotypes and alanine (A) is encoded by DENV-2 and DENV-4 genotypes) or at sites where more significant variation was observed (e.g. position 1928 of NS3-2 region). In such cases the choice of amino acid was based on the number of known or predicted T cell epitopes that included it; the amino acid more represented in the dataset was featured in the sequence. Although such an approach was generally well applicable, in several short regions of the alignment amino acid variation was too substantial to be represented by a single consensus sequence. For this reason three additional short sequences were included in DENV-Ag1: NS3-1A, NS3-3A and NS3-3B, each featuring a permutated sub-region of a larger sequence, that represented the consensus of remaining genotypes (FIG. 3A). Chosen regions were joined together and junction regions were optimized to remove any potential non-specific immunodominant epitopes that could appear at the junction sites (FIG. 4). DENV-Ag2 was created as a modified version of DENV-Ag1 in which its N-terminal 26 aa-long fragment (that included NS5-5 region and L1 linker) was replaced with a 47 aa-long sequence including 3 additional antigenic regions of NS-3 protein (NS3-4, NS3-5, and NS3-6). Such modification included several MHC class I epitopes that were predicted to induce more broad response to DENV compared to that induced by DENV-Ag1. Prediction of the MHC class I epitopes demonstrated that DENV-Ag2 should contain between 26 (minimum amount, predicted for HLA-A*01:01) and 55 (maximum amount, predicted for HLA-A*35:01) human epitopes per allele. The expected coverage of human population with DEN-Ag approximated with the allele coverage tool (IEDB)) predicted that both antigens should induce a protective effect against DENV in 86-100% of individuals from most geographic regions.

### Evaluation of the Immunogenicity of DENV-Ag1 in A129 Mice.

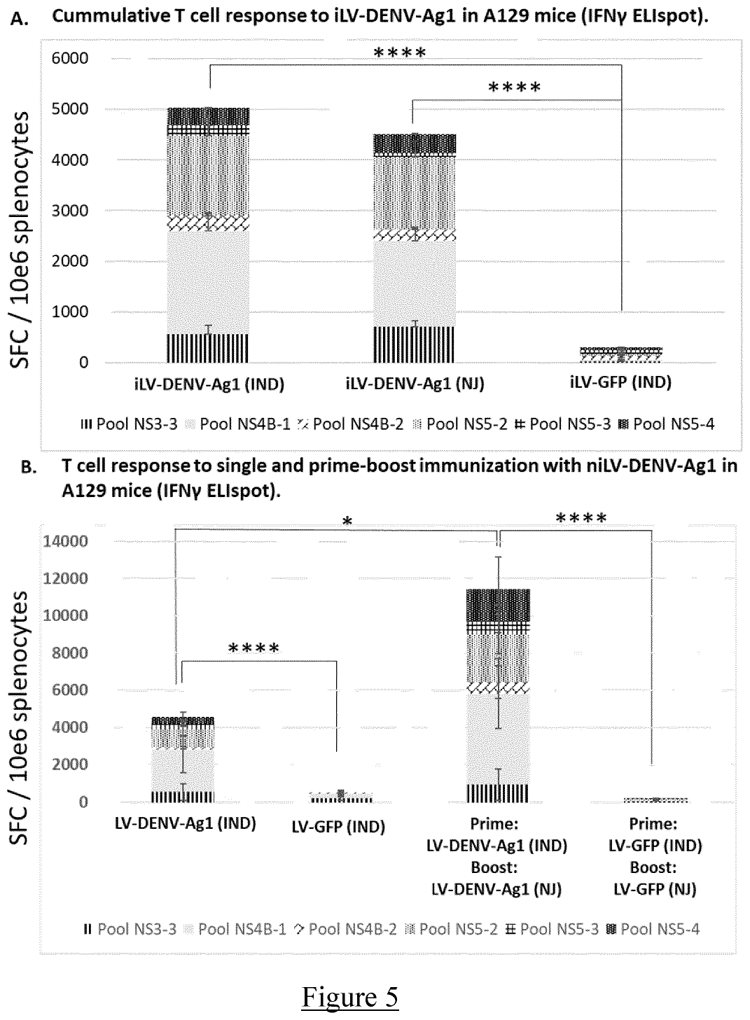

First, we measured activation of the T cell response induced by the integrative lentiviral vectors (iLV) iLV-DENV-Ag1(IND) and iLV-DENV-Ag1(NJ), pseudotyped with the glycoprotein (G) of vesicular stomatitis virus of either Indiana (IND) or New Jersey (NJ) serotype. Three groups of A129 mice (n=5) were immunized intra-muscularly (i.m.) with, respectively, 5×10⁷ TU of iLV-DENV-Ag1(IND), iLV-DENV-Ag1(NJ), or the control vector iLV-GFP(IND). Fourteen days post-immunization splenocytes were extracted and analyzed by Elispot for the secretion of IFNγ in response to re-stimulation with several peptide pools specific for DENV-Ag1. Immunization with both DENV-Ag1-expressing vectors have induced secretion of IFNγ by splenocytes stimulated with the antigen-specific peptides, with non-significant difference between vectors pseudotyped with VSV-IND or VSV-NJ (FIG. 5A).

To analyze T cell responses induced by DENV-Ag1 expressed in a context of a non-integrative LV vector (LV), two groups of A129 mice (n=6) were immunized i.m. with 3×10⁸ TU/mouse of either LV-DENV-Ag1(IND) or LV-GFP(IND), respectively. Antigen delivery by the single injection of LV (used at the 6-fold higher dose than the one of the integrative vector) has induced a comparable magnitude of T cell response (FIG. 5B), indicating that the non-integrative lentiviral vectors can be successfully used for the induction of the immune response against DENV-specific antigen, and corroborating our earlier published results (33). To compare the magnitude of responses induced by a single-dose vs. prime-boost immunization protocols, two groups of A129 mice (n=6) were immunized i.m. with a 7.5×10⁶ TU/mouse of either LV-DENV-Ag1(IND) or LV-GFP(IND). Two months later mice were boosted by another i.m. injection of 3×10⁸ TU/mouse of either LV-DENV-Ag1(NJ) or LV-GFP(NJ), respectively, and the analysis of T cell activity was carried 6 days after the second immunization. Implementation of a prime-boost immunization protocol has significantly increased the mean T cell response (by 2.4-fold).

### Detection of Mediators of T Cell Response (IFNγ, TNFα, IL2, and Lymphocyte Degranulation Marker CD107a) by the Intracellular Cytokine Staining.

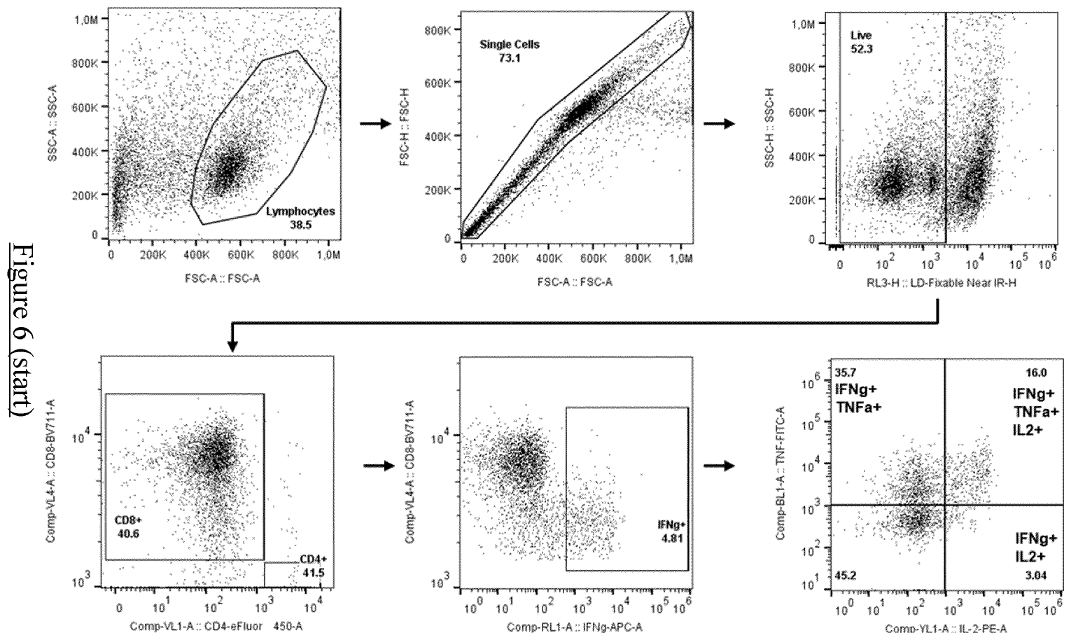

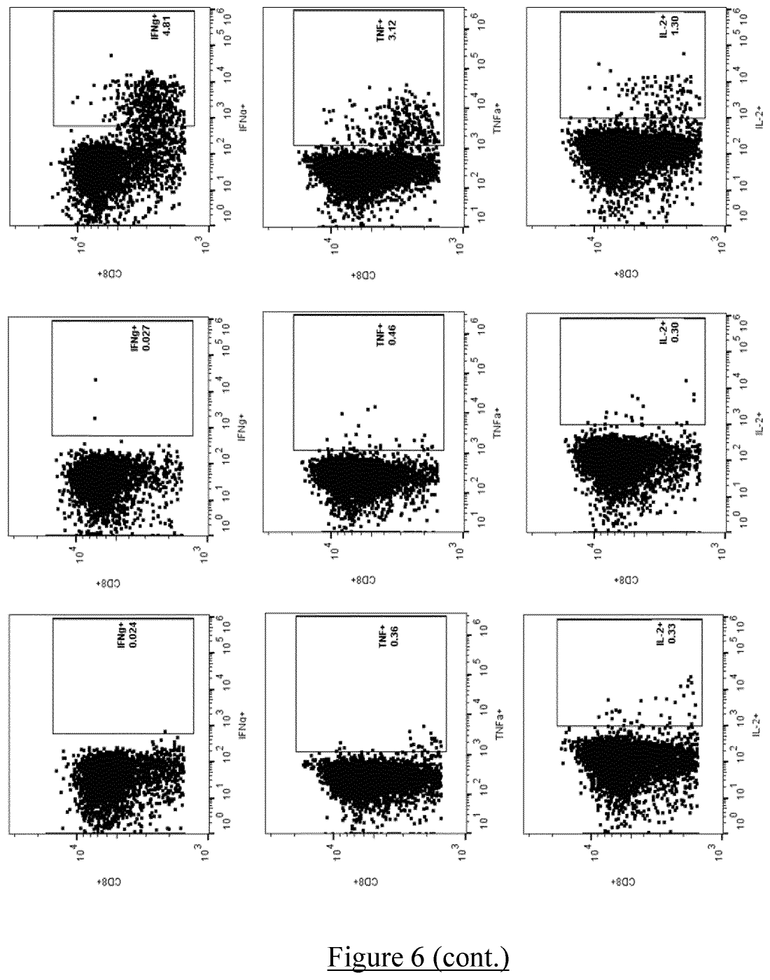

Cytokines released by antigen-experienced cytotoxic T lymphocytes are broadly accepted as an evidence of their targeted action against specific pathogens and several previous studies have correlated T cell immunity against DENV with the presence of DENV-specific T cells secreting IFNγ, TNFα, and IL2. Polypotent T cells (i.e. those that simultaneously secrete 2 or 3 cytokines in response to DENV) are thought to be particularly important for the antiviral response. We have investigated if immunization with LV-DENV-Ag1 vector induced cytokine production by antigen-specific cells and if the same population of T cells could simultaneously secrete several cytokines. Splenocytes from several IFNAR-BL6 mice immunized with 3×10⁸ TU of either LV-DENV-Ag1(IND) or LV-GFP(IND) vector were extracted 14 days post-infection and analyzed by the intracellular cytokine staining (ICS) for T cells secreting IFNγ, TNFα, IL2, and lymphocyte degranulation marker CD107a. During the procedure splenocytes from several mice immunized with the same vector were pooled and stimulated for 3 h with a pool of 11 DENV peptides, following by a 3 h incubation with Brefeldin A/Monensin (FIG. 6). Flow cytometry analysis of cytokine-stained cells has indicated that CD8+ T cells responded to antigen exposure/peptide stimulation by secretion of IFNγ, TNFα, and IL2. Moreover, a proportion of LV-DENV-Ag1-exposed/DENV peptides-stimulated cells co-expressed IFNγ and lymphocyte degranulation marker CD107a, indicating that such cells have target-specific cytotoxic properties and are able to mediate lysis of virus-infected cells. Polypotent CD8+ T cells simultaneously expressing three cytokines (IFNγ, TNFα, and IL2) have also been detected amongst splenocytes that were exposed to LV-DENV-Ag1 and stimulated with DENV-peptides, but not among those that were exposed to unrelated antigen (LV-GFP) or those that were stimulated with non-specific peptide (YF-C).

Single Dose Immunization Protocol: Protection of A129 Mice from DENV-1 and DENV-2 Infections.

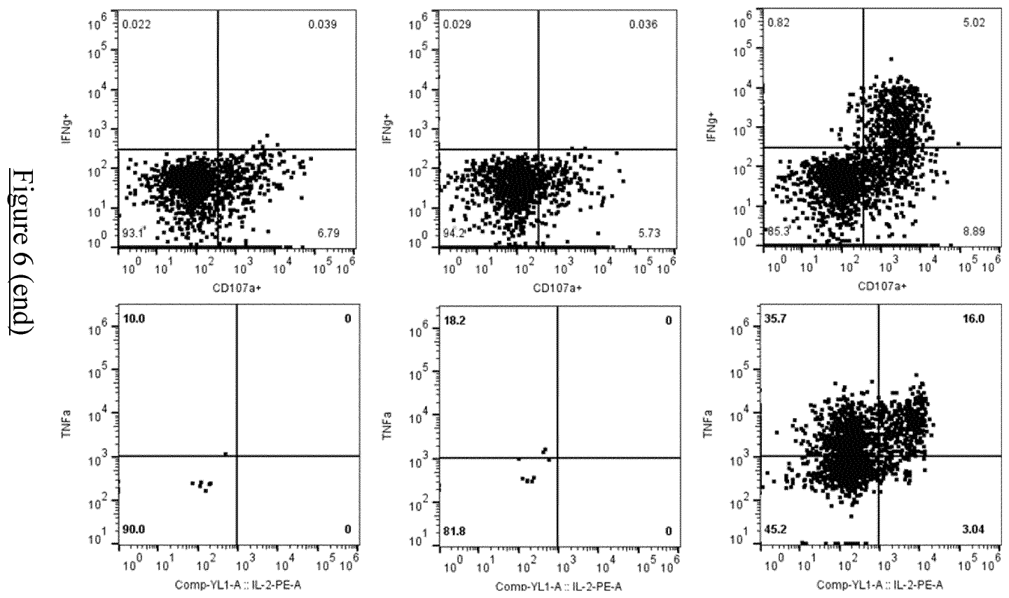

One group of A129 mice (n=10) was immunized intra-muscularly with 7.5×10⁶ TU/mouse of LV-DENV-Ag1(IND), and another group with the same dose of LV-GFP(IND). Twenty-seven days post-immunization each group was further divided in two equal sub-groups that were infected with either DENV-1 (1×10⁷ FFU/mouse) or DENV-2 (5×10⁵ FFU/mouse). The weight of infected mice was measured daily, and the blood samples were collected from the subgroups of several mice on days 1, 2, 3, 4 and 7-8 post-infection (p.i.) to monitor the level of viremia inplasma. Mice were sacrificed on days 7-8 p.i., when the increase of the weight indicated that they were recovering from infection. DENV-1 did not produce any symptoms in A129 mice, while mice infected with DENV-2 developed ruffled fur that became noticeable on day 1-2 p.i. and gradually became less evident during the progression of the recovery phase (around days 5-6, when mice started to regain weight). The mean weight of mice immunized with LV-DENV-Ag1(IND) and infected with either DENV-1 or DENV-2 was significantly higher than the weight of LV-GFP(IND)-immunized mice on d3-4 post-infection (FIGS. 7A and 7B).

Although viremia was detected in all groups of mice, the level of DENV-1 and DENV-2 viremia measured on days 1 to 4 post-infection was on average 20-30 times lower in mice immunized with DENV-specific vector compared to mice immunized with the control vector (FIGS. 7C and 7D). Furthermore, viremia in the groups immunized with LV-DEN-Ag1(IND) was resolved earlier than in the control groups: DENV-1 was not detectable in the plasma starting from day 6 p.i., and DENV-2 could not be detected after day 3 p.i. In contrast, in the plasma of mice immunized with LV-GFP(IND) both viruses were detectable up to 7-8 days post-infection. These results demonstrate that immunization of A129 mice with a single dose of LV-DENV-Ag1(IND) induces partial protection against DENV-1 and DENV-2 infections, significantly reducing the level of viremia as well as shortening its duration.

Single Dose Immunization Protocol: Protection of IFNAR-BL6 Mice from DENV-1, DENV-2, DENV-3 and DENV-4 Infections.

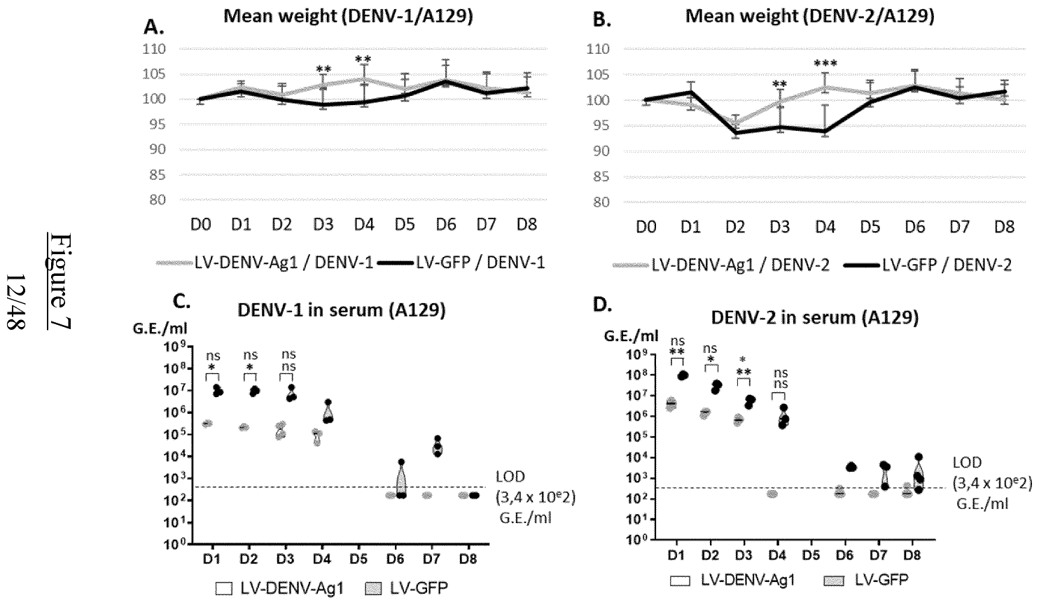

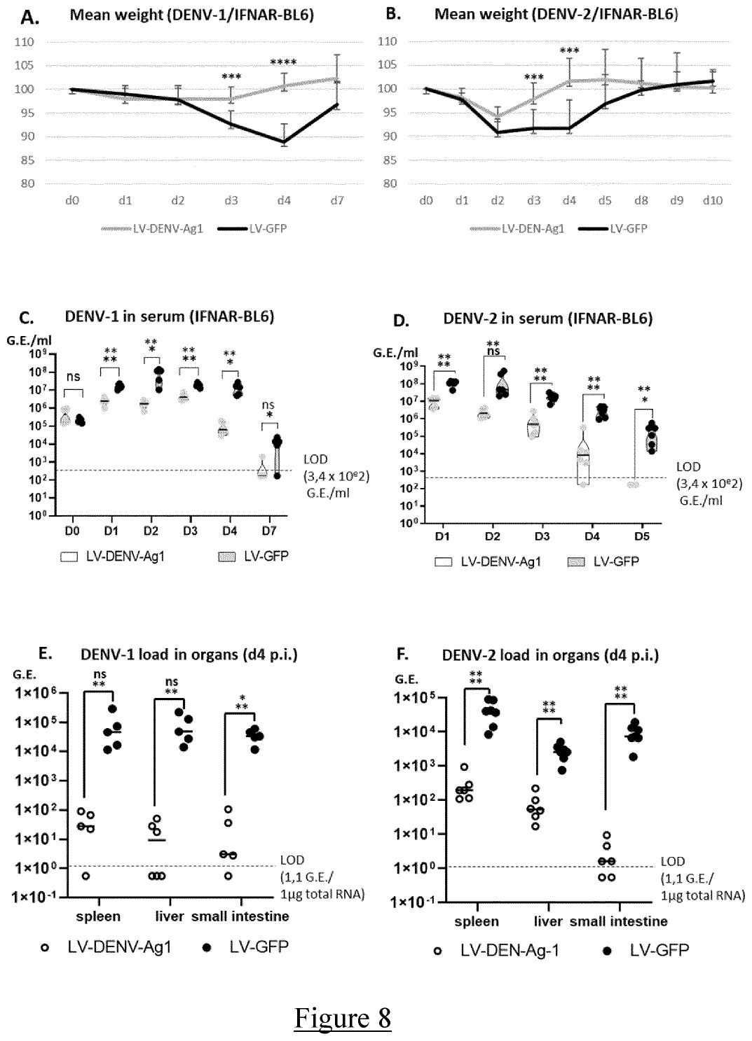

Immunization/protection studies were also performed in IFNAR-KO mice of C57BL/6 origin (IFNAR-BL6), that were previously shown to be more sensitive to experimental infections with various flaviviruses compared to mice of A129 lineage (34, and our unpublished data). To test the protection against DENV-1 and DENV-2, two groups of IFNAR-BL6 mice were immunized with 3×10⁸ TU/mouse of either LV-DENV-Ag1(IND) or LV-GFP(IND). One month later half of the mice from each immunized group were infected with DENV-1 (1×10⁷ FFU/mouse) and another half with DENV-2 (2×10⁶ FFU/mouse). No visible symptoms have been detected in any of the infected mice except for the weight loss that was observed in all groups during the first two days post-infection (FIGS. 8A and 8B). All mice immunized with LV-DENV-Ag1(IND) have regained weight between d3 and d4 post-infection (significant difference with the control mice), while the weight recovery of mice immunized with LV-GFP(IND) was delayed and generally occurred between d4 and d7-8 post-infection. Also, similar to the results obtained on A129 mice, DENV-1 and DENV-2 viremia was significantly lower in IFNAR-BL6 mice immunized with LV-DENV-Ag1(IND), starting from d1 and d2 post-infection, respectively (FIGS. 8C and 8D). Viremia levels also declined faster in mice immunized with DENV-Ag1-expressing vector: starting from d5 p.i. DENV-2 was undetectable in the plasma of such mice, while level of DENV-1 measured on d7 post-infection was significantly lower than corresponding viremia in control mice. Viral load detected in three different tissues (spleen, liver and small intestine) at day 4 post-infection was also significantly lower in mice immunized with LV-DENV-Ag1(IND) for both DENV-1 (FIG. 8E) and DENV-2 (FIG. 8F) infections. Altogether the immunization/protection experiments in the two lineages of IFNAR-KO mice produced similar results: faster weight recovery (d3-4 vs. d4-8), significantly lower viremia, faster viral clearance, and reduced viral presence in peripheral organs was observed in all mice immunized with LV-DENV-Ag1(IND).

To test the protection against DENV-3, two groups of mice (n=6) were immunized intramuscularly with 3×10⁸ TU/mouse of either LV-DENV-Ag1(IND) or LV-GFP(IND) and 2 months later (61 days p.i.) infected with 8×10⁶ FFU/mouse of DENV-3. Monitoring of viremia was performed in the subgroups of mice (n=3) on different days post-infection. Two independent experiments were performed in the same conditions to demonstrate statistical significance and the results were pooled after verification that no statistically significant differences were detected in the viremia levels and weight loss average between the LV-GFP control groups of the two experiments. Spleen samples were also collected from sacrificed animals on day 7 p.i. to determine viral load in the spleen. That organ was selected for the analysis because several studies showed that it has the highest level of DENV replication in laboratory infections of IFNAR-KO mice (35). Protection against DENV-4 was assessed in two groups of IFNAR-BL6 mice (n=6) that were immunized with 3×10⁸ TU/mouse of either LV-DENV-Ag1(IND) or LV-GFP(IND) and 1 month later infected with DENV-4 (1×10⁷ FFU/mouse). Weight of animals was measured daily and blood samples for monitoring of viremia were collected on different days post-infection. Samples of spleen, liver and small intestine were collected on day 7 to assess viral load in those organs.

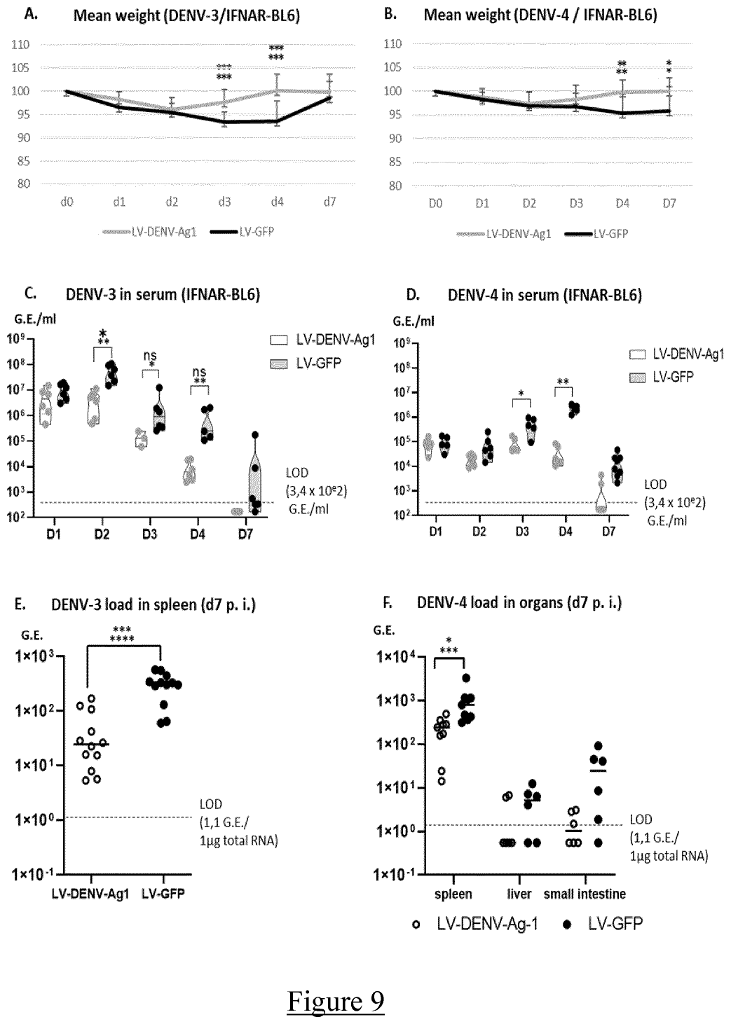

Similar to mice infected with DENV-1 and DENV-2, mice infected with DENV-3 or DENV-4 did not show any symptoms except for the weight loss that was observed during the first 2 days post-infection. The weight of mice immunized with LV-DENV-Ag1(IND) infected with DENV-3 was significantly higher than the weight of control mice on d3-4 post-infection (FIG. 9A), and viremia was significantly lower starting from d2 (FIG. 9C). On day 7 viremia was still detectable in 4 out of 6 mice that were immunized with LV-GFP, but not in any mice immunized with LV-DENV-Ag1(IND). Furthermore, viral load in the spleen of LV-DENV-Ag1(IND)-immunized mice detected on d7 was significantly lower than in mice of the control group (FIG. 9E).

DENV-4 infection resulted in significant weight difference between groups of mice immunized with LV-DENV-Ag1(IND) and LV-GFP(IND) observed on d4 and d7 post-infection (FIG. 9B). Mice immunized with LV-DENV-Ag1(IND) also had significantly lower viremia on d3 and d4 post-infection (FIG. 9D). Similarly to the results obtained with DENV-3 infection, at the end of the experiment viral RNA was detectable only in 2 animals out of 6 in the group immunized with LV-DENV-Ag1(IND), but in all animals of LV-GFP(IND)-immunized group. Significantly lower viral load had been detected in spleen of DENV-4 infected mice (FIG. 9F).

Prime-Boost Immunization Protocol: Protection of A129 Mice from DENV-2 Infection.

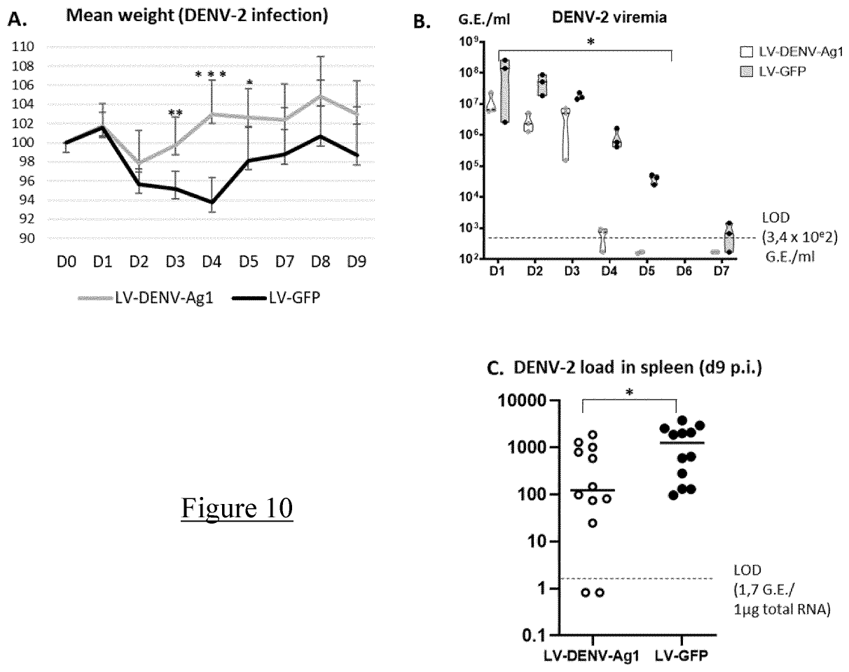

To evaluate the efficiency of a prime-boost immunization protocol for protection of IFNAR-KO mice against DENV infection, two groups of A129 mice (n=12) were immunized i.m. with 1×10⁷ TU/mouse of either LV-DENV-Ag1(IND) or LV-GFP(IND). Fifty-nine days post-immunization two groups were boosted with 2×10⁸ TU/mouse of LVs that expressed same antigens, but were pseudotyped with VSV-G of a different serotype, New Jersey (NJ), in order to avoid potential anti-vector immune response. Twenty-eight days after second immunization all mice were inoculated with 1×10⁷ FFU/mouse of DENV-2. Weight of animals was measured daily and blood samples were collected from the subgroups of mice on different days post-infection (FIG. 10). Animals were sacrificed on day 9 post-infection, after they have been gaining weight for two consecutive days. Similarly to the previous experiment that analyzed protection of A129 mice against DENV-2 by a single immunization with LV-DENV-Ag1(IND), all infected mice have initially lost weight during the first two days of infection (FIG. 10A) and developed ruffled fur.

However, mice immunized with LV-DENV-Ag1(IND) and LV-DENV-Ag1(NJ) started to regain weight earlier than the mice immunized with LV-GFP(IND)/LV-GFP (NJ) with the significant weight difference between the groups observed on d2-4 post-infection (FIG. 10A). The appearance of ruffled fur in infected animals has generally correlated with the weight loss and became less noticeable as soon as mice started to regain weight. Analysis of viremia by RT-qPCR demonstrated an approximately 10-fold lower viral load in serum of mice immunized with LVs expressing DENV-Ag1 on d1-2 p.i (FIG. 10B). Similar to the results of a single-dose immunization experiment, DENV-2 viremia in LV-DENV-Ag1(IND)-immunized mice has also diminished more quickly compared to viremia in control mice and was undetectable starting from d5 p.i. Viral load in the spleen (measured on day 9 p.i.) also was significantly lower in the LV-DEN-Ag1(IND)/LV-DENV-Ag1(NJ)-immunized group (FIG. 10C). Overall, results of this experiment are comparable to the results obtained with a single-dose immunization protocol (FIGS. 7B and D) and suggest that a prime-boost immunization does not provide a significant advantage for protection of A129 mice against DENV-2 infection.

### Mechanism of Protection: Depletion of CD8+ T Cells Results in Reduced Protection.

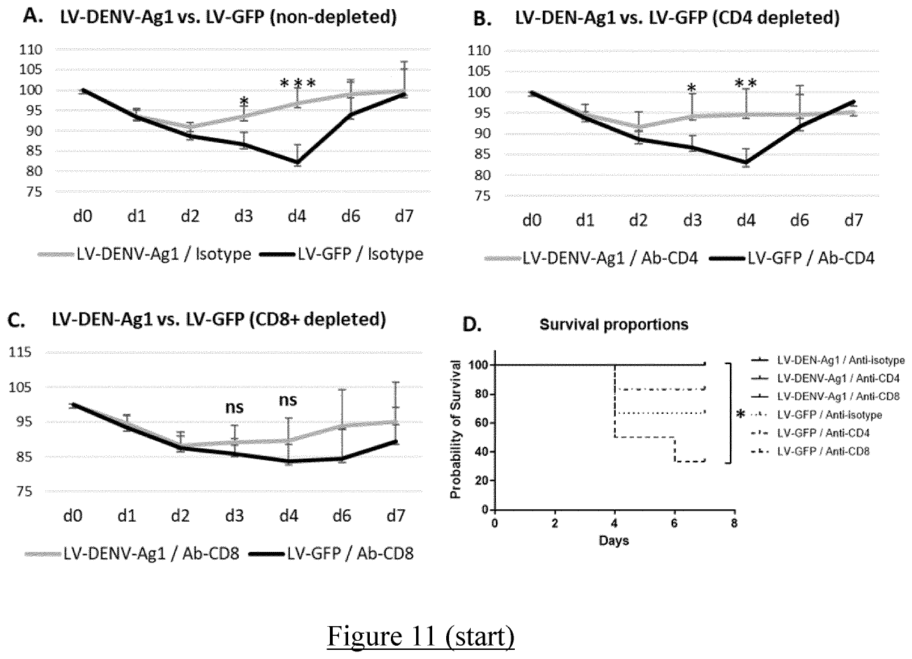

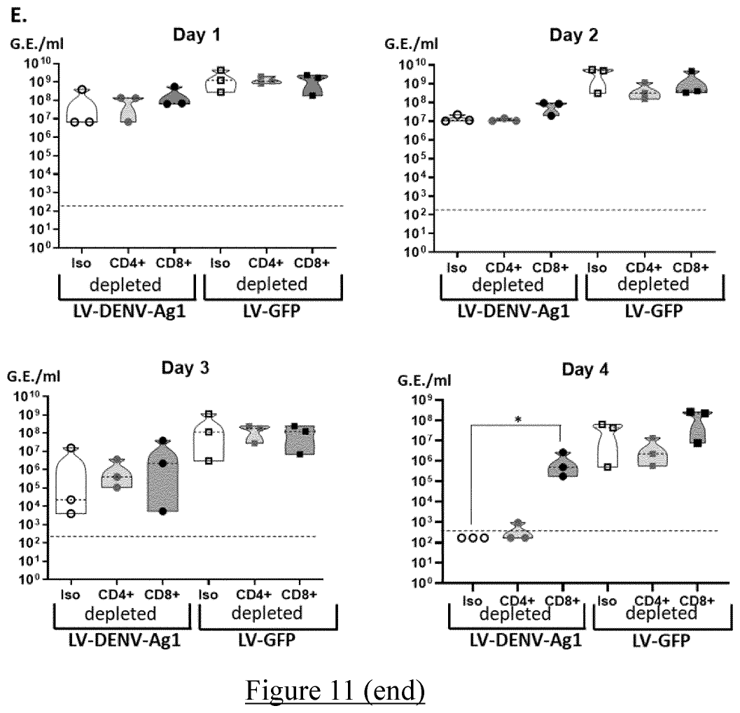

To analyze the role of different T lymphocyte populations (CD8+ and CD4+) in the protection against DENV infection induced by DENV-specific vector, a selective depletion of either CD8+ or CD4+ cells from IFNAR-BL6 mice immunized with LV-DENV-Ag1(IND) or with LV-GFP(IND) was carried out, prior to infection with DENV-2. First, two groups of mice were immunized intramuscularly with 3×10⁸ TU/mouse of either LV-DENV-Ag1(IND) or LV-GFP(IND) vector and 40 days later were boosted by the injection of same vectors at the same dose. Four days after boosting, each group was further divided into 3 subgroups that were injected intra-peritoneally with 250 μg/mouse of either anti-mouse CD8α antibody, anti-mouse CD4 antibody, or the IgG2b isotype control antibody. Second injection of the same antibodies was performed 3 days later, one day prior to infection. The following day mice were infected intravenously with 1×10⁷ FFU/mouse of DENV-2. The infection was monitored for seven days, and the measurement of animal weight were taken on days 1, 2, 3, 4, 6 and 7 post-infection. Blood samples for monitoring of the viremia were collected from the subgroups of mice on the same days. The efficiency of CD8+ and CD4+ T cell depletion has been verified by the cytometry of splenocytes extracted from euthanized animals on day 7. DENV-2 infection in mice pre-injected with IgG2b isotype control antibody has followed the same kinetics as seen previously in mice that were not subjected to antibody injections (FIGS. 11A and 8B). All mice infected by DENV-2 have lost weight during the first 2 days of infection, followed by the recovery phase that has started earlier in mice immunized by LV-DENV-Ag1(IND) vector (d2 p.i.) compare to mice immunized with LV-GFP(IND) vector (d4 p.i.). Viremia in animals immunized with DENV-specific vector was at all timepoints lower than viremia observed in mice immunized with the GFP-containing vector, with a clear drop in viremia level observed on d4 p.i. (FIG. 11E). Depletion of CD4+ T cells from the mice immunized either with LV-DENV-Ag1(IND) or LV-GFP(IND) vectors did not significantly alter the course of infection: the dynamics of weight loss and recovery, as well as the levels of viremia in mice that were depleted of CD4+ T cells were very similar to those seen in the corresponding groups of non-depleted mice (FIGS. 11A, B and E). In contrast, depletion of the CD8+ cells from either LV-DENV-Ag1(IND)- or LV-GFP(IND)-immunized groups has remarkably influenced the course of infection. Both groups demonstrated slower weight recovery and prolonged viremia compare to either non-depleted or CD4+-depleted groups (FIGS. 11C and E).

Remarkably, the fast reduction of viremia observed in LV-DENV-Ag1(IND)-immunized groups injected either with the isotype control or anti-CD4 antibodies on d4 did not occur in the group depleted of the CD8+ cells (FIG. 11E). Mortality was observed only in the groups immunized with the LV-GFP(IND) vector: 2 of 6 mice injected with isotype antibody (33%), 1 of 6 mice injected with anti-CD4 antibody (17%) and 4 of 6 mice injected with anti-CD8 antibody (67%) died from infection. Although the levels of viremia observed in 3 groups of LV-GFP(IND)-immunized mice were similar between days 1-3 p.i., viremia in the CD8+-depleted group was prolonged compared to two other groups from the day 4 of infection (FIG. 11D). Moreover, DENV-2 load measured in the spleen on day 7 was the highest in CD8+-depleted group compare to two other groups, and that group has also shown slowest weight recovery and lowest survival rate. These results suggest that the CD8+ T cell response plays a major role in LV-DENV-Ag1-induced protection against DENV challenge in IFNAR-KO mice, and that it helps to control infection at two different stages: At the early stage, DENV-Ag1-stimulated CD8+ cells are responsible for the initial reduction of viremia and faster viral clearance. Thus, appreciable differences in viremia (on d1-4 p.i.) and in weight (on d3-4 p.i.) are observed between the groups immunized with LV-DENV-Ag1 and LV-GFP, either depleted of CD4+ cells or not. In contrast, differences in viremia and weight between corresponding groups depleted of CD8+ cells are smaller and non-significant. Naïve CD8+ T cells (that are not specific for DENV in the beginning of infection) appear to be important for the control of infection at its late stage (d5-7), since depletion of CD8+ T cells prolongs viremia and delays weight recovery in both LV-DENV-Ag1- and LV-GFP-immunized groups, as well as increases mortality of LV-GFP-immunized mice.

### Polynucleotides and Lentiviral Vectors Expressing Non-Structural Antigens of ZIK Virus and YF Virus as Fusion Polypeptides

### Materials and Methods

### Antigen Design

For the design of ZIKV and YFV T-cell antigens the complete nucleotide sequences of 17 ZIKV strains (GenBank accession N^{o}s: KU955595.1, LC002520.1, KF268948.1, OL414716.1, HQ234500.1, KX377336.1, OK054351.1, MH119185.1, OQ661918.1, KY241712.1, ON209935.1, KY766069.1, KY014295.2, MF438286.1, KU922960.1, KU820897.5, KU509998.3) and of 19 strains of YFV (GenBank accession N^{o}s: JN620362.1, KF769015.1, DQ235229.1, MF004383.1, MW960207.1, JX898878.1, MF405338.1, HM582851.1, JF912187.1, JF912190.1, MF004382.1, JF912181.1, MW158361.1, U54798.1, KU978763.1, AY968064.1, KY861728.1, AF094612.1, KU921608.1) that represented different genetic lineages of each virus (64, 65) were retrieved from NCBI sequence database (66), aligned and used to construct phylogenetic tree with Mega 7 (67). Consensus sequences were inferred for major phylogenetic groups of ZIKV (African and Asian lineages) and YFV (South American, West African, and East-South African lineages) from the corresponding amino acid sequences using Consensus Maker software tool (68) in order to limit sequence diversity and identify conserved regions. MHC class I epitopes were predicted from the consensus sequences using Proteasomal cleavage/TAP transport/MHC-I binding combined predictor tool (69, 70) located at Immune Epitope Database (71) and netCTLpan predictor (72) located at DTU Bioinformatics server website (73). IEDB predictor was used to identify all 9- and 10-mer peptides presentable by 27 most prevalent Human Leukocyte Antigen (HLA) alleles (74), selecting those with the total positive score and a cut-off binding affinity IC50≤500 nM. Predictions with netCTLpan were performed for the same set of HLA alleles for all 9-mer peptides, and 100 epitopes per HLA allele with the best combined prediction score were retained. Epitopes predicted by the two methods were aligned to the consensus sequences of ZIKV and YFV using Blast (66) and plotted along the sequence length to identify the regions containing highest number of predicted MHC class I epitopes. Final selection of antigenic regions was done in a way to maximize inclusion of characterized human epitopes (retrieved from IEDB database) and the epitope-containing regions identify by predictions. Antigenic regions were joined together to create a single polypeptide and then prediction of T-cell epitopes was repeated to verify that no non-specific epitopes were created by joining of individual regions together and that all the epitopes located close to the junction sites were predicted to form correctly. In a case where joining of antigenic fragments has created a dominant non-specific MHC class I epitope for one of the 27 HLA alleles, such epitopes were eliminated by insertion of hydrophobic amino acid linkers at the junction sites. Additional rounds of epitope predictions were performed to confirm that all non-specific epitopes were eliminated.

### Production of Lentiviral Vectors

Sequences encoding poly-antigens of ZIKV and YFV (LV-ZIK, LV-YF-S and LV-YF-NS) were codon-optimized for the expression in mammalian cells and synthesized by GeneCust (France). Each antigen-coding sequence was inserted into pFLAPΔU3-β2m-WPRE vector between the beta 2 microglobulin (R2m) promoter and the Woodchuck Posttranscriptional Regulatory Element (mWPRE) that was previously mutated in order to improve the vector safety. Plasmids used for production of non-integrative LVs, including an antigen-containing transfer vector plasmid, a packaging plasmid NDK that encodes a mutated version of integrase protein (D64V) and an envelope plasmid that encodes G glycoprotein of VSV virus were purified using the NucleoBond Xtra Maxi EF Kit (Macherey Nagel), aliquoted and stored at −80° C. LV were produced in HEK-293T cells as described previously, and LV titer was determined by qPCR on LV-transduced HEK-293T cells that were treated with aphidicolin to prevent cell division (63, 38).

### Mice

Interferon-gamma receptor knockout mice (IFNAR-KO) that carry Ifnar1^{tm1Agt} allele on either 129 (A129) or C57BL/6J (IFNAR-BL6) genetic background, aged 6 to 16 weeks, were used in the experiments. Both mouse lineages belong to H-2^{b} MHC haplotype and thus have similar antigenic presentation and T-cell response. The initial assessment of the immunogenicity and protection efficiency of LV-ZIK, LV-YF-S and LV-YF-NS was performed in A129 mice because that lineage represent one of the established models for ZIKV infection (33-34). However, as our comparison of YFV infections in A129 and IFNAR-BL6 (unpublished) as well as published data (63, 34) indicated that mice of IFNAR-BL6 lineage are more susceptible to infections with ZIKV and YFV viruses, immunogenicity and protection studies were also performed on that mouse lineage. Mice were bred and maintained under specific pathogen-free conditions at animal facilities of Institut Pasteur and all experiments involving ZIKV and YFV infections were performed in the A3 animal facility. Animal experiments were performed in accordance with the French and European guidelines, following to approval by the Institute Pasteur Safety, Animal Care and Use Committee (CETEA no. DAP1800077) and Ministry of High Education and Research (APAFIS #18428-2019010717408411_v2).

Immunization and Inoculation with ZIKV and YFV

A129 and IFNAR-BL6 mice were immunized with 1-3×10⁸ TU/mouse (depending on the experiment) of all lentiviral vectors by intra-muscular (i.m.) injection of LV in a total volume of 50 μL, in the posterior muscle. Inoculations of mice with ZIKV and YFV were performed intraperitoneally in a total volume of 300 μl and inoculations doses (specified in the text) depended on the efficiency of viral propagation in Vero E6 cultures. The infectivity of the practicable doses was first verified in preliminary experiments in IFNAR-KO mice. Mice were monitored for signs of illness, such as lethargy, ruffled fur, hunched posture and neurological signs (partial paralysis, prostration, tremors, unsteady gait and/or falling) and their weight was recorded regularly. Mice were euthanized either in the case if they lost 20% of their initial weight, or if the 10% weight loss was accompanied by the appearance of neurological symptoms (i.e. abnormal movements and/or limb paralysis).

### Propagation and Titration of Viral Stocks

Asian strain Zika PF-13 (strain H/PF/2013; GenBank: KJ776791) was obtained through the DENFREE (FP7/2007-2013) consortium. The vaccine strain of YFV (17D-204, Stamaryl) was obtained from the commercial lot of vaccine purchased from the Institute Pasteur vaccination center. All viral stocks used for infection were produced and titrated in Vero E6 cells, essentially as described previously (63). Plaques produced by ZIKV and YFV were visualized by staining for 15 min with the Gram Crystal Violet solution (BD) diluted in H₂O (1:1), counted, and used to calculate the infectious virus titer that was expressed as a plaque number per milliliter of viral stock.

### ELISPOT Assay

The ELISPOT procedure was generally following the protocol supplied with ELISPOT kit for IFNγ detection (Mabtech AB, Nacka Strand, Sweden), except that 96-well PVDF plates (Millipore, Sigma) were activated by incubation with 35% ethanol, washed, and coated by the overnight incubation with 100 μl per well of 5 μg/ml rat anti-mouse IFNγ antibody (clone AN-18, BD Pharmingen). Splenocytes from immunized mice were added in triplicates at 1×10⁵ cells/well and stimulated for 18 h by the pools of antigen-specific peptides containing 2 μg/ml of each peptide. Negative controls (unstimulated splenocytes) and positive control (splenocytes stimulated by 2.5 μg/ml of concanavalin A) were also analyzed in triplicates for each animal. After incubation spots were revealed following a Mabtech AB Elispot kit protocol and counted on AID ELISpot Reader System ELR04 (Autoimmune Diagnostika GmbH, Strassberg, Germany). Average background signals from negative controls were subtracted from the average positive signals recorded for each splenocyte/peptide pool combination and results were expressed as a number of spot-forming cells per million of splenocytes.

### Cytometry

Analysis of intracellular cytokine secretion was performed following a previously described protocol (76). Briefly, splenocytes obtained by homogenization of spleens through 100 μm nylon filters (Cell Strainer, BD Bioscience) were plated at 4×10⁶ cells/well in 24-well plate and incubated for 6 h either with 10 μg/ml of pooled antigen-specific peptides or with equal amount of control non-specific peptide. Co-stimulatory monoclonal antibodies (mAbs) anti-CD28 and anti-CD49d (BD Biosciences) were added at that stage, at final concentration of 1 mg/mL each, as their presence was shown to increase the signals from low-affinity T-cells (77). During the last 3 h of incubation a mixture of Golgi Plug and Golgi Stop (BD Bioscience) containing PE-Cy7-anti-CD107a mAb (clone 1D4B, BioLegend) at 1:100 dilution of was added to the cells. Cells were washed with FACS buffer (PBS, containing 3% FBS and 1% EDTA) and incubated for 20 min at 4° C. with a mixture of FcγII/III receptor blocking anti-CD16/CD32 (clone 2.4G2), Near IR Live/Dead (Invitrogen), PerCP-Cy5.5-anti-CD3E (clone 145-2C11), eF450-anti-CD4 (clone RM4-5, eBioscience), and BV711-anti-CD8 (clone 53-6.7) mAbs (BD Biosciences or eBioscience). After incubation cells were washed, permeabilized by Cytofix/Cytoperm kit (BD Biosciences) for 20 min and then incubated with mAbs for cytokine staining: PE-anti-IL-2 (clone JES6-5H4), FITC-anti-TNF (MP6-XT22) and APC-anti-IFNγ (clone XMG1.2) (BD Biosciences), for 30 min at 4° C. Staining with rat anti-mouse isotype antibodies FITC-IgG1k, PE-IgG2bk and APC-IgG1k (BD Bioscience) was used as the control.

### Viremia and Viral Loads in Peripheral Organs

Mouse blood samples were collected at different timepoints during infection by bleeding from sub-mandibular vein directly into Microvette 500 K3E EDTA-containing tubes (Starstedt). Plasma samples that were clarified from blood cells by centrifugation and organ samples collected from euthanized animals were preserved at −80° C. until the RNA extraction to determine viremia and viral load in organs. RNA extraction and RT-qPCR analysis were performed as described previously (63). Analysis of viremia and organ load of ZIKV was performed using two-step RT-qPCR protocols adopted from Lanciotti et al. (29). YFV load in serum and peripheral organs of A129 mice was analyzed with RT-PCR protocol adopted from Bae et al. (30), and YFV load in serum and organs of IFNAR-BL6 mice with a protocol adopted from Fischer et al. (78). Standards for viral RNA quantification were obtained by in vitro transcription of cloned virus-specific DNA fragments that were amplified from stocks of ZIKV (primers ZIKV-F-CGGGATCCCGAGCCAAAAAGTCATATACTTG (SEQ ID NO. 213) and ZIKV-R-ACCGCTCGAGTCAGTTTCATGTCCTGTGTCATT (SEQ ID NO. 214)) and YFV (YFV-F-AACCCACACATGCAGGACAA (SEQ ID NO. 215) and YFV-R-GTTGCAGGTCAGCATCCACA (SEQ ID NO. 216)). The RT reactions were performed with Moloney murine leukemia virus reverse transcriptase (M-MLV) and virus-specific primers. The resulting cDNA was analyzed in duplicate qPCR run on a QuantStudio 12K Flex real-time PCR system (Applied Biosystems, Carlsbad, CA, USA), and the amount of viral RNA was determined from the standard curve reproduced for each RT-PCR run.

### Immunohistochemistry

Mouse brain and spleen samples were fixed in formalin for 72 hours and embedded in paraffin. Paraffin sections (5-μm thick) were stained with Hematoxylin and Eosin (H&E). Slides were scanned using the AxioScan Z1 (Zeiss) system and images were analyzed with the Zen 2.6 software.

### Statistical Analysis

Statistical analysis was performed as disclosed above in respect of the DENV experiments.

### Design and Refinement of ZIKV T Cell Antigens

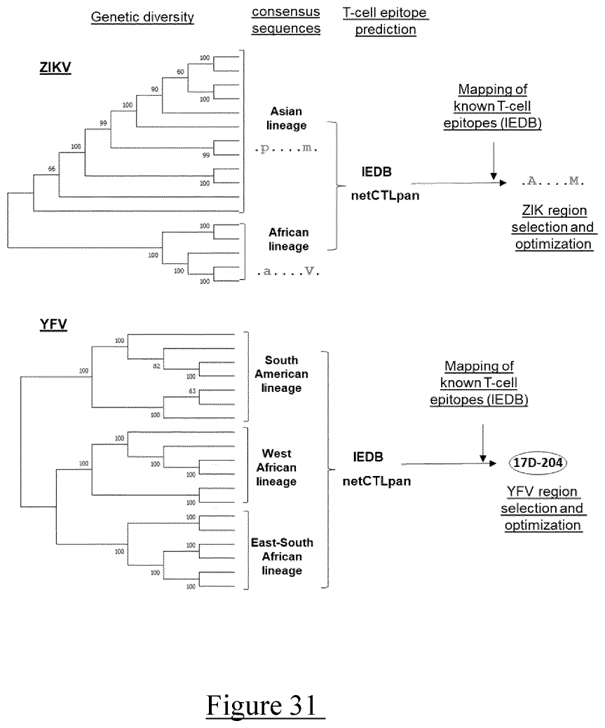

To create poly-antigens that could induce T-cell response against ZIKV and YFV, the genetic diversity of these viruses was first evaluated by inferring a phylogenetic tree, based on the stains representing main genetic lineages of each virus (FIG. 31).

For ZIKV, phylogenetic tree was based on the representatives strains of African genotype that originated from Senegal, Guinea and Nigeria (West Africa), Uganda and Central African republic (East Africa) (80), and Asian genotype including strains from Malaysia and India (ZB.1.0 lineage, South-Eastern/Southern Asia), Thailand (ZB.1.1 lineage, South-Eastern/Southern Asia), Singapore and Cambodia (ZB.1.2 lineage, South-Eastern/Southern Asia), French Polynesia and Haiti (ZB.2.0 lineage, Polynesia, Caribbean, South America), Mexico and Colombia (ZB.2.1 lineage, Central America), USA and Cuba (ZB.2.2 lineage, North America) (64).

A phylogenetic tree based on YFV strains included representatives of South American, West African and South-East African lineages. To find T-cell epitopes shared by different strains of ZIKV and YFV that could be presented by human HLA alleles, consensus amino acid sequences were inferred for the genetic lineages of each virus. The genetic diversity of ZIKV was represented by the consensus sequences of its two main genotypes (Asian and African), while diversity of YFV was summarized by three consensus sequences, each representing one phylogenetic lineage of that virus.

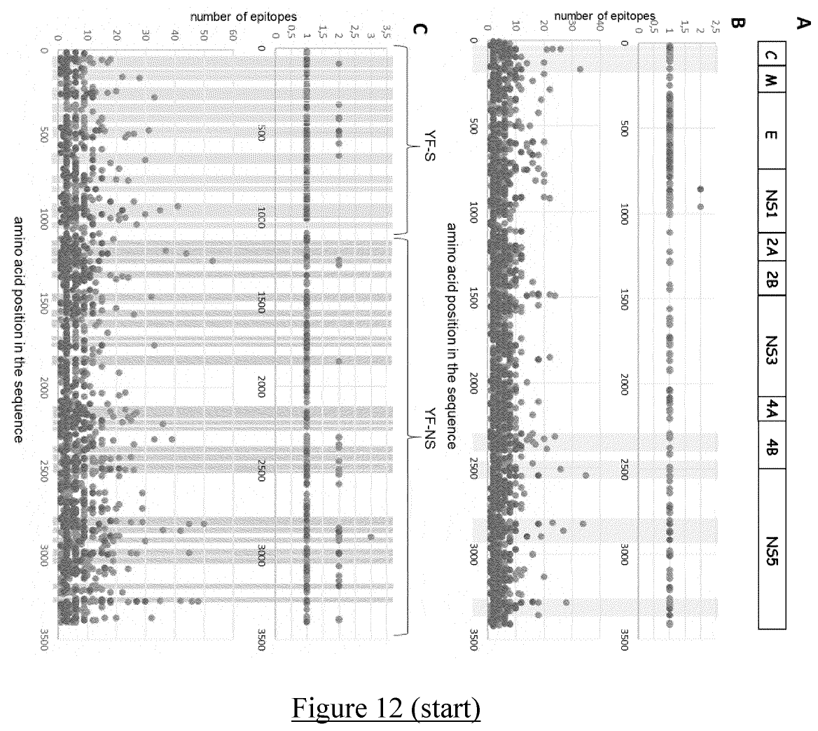

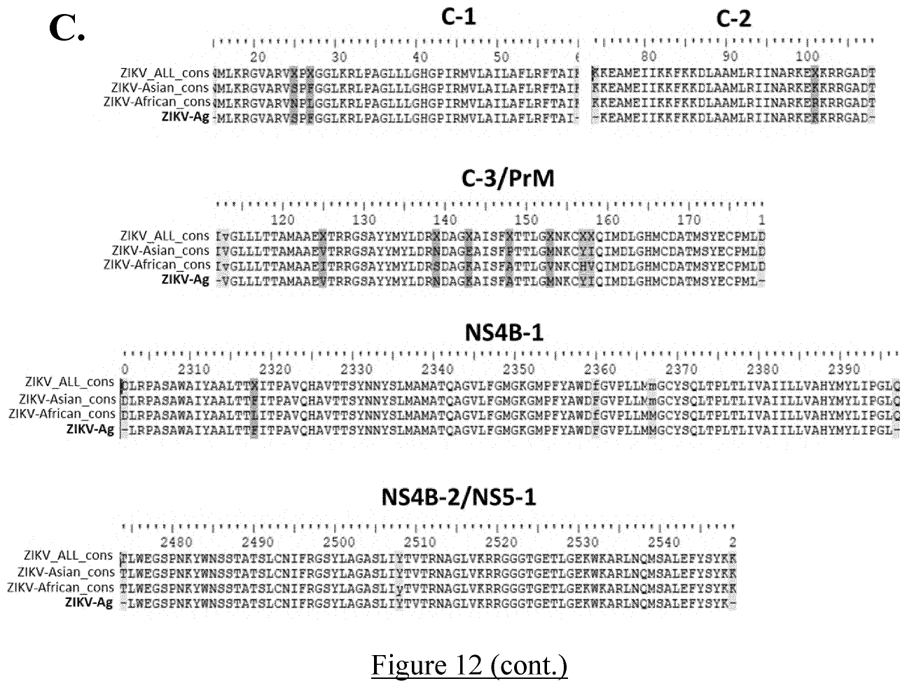

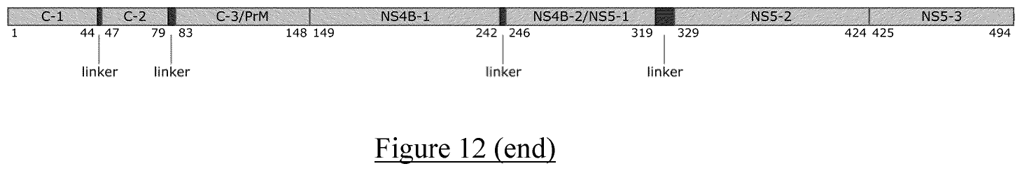

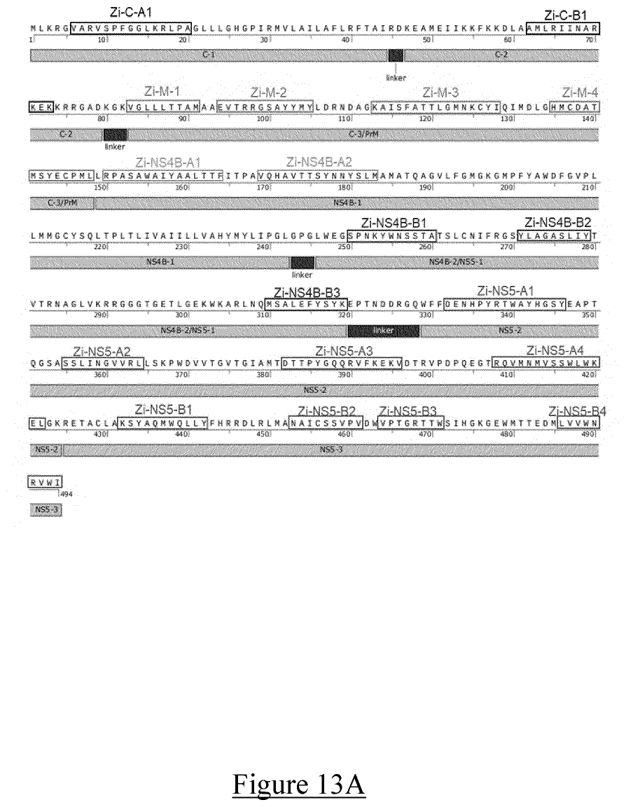

Sequences of 33 human MHC class I and 227 MHC class II ZIKV-specific epitopes, and 122 and 407 YFV-specific epitopes, respectively, annotated as positive in T-cell assays in IEDB database (71) were downloaded and matched to the alignment of the consensus ZIKV sequences of each virus to identify known T-cell immunogenic regions (FIG. 12). In order to assure that no potentially immunogenic region was missed due to the gaps in the current knowledge of ZIKV and YFV immunogenicity and to better define immunogenic regions, prediction of MHC class I epitopes presentable by 27 HLA alleles that are shared by 97% of global human population (74) was performed with the bioinformatic tools available at IEDB and DTU tech web sites (71, 73). The distribution of the predicted MHC class I epitopes was compared with distribution of verified epitopes, and several regions with the highest epitope density were selected to create poly-antigens specific for each virus (FIG. 13 and FIG. 13). The consensus sequences of two ZIKV genotypes representing phylogenetic lineages of Asian and African ZIKV are 97% similar and thus consensus sequence of Asian genotype that is more globally present, more diverse and also responsible for several large outbreaks of Zika disease, was selected as a master consensus sequence providing a base for ZIKV-specific antigen (further called “ZIK”). Two aa residues, E143K and P147A, in ZIKV alignment (FIG. 13) were converted to the consensus sequence of the African lineage because more human MHC class I epitopes presentable by a larger spectrum of HLA alleles were predicted from that sequence than from the corresponding sequence of Asian lineage. A first antigen (ZIK-Ag) was based on the conserved regions of ZIKV containing known and predicted clusters of T cell epitopes and was designed and optimized using a similar approach to the outlined above for design of DEN-Ag (FIG. 12). It included regions of C, PrM, NS4B, and NS5 proteins (FIG. 12). Regions from the structural proteins capsid (C) and pre-matrix (PrM) were included because those regions contained clusters of MHC class I epitopes (known or predicted) and the level of sequence homology between these regions and the corresponding regions of DENV was sufficiently low to avoid cross-reactive antibody responses that could result in ADE (FIG. 13). A second antigen represented the complete sequence of ZIKV-NS1 protein with a 20aa-long signal peptide derived from the E protein coding region added for the correct intracellular processing and targeting of NS1.

### Immunogenicity of Non-Integrative Lentiviral Vector Expressing ZIKV-Ag in A129 Mice.

To analyze T cell responses induced by ZIKV-Ag expressed from a non-integrative LV two groups of six mice each were immunized with a single dose 3×10⁸ TU/mouse of either LV-ZIKV-Ag(IND) or LV-GFP(IND). Splenocytes of immunized mice were collected 14 days after immunization and analyzed for production of IFNγ by Elispot assay using pools of region-specific peptides representing MHC class I epitopes of humans and A129 mice (H-2^{b} mice) (FIG. 14 and Table 1). The test has demonstrated that LV-ZIKV-Ag induced T cell response in A129 mice with the highest reactivity observed against NS5A, followed by NS5B and PrM regions of the antigen.

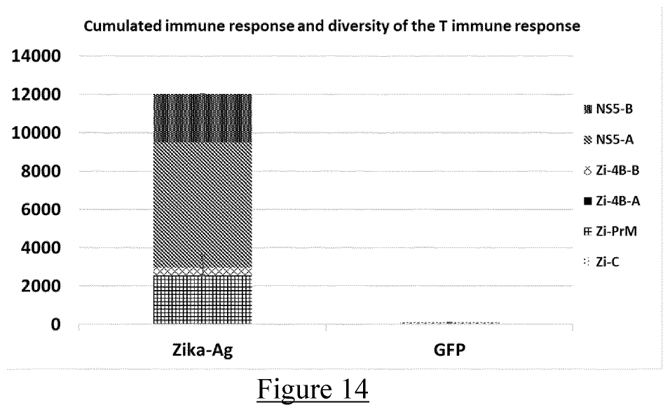

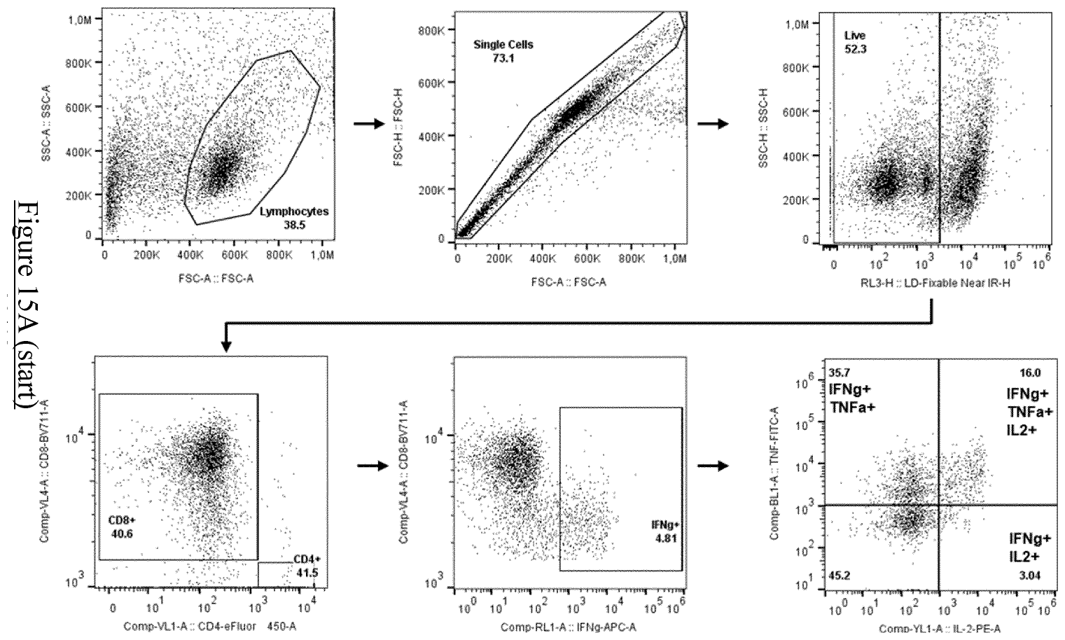

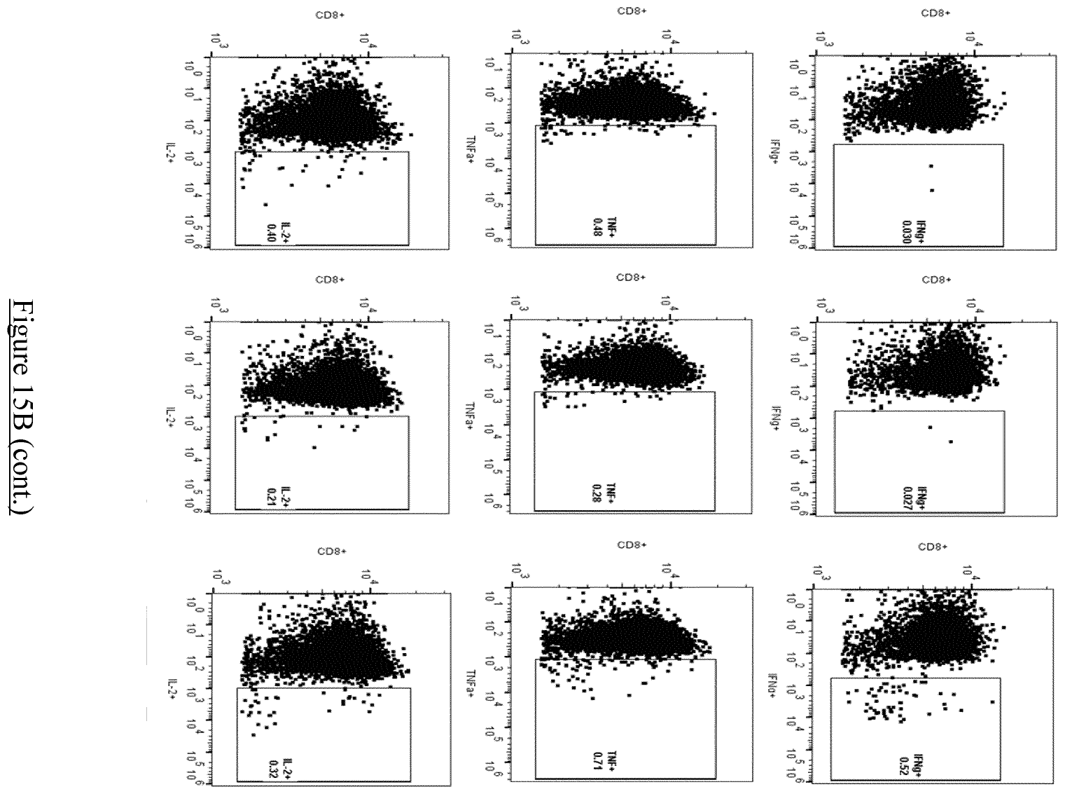

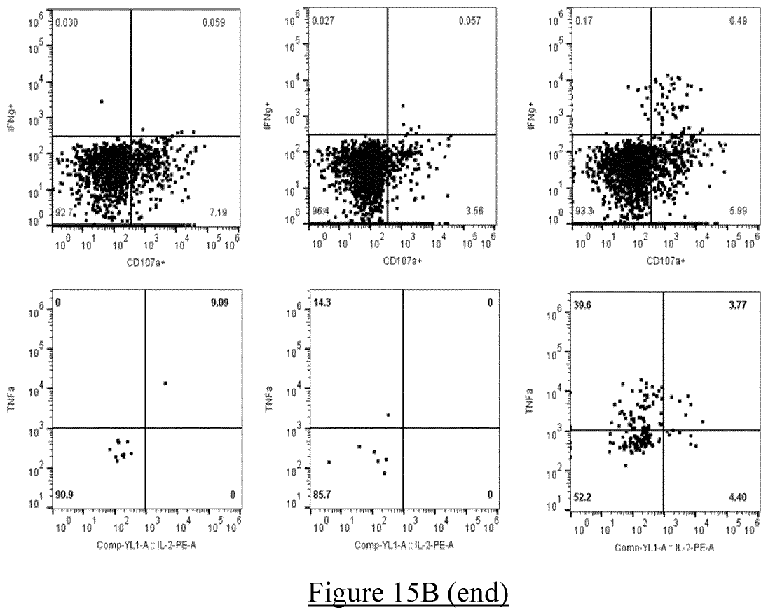

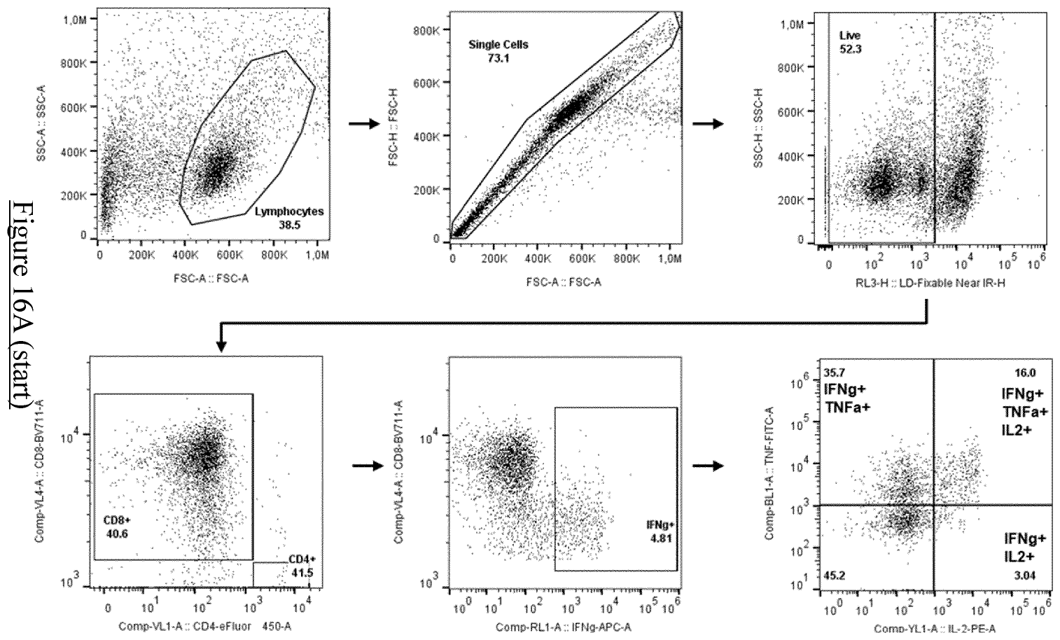

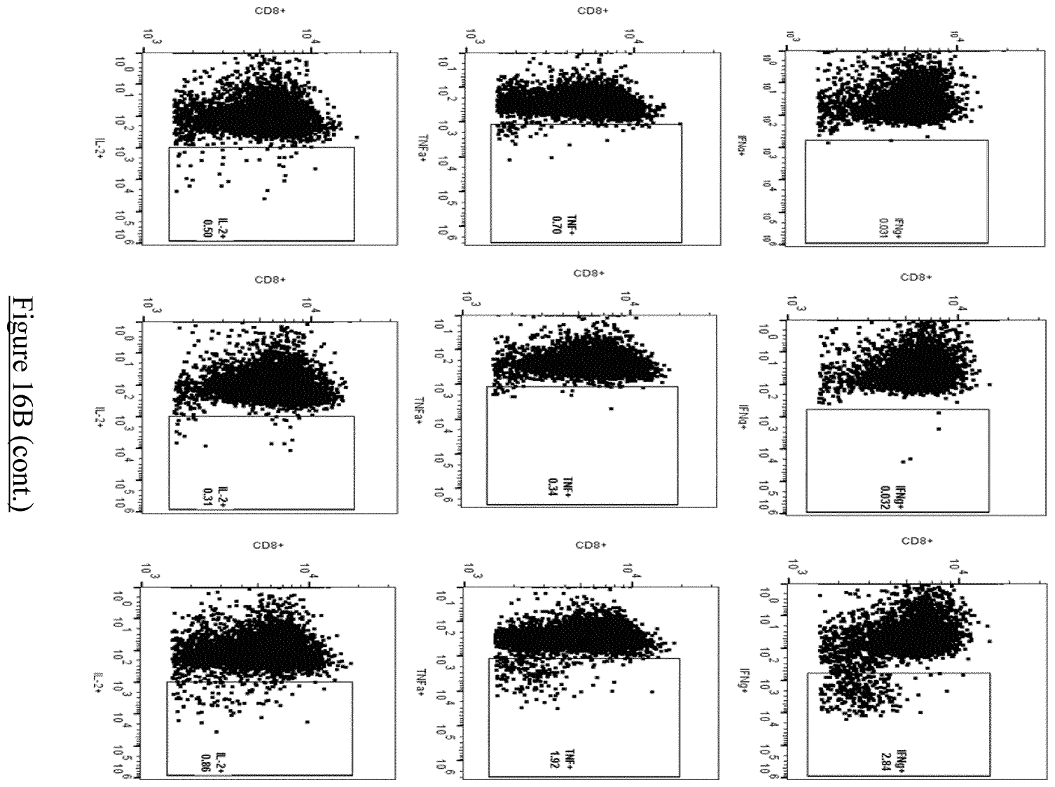

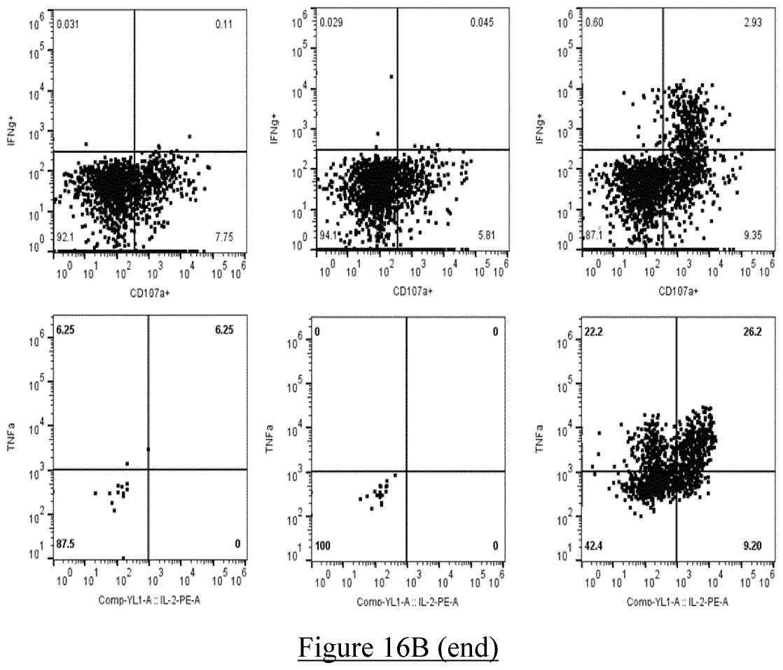

Immunogenicity of both LV-ZIKV-Ag and ZIKV-ZIKV-NS1 has also been evaluated in IFNAR-BL6 mice by the intracellular cytokine staining (FIG. 15 and FIG. 16, respectively). Splenocytes from IFNAR-BL6 mice immunized with 3×10⁸ TU of either LV-ZIKV-Ag, LV-ZIKV-NS1 or LV-GFP vector were extracted 14 days post-infection and analyzed for the secretion of IFNγ, TNFα, IL2, and lymphocyte degranulation marker CD107a. Flow cytometry analysis of cytokine-stained cells has demonstrated that CD8+ T cells responded to antigen exposure/peptide stimulation by secretion of IFNγ, TNFα, and IL2. A proportion of cells from mice immunized with LV expressing both ZIKV antigens co-expressed IFNγ and CD107a. Similar to what have been shown for LV expressing DENV-Ag1, polypotent CD8+ T cells simultaneously expressing three effector cytokines (IFNγ, TNFα, and IL2) have also been detected amongst splenocytes that were exposed to both ZIKV antigens (except that IL2 was not detected from CD8⁺ T cells from mice immunized with ZIKV-Ag) and re-stimulated with ZIKV-specific peptides, but not among those that were exposed to unrelated antigen (LV-GFP) or those that were stimulated with non-specific peptide (YF-C).

TABLE 1

| Peptide | Pool | SEQ ID | Peptide | | | |
|---|---|---|---|---|---|---|
| origin | name | NO. | name | Peptide sequence | Reactivity | REFERENCE |
| ZIK | Zi-C | 184 | Zi-C-A1 | VARVSPFGGLKRLPA | human (HLA-A*01:01), | IEDB, |
| antigen | | | | | human MHC class II, | netCTLpan |
| | | | | | mouse (H-2b, predicted) | |
| | | 185 | Zi-C-B1 | AMLRIINARKEK | mouse (H-2b, predicted) | netCTLpan |
| | Zi-PrM* | 186 | Zi-M-1 | VGLLLTTAM | mouse (H-2b, predicted) | IEDB |
| | | 187 | Zi-M-2 | EVTRRGSAYYMY | human, mouse (H-2b, | IEDB, |
| | | | | | predicted) | netCTLpan |
| | | 188 | Zi-M-3 | KAISFATTLGMNKCYI | mouse (H-2b, known and | IEDB |
| | | | | | predicted) | |
| | | 189 | Zi-M-4 | HMCDATMSYECPML | human (HLA-A*01:01), | IEDB, |
| | | | | | mouse (H-2b, known and | netCTLpan |
| | | | | | predicted) | |
| | Zi-NS4B-A | 190 | Zi- | RPASAWAIYAALTTF | human (HLA-B*07:02), | IEDB, |
| | | | NS4B- | | mouse (H-2b, predicted) | netCTLpan |
| | | | A1 | | | |
| | | 191 | Zi- | VQHAVTTSYNNYSLM | mouse (H-2b, predicted) | IEDB, |
| | | | NS4B- | | | netCTLpan |
| | | | A2 | | | |
| | Zi-NS4B-B | 192 | Zi- | SPNKYWNSSTA | human (HLA-B*07:02) | IEDB |
| | | | NS4B- | | | |
| | | | B1 | | | |
| | | 193 | Zi- | YLAGASLIY | human (HLA-A*01:01), | IEDB, |
| | | | NS4B- | | mouse (H-2b, predicted) | netCTLpan |
| | | | B2 | | | |
| | | 194 | Zi- | MSALEFYSYK | human (HLA-A*01:01), | IEDB, |
| | | | NS4B- | | mouse (H-2b, predicted) | netCTLpan |
| | | | B3 | | | |
| | Zi-NS5-A* | 195 | Zi-NS5- | DENHPYRTWAYHGSY | human (HLA-B*35:01), | IEDB, |
| | | | A1 | | mouse (H-2b, predicted) | netCTLpan |
| | | 196 | Zi-NS5- | SSLINGVVRL | mouse (H-2b, known and | IEDB, IEDB |
| | | | A2 | | predicted) | |
| | | 197 | Zi-NS5- | DTTPYGQQRVFKEKV | human (HLA-B*35:01) | IEDB |
| | | | A3 | | | |
| | | 198 | Zi-NS5- | RQVMNMVSSWLWKEL | mouse (H-2b, known) | IEDB |
| | | | A4 | | | |
| | Zi-NS5-B* | 199 | Zi-NS5- | KSYAQMWQLLY | human (HLA-A*01:01), | IEDB |
| | | | B1 | | mouse (H-2b, predicted) | |
| | | 200 | Zi-NS5- | NAICSSVPV | mouse (H-2b, predicted) | IEDB |
| | | | B2 | | | |
| | | 201 | Zi-NS5- | VPTGRTTW | human (HLA-B*07:02) | IEDB |
| | | | B3 | | | |
| | | 202 | Zi-NS5- | LVVWNRVWI | mouse (H-2b, predicted) | IEDB |
| | | | B4 | | | |
| YF-S | YF-S | 203 | YF-C | FIFFFLFNIL | mouse (H-2b, known/ | IEDB, |
| antigen | | | | | predicted) | netCTLpan |
| | | 204 | YF-M | NPFFAVTALTIAYLV | human MHC class II | IEDB, |
| | | | | | (known), mouse (H-2b, | netCTLpan |
| | | | | | predicted) | |
| | | 205 | YF-NS1 | TNGPWMQVPL | mouse (H-2b, known/ | IEDB, |
| | | | | | predicted) | netCTLpan |
| YF-NS | YF-NS* | 206 | YF- | AALHPFALLL | mouse (H-2b, known) | IEDB |
| antigen | | | NS2B | | | |
| | | 207 | YF- | IVNRNGEVI | mouse (H-2b, known/ | IEDB |
| | | | NS3-1 | | predicted) | |
| | | 208 | YF- | ATLTYRML | mouse (H-2b, known/ | IEDB |
| | | | NS3-2 | | predicted) | |
| | | 209 | YF- | VSGWNSITVM | mouse (H-2b, known/ | IEDB |
| | | | NS5-1 | | predicted) | |
| | | 210 | YF- | LALYLLLAL | mouse (H-2b, known/ | IEDB |
| | | | NS5-2 | | predicted) | |
| | | 211 | YF- | VMRGNHYAF | mouse (H-2b, known) | IEDB |
| | | | NS5-3 | | | |
| | | 212 | YF- | VMYNLWKMKT | mouse (H-2b, known) | IEDB |
| | | | NS5-4 | | | |

### Protection of A129 and IFNAR-BL6 Mice Against ZIKV Infection by LV-ZIKV-Ag and LV-ZIKV-NS1

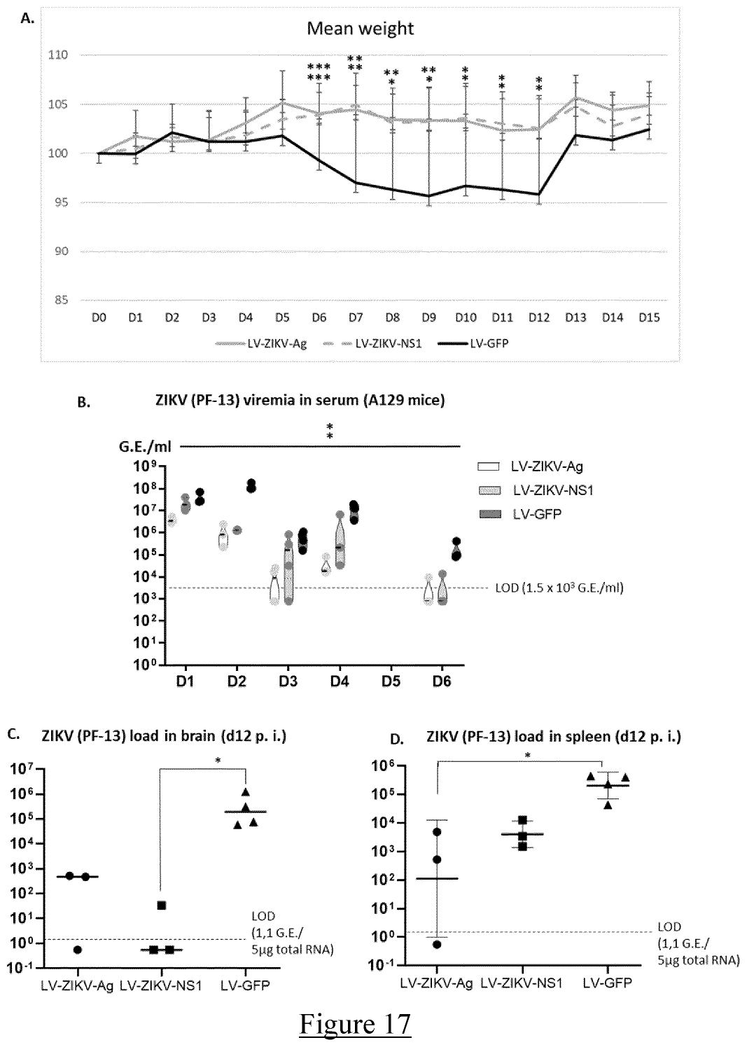

In the first experiment three groups of A129 mice were immunized, respectively, with LV-ZIK-Ag (1×10⁸ TU/mouse), LV-ZIK-NS1 (0.75×10⁸ TU/mouse) and LV-GFP (1×10⁸ TU/mouse) and one month later infected with ZIKV of “Asian” lineage (strain PF13), that has been responsible for large outbreaks of Zika disease in the Polynesia and South America. Similar to the infection of A129 mice with DENV, only a few visible symptoms were observed in the infected animals, such as ruffled fur and temporary weight loss (observed only in mice immunized with LV-GFP control vector). Significant differences in the weight of mice immunized with both ZIKV-specific vectors compared to mice immunized with LV-GFP vector were observed on days 6 to 12 post-infection (FIG. 17A). Viremia (measured on days 2, 4, and 6 post-infection) was detectable in all groups of infected mice, however viremia in mice immunized with ZIKV-specific vectors was approximately 100-fold lower than in the control mice on days 2 and 4 and was either undetectable or on the limit of detection at day 6, while it was still readily detectable in the LV-GFP-immunized mice (FIG. 17B). Moreover, viral load in peripheral organs (spleen and brain) measured on day 12 post-infection was also at least 100-fold lower than viral load in the control group of mice (FIGS. 17C and 17D).

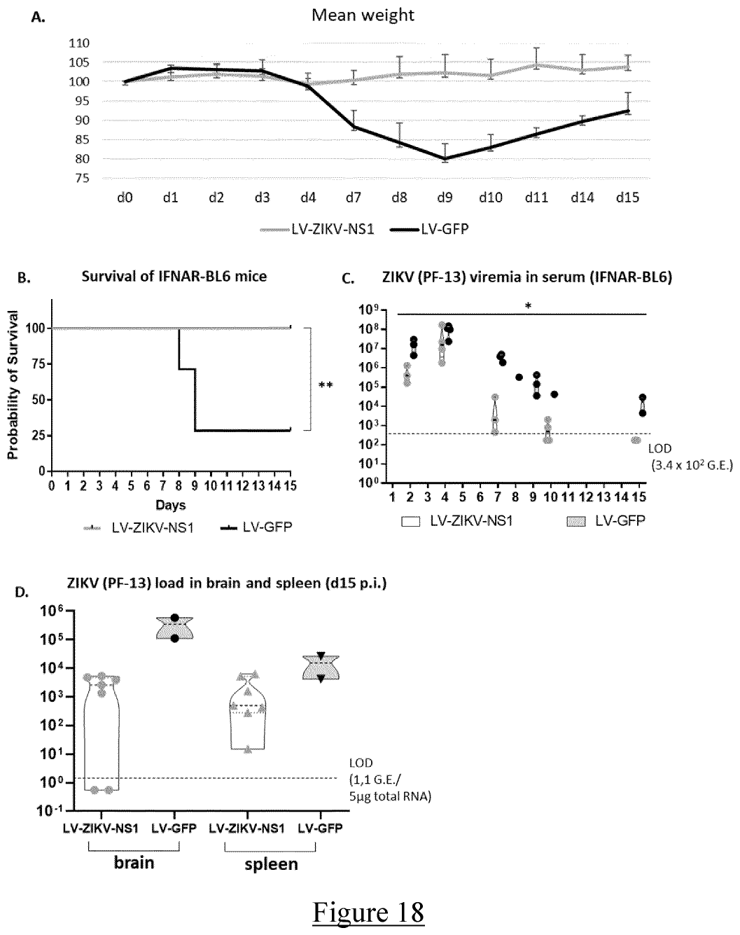

This experiment was repeated in IFNAR-BL6 mice that are more sensitive to ZIKV infection (63,34) and develop severe disease often associated with neurological symptoms and death. Mice immunized with LV-ZIK-NS1 (3×10⁸ TU/mouse) were compared to mice immunized with the same dose of LV-GFP vector that were inoculated i.p. one month later with 1×10³ PFU/mouse of ZIKV (PF-13). Immunization with LV-ZIKV-NS1 has completely protected mice against infection-induced symptoms and death: in the group immunized with LV-GFP neurological symptoms (weakness and flaccid paralysis of hind legs) were detected in all mice and 70% mortality was observed by 9 dpi, while no neurological symptoms and no mortality was observed in mice immunized with LV-ZIK-NS1 (FIG. 18B). Moreover, no significant weight loss was detected in mice immunized with LV-ZIK-NS1 vector (FIG. 18A). Similar to the protection experiment on A129 mice, viremia was detectable in both groups, but significantly lower viremia (>100-fold) was observed in the group immunized with LV-ZIKV-NS1 vector on days 7, 10 and 15 of infection (FIG. 18C). Viral load in the organs (spleen, brain and testes) of mice immunized with LV-ZIKV-NS1 was also lower than that of LV-GFP-immunized mice that have survived ZIKV infection (d15 post-infection) (FIG. 18D).

Altogether these data indicate that, similar to what has been seen with LV-DEN-Ag1 vector, ZIKV-specific vectors induce partial protection of mice, resulting in protection against the weight loss, lower viremia and lower viral load in the organs as well as (established at least for LV-ZIK-NS1 vector) protection against symptoms of ZIKV disease and death.

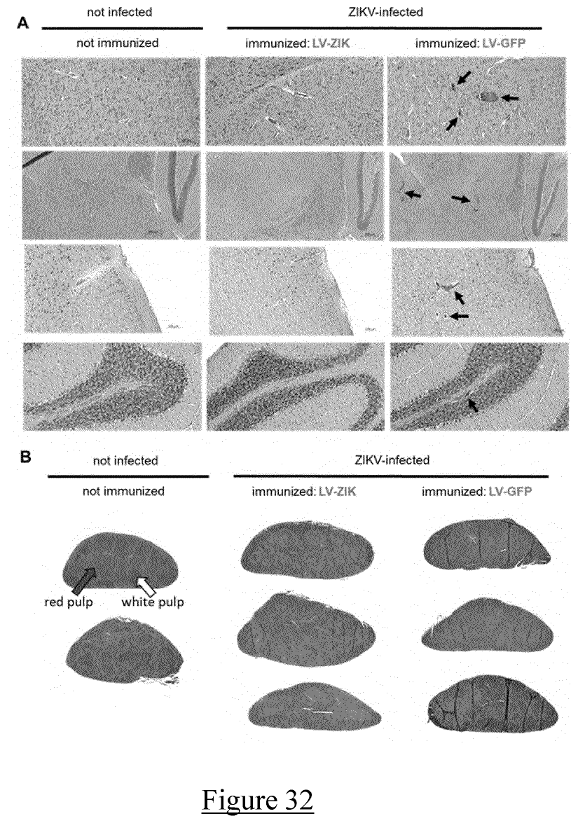

Histopathological analysis of the brain and spleen collected on 9 dpi from IFNAR-BL6 mice showed remarkable differences resulting from LV-ZIK vaccination. The brains from the LV-GFP-injected control mice displayed perivascular cuffing that was not observed in the untreated controls or the LV-ZIK-vaccinated ZIKV-inoculated mice (FIG. 32A). The spleens from the LV-GFP-injected control mice had very poor distinction of the white and red pulp zones compared to the spleens from untreated controls or the LV-ZIK-vaccinated ZIKV-inoculated mice (FIG. 32B).

Therefore, LV-ZIK induces a significant protection of IFNAR-KO mice, resulting in reduced weight loss, lower viremia, lower viral load and reduced pathology in the organs, as well as protection against ZIKV disease symptoms (weakness, paralysis of hind legs) and death.

### Design and Refinement of YFV T Cell Antigens

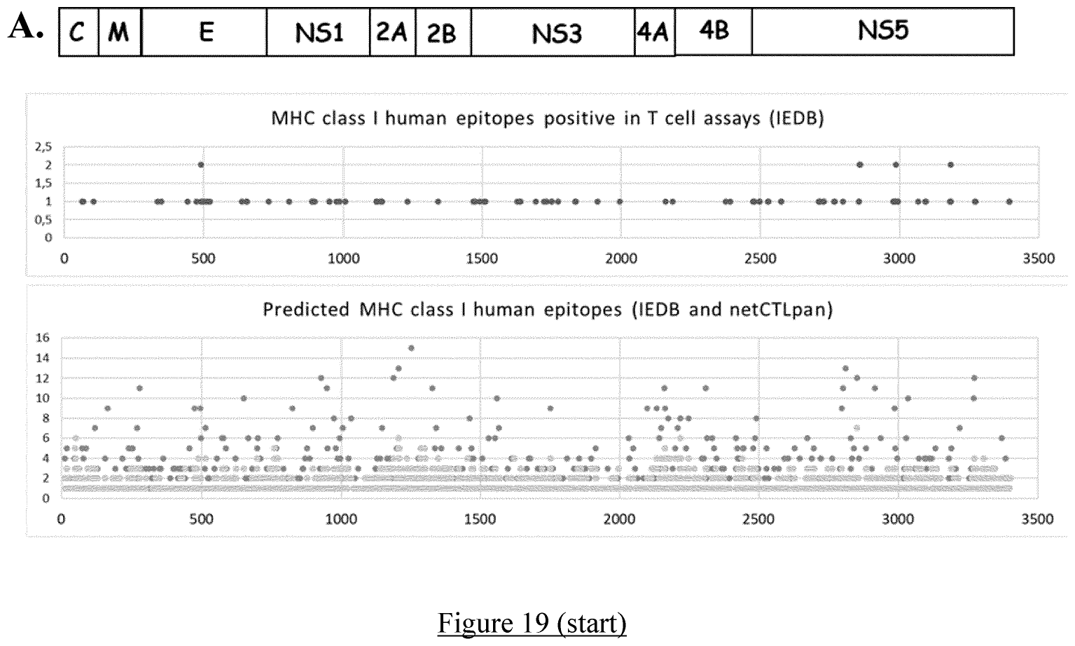

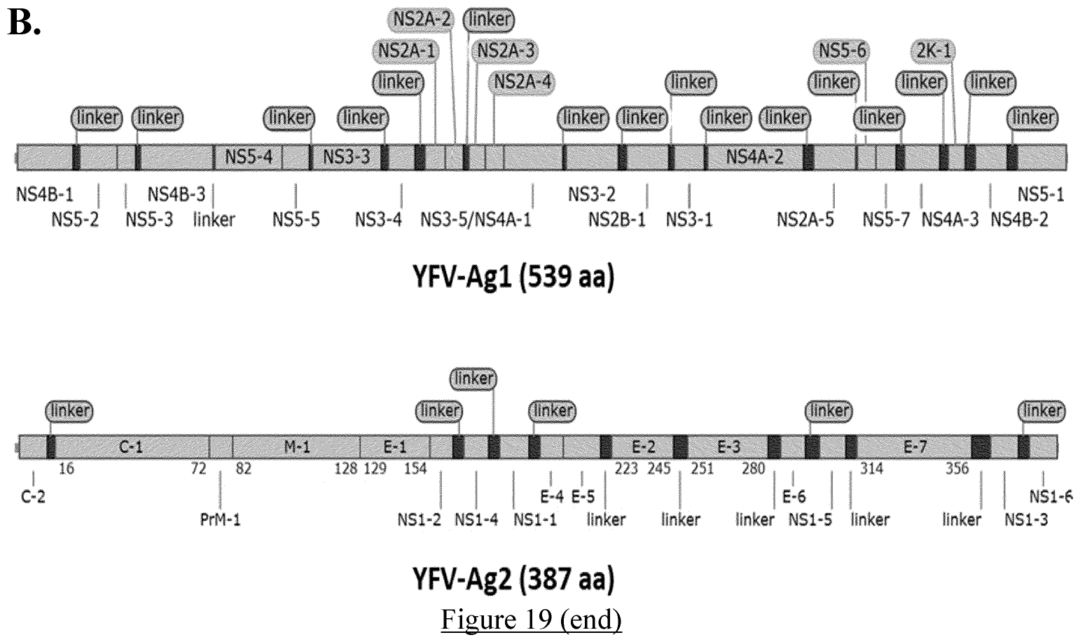

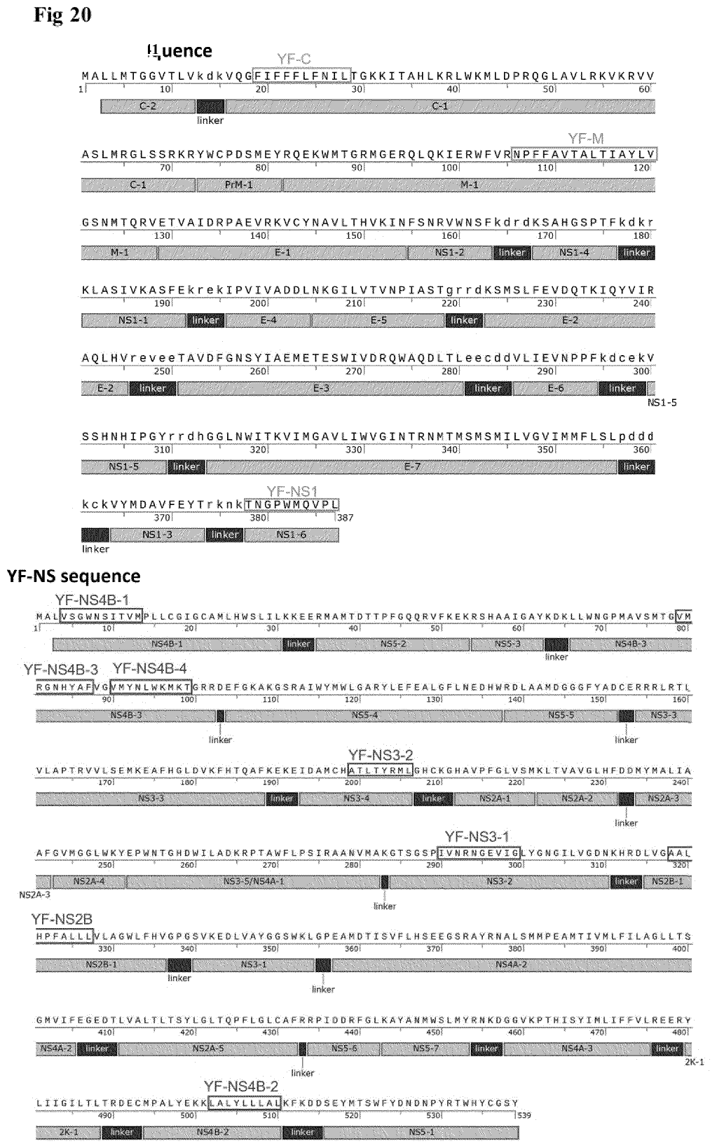

Two antigens were designed in order to develop candidate LV vaccine against YFV: the first (YFV-Ag1 or YFV-NS) included known and predicted T cell epitopes originating exclusively from the non-structural proteins NS2A, NS3B, NS3, NS4A, NS4B, and NS5, while the second antigen (YFV-Ag2 or YFV-S) has included T cell epitopes located in the structural proteins C, M, E and the secreted non-structural protein NS1. Both antigens were based on the sequence of currently used live-attenuated vaccine strain (17D-204) because that strain was used for the immunization and protection studies on IFNAR-KO mice. The antigenic regions chosen to compose YFV-Ag1 and YFV-Ag2 presented either individual MHC class I epitopes (both known and predicted) or clusters of such epitopes (FIG. 19A). The antigenic regions were arranged in a way to reduce the appearance of neo-epitopes and additional sequences (linkers) were designed to remove such epitopes if they were predicted to be formed, using the procedure outlined above for DENV-Ag design (FIG. 19B and FIG. 20).

### Immunogenicity of YFV Antigens for IFNAR(−/−) Mice

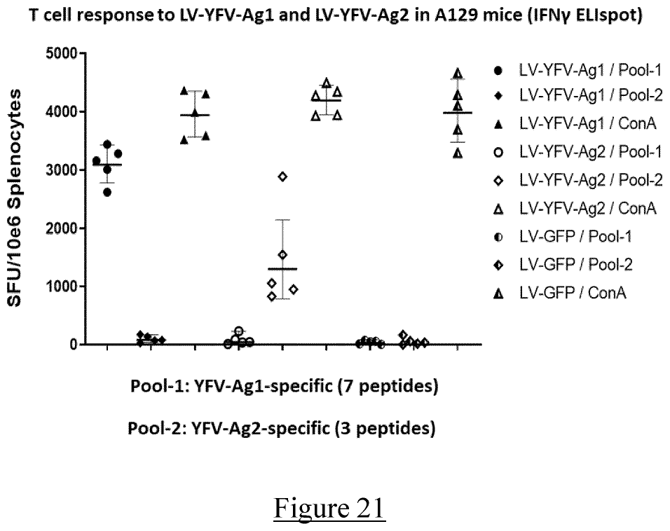

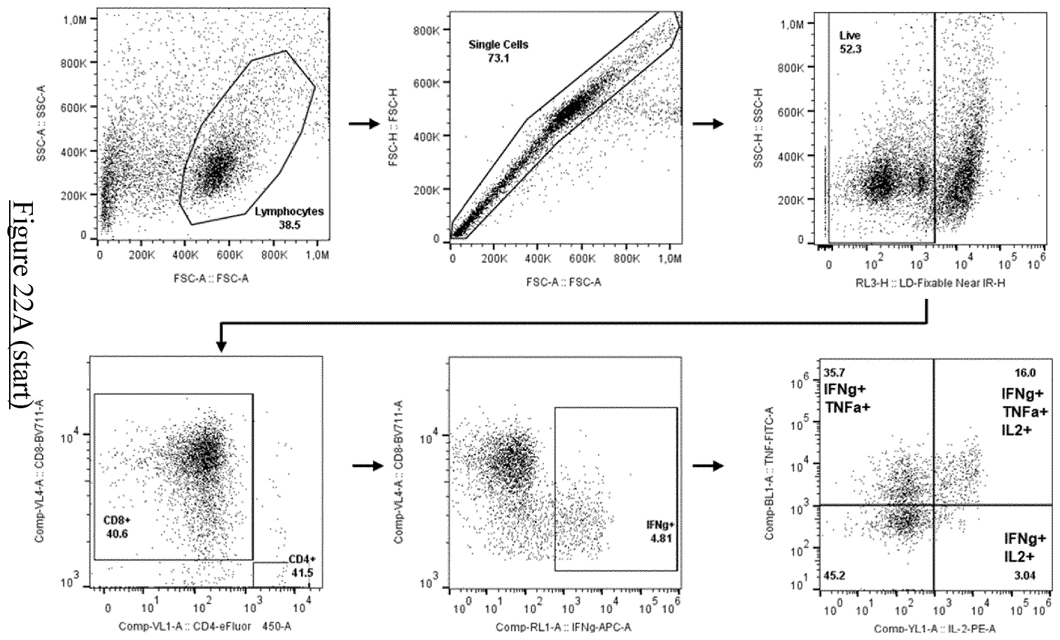

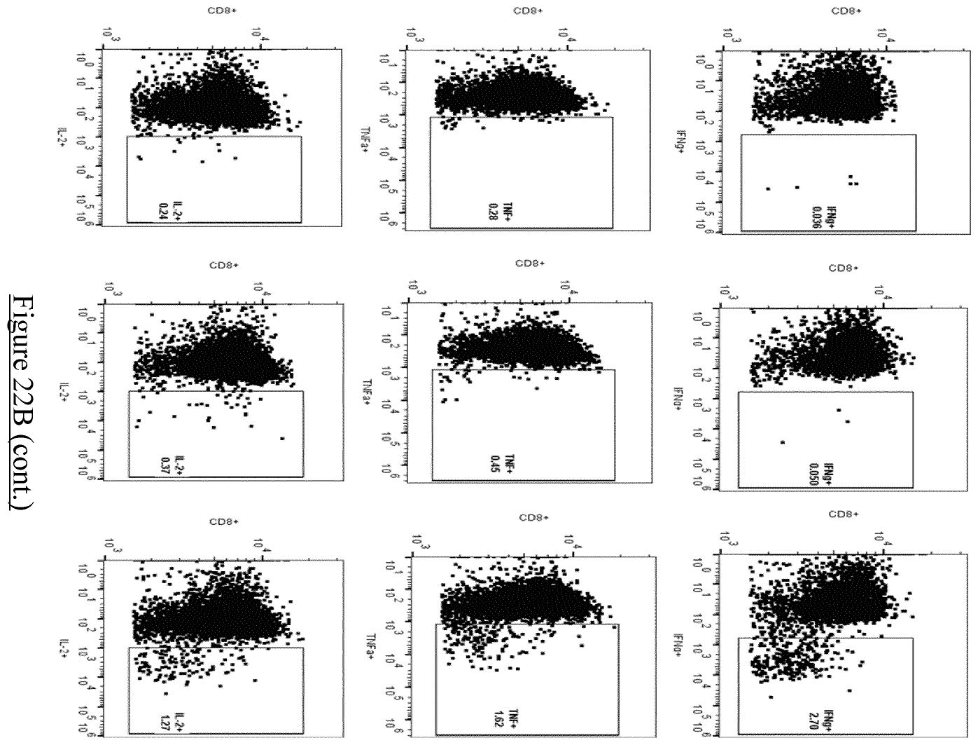

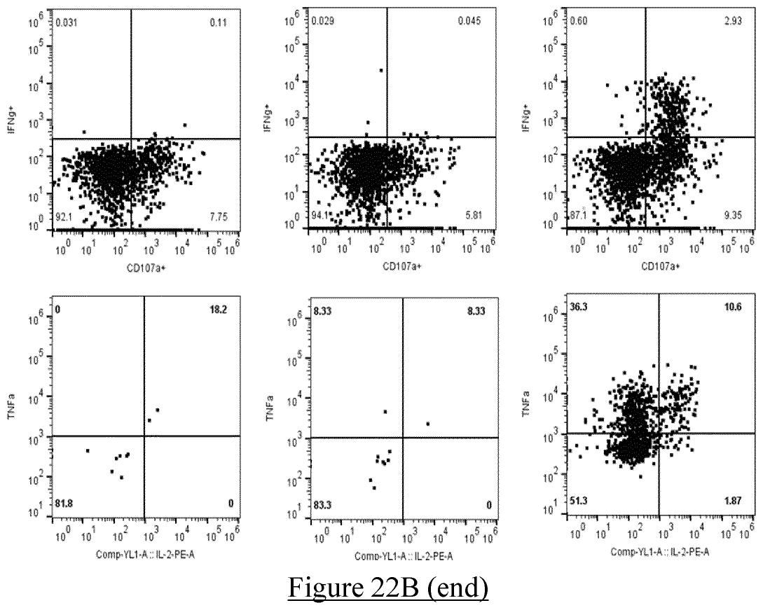

Analysis of the immunogenicity of LV-YFV-Ag1 and LV-YFV-Ag2 was performed in A129 mice. Three groups of mice were immunized with 3×10⁸ TU/mouse of either LV-YFV-Ag1, LV-YFV-Ag2, or LV-GFP and, 14 days post-immunization, splenocytes derived from immunized mice were re-stimulated ex-vivo with antigen-specific pools of peptides presenting selected MHC class I epitopes, and subjected to Elispot assay for IFNγ (FIG. 21). The analysis demonstrated that lentiviral vectors expressing both YFV-derived antigens were immunogenic and induced specific response in the immunized animals. Immunogenicity of LV-YFV-Ag1 has been additionally verified in IFNAR-BL6 mice using ICS (FIG. 22). Splenocytes from IFNAR-BL6 mice immunized with 3×10⁸ TU of either LV-YFV-Ag1 or LV-GFP vector were extracted 14 days post-infection and analyzed for the secretion of IFNγ, TNFα, IL2, and lymphocyte degranulation marker CD107a. Flow cytometry analysis of cytokine-stained cells has demonstrated that CD8+ T cells responded to antigen exposure/peptide stimulation by secretion of IFNγ, TNFα, and IL2 with a proportion of polypotent CD8+ T cells that simultaneously expressed all three cytokines. Similar to what has been observed with LV expressing antigens of DENV and ZIKV, cells co-expressing IFNγ and CD107a were also detected. No cytokine production was detected in splenocytes originating from mice immunized with LV-GFP and stimulated with YFV-Ag1-specific peptides, as well as in splenocytes that were immunized by LV-YFV-Ag1 but stimulated with a peptide not expressed from that antigen (YF-C), indicating that the cytokine production is specific.

### Protection of A129 Mice Against YFV Infection by LV-YFV-Ag1 and LV-YFV-Ag2

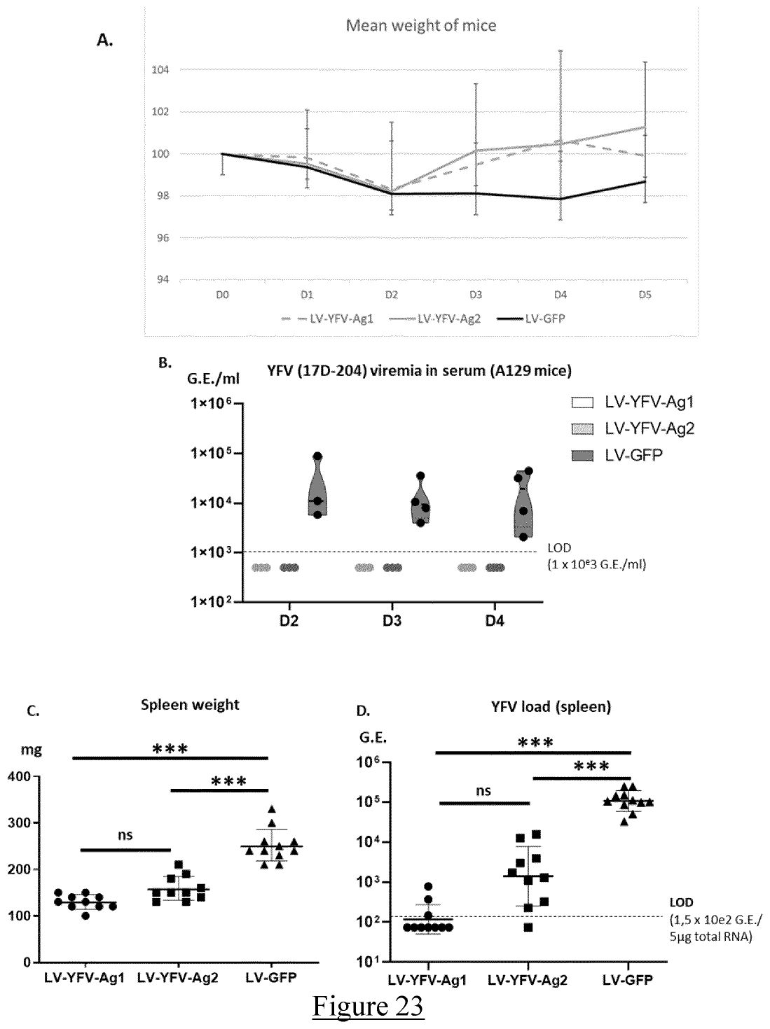

To analyze the protection against YFV, three groups of A129 mice were immunized, respectively, with 3×10⁸ TU/mouse of either LV-YFV-Ag1, LV-YFV-Ag2 or LV-GFP vector and in 1 month infected with 6×10⁶ PFU/mouse YFV (strain 17D-204). No weight loss or other apparent symptoms were noted in the infected animals (FIG. 23A). Viremia was analyzed on days 2, 3 and 4 post-infection (dpi), but was detectable only in mice immunized with the control vector LV-GFP (FIG. 23B). Analysis of spleens of infected mice indicated that YFV infection caused splenomegaly (increase in the size of spleen) in all mice immunized with LV-GFP, whereas mice immunized with both YFV-specific vectors had normal-size spleens (FIG. 23C). Furthermore, although viral presence was detectable in all the infected animals (in the spleen), it was significantly lower in the group of animals immunized with LV-YFV-Ag1 and LV-YFV-Ag2 compared to the control group (FIG. 23D).

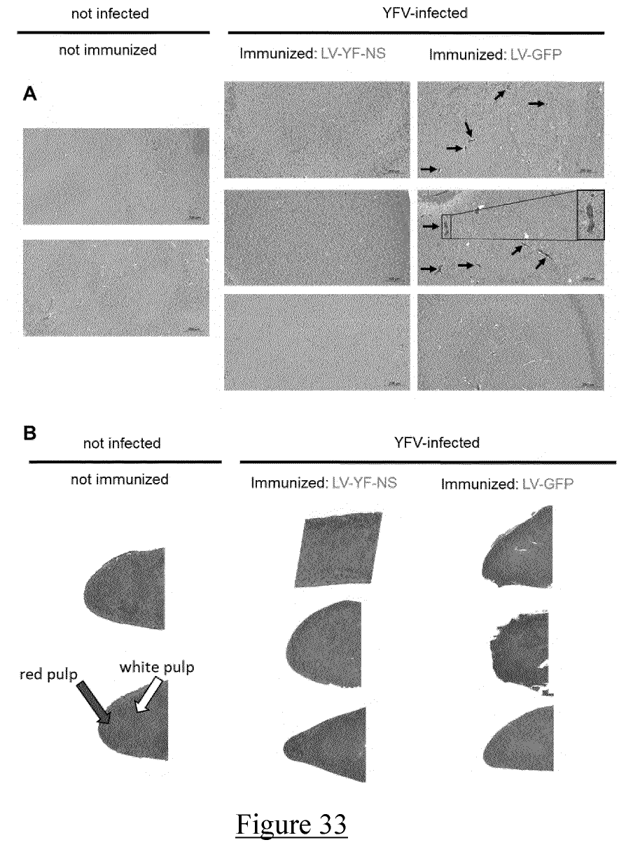

Protective potential of LV-YFV-NS was also assessed in the IFNAR-BL6 mice. Mice (n=5) were injected i.m. with 3×10⁸ TU/mouse of LV-YF-NS or LV-GFP. One month later, mice were inoculated with 5×10⁸ PFU/mouse of YFV (strain 17D-204). From 1 to 3 dpi, both groups of mice lost weight, however the mean weight of LV-YF-NS-immunized mice increased between 3 and 7 dpi, whereas mean weight of mice immunized with LV-GFP continued to decrease (FIG. 23E). Viremia measured 2 and 4 dpi was significantly lower (2.6×10² and 5.1×10³-fold differences between group's geometric means, respectively) in mice immunized with LV-YF-NS and could not be detected in these mice 7 dpi (FIG. 23F). In contrast, 7 dpi viremia was detectable in all the mice immunized with LV-GFP and 60% of them (3 of 5) were showing signs of severe disease, i.e., paralysis of the back legs, weakness and prostration, and reached humane endpoint of the experiment (FIG. 23G). Analysis of viral load in the brain, spleen and liver measured 7 dpi demonstrated significantly higher loads in all organs of mice immunized with LV-GFP (FIG. 23H). Histological analysis of spleen and brain of LV-GFP-injected control mice inoculated with YFV demonstrated poor contrast between white and red pulp in the spleen (FIG. 33A) and cuffing of the blood vessels in the brain (FIG. 33B), i.e. pathological changes that were also observed in LV-GFP-injected mice inoculated with ZIKV. In contrast, such changes were not observed in non-immunized non-infected mice, or in YFV-inoculated mice that were immunized with LV-YF-NS vector.

### Design of Bivalent DENV/ZIKV T Cell Antigens

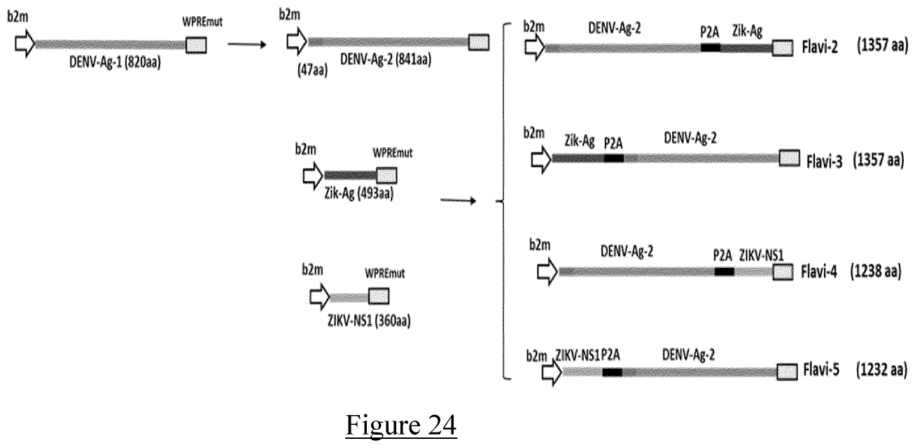

In order to facilitate the production and use of candidate prophylactic lentiviral vaccines against DENV and ZIKV, a set of bivalent antigens were constructed, that expressed DENV-Ag2 antigen in combination with one of the two ZIKV antigens (ZIKV-Ag or ZIKV-NS1) from a single construct. Thus, four antigenic constructs were created (FIG. 24): 1). Flavi-2 that express DENV-Ag2 followed by ZIKV-Ag ( ); 2). Flavi-3 that express ZIKV-Ag followed by DENV-Ag2; 3). Flavi-4 that express DENV-Ag2 followed by ZIKV-NS1 and 4). Flavi-5 that express ZIKV-NS1 followed by DENV-Ag2. In all the cases coding region of the first and second antigens were separated by the sequence of a self-cleaving polymerase P2A.

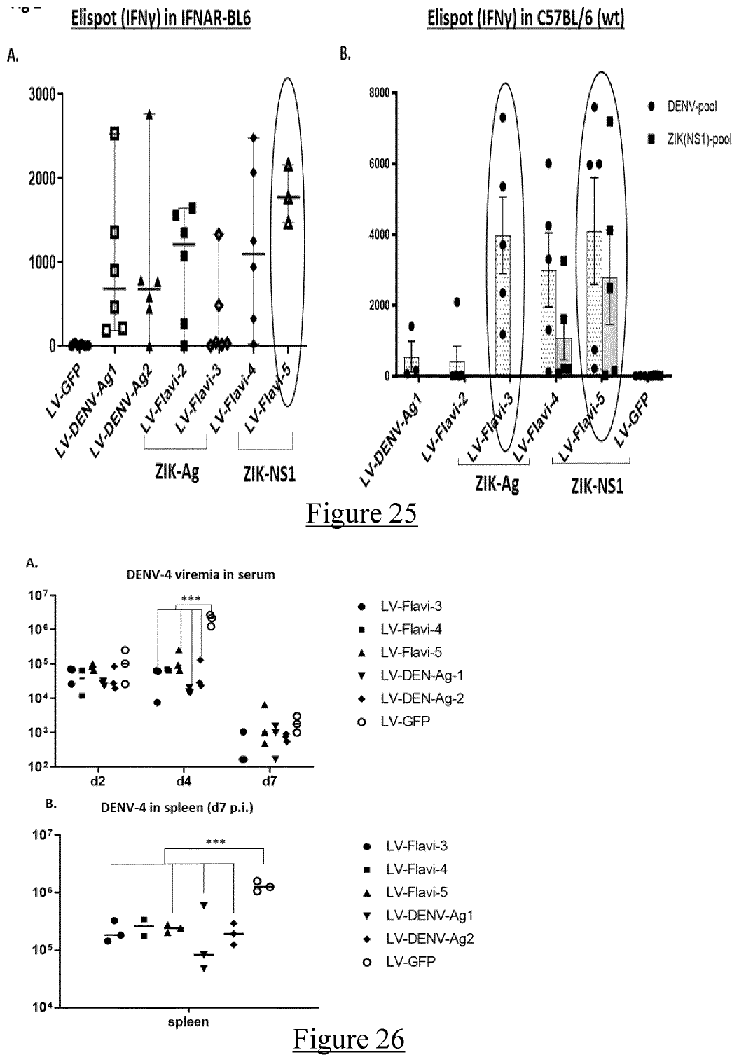

Immunogenicity of bivalent vectors was evaluated in IFNAR-BL6 mice and wild-type C57BL/6 mice to confirm that combining of DENV and ZIKV antigens in a single construct did not compromised immunogenicity and protection induced by the individual antigens. Groups of mice (n=3-5) were immunized i.m. with 1×10⁸ TU/mouse of each vector and T cell response was analyzed in splenocytes collected 14 days post-immunization, following re-stimulation with the pools of DENV-specific and ZIKV-specific peptides (FIGS. 25A and 25B). The results showed that all vectors were immunogenic and their immunogenicity was comparable or superior to that of vectors expressing original monovalent antigens.

Protection of IFNAR-BL6 Mice from DENV and ZIKV Infections by a Single-Dose Immunization with Bivalent DENV/ZIKV Vectors.

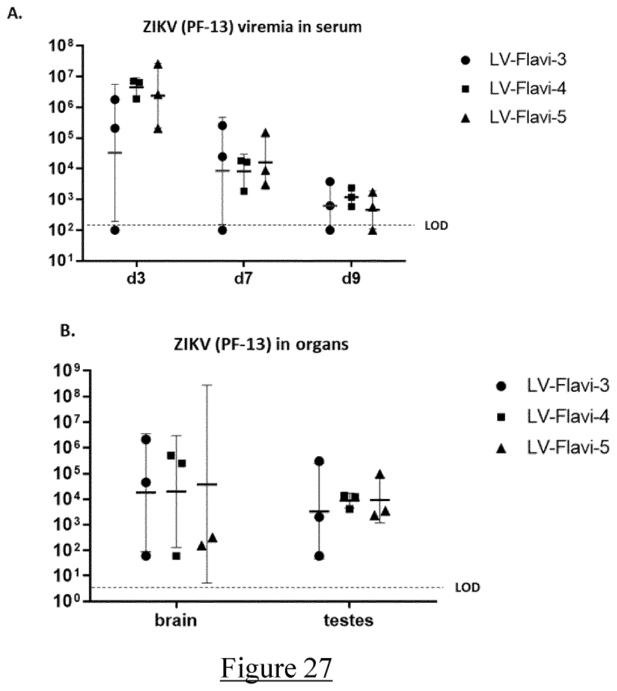

In the preliminary experiment, different “monovalent” and “bivalent” vectors were compared for the ability to protect against infection with one DENV serotype (DENV-4) (FIG. 26), and three bivalent DENV/ZIKV vectors (LV-Flavi-3, LV-Flavi-4 and LV-Flavi-5) were compared for the ability to protect against ZIKV infection (FIG. 27). No significant difference in the level of protection against DENV or ZIKV was seen between different vectors, suggesting that 1). Co-expression of DENV and ZIKV antigens from the same construct does not compromise protection against DENV; 2). Different bivalent DENV/ZIKV vectors induced similar level of protection against ZIKV. Based on the results of these protection experiments and immunogenicity tests described above, LV-Flavi-5 vector was selected for a more detailed analysis of protection against DENV and ZIKV.

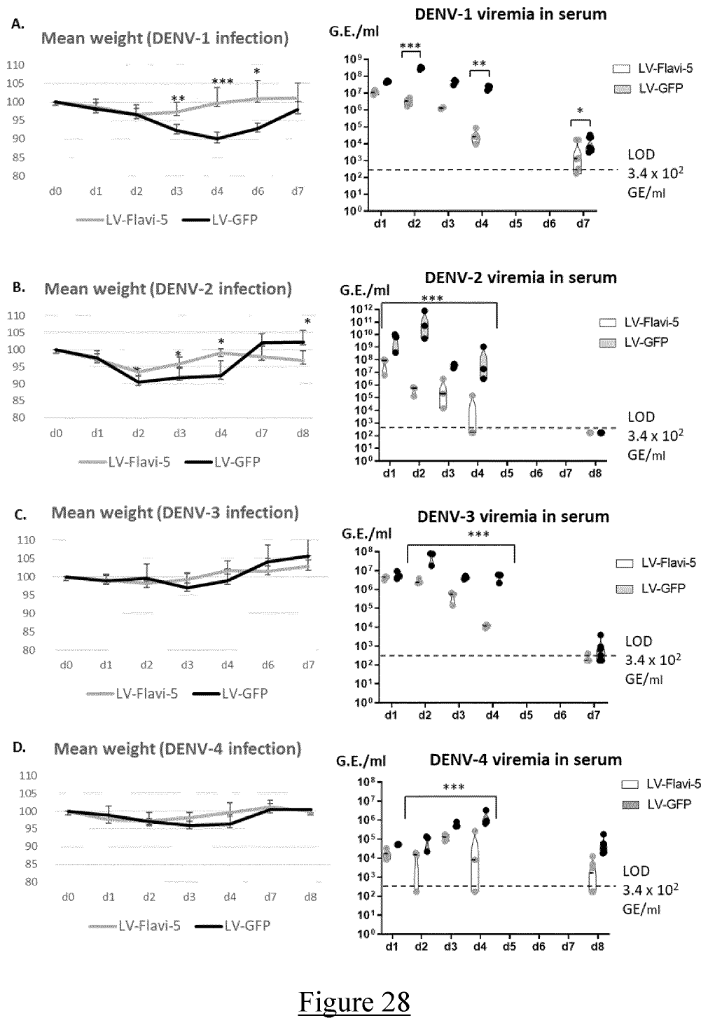

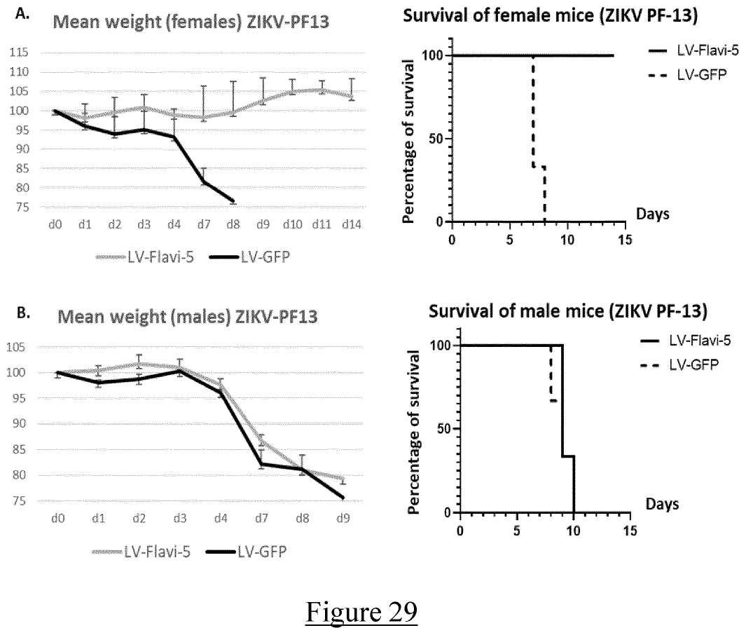

Protection provided by Flavi-5 bivalent vector against infections with 4 serotypes of DENV and ZIKV was re-evaluated in IFNAR-BL6 mice (FIG. 28). Groups of mice (n=6) were immunized with 3×10⁸ TU/mouse of either LV-Flavi-5 vector or the control LV-GFP vector, and 1 month post-immunization infected with DENV-1 (dose: 1×10⁷ PFU/mouse), DENV-2 (dose: 2×10⁶ PFU/mouse), DENV-3 (dose: 8×10⁶ PFU/mouse), DENV-4 (dose: 1×10⁷ PFU/mouse) or ZIKV (1×10³ PFU/mouse). The weight of mice was recorded regularly and viremia was analyzed at different days post-infection in the subgroups of infected mice. Immunization of IFNAR-BL6 with LV-Flavi-5 vector induced significant protection against all four DENV serotypes that was very similar to the level of protection achieved previously by immunization with LV-DEN-Ag1 vector. LV-Flavi-5 vector also efficiently protected female IFNAR-BL6 mice against ZIKV-induced weight loss and death, however, male mice (in which ZIKV infection is normally more pathogenic) were not protected, indicating that protection induced by the bivalent LV-Flavi-5 vector against ZIKV may be less efficient compared to the protection provided by monovalent vectors LV-ZIKV-Ag and LV-ZIK-NS1 (FIG. 29).

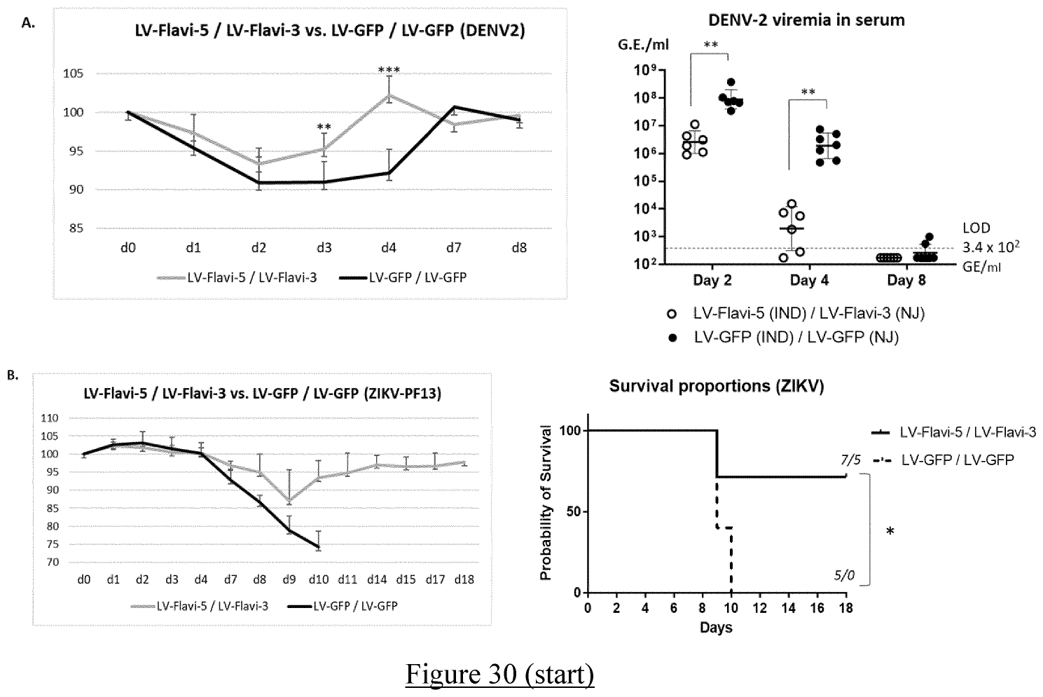

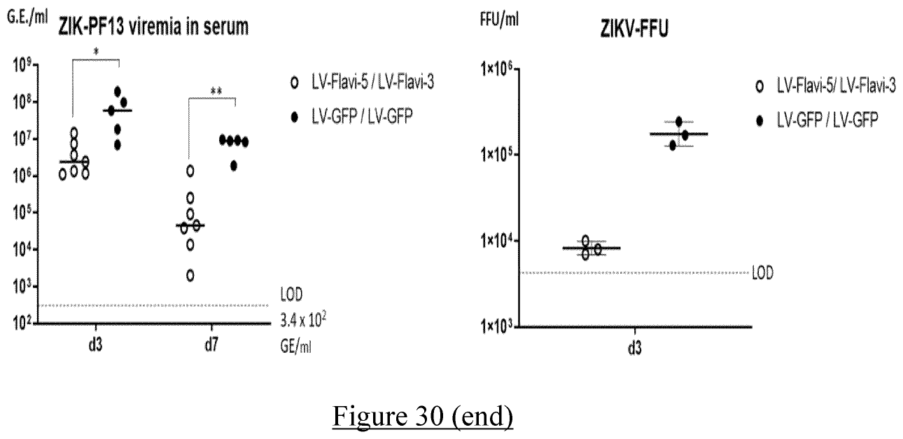

In attempt to improve the protection against ZIKV, a heterologous prime-boost strategy was employed, where IFNAR-BL6 mice were first immunized with 3×10⁸ TU/mouse of LV-Flavi-5 vector and in 1 month boosted with the same dose of LV-Flavi-3 vector. Although both vectors express same DENV antigen (DENV-Ag2), they express different ZIKV-specific antigens: ZIK-NS1 and ZIK-Ag, respectively. The protective effect of such prime-boost immunization was analyzed exclusively in male IFNAR-BL6 mice (to achieve the most robust possible conditions for viral challenge), that were after 1 month of second immunization infected with either DENV-2 or ZIKV. While the protection against DENV-2 induced by LV-Flavi-5/LV-Flavi-3 immunization was not significantly different from the protection induced by a single immunization with LV-Flavi-5 (FIG. 30A), protection against ZIKV has substantially improved: 70% of male mice immunized with LV-Flavi-5/LV-Flavi-3 survived ZIKV challenge while all mice immunized with LV-GFP have died (FIG. 30B). The protection also manifested itself in significantly lower viremia (10 to 100 fold) and lower presence of infectious virus in the plasma (150-fold) of infected mice immunized with LV-Flavi-5/LV-Flavi-3 vectors compare to controls. All in all these data suggest that “bivalent” DENV/ZIKV vector provides similar level of protection against DENV 1-4 compared to the “monovalent” DENV vector, but possibly lower protection against ZIKV. However, protection against ZIKV could be significantly improved by using consecutive immunization strategy with two bivalent vectors (LV-Flavi-5 and LV-Flavi-3) that express different ZIKV-specific antigens.

### REFERENCES

1. Guzman M G, Harris E. Dengue. Lancet. 2015 Jan. 31; 385(9966):453-65.
2. Ngono A E, Shresta S. Immune Response to Dengue and Zika. Annu Rev Immunol. 2018 Apr. 26; 36:279-308.
3. Wilken L, Rimmelzwaan G F. Adaptive Immunity to Dengue Virus: Slippery Slope or Solid Ground for Rational Vaccine Design?Pathogens. 2020 Jun. 15; 9(6):470.
4. Rothman A L. Immunity to dengue virus: a tale of original antigenic sin and tropical cytokine storms. Nat Rev Immunol. 2011 Jul. 15; 11(8):532-43.
5. Weiskopf D, Angelo M A, de Azeredo E L, Sidney J, Greenbaum J A, Fernando A N, Broadwater A, Kolla R V, De Silva A D, de Silva A M, Mattia K A, Doranz B J, Grey H M, Shresta S, Peters B, Sette A. Comprehensive analysis of dengue virus-specific responses supports an HLA-linked protective role for CD8+ T cells. Proc Natl Acad Sci USA. 2013 May 28; 110(22):E2046-53.
6. Weiskopf D, Angelo M A, Bangs D J, Sidney J, Paul S, Peters B, de Silva A D, Lindow J C, Diehl S A, Whitehead S, Durbin A, Kirkpatrick B, Sette A. The human CD8+ T cell responses induced by a live attenuated tetravalent dengue vaccine are directed against highly conserved epitopes. J Virol. 2015 January; 89(1):120-8.
7. Weiskopf D, Cerpas C, Angelo M A, Bangs D J, Sidney J, Paul S, Peters B, Sanches F P, Silvera C G, Costa P R, Kallas E G, Gresh L, de Silva A D, Balmaseda A, Harris E, Sette A.

Human CD8+ T-Cell Responses Against the 4 Dengue Virus Serotypes Are Associated With Distinct Patterns of Protein Targets. J Infect Dis. 2015 Dec. 1; 212(11):1743-51.
8. Elong Ngono A, Chen H W, Tang W W, Joo Y, King K, Weiskopf D, Sidney J, Sette A, Shresta S. Protective Role of Cross-Reactive CD8 T Cells Against Dengue Virus Infection. EBioMedicine. 2016 November; 13:284-293.
9. Yauch L E, Zellweger R M, Kotturi M F, Qutubuddin A, Sidney J, Peters B, Prestwood T R, Sette A, Shresta S. A protective role for dengue virus-specific CD8+ T cells. J Immunol. 2009 Apr. 15; 182(8):4865-73.
10. Zellweger R M, Eddy W E, Tang W W, Miller R, Shresta S. CD8+ T cells prevent antigen-induced antibody-dependent enhancement of dengue disease in mice. J Immunol. 2014 Oct. 15; 193(8):4117-24.
11. Zellweger R M, Tang W W, Eddy W E, King K, Sanchez M C, Shresta S. CD8+ T Cells Can Mediate Short-Term Protection against Heterotypic Dengue Virus Reinfection in Mice. J Virol. 2015 June; 89(12):6494-505.
12. Roth C, Delgado F G, Simon-Lorière E, Sakuntabhai A. Immune Responses to Dengue and Zika Viruses-Guidance for T Cell Vaccine Development. Int J Environ Res Public Health. 2018 Feb. 23; 15(2):385.
13. Ku M W, Charneau P, Majlessi L. Use of lentiviral vectors in vaccination. Expert Rev Vaccines. 2021 December; 20(12):1571-1586.
14. Ku M W, Authié P, Bourgine M, Anna F, Noirat A, Moncoq F, Vesin B, Nevo F, Lopez J, Souque P, Blanc C, Fert I, Chardenoux S, Lafosse L, Cussigh D, Hardy D, Nemirov K, Guinet F, Langa Vives F, Majlessi L, Charneau P. Brain cross-protection against SARS-CoV-2 variants by a lentiviral vaccine in new transgenic mice. EMBO Mol Med. 2021 Dec. 7; 13(12):e14459.
15. Ku M W, Bourgine M, Authié P, Lopez J, Nemirov K, Moncoq F, Noirat A, Vesin B, Nevo F, Blanc C, Souque P, Tabbal H, Simon E, Hardy D, Le Dudal M, Guinet F, Fiette L, Mouquet H, Anna F, Martin A, Escriou N, Majlessi L, Charneau P. Intranasal vaccination with a lentiviral vector protects against SARS-CoV-2 in preclinical animal models. Cell Host Microbe. 2021 Feb. 10; 29(2):236-249.
16. National Center for Biotechnology Information (NCBI)[Internet]. Bethesda (MD): National Library of Medicine (US), National Center for Biotechnology Information; [1988]-[cited 2022 Mar. 27]. Available from: https://www.ncbi.nlm.nih.gov/
17. Clustal W/Clustal X: Multiple alignment of nucleic acid and protein sequences. Available from: Clustal W and Clustal X Multiple Sequence Alignment
18. Kumar S, Stecher G, Tamura K. MEGA7: Molecular Evolutionary Genetics Analysis Version 7.0 for Bigger Datasets. Mol Biol Evol. 2016 July; 33(7):1870-4.
19. MAFFT version 7: Multiple alignment program for amino acid or nucleotide sequences. Available from: MAFFT—a multiple sequence alignment program (cbrc.ip).
20. Hall, T. A. (1999) BioEdit: A User-Friendly Biological Sequence Alignment Editor and Analysis Program for Windows 95/98/N T. Nucleic Acids Symposium Series, 41, 95-98.
21. Immune Epitope Database and Analysis Resource. Available from: IEDB.org: Free epitope database and prediction resource.
22. Paul S, Weiskopf D, Angelo M A, Sidney J, Peters B, Sette A. HLA class I alleles are associated with peptide-binding repertoires of different size, affinity, and immunogenicity. J Immunol. 2013 Dec. 15; 191(12):5831-9.
23. HLA allele frequencies and reference sets with maximal population coverage—IEDB Solutions Center.
24. Selecting thresholds (cut-offs) for MHC class I and II binding predictions—IEDB Solutions Center.
25. Stranzl T, Larsen M V, Lundegaard C, Nielsen M. NetCTLpan: pan-specific MHC class I pathway epitope predictions. Immunogenetics. 2010 June; 62(6):357-68.
26. HIV sequence database: Consensus Maker. Available from: Consensus Maker (lanl.qov).
27. Zennou V, Petit C, Guetard D, Nerhbass U, Montagnier L, Charneau P. HIV-1 genome nuclear import is mediated by a central DNA flap. Cell. 2000 Apr. 14; 101(2):173-85.
28. Gurukumar K R, Priyadarshini D, Patil J A, Bhagat A, Singh A, Shah P S, Cecilia D. Development of real time PCR for detection and quantitation of Dengue Viruses. Virol J. 2009 Jan. 23; 6:10.
29. Lanciotti R S, Kosoy O L, Laven J J, Velez J O, Lambert A J, Johnson A J, Stanfield S M, Duffy M R. Genetic and serologic properties of Zika virus associated with an epidemic, Yap State, Micronesia, 2007. Emerg Infect Dis. 2008 August; 14(8):1232-9.
30. Bae H G, Nitsche A, Teichmann A, Biel S S, Niedrig M. Detection of yellow fever virus: a comparison of quantitative real-time PCR and plaque assay. J Virol Methods. 2003 Jun. 30; 110(2):185-91.
31. Elong Ngono A, Chen H W, Tang W W, Joo Y, King K, Weiskopf D, Sidney J, Sette A, Shresta S. Protective Role of Cross-Reactive CD8 T Cells Against Dengue Virus Infection. EBioMedicine. 2016 November; 13:284-293.
32. Backert L, Kohlbacher O. Immunoinformatics and epitope prediction in the age of genomic medicine. Genome Med. 2015; 7:119.
33. Ku M W, Anna F, Souque P, Petres S, Prot M, Simon-Loriere E, Charneau P, Bourgine M. A Single Dose of NILV-Based Vaccine Provides Rapid and Durable Protection against Zika Virus. Mol Ther. 2020 Aug. 5; 28(8):1772-1782.
34. Manet C, Simon-Loriere E, Jouvion G, Hardy D, Prot M, Conquet L, Flamand M, Panthier J J, Sakuntabhai A, Montagutelli X. Genetic Diversity of Collaborative Cross Mice Controls Viral Replication, Clinical Severity, and Brain Pathology Induced by Zika Virus Infection, Independently of Oas1b. J Virol. 2020 Jan. 17; 94(3):e01034-19.
35. Prestwood T R, Morar M M, Zellweger R M, Miller R, May M M, Yauch L E, Lada S M, Shresta S. Gamma interferon (IFN-γ) receptor restricts systemic dengue virus replication and prevents paralysis in IFN-α/p receptor-deficient mice. J Virol. 2012 December; 86(23):12561-70.
36. Sidney J, Peters B, Frahm N, Brander C, Sette A. HLA class I supertypes: A revised and updated classification. BMC Immunol. 2008; 9(1):1471-2172.)
37. Weiskopf D, Angelo M A, de Azeredo E L, Sidney J, Greenbaum J A, Fernando A N, Broadwater A, Kolla R V, De Silva A D, de Silva A M, Mattia K A, Doranz B J, Grey H M, Shresta S, Peters B, Sette A. Comprehensive analysis of dengue virus-specific responses supports an HLA-linked protective role for CD8+ T cells. PNAS. 2013; 110(22):E2046-53.
38. Zennou, V., Petit, C., Guetard, D., Nerhbass, U., Montagnier, L., and Charneau, P. (2000). HIV-1 genome nuclear import is mediated by a central DNA flap. Cell 101, 173-185.
39 Firat H. et al. The Journal of Gene Medicine 2002; 4: 38-45.
40. VandenDriessche T, Thorrez L, Naldini L, Follenzi A, Moons L, Berneman Z, Collen D, Chuah M K. Lentiviral vectors containing the human immunodeficiency virus type-1 central polypurine tract can efficiently transduce nondividing hepatocytes and antigen-presenting cells in vivo. Blood. 2002 Aug. 1; 100(3):813-22.
41. Sakuma T, Barry M A, Ikeda Y. Lentiviral vectors: basic to translational. Biochem J. 2012 May 1; 443(3):603-18. doi: 10.1042/BJ20120146. PMID: 22507128.—Jones S, Peng P D, Yang S, Hsu C, Cohen C J, Zhao Y, Abad J, Zheng Z, Rosenberg S A, Morgan R A. Lentiviral vector design for optimal T cell receptor gene expression in the transduction of peripheral blood lymphocytes and tumor-infiltrating lymphocytes. Hum Gene Ther. 2009 June; 20(6):630-40.
42. Cockrell A S, Kafri T. Gene delivery by lentivirus vectors. Mol Biotechnol. 2007 July; 36(3):184-204.
43. Ikeda, Y., Takeuchi, Y., Martin, F. et al. Continuous high-titer HIV-1 vector production. Nat Biotechnol 21, 569-572 (2003).
44. Cockrell A S, Ma H, Fu K, McCown T J, Kafri T. A trans-lentiviral packaging cell line for high-titer conditional self-inactivating HIV-1 vectors. Mol Ther. 2006 August; 14(2):276-84.
45. Xu K, Ma H, McCown T J, Verma I M, Kafri T. Generation of a stable cell line producing high-titer self-inactivating lentiviral vectors. Mol Ther. 2001 January; 3(1):97-104.
46. Kafri T, van Praag H, Ouyang L, Gage F H, Verma I M. A packaging cell line for lentivirus vectors. J Virol. 1999 January; 73(1):576-84.
47. Zufferey R, Donello J E, Trono D, Hope T J. Woodchuck hepatitis virus posttranscriptional regulatory element enhances expression of transgenes delivered by retroviral vectors. J Virol. 1999 April; 73(4):2886-92.
48. Park F, Kay M A. Modified HIV-1 based lentiviral vectors have an effect on viral transduction efficiency and gene expression in vitro and in vivo. Mol Ther. 2001 September; 4(3):164-73.
49. Garg H, Mehmetoglu-Gurbuz T, Joshi A. Recent Advances in Zika Virus Vaccines. Viruses (2018) 10(11). Epub 2018/11/18. doi: 10.3390/v10110631.
50. Ghaffar K A, Ng L F P, Renia L. Fast Tracks and Roadblocks for Zika Vaccines. Vaccines (2018) 6(4). Epub 2018/11/25. doi: 10.3390/vaccines6040077.
51. Bardina S V, Bunduc P, Tripathi S, Duehr J, Frere J J, Brown J A, et al. Enhancement of Zika Virus Pathogenesis by Preexisting Antiflavivirus Immunity. Science (2017) 356(6334):175-80. Epub 2017/04/01. doi: 10.1126/science.aal4365.
52. Miner J J, Diamond M S. Dengue Antibodies, Then Zika: A Fatal Sequence in Mice. Immunity (2017) 46(5):771-3. Epub 2017/05/18. doi: 10.1016/j.immuni.2017.04.023.
53. Dejnirattisai W, Supasa P, Wongwiwat W, Rouvinski A, Barba-Spaeth G, Duangchinda T, et al. Dengue Virus Sero-Cross-Reactivity Drives Antibody-Dependent Enhancement of Infection with Zika Virus. Nat Immunol (2016) 17(9):1102-8. Epub 2016/06/25. doi: 10.1038/ni.3515.
54. Londono-Renteria B, Troupin A, Cardenas J C, Hall A, Perez O G, Cardenas L, et al. A Relevant in Vitro Human Model for the Study of Zika Virus Antibody-Dependent Enhancement. J Gen Virol (2017) 98(7):1702-12. Epub 2017/07/12. doi: 10.1099/jgv.0.000833.
55. Fowler A M, Tang W W, Young M P, Mamidi A, Viramontes K M, McCauley M D, et al. Maternally Acquired Zika Antibodies Enhance Dengue Disease Severity in Mice. Cell host & microbe (2018) 24(5):743-50.e5. Epub 2018/11/16. doi: 10.1016/j.chom.2018.09.015.
56. Zimmerman M G, Quicke K M, O'Neal J T, Arora N, Machiah D, Priyamvada L, et al. Cross-Reactive Dengue Virus Antibodies Augment Zika Virus Infection of Human Placental Macrophages. Cell host & microbe (2018) 24(5):731-42.e6. Epub 2018/11/16. doi: 10.1016/j.chom.2018.10.008.
57. Waggoner J J, Rojas A, Pinsky B A. Yellow Fever Virus: Diagnostics for a Persistent Arboviral Threat. J Clin Microbiol (2018) 56(10). Epub 2018/07/20. doi: 10.1128/jcm.00827-18.
58. Douam F, Ploss A. Yellow Fever Virus: Knowledge Gaps Impeding the Fight against an Old Foe. Trends in microbiology (2018) 26(11):913-28. Epub 2018/06/24. doi: 10.1016/j.tim.2018.05.012.
59. Ishikawa T, Yamanaka A, Konishi E. A Review of Successful Flavivirus Vaccines and the Problems with Those Flaviviruses for Which Vaccines Are Not yet Available. Vaccine (2014) 32(12):1326-37. Epub 2014/02/04. doi: 10.1016/j.vaccine.2014.01.040.
60. Possas C, Lourenco-de-Oliveira R, Tauil P L, Pinheiro F P, Pissinatti A, Cunha R V D, et al. Yellow Fever Outbreak in Brazil: The Puzzle of Rapid Viral Spread and Challenges for Immunisation. Memorias do Instituto Oswaldo Cruz (2018) 113(10):e180278. Epub 2018/11/15. doi: 10.1590/0074-02760180278.
61. Monath T P, Fowler E, Johnson C T, Balser J, Morin M J, Sisti M, et al. An Inactivated Cell-Culture Vaccine against Yellow Fever. N Engl J Med (2011) 364(14):1326-33. Epub 2011/04/08. doi: 10.1056/NEJMoa1009303.
62. Nemirov K, Bourgine M, Anna F, Wei Y, Charneau P, Majlessi L. Lentiviral Vectors as a Vaccine Platform against Infectious Diseases. Pharmaceutics (2023) 15(3):846.
63. Nemirov K, Authié P, Souque P, Moncoq F, Noirat A, Blanc C, et al. Preclinical Proof of Concept of a Tetravalent Lentiviral T-Cell Vaccine against Dengue Viruses. Frontiers in Immunology (2023) 14. doi: 10.3389/fimmu.2023.1208041.
64. Seabra S G, Libin P J K, Theys K, Zhukova A, Potter B I, Nebenzahl-Guimaraes H, et al. Genome-Wide Diversity of Zika Virus: Exploring Spatio-Temporal Dynamics to Guide a New Nomenclature Proposal. Virus evolution (2022) 8(1). doi: 10.1093/ve/veac029.
65. Beasley D W, McAuley A J, Bente D A. Yellow Fever Virus: Genetic and Phenotypic Diversity and Implications for Detection, Prevention and Therapy. Antiviral Res (2015) 115:48-70. Epub 2014/12/30. doi: 10.1016/j.antiviral.2014.12.010.
66. Sayers E W, Bolton E E, Brister J R, Canese K, Chan J, Comeau D C, et al. Database Resources of the National Center for Biotechnology Information. Nucleic Acids Res (2022) 50(D1):D20-d6. Epub 2021/12/02. doi: 10.1093/nar/gkab1112.
67. Agrawal V K, Prusty B S K, Reddy C S, Mohan Reddy G K, Agrawal R K, Sekher Srinivasarao Bandaru V C. Clinical Profile and Predictors of Severe Dengue Disease: A Study from South India. Caspian journal of internal medicine (2018) 9(4):334-40. Epub 2018/12/05. doi: 10.22088/cjim.9.4.334.
68. Los Alamos Hiv Database (2018). Available from: http://www.hiv.lanl.qov.
69. Tenzer S, Peters B, Bulik S, Schoor O, Lemmel C, Schatz M M, et al. Modeling the Mhc Class I Pathway by Combining Predictions of Proteasomal Cleavage, Tap Transport and Mhc Class I Binding. Cell Mol Life Sci (2005) 62(9):1025-37. Epub 2005/05/04. doi: 10.1007/s00018-005-4528-2.
70. Peters B, Bulik S, Tampe R, Van Endert P M, Holzhutter H G. Identifying Mhc Class I Epitopes by Predicting the Tap Transport Efficiency of Epitope Precursors. J Immunol (2003) 171(4):1741-9. Epub 2003/08/07. doi: 10.4049/jimmunol.171.4.1741.
71. Immune Epitope Database & Tools [Internet]. (2018). Available from: https://www.iedb.org/.
72. Stranzl T, Larsen M V, Lundegaard C, Nielsen M. Netctlpan: Pan-Specific Mhc Class I Pathway Epitope Predictions. Immunogenetics (2010) 62(6):357-68. Epub 2010/04/10. doi: 10.1007/s00251-010-0441-4.
73. Dtu Health Tech: Bioinformatic Tools and Services [cited 2018-2023]. Available from: https://services.healthtech.dtu.dk/.
74. Paul S, Weiskopf D, Angelo M A, Sidney J, Peters B, Sette A. Hla Class I Alleles Are Associated with Peptide-Binding Repertoires of Different Size, Affinity, and Immunogenicity. J Immunol (2013) 191(12):5831-9. Epub 2013/11/06. doi: 10.4049/jimmunol.1302101.
75. Zellweger R M, Prestwood T R, Shresta S. Enhanced Infection of Liver Sinusoidal Endothelial Cells in a Mouse Model of Antibody-Induced Severe Dengue Disease. Cell host & microbe (2010) 7(2):128-39. Epub 2010/02/16. doi: 10.1016/j.chom.2010.01.004.
76. Lopez J, Anna F, Authie P, Pawlik A, Ku M W, Blanc C, et al. A Lentiviral Vector Encoding Fusion of Light Invariant Chain and Mycobacterial Antigens Induces Protective Cd4(+) T cell Immunity. Cell Rep (2022) 40(4):111142. Epub 2022/07/30. doi: 10.1016/j.celrep.2022.111142.
77. Maecker H T. Multiparameter Flow Cytometry Monitoring of T Cell Responses. Methods Mol Biol (2009) 485:375-91. Epub 2008/11/21. doi: 10.1007/978-1-59745-170-3_25.
78. Fischer C, Torres M C, Patel P, Moreira-Soto A, Gould E A, Charrel R N, et al. Lineage-Specific Real-Time Rt-Pcr for Yellow Fever Virus Outbreak Surveillance, Brazil. Emerging infectious diseases (2017) 23(11). Epub 2017/09/28. doi: 10.3201/eid2311.171131.
79. Graphpad Prism. GraphPad Software. version 9.5.0 for Windows ed. San Diego, California USA: GraphPad Software.
80. Collins N D, Widen S G, Li L, Swetnam D M, Shi P-Y, Tesh R B, et al. Inter- and Intra-Lineage Genetic Diversity of Wild-Type Zika Viruses Reveals Both Common and Distinctive Nucleotide Variants and Clusters of Genomic Diversity. Emerging microbes & infections (2019) 8(1):1126-38. doi: 10.1080/22221751.2019.1645572.

1. A recombinant polynucleotide comprising at least one polynucleotide encoding a fusion polypeptide which comprises MHC class I T-cell epitopes suitable to elicit a T cell response, wherein the MHC class I T-cell epitopes originate from a plurality of non-structural or structural antigens wherein the antigens are from at least one flavivirus selected from the group of Dengue virus (DENV), ZIKA virus (ZIKV) and Yellow Fever virus (YFV) and wherein the MHC class I T-cell epitopes are assembled together through junction regions that are devoid of non-specific neoepitopes.

2. A recombinant polynucleotide according to claim 1, comprising a first polynucleotide encoding a first fusion polypeptide which comprises MHC class I T-cell epitopes originating from more than one non-structural DENV proteins and forming an assembled DENV-based consensus antigen of DENV-1, DENV-2, DENV-3 and DENV-4 strains.

3. The recombinant polynucleotide according to claim 1 wherein the MHC class I T-cell epitopes originate from at least 2 antigens of DENV selected from the group of the NS3, NS4A, NS4B and NS5 antigens.

4. The recombinant polynucleotide according to claim 1, wherein the junction regions include hydrophobic amino acid linkers and are devoid of sequences encoding non-specific immunodominant epitopes.

5. The recombinant polynucleotide according to claim 1 wherein the assembled polynucleotide comprises the polynucleotides encoding the MHC class I T-cell epitopes of SEQ ID No. 2, SEQ ID No. 4, SEQ ID No. 6, SEQ ID No. 8, SEQ ID No. 10, SEQ ID No. 12, SEQ ID No. 14, SEQ ID No. 16, SEQ ID No. 18, SEQ ID No. 20, SEQ ID No. 22, SEQ ID No. 24, SEQ ID No. 26 and SEQ ID No. 28 or a variant thereof which is devoid of SEQ ID No. 2.

6. The recombinant polynucleotide according to claim 1 which is selected from:
a. a recombinant polynucleotide which comprises a polynucleotides encoding the MHC class I T-cell epitopes of the following sequences, arranged from 5′ to 3′ in the recombinant polynucleotide in accordance with the following order: SEQ ID No. 2, SEQ ID No. 4, SEQ ID No. 6, SEQ ID No. 8, SEQ ID No. 10, SEQ ID No. 12, SEQ ID No. 14, SEQ ID No. 16, SEQ ID No. 18, SEQ ID No. 20, SEQ ID No. 22, SEQ ID No. 24, SEQ ID No. 26 and SEQ ID No. 28 and wherein the junction regions between said polynucleotides encoding the MHC class I T-cell epitopes are devoid of sequences encoding non-specific immunodominant epitopes or,
b. the recombinant polynucleotide which comprises the polynucleotides encoding the MHC class I T-cell epitopes of the following sequences, arranged from 5′ to 3′ in the recombinant polynucleotide in accordance with the following order: SEQ ID No. 4, SEQ ID No. 6, SEQ ID No. 8, SEQ ID No. 10, SEQ ID No. 12, SEQ ID No. 14, SEQ ID No. 16, SEQ ID No. 18, SEQ ID No. 20, SEQ ID No. 22, SEQ ID No. 24, SEQ ID No. 26, SEQ ID No. 28, SEQ ID No. 32, SEQ ID No. 34 and SEQ ID No. 36 and wherein the junction regions between said polynucleotides encoding the MHC class I T-cell epitopes are devoid of sequences encoding non-specific immunodominant epitopes, in particular the recombinant polynucleotide wherein an amino acid linker sequence is inserted as a junction region between all consecutive above sequences except between SEQ ID No. 8 and SEQ ID No. 10, between SEQ ID No. 16 and SEQ ID No. 18, between SEQ ID No. 18 and SEQ ID No. 20 and between SEQ ID No. 34 and SEQ ID No. 36

7. The recombinant polynucleotide according to claim 1, wherein the MHC class I T-cell epitopes originate from at least 2 antigens of ZIKV selected from the group of the NS4B and NS5 antigens and further MHC class I T-cell epitopes originate from at least 2 antigens of ZIKV selected from the group of the C and PrM antigens, wherein the MHC class I T-cell epitopes are assembled together through junction regions that are devoid of non-specific neoepitopes.

8. The recombinant polynucleotide according to claim 1, wherein the polynucleotides encoding the MHC class I T-cell epitopes are arranged from 5′ to 3′ in the recombinant polynucleotide in accordance with the following order: SEQ ID No. 40, SEQ ID No. 42, SEQ ID No. 44, SEQ ID No. 46, SEQ ID No. 48, SEQ ID No. 50 and SEQ ID No. 52 and wherein the junction regions between said polynucleotides encoding the MHC class I T-cell epitopes are devoid of sequences encoding non-specific immunodominant epitopes, in particular the recombinant polynucleotide wherein an amino acid linker sequence is inserted as a junction region between all consecutive above sequences, except between SEQ ID No. 44 and SEQ ID No. 46 and between SEQ ID No. 50 and SEQ ID No. 52.

9. The recombinant polynucleotide according to claim 1, wherein the MHC class I T-cell epitopes originate from at least 1 antigen of YFV selected from the group of the NS-1, NS-2A, NS2B, NS-3, NS-4 and NS4B and NS5 antigens and further MHC class I T-cell epitopes originate from at least 2 antigens of YFV selected from the group of C, M and E antigens, wherein the MHC class I T-cell epitopes are assembled together through junction regions that are devoid of non-specific neoepitopes.

10. The recombinant polynucleotide according to claim 1, which is selected from:
a. a polynucleotide which comprises polynucleotides encoding the MHC class I T-cell epitopes arranged from 5′ to 3′ in the recombinant polynucleotide in accordance with the following order: SEQ ID No. 58, SEQ ID No. 60, SEQ ID No. 62, SEQ ID No. 64, SEQ ID No. 66, SEQ ID No. 68, SEQ ID No. 70, SEQ ID No. 72, SEQ ID No. 74, SEQ ID No. 76, SEQ ID No. 78, SEQ ID No. 80, SEQ ID No. 82, SEQ ID No. 84, SEQ ID No 86, SEQ ID No. 88, SEQ ID No. 90, SEQ ID No. 92, SEQ ID No. 94, SEQ ID No. 96, SEQ ID No. 98, SEQ ID No. 100, SEQ ID No. 102 and SEQ ID No. 104 and wherein the junction regions between said polynucleotides encoding the MHC class I T-cell epitopes are devoid of sequences encoding non-specific immunodominant epitopes, in particular the recombinant polynucleotide wherein an amino acid linker sequence is inserted as a junction region between all consecutive above sequences except between SEQ ID No. 60 and SEQ ID No. 62, between SEQ ID No. 66 and SEQ ID No. 68, between SEQ ID No. 74 and SEQ ID No. 76, between SEQ ID No. 78 and SEQ ID No. 80 and SEQ ID No. 82 and between SEQ ID No. 94 and SEQ ID No. 96, or
b. a polynucleotide which comprises polynucleotides encoding the MHC class I T-cell epitopes arranged from 5′ to 3′ in the recombinant polynucleotide in accordance with the following order: SEQ ID No. 108, SEQ ID No. 110, SEQ ID No. 112, SEQ ID No. 114, SEQ ID No. 116, SEQ ID No. 118, SEQ ID No. 120, SEQ ID No. 122, SEQ ID No. 124, SEQ ID No. 126, SEQ ID No. 128, SEQ ID No. 130, SEQ ID No. 132, SEQ ID No. 134, SEQ ID No. 136, SEQ ID No. 138 and SEQ ID No. 140 and wherein the junction regions between said polynucleotides encoding the MHC class I T-cell epitopes are devoid of sequences encoding non-specific immunodominant epitopes.

11. The recombinant polynucleotide according to claim 1, which further contains a sequence encoding a signal peptide at its 5′-end.

12. The recombinant polynucleotide according to claim 1, whose sequence consists of SEQ ID No. 29 or SEQ ID No. 37.

13. The recombinant polynucleotide according to claim 1 wherein the sequence of the polynucleotide is modified with respect to the sequence of SEQ ID No. 29 or SEQ ID No. 37, wherein the modification consists of a point mutation, substitution or deletion of nucleotides, and the modified sequence encodes a fusion polypeptide that has at least 90% sequence identity with the sequence of the original fusion polypeptide.

14. The recombinant polynucleotide according to claim 1, which comprises (i) a first polynucleotide encoding a first fusion polypeptide comprising MHC class I T-cell epitopes originating from structural and from non-structural DENV proteins and forming an assembled DENV-based antigen and (ii) a second polynucleotide encoding a second fusion polypeptide comprising MHC class I T-cell epitopes originating from structural and from non-structural ZIKV proteins and forming an assembled ZIKV-based antigen and/or (iii) a third polynucleotide encoding a second fusion polypeptide comprising MHC class I T-cell epitopes originating from structural and/or from non-structural YFV proteins and forming an assembled YFV-based antigen, wherein the first and, when present, the second and third polynucleotides are operably linked in an expression cassette and are separated by a sequence encoding a self-cleavage peptide optionally associated with a spacer sequence.

15. A recombinant polyepitopic polypeptide whose amino acid sequence consists of SEQ ID No. 30, SEQ ID No. 38, SEQ ID No. 54, SEQ ID No. 56, SEQ ID No. 106, SEQ ID No. 142, or a variant thereof obtained by deletion or by point mutation of one or more amino acid residues and wherein the variant sequence has at least 90% sequence identity.

16. A recombinant lentiviral vector genome comprising the recombinant polynucleotide according to claim 1.

17. The recombinant lentiviral vector genome according to claim 16, wherein said genome is the insert obtained from a pFLAP vector plasmid selected from the group of pFlap-beta2m-DENV-Ag1-WPREm of SEQ ID No. 151, pFlap-beta2m-DENV-Ag2-WPREm of SEQ ID No. 152, pFlap-beta2m-ZIKV-Ag-WPREm of SEQ ID No. 153, pFlap-beta2m-ZIKV-NS1-WPREm of SEQ ID No. 154, pFlap-beta2m-YFV-Ag1-WPREm of SEQ ID No. 155, pFlap-beta2m-YFV-Ag2-WPREm of SEQ ID No. 156, pFlap-beta2m-DENV-Ag2_ZIKV-Ag-WPREm_(Flavi-2) of SEQ ID No. 157, pFlap-beta2m-ZIKV-Ag_DENV-Ag2-WPREm_(Flavi-3) of SEQ ID No. 158, pFlap-beta2m-DENV-Ag2_ZIKV-NS1-WPREm_(Flavi-4) of SEQ ID No. 159, and pFlap-beta2m-ZIKV-NS1-DENV-Ag2-WPREm_(Flavi-5) of SEQ ID No. 160.

18. A recombinant lentiviral vector particle which comprises the recombinant lentiviral vector genome according to claim 1.

19. The recombinant lentiviral vector particle according to claim 18, wherein said recombinant lentiviral vector particle is a recombinant replication-incompetent pseudotyped lentiviral vector particle.

20. A mammalian host cell transfected with the recombinant polynucleotide according to claim 1.

21. A composition suitable for administration to a human host, comprising the recombinant polynucleotide according to claim 1 together with one or more pharmaceutically acceptable excipient(s) suitable for administration to the human host and optionally an adjuvant.

22. A method for the treatment of a patient with a flavivirus infection selected from the group of Dengue virus (DENV), ZIKA virus (ZIKV) and Yellow Fever virus (YFV) comprising the administration of an effective amount of the composition of claim 21 to the patient.

23. The method of claim 22, wherein the DENV is and DENV-4.

24. A The method of claim 22, wherein the composition is comprised in a multivalent vaccine.

25. The method of claim 22, wherein the administration comprises a prime-boost administration regimen of the composition.
